(19) United States
(12) Patent Application Publication
(10) Pub. No.: US 2016/0318933 A1
(43) Pub. Date: Nov. 3, 2016

(54) FUSED PYRAZOLE DERIVATIVE

(71) Applicant: SUMITOMO DAINIPPON PHARMA CO., LTD., Osaka-shi, Osaka (JP)

(72) Inventors: Hidefumi YOSHINAGA, Osaka (JP); Yoshiharu URUNO, Osaka (JP); Kiyoto SAWAMURA, Osaka (JP); Nana GOTO, Osaka (JP); Yohei IKUMA, Osaka (JP)

(73) Assignee: Sumitomo Dainippon Pharma Co., Ltd., Osaka (JP)

(21) Appl. No.: 15/029,692

(22) PCT Filed: Oct. 22, 2014
(86) PCT No.: PCT/JP2014/078103

§ 371 Date: Jul. 19, 2016

(30) Foreign Application Priority Data
Oct. 23, 2013 (JP) 2013-220037

(51) Int. Cl. C07D 487/04; C07D 519/00

(52) U.S. Cl. CPC C07D 487/04; C07D 519/00

(57) ABSTRACT

The present invention provides a cyclic aminomethyl pyrimidine derivative including a pharmaceutically acceptable salt thereof with high selectivity for dopamine D₄ receptor, which is useful for treating a disease such as attention deficit hyperactivity disorder. Specifically, the present invention relates no a compound of formula (1) or a pharmaceutically acceptable salt thereof, wherein n and m are independently 1 or 2; W¹, W³, and W⁴ are independently single bond or optionally-substituted C₁₋₄ alkylene group; W² is optionally-substituted C₁₋₄ alkylene group; R¹ and R² are independently hydrogen atom, etc.; R³ is hydrogen atom, halogen atom, etc.; X¹ and X² are independently single bond, oxygen atom, etc.; ring Q¹ is optionally-substituted 5- to 10-membered heteroaryl group, etc.; ring Q² is optionally-substituted 6-membered heteroaryl group, etc.

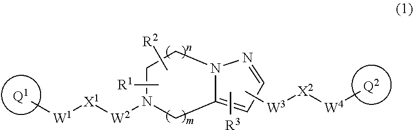

### TECHNICAL FIELD

The present invention relates to a fused pyrazole derivative including a salt thereof which has a selective dopamine D₄ receptor agonistic effect as well as a medicament for treating a central nervous system disease comprising the derivative as an active ingredient.

### BACKGROUND ART

Dopamine D₄ receptor is one of G protein-coupled receptors (GPCRs), and is highly expressed in frontal association area associated with attention behavior and cognitive function. Hence, a dopamine D₄ receptor agonist is expected to be used for treating a central nervous system disease related to higher brain function such as attention deficit hyperactivity disorder (ADHD). ADHD is one of developmental disorders accompanying inattention, hyperactivity, and impulsivity as a predominant symptom, which appears in childhood. Also, it is known that the predominant symptom of ADHD persists into adulthood. As a first-choice drug for treating ADHD, methylphenidate which is one of central nervous system stimulants has been used. Methylphenidate exhibits a fast-acting therapeutic effect, which is thought to be produced by the regulation of dopamine transporter function associated with the release of dopamine that is a neurotransmitter. However, methylphenidate is at risk for drug dependence or drug abuse, and also at risk for cardiovascular side effects such as palpitation, tachycardia, and blood-pressure variation. Thus, as a medicament for treating ADHD which is at low risk for drug dependence, a selective noradrenaline reuptake inhibitor, atomoxetine which is one of non-central nervous system stimulants has been used. However, atomoxetine requires an adequate period after the administration to exert its therapeutic effect. Accordingly, it has been desired to develop a medicament for treating ADHD, which has a reduced risk for drug dependence and also a reduced risk for cardiovascular side effects, and exhibits a fast-acting therapeutic effect.

It has been reported that ADHD patients have mutations in dopamine transporter genes or dopamine D₄ receptor genes (e.g. Non-Patent Reference 1). Also, it has been reported that children with gene polymorphism in a seven times repeating sequence of 48 bp within the third exon of dopamine D₄ receptor genes have the delayed development of cerebral cortex (e.g. Non-Patent Reference 3). In addition, it has been reported that a dopamine D₄ receptor is highly expressed in frontal association area associated with attention behavior and cognitive function (e.g. Non-Patent Reference 2). Hence, it has been thought that the dopamine D₄ receptor is associated with attention and cognitive functions. In addition, it is well known that the dopamine D₄ receptor is not expressed in nucleus accumbens associated with drug dependence.

Accordingly, a drug which exhibits a selective dopamine D₄ receptor agonistic effect has been expected as a medicament for treating a central nervous system disease related to dopaminergic nerves, especially a medicament for treating ADHD which exhibits a fast-acting therapeutic effect with reduced side effects such as drug dependence.

Patent Document 1 discloses that the compound of the following formula can modulate the activity of metabotropic glutamate receptor 5 (mGluR5), and thus is useful in the treatment, prevention, and/or control of various disorders such as neurological disorder:

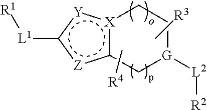

wherein R¹ is aryl, heteroaryl, etc.;

R² is aryl, heteroaryl, etc.;

R³ and R⁴ are independently hydrogen, halogen, lower alkyl, etc.;

L¹ is a bond, —O—, —CR⁵R⁶—, etc.;

L² is a bond, —O—, —CR⁵R⁶—, etc.;

X is C or N;

Y is O, S, N, etc.;

Z is O, S, N, etc.;

R⁵ and R⁶ are independently hydrogen, halogen, or lower alkyl, or CR⁵R⁶ is C═O; or R⁵ and R⁶ may be combined with the carbon atom to which they are attached to form 3- to 7-membered cycloalkyl;

G is N or CH;

o is 0, 1, or 2; and

p is 1 or 2.

However, Patent Reference 1 does not specifically disclose a fused pyrazole derivative.

### PRIOR ART DOCUMENTS

### Patent Documents

Patent Reference 1: JP 2012-522793

### Non-Patent Documents

Non-Patent Reference 1: Biological Psychiatry 2005, 57, 1313.
Non-Patent Reference 2: Archives of General Psychiatry, 2007, 64, 921.
Non-Patent Reference 3: The Journal of Pharmacology Experimental Therapeutics, 1997, 282, 1020.

### SUMMARY OF INVENTION

### Problem to be Solved by the Invention

An object of the present invention is to provide a novel selective dopamine D₄ receptor agonist useful as a medicament for treating a central nervous system disease.

### Means for Solving the Problems

The present inventors have extensively studied to reach the above object, and then have found that a compound of the following formula (1) or a pharmaceutically acceptable salt thereof (hereinafter referred to as “the present compound”, as necessary) exhibits a remarkable selective dopamine D₄ receptor agonistic effect. Based upon the new findings, the present invention has been completed.

The present invention provides inventions of various embodiments described below.

Term [1]

A compound of formula (1):

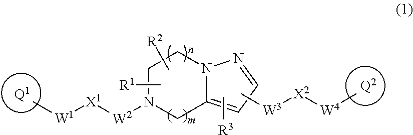

or a pharmaceutically acceptable salt thereof, wherein

n and m are independently 1 or 2;

W¹, W³, and W⁴ are independently single bond or optionally-substituted C₁₋₄ alkylene group;

W² is C₁₋₄ alkylene group;

R¹ and R² are independently hydrogen atom, halogen atom, or optionally-substituted C₁₋₆ alkyl group, or R¹ and R² may be combined with the carbon atom(s) to which they are attached to form 3- to 8-membered cycloalkane ring;

R³ is hydrogen atom, halogen atom, cyano group, optionally-substituted C₁₋₆ alkyl group, optionally-substituted C₁₋₆ alkoxy group, optionally-substituted C₁₋₆ alkylcarbonyl group, or optionally-substituted aminocarbonyl group;

X¹ and X² are independently single bond, oxygen atom, sulfur atom, —C(O)—, —NR⁴⁰—, or —C(O)NR⁴⁰—, wherein said R⁴⁰ is hydrogen atom or C₁₋₆ alkyl group;

ring Q¹ is optionally-substituted C₆₋₁₀ aryl group, optionally-substituted 5- to 10-membered heteroaryl group, optionally-substituted C₅₋₁₀ cycloalkyl group, or optionally-substituted 5- to 10-membered cyclic amino group; and

ring Q² is optionally-substituted phenyl group, optionally-substituted 6-membered heteroaryl group, optionally-substituted 5- or 6-membered saturated heterocyclyl group, or optionally-substituted 5- or 6-membered cyclic amino group.

Term [2]

The compound according to term [1] or a pharmaceutically acceptable salt thereof, wherein

n and m are independently 1 or 2;

W¹, W³, and W⁴ are independently single bond or C₁₋₄ alkylene group which may be optionally substituted with the same or different 1 or 2 halogen atoms;

W² is C₁₋₄ alkylene group;

R¹ and R² are independently hydrogen atom, halogen atom, or C₁₋₆ alkyl group which may be optionally substituted with the same or different 1 to 3 halogen atoms, or R¹ and R² may be combined with the carbon atom(s) to which they are attached to form 3- to 8-membered cycloalkane ring;

R³ is

(1) hydrogen atom,
(2) halogen atom,
(3) cyano group,
(4) C₁₋₆ alkyl group which may be optionally substituted with the same or different 1 to 3 halogen atoms,
(5) C₁₋₆ alkoxy group which may be optionally substituted with the same or different 1 to 3 halogen atoms,
(6) C₁₋₆ alkylcarbonyl group which may be optionally substituted with the same or different 1 to 3 halogen atoms, or
(7) aminocarbonyl group wherein the amino moiety thereof may be optionally substituted with the same or different 1 or 2 groups selected independently from the group consisting of C₁₋₆ alkyl and C₃₋₇ cycloalkyl group;

X¹ and X² are independently single bond, oxygen atom, sulfur atom, —C(O)—, —NR⁴⁰—, or —C(O)NR⁴⁰—, wherein said R⁴⁰ is hydrogen atom or C₁₋₆ alkyl group;

ring Q¹ is

(8) C₆₋₁₀ aryl group which may be optionally substituted with the same or different 1 to 4 groups selected independently from the group consisting of

(a) halogen atom,

(b) C₁₋₆ alkyl group which may be optionally substituted with the same or different 1 to 3 groups selected independently from the group consisting of halogen atom and hydroxy group,

(c) C₁₋₆ alkoxy group which may be optionally substituted with the same or different 1 to 3 halogen atoms,

(d) cyano group, and

(e) amino group which may be optionally substituted with the same or different 1 or 2 groups selected independently from the group consisting of C₁₋₆ alkyl group and C₃₋₇ cycloalkyl group,

(9) 5- to 10-membered heteroaryl group which may be optionally substituted with the same or different 1 to 4 groups selected independently from the group consisting of (a) to (e) defined in the above (8),
(10) C₅₋₁₀ cycloalkyl group which may be optionally substituted with the same or different 1 to 4 groups selected independently from the group consisting of (a) to (e) defined in the above (8), or
(11) 5- to 10-membered cyclic amino group which may be optionally substituted with the same or different 1 to 4 groups selected independently from the group consisting of (a) to (e) defined in the above (8); and

ring Q² is

(12) phenyl group which may be optionally substituted with the same or different 1 to 4 groups selected independently from the group consisting of (a) to (e) defined in the above (8),
(13) 6-membered heteroaryl group which may be optionally substituted with the same or different 1 to 4 groups selected independently from the group consisting of (a) to (e) defined in the above (8),
(14) 5- or 6-membered saturated heterocyclyl group which may be optionally substituted with the same or different 1 to 4 groups selected independently from the group consisting of (a) to (e) defined in the above (8), or
(15) 5- or 6-membered cyclic amino group which may be optionally substituted with the same or different 1 to 4 groups selected independently from the group consisting of (a) to (e) defined in the above (8).

Term [3]

The compound according to term [1] or [2] or a pharmaceutically acceptable salt thereof, wherein W³, X¹, and X² are single bond.

Term [4]

The compound according to term [1] or [2] or a pharmaceutically acceptable salt thereof, wherein the compound is a compound of formula (1a):

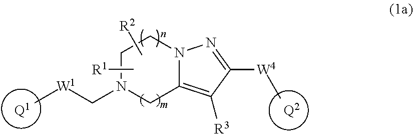

wherein n, m, W¹, W⁴, R¹, R², R³, ring Q¹, and ring Q² are as defined in term [1] or [2]

Term [5]

The compound of term [4] or a pharmaceutically acceptable salt thereof, wherein

n and m are independently 1 or 2;

W¹ and W⁴ are independently single bond or C₁₋₄ alkylene group which may be optionally substituted with the same or different 1 or 2 halogen atoms;

R¹ and R² are independently hydrogen atom, halogen atom, or C₁₋₆ alkyl group which may be optionally substituted with the same or different 1 to 3 halogen atoms, or R¹ and R² may be combined with the carbon atom(s) to which they are attached to form 3- to 8-membered cycloalkane ring;

R³ is

(1) hydrogen atom,
(2) halogen atom,
(3) cyano group,
(4) C₁₋₆ alkyl group which may be optionally substituted with the same or different 1 to 3 halogen atoms, or
(5) C₁₋₆ alkoxy group which may be optionally substituted with the same or different 1 to 3 halogen atoms;

ring Q¹ is

(6) 5- to 10-membered heteroaryl group which may be optionally substituted with the same or different 1 to 4 groups selected independently from the group consisting of

(a) halogen atom,

(b) C₁₋₆ alkyl group which may be optionally substituted with the same or different 1 to 3 groups selected independently from the group consisting of halogen atom and hydroxy group,

(c) C₁₋₆ alkoxy group which may be optionally substituted with the same or different 1 to 3 halogen atoms,

(d) cyano group, and

(e) amino group which may be optionally substituted with the same or different 1 or 2 groups selected independently from the group consisting of C₁₋₆ alkyl group and C₃₋₇ cycloalkyl group,

(7) C₆₋₁₀ aryl group which may be optionally substituted with the same or different 1 to 4 groups selected independently from the group consisting of (a) to (e) defined in the above (6), or
(8) C₅₋₁₀ cycloalkyl group which may be optionally substituted with the same or different 1 to 4 groups selected independently from the group consisting of (a) to (e) defined in the above (6);

ring Q² is

(9) phenyl group which may be optionally substituted with the same or different 1 to 4 groups selected independently from the group consisting of (a) to (e) defined in the above (6),
(10) 6-membered heteroaryl group which may be optionally substituted with the same or different 1 to 4 groups selected independently from the group consisting of (a) to (e) defined in the above (6), or
(11) 5- or 6-membered saturated heterocyclyl group which may be optionally substituted with the same or different 1 to 4 groups selected independently from the group consisting of (a) to (e) defined in the above (6).

Term [6]

The compound according to term [5] or a pharmaceutically acceptable salt thereof, wherein the ring Q² is

(1) phenyl group which may be optionally substituted with the same or different 1 to 4 groups selected independently from the group consisting of

(a) halogen atom,

(b) C₁₋₆ alkyl group which may be optionally substituted with the same or different 1 to 3 halogen atoms,

(c) C₁₋₆ alkoxy group which may be optionally substituted with the same or different 1 to 3 halogen atoms,

(d) cyano group, and

(e) amino group which may be optionally substituted with the same or different 1 or 2 groups selected independently from the group consisting of C₁₋₆ alkyl group and C₃₋₇ cycloalkyl group, or

(2) 6-membered heteroaryl group containing 1 to 3 nitrogen atoms which may be optionally substituted with 1 to 4 groups selected independently from the group consisting of (a) to (e) defined in the above (1).

Term [7]

The compound according to any one of terms [4] to [6] or a pharmaceutically acceptable salt thereof, wherein

n is 1 or 2;

m is 1;

both W¹ and W⁴ are single bond;

R¹, R², and R³ are independently hydrogen atom, halogen atom, or C₁₋₆ alkyl group which may be optionally substituted with the same or different 1 to 3 halogen atoms;

ring Q¹ is

(1) 5- to 10-membered heteroaryl group containing 1 to 3 nitrogen atoms which may be optionally substituted with the same or different 1 to 4 groups selected independently from the group consisting of

(a) halogen atom,

(b) C₁₋₆ alkyl group which may be optionally substituted with the same or different 1 to 3 halogen atoms,

(c) C₁₋₆ alkoxy group which may be optionally substituted with the same or different 1 to 3 halogen atoms,

(d) cyano group, and

(e) amino group which may be optionally substituted with the same or different 1 or 2 groups selected independently from the group consisting of C₁₋₆ alkyl group and C₃₋₇ cycloalkyl group, or

(2) C₆₋₁₀ aryl group which may be optionally substituted with the same or different 1 to 4 groups selected independently from the group consisting of (a) to (e) defined in the above (1);

ring Q² is

(3) pyridyl group which may be optionally substituted with the same or different 1 to 4 groups selected independently from the group consisting of (a) to (e) defined in the above (1), or
(4) phenyl group which may be optionally substituted with the same or different 1 to 4 groups selected independently from the group consisting of (a) to (e) defined in the above (1).

Term [8]

The compound according to any one of terms [4] to [7] or a pharmaceutically acceptable salt thereof, wherein ring Q¹ is 5- to 10-membered heteroaryl group containing 1 to 3 nitrogen atoms which may be optionally substituted with the same or different 1 to 4 groups selected independently from the group consisting of

(a) halogen atom,

(b) C₁₋₆ alkyl group which may be optionally substituted with the same or different 1 to 3 groups selected independently from the group consisting of halogen atom and hydroxy group,

(c) C₁₋₆ alkoxy group which may be optionally substituted with the same or different 1 to 3 halogen atoms,

(d) cyano group, and

(e) amino group which may be optionally substituted with the same or different 1 or 2 groups selected independently from the group consisting of C₁₋₆ alkyl group and C₃₋₇ cycloalkyl group.

Term [9]

The compound according to any one of terms [4] to [7] or a pharmaceutically acceptable salt thereof, wherein ring Q¹ is

(1) 6-membered heteroaryl group containing 1 to 3 nitrogen atoms which may be optionally substituted with the same or different 1 to 4 groups selected independently from the group consisting of

(a) halogen atom,

(b) C₁₋₆ alkyl group which may be optionally substituted with the same or different 1 to 3 halogen atoms,

(c) C₁₋₆ alkoxy group which may be optionally substituted with the same or different 1 to 3 halogen atoms,

(d) cyano group, and

(e) amino group which may be optionally substituted with the same or different 1 or 2 groups selected independently from the group consisting of C₁₋₆ alkyl group and C₃₋₇ cycloalkyl group, or

(2) phenyl group which may be optionally substituted with the same or different 1 to 4 groups selected independently from the group consisting of (a) to (e) defined the above (1).

Term [10]

The compound according to any one of terms [4] to [8] or a pharmaceutically acceptable salt thereof, wherein ring Q¹ is a group of the following formula (2a) or (2b):

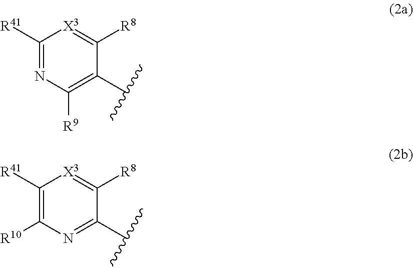

wherein X³ is N or CR⁷;

R⁴¹ is halogen atom or C₁₋₆ alkyl group which may be optionally substituted with the same or different 1 to 3 groups selected independently from the group consisting of halogen atom and hydroxy group;

R⁷, R⁸, R⁹, and R¹⁰ are independently hydrogen atom, halogen atom, C₁₋₆ alkyl group which may be optionally substituted with the same or different 1 to 3 halogen atoms, or amino group which may be optionally substituted with the same or different 1 or 2 C₁₋₆ alkyl groups;

or R⁴¹ and R¹⁰, or R⁴¹ and R⁷ may be combined with the carbon atom(s) to which they are attached to form 5- to 8-membered cycloalkane ring or 5- to 8-membered cycloalkene ring.

Term [11]

The compound according to any one of terms [4] to [10] or a pharmaceutically acceptable salt thereof, wherein ring Q² is a group of the following formula (3):

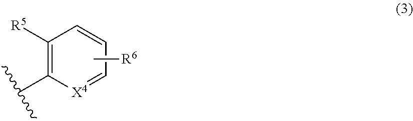

wherein X⁴ is N or CH;

R⁵ is halogen atom, C₁₋₆ alkyl group which may be optionally substituted with the same or different 1 to 3 halogen atoms, or C₁₋₆ alkoxy group which may be optionally substituted with the same or different 1 to 3 halogen atoms;

R⁶ is hydrogen atom, halogen atom, C₁₋₆ alkyl group which may be optionally substituted with the same or different 1 to 3 halogen atoms, or C₁₋₆ alkoxy group which may be optionally substituted with the same or different 1 to 3 halogen atoms.

Term [12]

The compound according to term [11] or a pharmaceutically acceptable salt thereof, wherein X⁴ is N.

Term [13]

The compound according to any one of terms [1] to [12] or a pharmaceutically acceptable salt thereof, wherein both R¹ and R² are hydrogen atom.

Term [14]

The compound according to term [1] or a pharmaceutically acceptable salt thereof, wherein the compound is a compound of formula (1b):

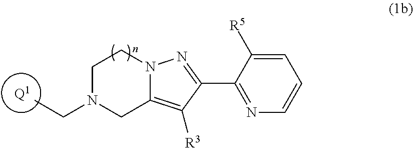

wherein n is 1 or 2;

ring Q¹ is a group of the following formula (2c) or (2d):

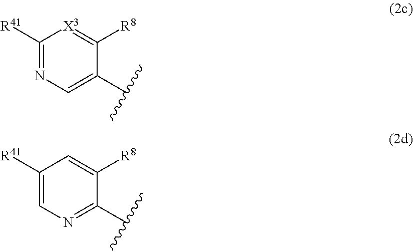

wherein X³ is N or CH;
R⁴¹ is halogen atom or C₁₋₆ alkyl group which may be optionally substituted with the same or different 1 to 3 halogen atoms; and
R⁸ is hydrogen atom, halogen atom, or C₁₋₆ alkyl group which may be optionally substituted with the same or different 1 to 3 halogen atoms;

R³ is hydrogen atom, halogen atom, or C₁₋₆ alkyl group which may be optionally substituted with the same or different 1 to 3 halogen atoms; and

R⁵ is halogen atom or C₁₋₆ alkyl group which may be optionally substituted with the same or different 1 to 3 halogen atoms.

Term [15]

The compound according to term [14] or a pharmaceutically acceptable salt thereof, wherein the ring Q¹ is a group of formula (2c).

Term [16]

The compound according to term [15] or a pharmaceutically acceptable salt thereof, wherein X³ is CH.

Term [17]

The compound according to term [15] or a pharmaceutically acceptable salt thereof, wherein X³ is N.

Term [18]

The compound according to term [14] or a pharmaceutically acceptable salt thereof, wherein the ring Q is a group of formula (2d).

Term [19]

The compound according to any one of terms [1] to [18] or a pharmaceutically acceptable salt thereof, wherein

n is 1; and

R³ is hydrogen atom or C₁₋₆ alkyl group.

Term [20]

The compound according to any one of terms [10] to [19] or a pharmaceutically acceptable salt thereof, wherein R⁶ is hydrogen atom.

Term [21]

The compound according to any one of terms [10] to [20] or a pharmaceutically acceptable salt thereof, wherein R⁴¹ is C₁₋₄ alkyl group substituted with 1 to 3 fluorine atoms.

Term [22]

A compound selected from the group consisting of the following formulae:

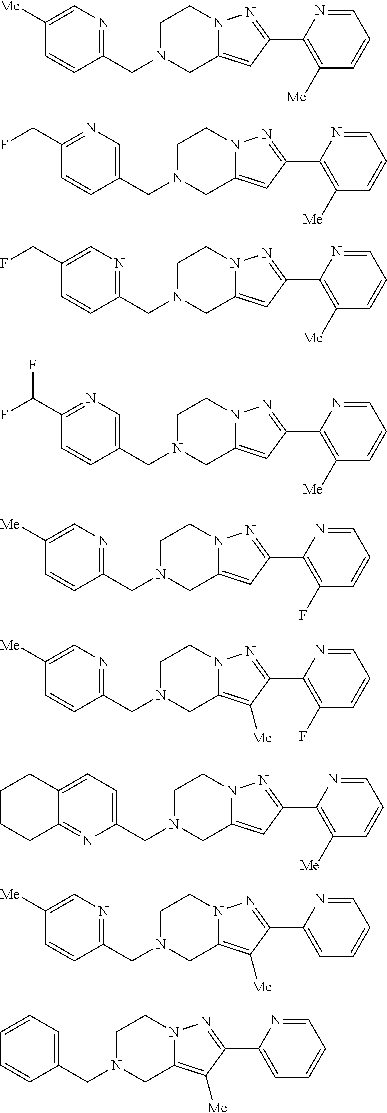

or a pharmaceutically acceptable salt thereof.

Term [23]

A pharmaceutical product comprising the compound according to any one of terms [1] to [22] or a pharmaceutically acceptable salt thereof as an active ingredient.

Term [24]

A medicament for treating attention deficit hyperactivity disorder, comprising the compound according to any one of terms [1] to [22] or a pharmaceutically acceptable salt thereof as an active ingredient.

Term [25]

The medicament according to term [24], wherein the attention deficit hyperactivity disorder is a disorder with inattention as a predominant symptom.

Term [26]

The medicament according to term [24], wherein the attention deficit hyperactivity disorder is a disorder with hyperactivity as a predominant symptom.

Term [27]

The medicament according to term [24], wherein the attention deficit hyperactivity disorder is a disorder with impulsivity as a predominant symptom.

Term [28]

A medicament for treating autistic spectrum disorder, comprising the compound according to any one of terms [1] to [22] or a pharmaceutically acceptable salt thereof as an active ingredient.

Term [29]

The medicament according to term [28], wherein the autistic spectrum disorder is a disorder with persistent deficits in social communication and social interaction as a predominant symptom.

Term [30]

The medicament according to term [28], wherein the autistic spectrum disorder is a disorder with restricted repetitive behaviors, interests, or activities.

Term [31]

A method for treating a central nervous system disease selected from the group consisting of attention deficit hyperactivity disorder, autistic spectrum disorder, schizophrenia, mood disorder, and cognitive dysfunction, which comprises administering a therapeutically effective amount of the compound according to any one of terms [1] to [22] or a pharmaceutically acceptable salt thereof to a patient in need thereof.

Term [32]

Use of the compound according to any one of terms [1] to [22] or a pharmaceutically acceptable salt thereof in the manufacture of a medicament for treating a central nervous system disease selected from the group consisting of attention deficit hyperactivity disorder, autistic spectrum disorder, schizophrenia, mood disorder, and cognitive dysfunction.

Term [33]

The compound according to any one of terms [1] to [22] or a pharmaceutically acceptable salt thereof for use in treating a central nervous system disease selected from the group consisting of attention deficit hyperactivity disorder, autistic spectrum disorder, schizophrenia, mood disorder, and cognitive dysfunction.

### Effects of the Invention

The present compound exhibits a potent effect on the dopamine D₄ receptor. In addition, the present compound has high bioavailability after oral administration and good brain penetration, and is also at low risk for hepatotoxicity. Hence, the present compound is useful as a highly-safe and potent medicament for treating a central nervous system disease, which has no drug dependence and a reduced risk for a cardiovascular side effect, and exhibits a fast-acting pharmaceutical effect in a small dose (e.g. a medicament for treating attention deficit hyperactivity disorder).

### DESCRIPTION OF EMBODIMENTS

Hereinafter, the present invention is explained in detail. The number of carbon atoms in the definition of the “substituent” used herein may be expressed as, for example, the term “C₁₋₆”. Specifically, the term “C₁₋₆ alkyl” is used for the same meaning as alkyl group having 1 to 6 carbon atoms.

Specific examples of “halogen atom” used herein include fluorine atom, chlorine atom, bromine atom, and iodine atom.

The term “C₁₋₆ alkyl group” used herein means a straight or branched, saturated hydrocarbon group having 1 to 6 carbon atoms. Preferred examples thereof include “C₁₋₄ alkyl group”. Specific examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, and 2-ethylbutyl.

The term “C₁₋₄ alkylene group” used herein means a straight or branched, divalent saturated hydrocarbon group having 1 to 4 carbon atoms, or a divalent saturated hydrocarbon group containing a cyclic structure having 3 to 4 carbon atoms.

Specific examples of the straight or branched “C₁₋₄ alkylene group” include methylene, ethylene, propyl, propylene, butylene, 1-methylmethylene, 1-ethylmethylene, 1-propylmethylene, 1-methylethylene, 2-methylethylene, and 1-ethylethylene. Preferred examples thereof include methylene and ethylene.

Specific examples of the “C₁₋₄ alkylene group” containing a cyclic structure include the following groups:

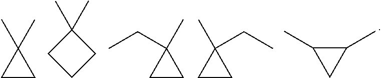

The term “C₁₋₆ alkoxy group” used herein means “C₁₋₆ alkyl-O— group” wherein the “C₁₋₆ alkyl” moiety thereof is as defined in the above “C₁₋₆ alkyl”. Preferred examples thereof include “C₁₋₄ alkoxy group”. Specific examples of the “C₁₋₆ alkoxy group” include methoxy, ethoxy, propoxy, isopropoxy, butoxy, isobutoxy, sec-butoxy, and tert-butoxy.

The “C₁₋₆ alkyl” moiety of “C₁₋₆ alkylcarbonyl group” used herein is as defined in the above “C₁₋₆ alkyl”. Preferred examples thereof include “C₁₋₄ alkylcarbonyl group”. Specific examples of the “C₁₋₆ alkylcarbonyl group” include methylcarbonyl, ethylcarbonyl, propylcarbonyl, isopropylcarbonyl, pentylcarbonyl, isobutylcarbonyl, and butylcarbonyl.

The term “aminocarbonyl group” used herein means a formyl group wherein hydrogen atom is substituted with amino group.

The term “C₃₋₁₀ cycloalkyl group” used herein means a 3- to 10-membered monocyclic or polycyclic, saturated or partially-unsaturated hydrocarbon group. Preferred examples thereof include “C₃₋₆ cycloalkyl group” and “C₅₋₁₀ cycloalkyl group”. Specific examples of the “C₃₋₁₀ cycloalkyl group” include cyclopropyl, cyclobutyl, cyclopentyl, cyclohexyl, cycloheptyl, cyclopentenyl, cyclohexenyl, decalinyl, adamantyl, and norbornyl.

The “C₃₋₁₀ cycloalkyl group” may be combined with phenyl or 3- or 6-membered heteroaryl to form a fused ring. When the cycloalkyl is fused with an aromatic ring (i.e. phenyl or 5- or 6-membered heteroaryl) to form a polycyclic “C₃₋₁₀ cycloalkyl group”, the binding position of the fused ring group is limited to one of the carbon atoms in the cycloalkyl ring. Specific examples thereof include the groups of the following formulae. Examples of substituents for the phenyl and 5- or 6-membered heteroaryl include the substituents in the “optionally-substituted C₆₋₁₀ aryl group” and “optionally-substituted heteroaryl group”

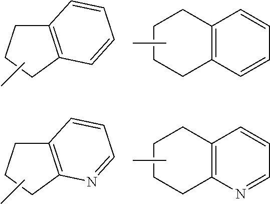

The term “3- to 8-membered/5- to 8-membered cycloalkane ring” used herein means a 3- to 8-membered/5- to 8-membered monocyclic saturated hydrocarbon ring. Preferred examples thereof include 5- or 6-membered saturated hydrocarbon ring. Specific examples of the “3- to 8-membered/5- to 8-membered cycloalkane ring” include cyclopropane ring, cyclobutane ring, cyclopentane ring, cyclohexane ring, cycloheptane ring, and cyclooctane ring.

The term “5- to 8-membered cycloalkene ring” used herein means a 5- to 8-membered monocyclic partially-unsaturated hydrocarbon ring. Preferred examples thereof include 5- or 6-membered partially-unsaturated hydrocarbon ring. Specific examples of the “5- to 8-membered cycloalkene ring” include cyclopentene ring, cyclohexene ring, cycloheptene ring, cycloheptadiene ring, and cyclooctene ring.

The term “C₆₋₁₀ aryl group” used herein means an aromatic hydrocarbon group having 6 to 10 carbon atoms. Preferred examples thereof include “C₆ aryl group” (phenyl) Specific examples of the “C₆₋₁₀ aryl group” include phenyl, 1-naphthyl, and 2-naphthyl.

The “C₆₋₁₀ aryl group” also encompasses a fused ring group of phenyl with a 5- to 7-membered ring containing the same or different one or more (e.g. 1 to 4) heteroatoms selected from the group consisting of nitrogen atom, sulfur atom, and oxygen atom or a 5- to 7-membered saturated or partially-unsaturated hydrocarbon ring (e.g. cyclopentane, cyclopentene, or cyclohexane). When the aromatic ring is fused with a non-aromatic ring to form a polycyclic “C₆₋₁₀ aryl group”, the binding position of the fused ring group is limited to one of the carbons in the aromatic ring. Specific examples thereof include the groups of the following formulae:

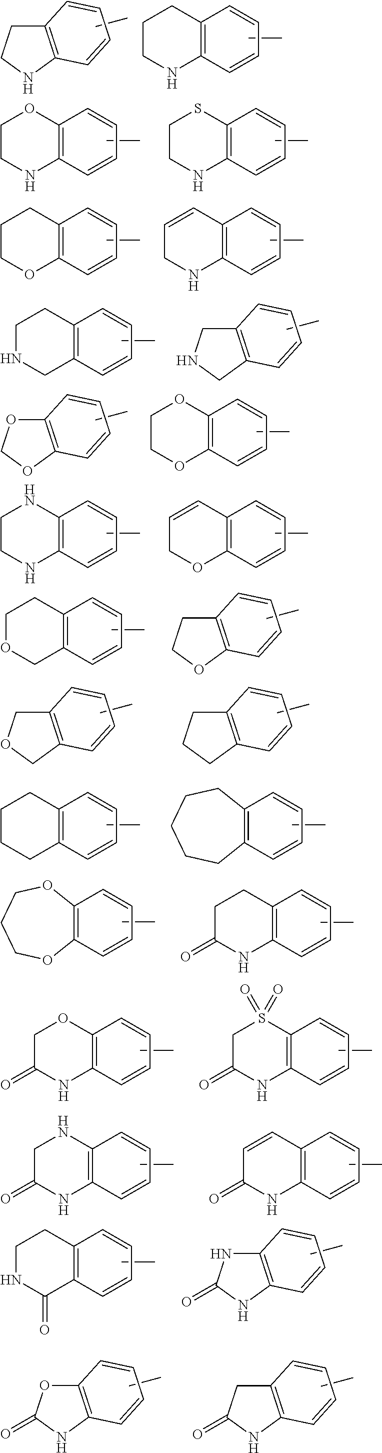

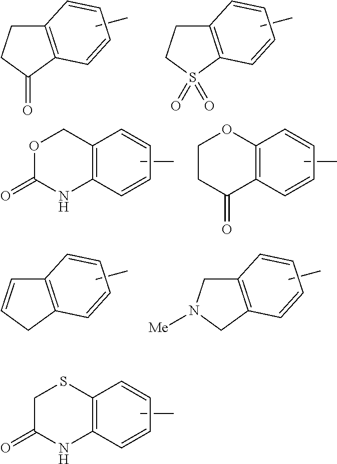

Examples of the term “heteroaryl group” used herein include a 5- to 10-membered monocyclic or polycyclic aromatic group which contains the same or different one or more (e.g. 1 to 4) heteroatoms selected from the group consisting of nitrogen atom, sulfur atom, and oxygen atom. The “polycyclic heteroaryl group” is preferably a di- or tri-cyclic group, and more preferably a di-cyclic group. The polycyclic heteroaryl group also encompasses a fused ring group of a monocyclic heteroaryl group mentioned above with an aromatic group (such as benzene and pyridine) or a non-aromatic ring (such as cyclohexyl and piperidine). Specific examples of the “heteroaryl group” include the groups of the following formulae:

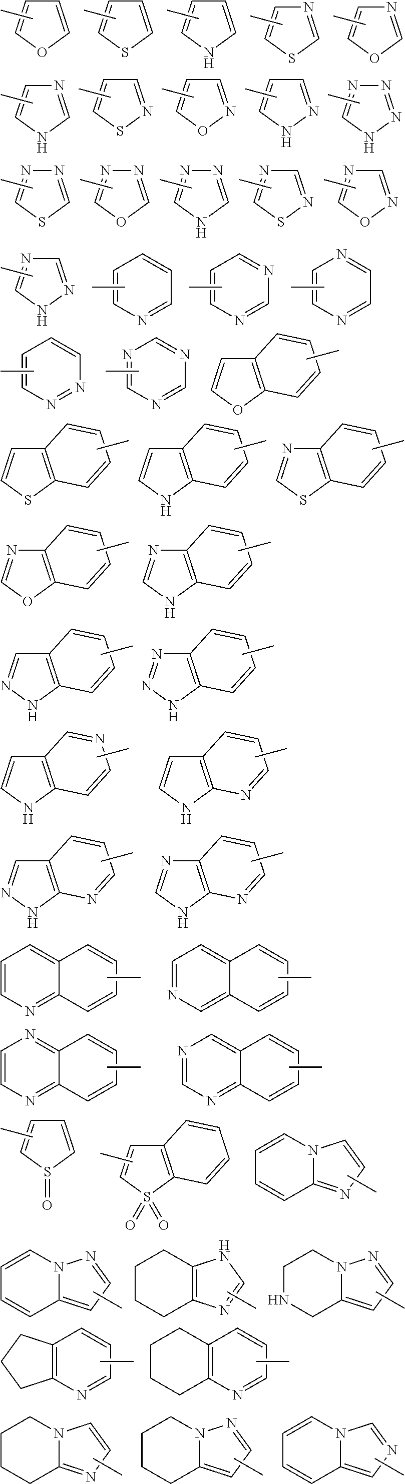

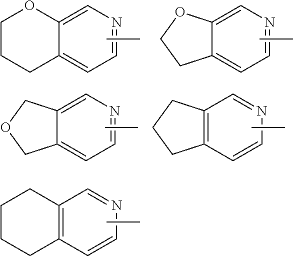

The “5- to 10-membered heteroaryl group” as ring Q¹ is preferably a 5- to 10-membered heteroaryl group containing 1 to 3 nitrogen atoms, more preferably the groups of the following formulae:

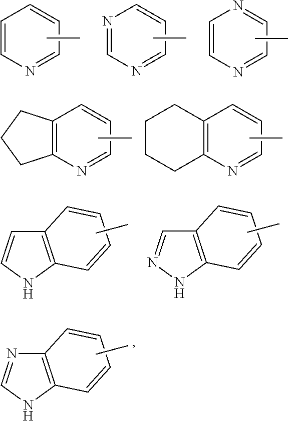

and most preferably the groups of the following formulae:

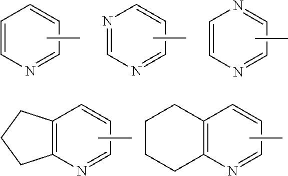

Specific examples of the “6-membered heteroaryl group” as ring Q² include pyridyl, pyrimidinyl, pyridazinyl, pyrazinyl, and triazinyl, preferably pyridyl and pyrimidinyl, and more preferably pyridyl.

The bond across a ring in the above formulae means that a “group” is linked at any replaceable position in the ring. For example, when a grout is the heteroaryl group of the following formula:

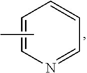

the group means 2-pyridyl group, 3-pyridyl group, or 4-pyridyl group.

Furthermore, when a “heteroaryl group” is a polycyclic group, for example, the group of the following formula:

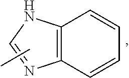

the group may be 1-benzimidazolyl, 2-benzimidazolyl, or 4-, 5-, 6- or 7-benzimidazolyl.

When an aromatic ring is fused with a non-aromatic ring (such as cyclohexane ring and piperidine ring) to form a polycyclic heteroaryl group, the binding position of the fused ring group is limited to one of the carbons in the aromatic ring. For example, when the “polycyclic heteroaryl group” is the group of the following formula:

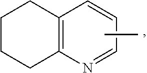

the bond means that a group is linked at 2-, 3-, or 4-position.

Examples of the term “saturated heterocyclyl group” include a 4- to 10-membered monocyclic or polycyclic saturated heterocyclyl group containing the same or different 1 to 3 heteroatoms selected from the group consisting of nitrogen atom, oxygen atom, and sulfur atom. Each of the nitrogen atom, oxygen atom, and sulfur atom composes the heterocycle. The heterocyclyl group may be saturated or partially-unsaturated. The group is preferably a saturated heterocyclyl group, and more preferably a 5- or 6-membered saturated heterocyclyl group. Specific examples of the heterocyclyl group include pyranyl, dihydropyranyl, tetrahydropyranyl, tetrahydrofuryl, pyrrolidinyl, imidazolidinyl, piperidinyl, morpholinyl, thiomorpholinyl, dioxothiomorpholinyl, hexamethyleneiminyl, oxazolidinyl, thiazolidinyl, imidazolidinyl, tetrahydrofuranyl, oxooxazolidyl, dioxooxazolidinyl, dioxothiazolidinyl, tetrahydropyranyl, 5-oxo-1,2,4-oxadiazol-3-yl, 5-oxo-1,2,4-thiadiazol-3-yl, and 5-thioxo-1,2,4-oxadiazol-3-yl. The nitrogen atom in the ring is not the binding position in the “group”. In other words, the group does not encompass groups such as 1-pyrrolidino group.

The “4- to 6-membered saturated heterocyclyl group” also encompasses a saturated bicyclo ring group and a saturated spiro ring group which include “4- to 6-membered saturated hetero ring” as the basic skeleton of the group. Specific examples thereof include the following “groups”

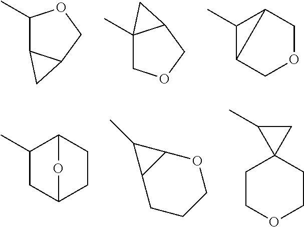

The “saturated heterocyclyl group” may be combined with phenyl or a 5- or 6-membered heteroaryl to form a fused ring. For example, the group also encompasses fused ring groups of the above 4- to 6-membered saturated heterocyclyl group with phenyl or 5- or 6-membered heteroaryl. Specific examples thereof include dihydroindolyi, dihydroisoindolyl, dihydropurinyl, dihydrothiazolopyrimidinyl, dihydrobenzodioxanyl, isoindolinyl, tetrahydroquinolinyl, decahydroquinolinyl, tetrahydroisoquinolinyl, decahydroisoquinolinyl, tetrahydronaphthyridinyl, and tetrahydropyridoazepinyl. Examples of substituents for the phenyl and 5- or 6-membered heteroaryl include the substituents in the “optionally-substituted C₆₋₁₀ aryl group” and “optionally-substituted heteroaryl group”.

The term “5- to 10-membered cyclic amino group” used herein means a 5- to 10-membered monocyclic or polycyclic amino group. The nitrogen atom in the ring is the direct binding position in the “group”, and is preferably 5- to 7-membered group. Specific examples thereof include azetidino, pyrrolidino, piperidino, morpholino, thiomorpholino, thiomorpholinooxide, thiomorpholinodioxide, and piperazino. The group also encompasses a cyclic amino group including a partially-unsaturated bond or bonds therein.

The “5- to 10-membered cyclic amino group” may be combined with phenyl or 5- or 6-membered monocyclic heteroaryl to form a fused ring. Specific examples thereof include the “groups” of the following formulae. Examples of substituents for the phenyl or 5- or 6-heteroaryl include the substituents in the “optionally-substituted C₆-10 aryl group” and “optionally-substituted heteroaryl group”

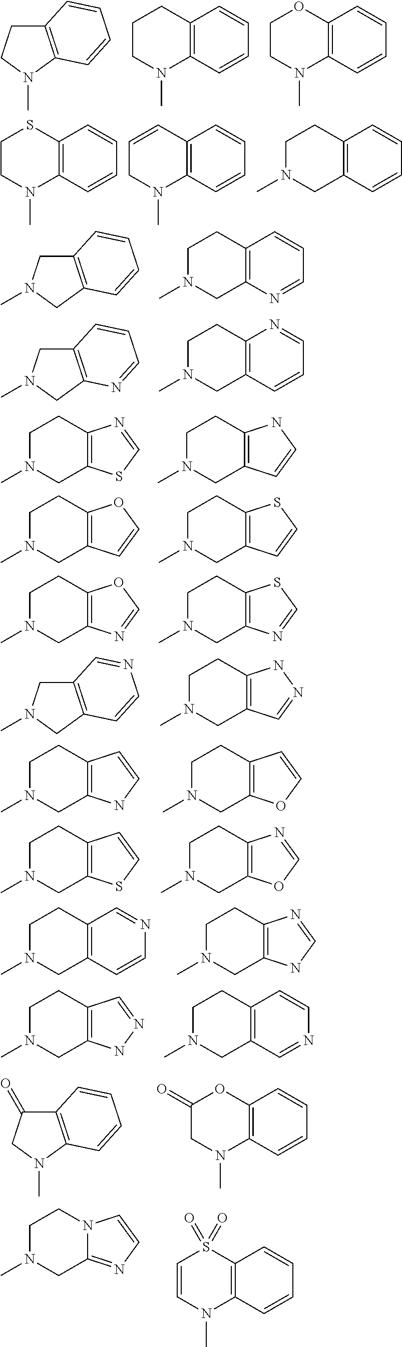

Unless otherwise specified, a substituent or substituents used in the definition “optionally-substituted” may be substituted at any replaceable positions, and the number of substituents is not limited as long as it is within replaceable range. For example, when optionally-substituted C₁₋₆ alkyl group is methyl group, the replaceable number thereof is 1 to 3. When optionally-substituted C₆₋₁₀ aryl group is phenyl group, the replaceable number thereof is 1 to 5. Also, when multiple substituted groups are present, they may be the same or different. In addition, unless otherwise specified, the definition of each group is also applied in another group including said group as a part thereof or a substituent thereof.

Examples of substituents in the “optionally-substituted C₁₋₄ alkylene group” include hydroxy group, halogen atom, C₃₋₇ cycloalkyl group, and C₁₋₆ alkoxy group. Preferred examples thereof include fluorine atom.

Examples of substituents in the “optionally-substituted C₁₋₆ alkyl group”, “optionally-substituted C₁₋₆ alkoxy group”, and “optionally-substituted C₁₋₆ alkylcarbonyl group” include

(1) halogen atom,
(2) C₃₋₇cycloalkyl group,
(3) C₁₋₆ alkoxy group which may be optionally substituted with the same or different 1 to 3 halogen atoms,
(4) cyano group,
(5) amino group which may be optionally substituted with the same or different 1 or 2 groups selected independently from the group consisting of C₁₋₆ alkyl group and C₃₋₇ cycloalkyl group,
(6) hydroxy group,
(7) C₁₋₆ alkoxycarbonyl group, and
(8) aminocarbonyl group wherein the amino moiety thereof may be optionally substituted with the same or different 1 or 2 groups selected independently from the group consisting of C₁₋₆ alkyl and C₃₋₇ cycloalkyl group.

Preferred examples thereof include fluorine atom and C₁₋₆ alkoxy group.

Examples of substituents in the “optionally-substituted aryl group”, “optionally-substituted heteroaryl group”, “optionally-substituted saturated heterocyclyl group”, “optionally-substituted cyclic amino group”, and “optionally-substituted cycloalkyl group” include

(1) halogen atom,
(2) C₁₋₆ alkyl group which may be optionally substituted with the same or different 1 to 3 halogen atoms,
(3) C₁₋₆ alkoxy group which may be optionally substituted with the same or different 1 to 3 halogen atoms,
(4) cyano group,
(5) amino group which may be optionally substituted with the same or different 1 or 2 groups selected independently from the group consisting of C₁₋₆ alkyl group and C₃₋₇ cycloalkyl group,
(6) hydroxy group,
(7) C₁₋₆ alkoxycarbonyl group, and
(8) aminocarbonyl group wherein the amino moiety thereof may be optionally substituted with the same or different 1 or 2 groups selected independently from the group consisting of C₁₋₆ alkyl and C₃₋₇ cycloalkyl group.

Preferred examples thereof include halogen atom, C₁₋₆ alkyl group, C₁₋₆ alkoxy group, cyano group, and amino group which may be optionally substituted with the same or different 1 or 2 groups selected independently from the group consisting of C₁₋₆ alkyl group and C₃₋₇ cycloalkyl group.

Examples of substituents in the “optionally-substituted amino group” and “optionally-substituted aminocarbonyl group” include the same or different 1 or 2 groups selected independently from the group consisting of

(1) C₁₋₆ alkyl group which may be optionally substituted with

(a) 1 to 3 halogen atoms,

(b) cyano group,

(c) hydroxy group,

(d) C₁₋₆ alkoxy group which may be optionally substituted with the same or different 1 to 3 halogen atoms, or

(e) C₃₋₇ cycloalkyl group which may be optionally substituted with the same or different 1 to 3 halogen atoms or C₁₋₆ alkyl group;

(2) C₃₋₇ cycloalkyl group which may be optionally substituted with C₁₋₆ alkyl, C₁₋₆ alkoxy, or the same or different 1 to 3 halogen atoms;
(3) phenyl group which may be optionally substituted with the same or different 1 to 4 groups selected independently from the group consisting of

(a) halogen atom,

(b) cyano group,

(c) C₁₋₆ alkyl group which may be optionally substituted with the same or different 1 to 3 halogen atoms, and

(d) C₁₋₆ alkoxy group which may be optionally substituted with the same or different 1 to 3 halogen atoms;

(4) 5- or 6-membered heteroaryl group which may be optionally substituted with the same or different 1 to 4 groups selected independently from (a) to (d) defined in the above (3); and
(5) 5- or 6-membered saturated heterocyclyl group which may be optionally substituted with the same or different 1 to 4 groups selected independently from (a) to (d) defined in the above (3).

The phrase “R¹ and R² may be combined with the carbon atom(s) to which they are attached to form 3- to 8-membered cycloalkane ring” means that (1) R¹ and R² are combined with the same carbon atom to form 3- to 8-membered spirocycloalkane ring; and (2) R¹ and R² are combined with the different adjacent carbon atoms to form 3- to 8-membered fused cycloalkane ring.

The present compound may be in the forms of a hydrate and/or a solvate. Thus, the present compound also encompasses hydrate and/or solvate such as ethanol solvate. Furthermore, the present compound encompasses all types of crystal forms of the present compound.

Specific examples of the pharmaceutically acceptable salt of the compound of formula (1) (hereinafter referred to as “compound (1)”, as necessary) include an inorganic acid salt such as hydrochloride, hydrobromate, sulfate, phosphate, and nitrate; and an organic acid salt such as acetate, propionate, oxalate, succinate, lactate, malate, tartrate, citrate, maleate, fumarate, methanesulfonate, p-toluenesulfonate, benzenesulfonate, and ascorbate.

The compound of formula (1) may be in the form of a tautomer. Thus, the present compound also encompasses the tautomer of the compound of formula (1).

The compound of formula (1) may contain one or more asymmetric carbon atoms. Thus, the present compound encompasses not only racemic forms of the compound of formula (1) but also optically-active forms thereof. When the compound of formula (1) contains two or more asymmetric carbon atoms, the compound can result in various stereoisomerisms. Thus, the present compound also encompasses the sterecisomer of the compound and a mixture or isolate thereof.

Also, the present compound encompasses the compound of formula (1) wherein one or more of ¹H are substituted with ²H(D) (i.e. deuterated form).

Hereinafter, the process of preparing the present compound is illustrated with some examples, but the invention should not be limited thereto. Also, some terms herein may be defined by the following abbreviations for the sake of simplicity.

Boc group: tert-butoxycarbonyl group

Cbz group: benzyloxycarbonyl group

Alloc group: allyloxycarbonyl group

Fmoc group: 9-fluorenylmethyloxycarbonyl group

THF: tetrahydrofuran

DMF: N, N-dimethylformamide

Preparations

The present compound can be prepared according to, for example, the following processes of Preparations 1-7. The processes may be optionally modified by those skilled in the organic synthesis field. As appropriate, each compound used as a starting material may be used in the salt form.

In the following processes, besides the case where the use of protective groups is specified, any functional groups other than reaction sites may be optionally protected and then deprotected after the reaction or reactions are completed to give the desired compound, when the functional groups can be changed under the reaction condition or can be inappropriate for carrying out the reaction. The protective group includes any conventional groups described in various literatures, for example, T. W. Greene and P. G. M. Wuts, “Protective Groups in Organic Synthesis”, 3rd Ed., John Wiley and Sons, inc., New York (1999). More specifically, specific examples of the protective groups for amino group include benzyloxycarbonyl, tert-butoxycarbonyl, acetyl, and benzyl, and specific examples of the protective groups for hydroxy group include trialkylsilyl, acetyl, and benzyl.

The protecting groups can be introduced and cleaved according to commonly-used methods in synthetic organic chemistry (e.g. the method described in T. W. Greene and P. G. M. Wuts, “Protective Groups in Organic Synthesis”, 3rd Ed., John Wiley and Sons, inc., New York (1999)) and similar methods thereto.

Preparation 1

The compound of formula (1) is prepared according to, for example, the following process:

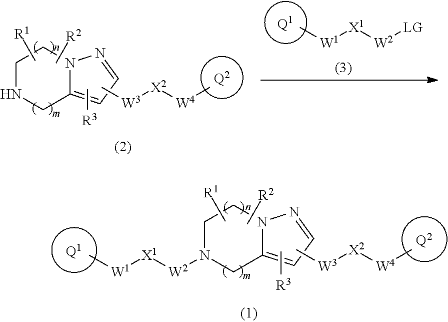

wherein m, n, W¹, W², W³, W⁴, R¹, R², R³, X¹, X², ring Q¹, and ring Q² are as defined in the above term [1], LG is a leaving group (e.g. iodine atom, bromine atom, chlorine atom, and substituted sulfonyl group (e.g. methanesulfonyl group and p-toluenesulfonyl group)).

Compound (1) can be prepared by reacting compound (2) with compound (3) in an appropriate inert solvent. The reaction may be carried out in the presence of a base and/or a phase-transfer catalyst, as appropriate. The reaction temperature is typically a temperature of about −20° C. to the boiling point of the used solvent. The reaction time is typically in the range from 10 minutes to 48 hours, which may vary according to various conditions such as a reaction temperature, a base, a starting material, and a solvent to be used.

Specific examples of the base include an organic base such as triethylamine, diisopropylethylamine, and pyridine; an inorganic base such as potassium carbonate, sodium carbonate, cesium carbonate, potassium hydrogen carbonate, sodium hydrogen carbonate, potassium dihydrogen phosphate, dipotassium hydrogen phosphate, potassium phosphate, sodium dihydrogen phosphate, disodium hydrogen phosphate, sodium phosphate, potassium hydroxide, sodium hydroxide, and sodium hydride; and a metal alkoxide such as sodium methoxide and potassium tert-butoxide.

Specific examples of the phase-transfer catalyst include tetrabutylammonium hydrogen sulfate.

Specific examples of the inert solvent include a halogenated hydrocarbon such as chloroform and dichloromethane; an aromatic hydrocarbon such as benzene and toluene; an ether-type solvent such as diethyl ether, tetrahydrofuran (THF), and 1,4-dioxane; a lower alcohol such as methanol, ethanol, and 2-propanol; an aprotic polar solvent such as acetonitrile, acetone, methyl ethyl ketone, dimethylformamide, N-methyl-2-pyrrolidinone, and dimethylsulfoxide; and a mixture thereof.

Preparation 2

Among the compound of formula (1), the compound of formula (1b) is prepared according to, for example, the following process:

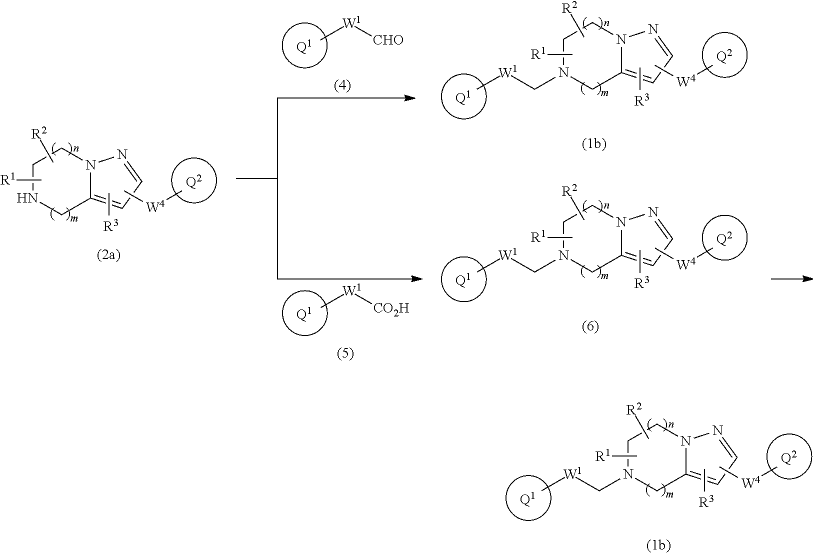

wherein m, n, W¹, W⁴, R¹, R², R³, ring Q¹, and ring Q² are as defined in the above term [1].

Compound (1b) can be prepared by the reductive amination of compound (2a) and the aldehyde compound of formula (4) with a reducing agent in an appropriate inert solvent. The reaction may be carried out in the presence of a base or an acid, as appropriate. The reaction temperature is typically a temperature of about −20° C. to the boiling point of the used solvent. The reaction time is typically in the range from 10 minutes to 48 hours, which may vary according to various conditions such as a reaction temperature, a reducing agent, a starting material, and a solvent to be used.

Specific examples of the reducing agent include a complex hydride such as sodium triacetoxyborohydride, lithium aluminum hydride, sodium borohydride, and sodium cyanoborohydride; and a borane complex (e.g. borane-dimethylsulfide complex and borane-tetrahydrofuran complex).

Specific examples of the base include an organic base such as triethylamine, diisopropylethylamine, and pyridine; an inorganic base such as potassium carbonate, sodium carbonate, cesium carbonate, potassium hydrogen carbonate, sodium hydrogen carbonate, potassium dihydrogen phosphate, dipotassium hydrogen phosphate, potassium phosphate, sodium dihydrogen phosphate, disodium hydrogen phosphate, sodium phosphate, potassium hydroxide, sodium hydroxide, and sodium hydride; and a metal alkoxide such as sodium methoxide and potassium tert-butoxide.

Specific examples of the acid include an organic acid such as acetic acid, trifluoroacetic acid, and methanesulfonic acid; and an inorganic acid such as hydrochloric acid and sulfuric acid.

Specific examples of the solvent include water; acetonitrile; a halogenated hydrocarbon such as chloroform and dichloromethane; an aromatic hydrocarbon such as benzene and toluene; an ether-type solvent such as 1,2-dimethoxyethane, tetrahydrofuran, and 1,4-dioxane; an alcohol-type solvent such as methanol, ethanol, and 2-propanol; an aprotic polar solvent such as dimethylformamide and N-methyl-2-pyrrolidinone; and a mixture thereof.

Compound (1b) can be also prepared by reacting compound (6) with a reducing agent in an inert solvent. The reaction temperature is typically a temperature of about −20° C. to the boiling point of the used solvent. The reaction time is typically in the range from 10 minutes to 48 hours, which may vary according to various conditions such as a reaction temperature, a condensing agent, a starting material, and a solvent to be used.

Specific examples of the reducing agent include lithium aluminum hydride and a borane complex (e.g. borane-dimethylsulfide complex or borane-tetrahydrofuran complex).

Specific examples of the inert solvent include an ether-type solvent such as tetrahydrofuran and 1,4-dioxane; and a mixture thereof.

Compound (6) can be prepared by reacting compound (2a) with the carboxylic acid of formula (5) in an inert solvent in the presence of a condensing agent. Furthermore, the reaction may be carried out in the presence of a base. The reaction temperature is typically a temperature of about −20° C. to the boiling point of the used solvent. The reaction time is typically in the range from 10 minutes to 48 hours, which may vary according to various conditions such as a reaction temperature, a condensing agent, a starting material, and a solvent to be used.

Compound (6) can be also prepared by reacting compound (2a) with an acid halide or an acid anhydride derived from compound (5) in an inert solvent in the presence of a base. The reaction temperature is typically a temperature of about −20° C. to the boiling point of the used solvent. The reaction time is typically in the range from 10 minutes to 48 hours, which may vary according to various conditions such as a reaction temperature, a condensing agent, a starting material, and a solvent to be used.

Specific examples of the condensing agent include dicyclohexylcarbodiimide (DCC), diisopropylcarbodiimide (DIPC), 1-ethyl-3-(3-dimethylaminopropyl)-carbodiimide (WSC), benzotriazol-1-yl-tris(dimethylamino)phosphonium hexafluorophosphate (BOP), diphenylphosphonyldiamide (DPPA), N,N-carbonyldiimidazole (CDI), and benzotriazol-1-yl-N,N,N′,N′-tetramethyluronium hexafluorophosphate (HBTU). As appropriate, the reaction may be carried out with the addition of an additive such as N-hydroxysuccinimide (HOSu), 1-hydroxybenzotriazole (HOBt), and 3-hydroxy-4-oxo-3,4-dihydro-1,2,3-benzotriazine (HOOBt).

Specific examples of the base include an organic base such as triethylamine, diisopropylethylamine, and pyridine; an inorganic base such as potassium carbonate, sodium carbonate, cesium carbonate, potassium hydrogen carbonate, sodium hydrogen carbonate, potassium dihydrogen phosphate, dipotassium hydrogen phosphate, potassium phosphate, sodium dihydrogen phosphate, disodium hydrogen phosphate, sodium phosphate, potassium hydroxide, sodium hydroxide, and sodium hydride; and a metal alkoxide such as sodium methoxide and potassium tert-butoxide.

Specific examples of the inert solvent include a halogenated hydrocarbon such as chloroform and dichloromethane; an aromatic hydrocarbon such as benzene and toluene; an ether-type solvent such as diethyl ether, tetrahydrofuran (THF), and 1,4-dioxane; an aprotic polar solvent such as acetonitrile, acetone, methyl ethyl ketone, dimethylformamide, N-methyl-2-pyrrolidinone, and dimethylsulfoxide; and a mixture thereof.

Preparation 3

Among the compound of formula (2), the compound of formula (2b) is prepared according to, for example, the following process:

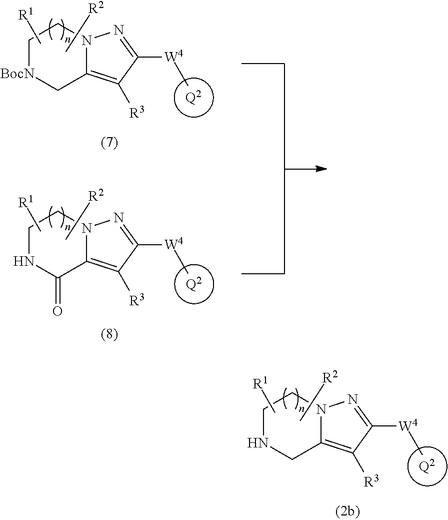

wherein n, R¹, R², R³, W⁴, and ring Q² are as defined in the above term [1].

Compound (2b) is prepared by reacting compound 7 with an acid (e.g. an inorganic acid such as hydrochloric acid and sulfuric acid, and an organic acid such as trifluoroacetic acid) in an appropriate inert solvent. The reaction temperature is typically a temperature of about −20° C. to the boiling point of the used solvent. The reaction time is typically in the range from 10 minutes to 48 hours, which may vary according to various conditions such as a reaction temperature, an acid, a starting material, and a solvent to be used.

Specific examples of the inert solvent include a halogenated hydrocarbon such as chloroform and dichloromethane; an aromatic hydrocarbon such as benzene and toluene; an ether-type solvent such as diethyl ether, tetrahydrofuran, 1,4-dioxane, and 1,2-dimethoxyethane; a lower alcohol such as methanol, ethanol, and 2-propanol; an aprotic polar solvent such as acetonitrile, dimethylformamide, N-methyl-2-pyrrolidinone, and dimethylsulfoxide; and a mixture thereof.

Compound (2b) is also prepared by reacting compound (8) with a reducing agent. The reaction temperature is typically a temperature of about −20° C. to the boiling point of the used solvent. The reaction time is typically in the range from 10 minutes to 48 hours, which may vary according to various conditions such as a reaction temperature, a condensing agent, a starting atrial, and a solvent to be used.

Specific examples of the reducing agent include lithium aluminum hydride and a borane complex (e.g. borane-dimethylsulfide complex or borane-tetrahydrofuran complex).

Specific examples of the inert solvent include an ether-type solvent such as tetrahydrofuran and 1,4-dioxane, and a mixture thereof.

Preparation 4

Among the compound of formula (7), the compounds of formulae (7b) and (7c) are prepared according to, for example, the following process:

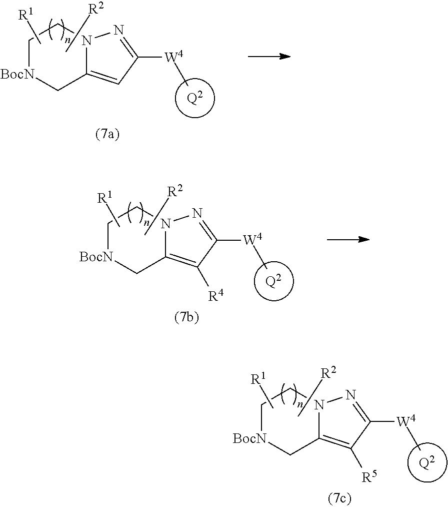

wherein n, R¹, R², W⁴, and ring Q² are as defined in the above term [1], R⁴ is halogen atom, and R⁵ is C₁₋₆ alkyl group.

Compound (7b) is prepared by reacting compound (7a) with a halogenating agent such as N-bromosuccinimide, N-chlorosuccinimide, and 1-chloromethyl-4-fluoro-1,4-diazoniabicyclo[2.2.2]octane bis(tetrafluoroborate) in an appropriate inert solvent. The reaction temperature is typically a temperature of about −20° C. to the boiling point of the used solvent. The reaction time is typically in the range from 10 minutes to 48 hours, which may vary according to various conditions such as a reaction temperature, a halogenating agent, a starting material, and a solvent to be used.

Specific examples of the inert solvent include a halogenated hydrocarbon such as chloroform and dichloromethane; an ether-type solvent such as diethyl ether, tetrahydrofuran, 1,4-dioxane, and 1,2-dimethoxyethane; an aprotic polar solvent such as dimethylformamide and N-methyl-2-pyrrolidinone; and a mixture thereof.

Compound (7c) is prepared by the coupling reaction of compound (7b) with an agent, for example, an organozinc compound such as dimethyl zinc or an organoboron compound such as trimethylboroxine in an appropriate inert solvent in the presence of a transition metal catalyst. The reaction may be carried out in the presence of a ligand, a base, an additive, etc., as appropriate. The reaction temperature is typically a temperature of −10° C. to the boiling point of the used solvent.

Specific examples of the transition metal catalyst include palladium (II) acetate, palladium (II) chloride, tris(dibenzylideneacetone)dipalladium (0), tetrakis(triphenylphosphine)palladium (0), bis(triphenylphosphine)palladium (II) chloride, dichlorobis(tri-O-tolylphosphine)palladium (II), bis(tri-tert-butylphosphine)palladium (0), and [1,1′-bis(diphenylphosphino)ferrocene]dichloropalladium (II).

Specific examples of the ligand include triphenylphosphine, tri-O-tolylphosphine, tri-tert-butylphosphine, tri-2-furylphosphine, tricyclohexylphosphine, triphenylarsine, 1,1′-bis(diphenylphosphino)ferrocene, 2-dicyclohexylphosphino-2′,6′-dimethoxybiphenyl, and 2-dicyclohexylphosphino-2′,4′,6′-triisopropylbiphenyl.

Specific examples of the base include an organic base such as triethylamine and diisopropylethylamine; and an inorganic base such as sodium carbonate, sodium hydrogen carbonate, potassium carbonate, cesium carbonate, and potassium phosphate.

Specific examples of the additive include an inorganic salt such as lithium chloride, cesium fluoride, copper (I) iodide, and copper (I) bromide.

Alternatively, compound (7c) can be prepared by reacting compound (7b) with alkyllithium such as n-butyllithium, and then alkyl halide such as methyl iodide.

Preparation 5

Among the compound of formula (7), the compound of formula (7d) is prepared by according to, for example, the following process:

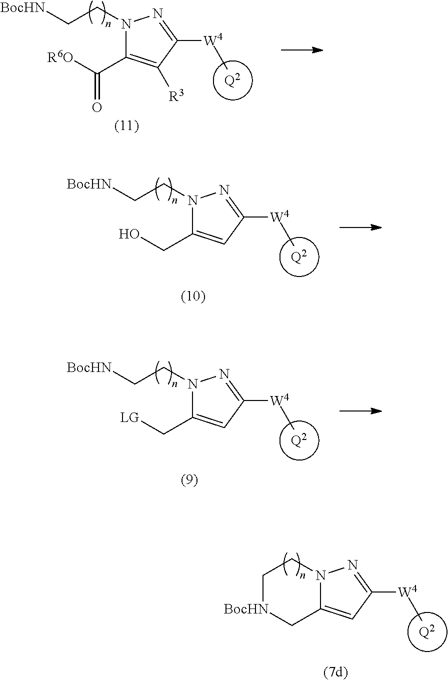

wherein n, W⁴, and ring Q are as defined in the above term [1], R⁶ is optionally-substituted C₁₋₄ alkyl group, and LG is a leaving group (e.g. iodine atom, bromine atom, chlorine atom, and substituted sulfonyl group (e.g. methanesulfonyl group and p-toluenesulfonyl group)).

Compound (7d) is prepared by reacting compound (9) with a base in an appropriate inert solvent. The reaction may be carried out in the presence of a phase-transfer catalyst, as appropriate. The reaction temperature is typically a temperature of about −20° C. to the boiling point of the used solvent. The reaction time is typically in the range from 10 minutes to 48 hours, which may vary according to various conditions such as a reaction temperature, a base, a starting material, and a solvent to be used.

Specific examples of the base include an organic base such as triethylamine, diisopropylethylamine, and pyridine; an inorganic base such as potassium carbonate, sodium carbonate, cesium carbonate, potassium hydrogen carbonate, sodium hydrogen carbonate, potassium dihydrogen phosphate, dipotassium hydrogen phosphate, potassium phosphate, sodium dihydrogen phosphate, disodium hydrogen phosphate, sodium phosphate, potassium hydroxide, sodium hydroxide, and sodium hydride; and a metal alkoxide such as sodium methoxide and potassium tert-butoxide.

Specific examples of the phase-transfer catalyst include tetrabutylammonium hydrogen sulfate.

Specific examples of the inert solvent include a halogenated hydrocarbon such as chloroform and dichloromethane; an aromatic hydrocarbon such as benzene and toluene; an ether-type solvent such as diethyl ether, tetrahydrofuran (THF), and 1,4-dioxane; a lower alcohol such as methanol, ethanol, and 2-propanol; an aprotic polar solvent such as acetonitrile, acetone, methyl ethyl ketone, dimethylformamide, N-methyl-2-pyrrolidinone, and dimethylsulfoxide; and a mixture thereof.

Compound (9) is prepared by converting hydroxyl group in compound (10) to halogen atom or substituted sulfonyloxy group such as p-toluenesulfonyl group and methanesulfonyl group in an appropriate inert solvent according to a conventional method.

For example, compound (9) wherein LG is halogen atom is prepared by reacting compound (10) with carbon tetrachloride or carbon tetrabromide in an appropriate inert solvent in the presence of triphenylphosphine.

Also, compound (9) wherein LG is substituted sulfonyloxy group is prepared by reacting compound (10) with p-toluenesulfonyl chloride or methanesulfonyl chloride in an inert solvent in the presence of a base. The reaction temperature is typically a temperature of about −20° C. to the boiling point of the used solvent. The reaction time is typically in the range from 10 minutes to 48 hours, which may vary according to various conditions such as a reaction temperature, a base, a starting material, and a solvent to be used.

Specific examples of the inert solvent include a halogenated solvent such as chloroform and dichloromethane; an ether-type solvent such as diethyl ether, tetrahydrofuran, 1,4-dioxane, and 1,2-dimethoxyethane; an aprotic polar solvent such as acetonitrile, dimethylformamide, N-methyl-2-pyrrolidinone, and dimethylsulfoxide; and a mixture thereof.

Specific examples of the base include an organic base such as triethylamine and pyridine, and an inorganic base such as potassium carbonate and sodium hydroxide.

Also, compound (9) wherein LG is halogen atom is prepared by reacting compound (9) wherein LG is substituted sulfonyloxy group with lithium bromide or lithium chloride in an inert solvent.

Compound (10) is prepared by reacting compound (11) with a reducing agent. The reaction temperature is typically a temperature of about −20° C. to the boiling point of the used solvent. The reaction time is typically in the range from 10 minutes to 48 hours, which may vary according to various conditions such as a reaction temperature, a condensing agent, a starting material, and a solvent to be used.

Specific examples of the reducing agent include lithium aluminum hydride and a borane complex (e.g. borane-dimethylsulfide complex or borane-tetrahydrofuran complex).

Specific examples of the inert solvent include an ether-type solvent such as tetrahydrofuran and 1,4-dioxane, and a mixture thereof.

Preparation 6

Among the compound of formula (8), the compound of formula (8a) is prepared according to, for example, the following process:

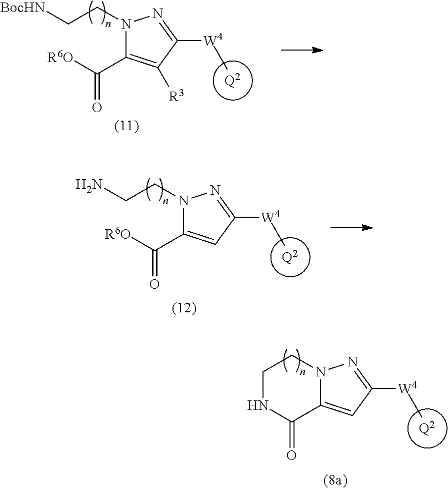

wherein n, W⁴, and ring Q² are as defined in the above term [1], and R⁶ is optionally-substituted C₁₋₄ alkyl group.

Compound (8a) is prepared by reacting compound (12) with a base (e.g. an inorganic base such as potassium carbonate and sodium carbonate; and an organic base such as triethylamine and pyridine) or an acid (e.g. an inorganic acid such as hydrochloric acid and sulfuric acid; and an organic acid such as trifluoroacetic acid). The reaction temperature is typically a temperature of about −20° C. to the boiling point of the used solvent. The reaction time is typically in the range from 10 minutes to 48 hours, which may vary according to various conditions such as a reaction temperature, an acid, a starting material, and a solvent to be used.

Specific examples of the inert solvent include a halogenated hydrocarbon such as chloroform and dichloromethane; an aromatic hydrocarbon such as benzene and toluene; an ether-type solvent such as diethyl ether, tetrahydrofuran, 1,4-dioxane, and 1,2-dimethoxyethane; a lower alcohol such as methanol, ethanol, and 2-propanol; an aprotic polar solvent such as acetonitrile, dimethylformamide, N-methyl-2-pyrrolidinone, and dimethylsulfoxide; and a mixture thereof.

Compound (12) is prepared by reacting compound (11) with an acid (e.g. an inorganic acid such as hydrochloric acid and sulfuric acid, and an organic acid such as trifluoroacetic acid) in an appropriate inert solvent. The reaction temperature is typically a temperature of about −20° C. to the boiling point of the used solvent. The reaction time is typically in the range from 10 minutes to 48 hours, which may vary according to various conditions such as a reaction temperature, an acid, a starting material, and a solvent to be used.

Specific examples of the inert solvent include a halogenated hydrocarbon such as chloroform and dichloromethane; an aromatic hydrocarbon such as benzene and toluene; an ether-type solvent such as diethyl ether, tetrahydrofuran, 1,4-dioxane, and 1,2-dimethoxyethane; a lower alcohol such as methanol, ethanol, and 2-propanol; an aprotic polar solvent such as acetonitrile, dimethylformamide, N-methyl-2-pyrrolidinone, and dimethylsulfoxide; and a mixture thereof.

Preparation 7

The compound of formula (11) is prepared according to, for example, the following process:

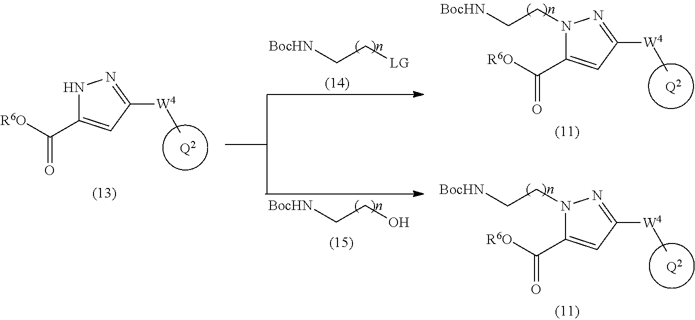

wherein n, W⁴, and ring Q² are as defined in the above term [1], R⁶ is optionally-substituted C₁₋₄ alkyl group, and LG is a leaving group (e.g. iodine atom, bromine atom, chlorine atom, and substituted sulfonyl group (e.g. methanesulfonyl group and p-toluenesulfonyl group)).

Compound (11) is prepared by reacting compound (13) with compound (14) in an appropriate inert solvent. The reaction may be carried out in the presence of a base and/or a phase-transfer catalyst, as appropriate. The reaction temperature is typically a temperature of about −20° C. to the boiling point of the used solvent. The reaction time is typically in the range from 10 minutes to 48 hours, which may vary according to various conditions such as a reaction temperature, a base, a starting material, and a solvent to be used.

Specific examples of the base include an organic base such as triethylamine, diisopropylethylamine, and pyridine; an inorganic base such as potassium carbonate, sodium carbonate, cesium carbonate, potassium hydrogen carbonate, sodium hydrogen carbonate, potassium dihydrogen phosphate, dipotassium hydrogen phosphate, potassium phosphate, sodium dihydrogen phosphate, disodium hydrogen phosphate, sodium phosphate, potassium hydroxide, sodium hydroxide, and sodium hydride; and a metal alkoxide such as sodium methoxide and potassium tert-butoxide.

Specific examples of the phase-transfer catalyst include tetrabutylammonium hydrogen sulfate.

Specific examples of the inert solvent include a halogenated hydrocarbon such as chloroform and dichloromethane; an aromatic hydrocarbon such as benzene and toluene; an ether-type solvent such as diethyl ether, tetrahydrofuran (THF), and 1,4-dioxane; a lower alcohol such as methanol, ethanol, and 2-propanol; an aprotic polar solvent such as acetonitrile, acetone, methyl ethyl ketone, dimethylformamide, N-methyl-2-pyrrolidinone, and dimethylsulfoxide; and a mixture thereof.

Compound (11) is prepared by the Mitsunobu reaction of compound (13) with compound (15) in an appropriate inert solvent according to a conventional method. Specifically, the reaction can be carried out in the co-presence of triphenylphosphine and a Mitsunobu reagent such as diethyl azodicarboxylate and diisopropyl azodicarboxylate or using a cyanomethylenephosphorane reagent. The reaction temperature is typically a temperature of about −20° C. to the boiling point of the used solvent. The reaction time is typically in the range from 10 minutes to 48 hours, which may vary according to various conditions such as a reaction temperature, a base, a starting material, and a solvent to be used.

Specific examples of the inert solvent include an aromatic hydrocarbon such as benzene and toluene; an ether-type solvent such as diethyl ether, tetrahydrofuran (THF), and 1,4-dioxane; and a mixture thereof.

Preparation 8

The compound of formula (13) is prepared according to, for example, the following process:

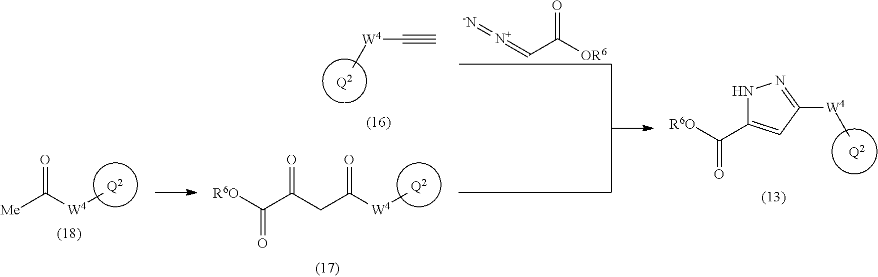

wherein W⁴ and ring Q² are as defined in the above term [1], and R⁶ is optionally-substituted C₁₋₄ alkyl group.

Compound (13) is prepared by reacting compound (16) with diazoacetate such as ethyl diazoacetate in an appropriate inert solvent. For example, compound (13) is prepared by reacting compound (16) with a base such as n-butyllithium, and then diazoacetate in all inert solvent such as tetrahydrofuran and toluene. Also, the reaction may be carried out in the presence of zinc trifluoromethanesulfonate and a base such as triethylamine as an additive, as appropriate. The reaction temperature is typically a temperature of about −20° C. to the boiling point of the used solvent. The reaction time is typically in the range from 10 minutes to 48 hours, which may vary according to various conditions such as a reaction temperature, a base, a starting material, and a solvent to be used.

Specific examples of the inert solvent include a halogenated hydrocarbon such as chloroform and dichloromethane; an aromatic hydrocarbon such as benzene and toluene; an ether-type solvent such as diethyl ether, tetrahydrofuran (THF), and 1,4-dioxane; an aprotic polar solvent such as dimethylformamide and N-methyl-2-pyrrolidinone; and a mixture thereof.

Compound (13) is also prepared by reacting compound (17) with hydrazine. The reaction temperature is typically a temperature of about −20° C. to the boiling point of the used solvent. The reaction time is typically in the range from 10 minutes to 48 hours, which may vary according to various conditions such as a reaction temperature, a starting material, and a solvent to be used.

Specific examples of the solvent include an aromatic hydrocarbon such as benzene and toluene; an ether-type solvent such as diethyl ether, tetrahydrofuran (THF), and 1,4-dioxane; a lower alcohol such as methanol, ethanol, and 2-propanol; an aprotic polar solvent such as dimethylformamide and N-methyl-2-pyrrolidinone; and a mixture thereof.

Compound 17 is prepared by reacting compound (18) with oxalate such as diethyl oxalate in the presence of a base. The reaction temperature is typically a temperature of about −20° C. to the boiling point of the used solvent. The reaction time is typically in the range from 10 minutes to 48 hours, which may vary according to various conditions such as a reaction temperature, a base, a starting material, and a solvent to be used. Specific examples of the base include sodium, sodium ethoxide, lithium hexamethylenedisilazane, sodium hydride, potassium tert-butoxide, and lithium diisopropylamide.

Specific examples of the solvent include an aromatic hydrocarbon such as benzene and toluene; an ether-type solvent such as diethyl ether, tetrahydrofuran (THF), and 1,4-dioxane; a lower alcohol such as methanol, ethanol, and 2-propanol; an aprotic polar solvent such as dimethylformamide and N-methyl-2-pyrrolidinone; and a mixture thereof.

Preparation 9

Among the compound of formula (2), the compound of formula (2c) is prepared according to, for example, the following process:

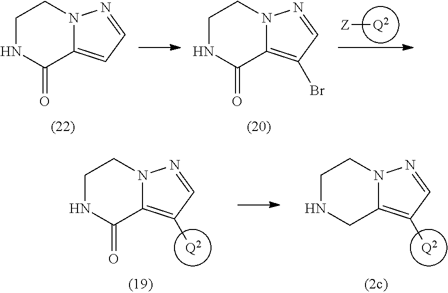

wherein ring Q² is as defined in the above term [1], and Z is boronic acid group (—B(OH)₂), boronate group (such as pinacol boronate group), organotin group (such as —Sn(n-Bu)₄), zinc halide (such as ZnCl and ZnBr), or magnesium halide (such as MgCl and MgBr).

Compound (2c) is also prepared by reacting compound (19) with a reducing agent. The reaction temperature is typically a temperature of about −20° C. to the boiling point of the used solvent. The reaction time is typically in the range from 10 minutes to 48 hours, which may vary according to various conditions such as a reaction temperature, a condensing agent, a starting material, and a solvent to be used.

Specific examples of the reducing agent include lithium aluminum hydride and a borane complex (e.g. borane-dimethylsulfide complex or borane-tetrahydrofuran complex).

Specific examples of the inert solvent include an ether-type solvent such as tetrahydrofuran and 1,4-dioxane, and a mixture thereof.

Compound (19) is prepared by the coupling reaction of compound (20) with compound (21) in an appropriate inert solvent in the presence of a transition metal catalyst. The reaction may be carried out in the presence of a ligand, a base, an additive, etc., as appropriate. The reaction temperature is typically a temperature of −10° C. to the boiling point of the used solvent. The reaction time is typically in the range from 10 minutes to 48 hours, which may vary according to various conditions such as a reaction temperature, a transition metal catalyst, a starting material, and a solvent to be used.

Specific examples of the transition metal catalyst include palladium (II) acetate, palladium (II) chloride, tris(dibenzylideneacetone)dipalladium (0), tetrakis(triphenylphosphine)palladium (0), bis(triphenylphosphine)palladium (II) chloride, dichlorobis(tri-O-tolylphosphine)palladium (II), bis(tri-tert-butylphosphine)palladium (0), and [1,1′-bis(diphenylphosphino)ferrocene]dichloropalladium (II).

Specific examples of the ligand include triphenylphosphine, tri-O-tolylphosphine, tri-tert-butylphosphine, tri-2-furylphosphine, tricyclohexylphosphine, triphenylarsine, 1,1′-bis(diphenylphosphino)ferrocene, 2-dicyclohexylphosphino-2′,6′-dimethoxybiphenyl, and 2-dicyclohexylphosphino-2′,4′,6′-triisopropylbiphenyl.

Specific examples of the base include an organic base such as triethylamine and diisopropylethylamine; and an inorganic base such as sodium carbonate, sodium hydrogen carbonate, potassium carbonate, cesium carbonate, and potassium phosphate.

Specific examples of the additive include an inorganic salt such as lithium chloride, cesium fluoride, copper (I) iodide, and copper (I) bromide.

Specific examples of the inert solvent include water; acetonitrile; a halogenated hydrocarbon such as chloroform and dichloromethane; an aromatic hydrocarbon such as benzene and toluene; an ether-type solvent such as 1,2-dimethoxyethane, tetrahydrofuran, and 1,4-dioxane; a lower alcohol such as methanol, ethanol, and 2-propanol; an aprotic polar solvent such as dimethylformamide and N-methyl-2-pyrrolidinone; and a mixture thereof.

Compound (20) is prepared by reacting compound (22) with a brominating agent such as N-bromosuccinimide in an appropriate inert solvent. The reaction temperature is typically a temperature of −20° C. to the boiling point of the used solvent. The reaction time is typically in the range from 10 minutes to 48 hours, which may vary according to various conditions such as a reaction temperature, a brominating agent, a starting material, and a solvent to be used.

Specific examples of the inert solvent include a halogenated hydrocarbon such as chloroform and dichloromethane; an ether-type solvent such as diethyl ether, tetrahydrofuran, 1,4-dioxane, and 1,2-dimethoxyethane; an aprotic polar solvent such as dimethylformamide and N-methyl-2-pyrrolidinone; and a mixture thereof.

The intermediates and desired compounds in the above preparation processes may be isolated and purified by a conventional purification method in organic synthetic chemistry such as neutralization, filtration, extraction, washing, drying, concentration, recrystallization, and each type of chromatography. The intermediates may be also used in the next reaction without any specific purification.

An optically-active product of the present compound can be prepared from an optically-active starting material or intermediate, or by the optical resolution of the racemate of a final product. The optical resolution method includes a physical separation method with optically-active column, and a chemical separation method such as a fractional crystallization method. A diastereomer of the present compound can be prepared by, for example, a fractional crystallization method.

The pharmaceutically acceptable salt of the compound of formula (1) can be prepared by, for example, mixing the compound of formula (1) with a pharmaceutically acceptable acid in a solvent such as water, methanol, ethanol, and acetone.

The present compound is a dopamine D₄ receptor agonist, and thus can be used for treating a central nervous system disease with a symptom similar to ADHD, for example, autistic spectrum disorder (autistic spectrum disorder defined in Diagnostic and Statistical Manual of Mental Disorders, 5th Edition (DSM-V), which is classified as autism, Asperger's syndrome, atypical pervasive developmental disorder, and childhood disintegrative disorder in previous DSM-IV) as well as schizophrenia, mood disorder, and cognitive dysfunction with an ADHD-like symptom. The present compound may be used in combination with a central nervous system stimulant such as methylphenidate, a selective noradrenaline reuptake inhibitor such as atomoxetine, and each type of medicament for treating schizophrenia.

One of hypotheses implicated in the pathogenesis of autistic spectrum disorder is assumed to be a lack of the conformity in the network of nerves caused by the imbalance between excitatory and inhibitory neurotransmitters in the cerebral cortex. Then, it has been demonstrated that the imbalance can be improved by amplifying γ wave which is a brain wave in high-frequency zone. Furthermore, it has been already reported that a dopamine D₄ receptor agonist can amplify γ wave in the cerebral cortex.

Also, it has been reported that oxytocin which is a hormone produced in the hypothalamus is involved in social cognition. That is, the reports suggest that oxytocin is associated with autism. The dopamine D₄ receptor is highly expressed in oxytocin-producing neurons which are expressed in hypothalamic paraventricular nuclei, and thus a dopamine D₄ receptor agonist is expected to enhance the release of oxytocin in the brain with the activation of oxytocin-producing neurons.

Accordingly, the dopamine D₄ receptor agonist can be used as a medicament for treating autistic spectrum disorder by the amplification of γ wave in the cerebral cortex, or the enhancement of the release of oxytocin in hypothalamus.

The present compound is preferably used for the treatment of ADHD and autistic spectrum disorder.

The present compound is preferably used for the treatment of AHD, in particular, ADHD with inattention, hyperactivity, and impulsivity as a predominant symptom.

The present compound is preferably used for the treatment of autistic spectrum disorder, in particular, autistic spectrum disorder with a persistent deficit in social communication and social interaction, and restricted repetitive behaviors, interests, or activities as a predominant symptom.

When a medicinal compound is taken into the body, the compound is metabolized to change its chemical structure, and is converted to a reactive intermediate, i.e. a reactive metabolite. The reactive metabolite might result in any toxicity (e.g. hepatotoxicity, allergy, tissue necrosis, mutagenicity, and carcinogenicity). As one of simple tests for evaluating toxic risks from the reactive metabolite, the glutathione (GSH) trapping test with dansyl glutathione (dGSH) may be used. In the test, when compounds with a high level of covalent binding to dGSH are exposed systemically, the above toxic risks are increased.

On the other hand, the present compound has an extremely low level of covalent binding to dGSH as shown in Test Example 4, and thus is at low risk for hepatotoxicity, etc. As a result, the present compound is expected to be administered safely over a long period.

The present compound may be administered orally or parenterally. The present compound may be orally-administered in the commonly-used dosage forms. The present compound may be parenterally-administered in the forms of a topical preparation, an injectable preparation, a transdermal preparation, and a transnasal preparation. Examples of the preparation for oral or rectal administration include a capsule, a tablet, a pill, a powder, a cachet, a suppository, and a solution. Examples of the injectable preparation include a sterile solution and a suspension. Examples of the topical preparation include a cream, an ointment, a lotion, and a transdermal preparation (e.g. a conventional patch and a matrix).

The above dosage form can be formulated using pharmaceutically acceptable excipient and additive in a conventional manner. Examples of the pharmaceutically acceptable excipient and additive include a carrier, a binder, a perfume, a buffer, a thickener, a colorant a stabilizer, an emulsifier, a dispersant, a suspending agent, and a preservative agent.

Examples of the pharmaceutically acceptable carrier include magnesium carbonate, magnesium stearate, talc, sucrose, lactose, pectin, dextrin, starch, gelatin, tragacanth, methylcellulose, sodium carboxymethyl cellulose, low-melting-point wax, and cacao butter. The capsule may be formulated by filling a capsule with the present compound together with a pharmaceutically acceptable carrier. The present compound may be mixed with a pharmaceutically acceptable excipient or without any excipient to be put into a capsule. The cachet may be also formulated in a similar manner thereto.

Examples of the injectable solution include a solution, a suspension, and an emulsion. For example, examples thereof include an aqueous solution and a water-propylene glycol solution. The solution may contain water, and may be also prepared in the form of a polyethylene glycol solution and/or a propylene glycol solution. The solution suitable for oral administration can be prepared by adding the present compound to water together with a colorant, a perfume, a stabilizing agent, a sweetening agent, a solubilizer, and a thickener, as appropriate. The solution suitable for oral administration can be also prepared by adding the present compound to water together with a dispersant to thicken the solution. Examples of the thickener include pharmaceutically acceptable natural or synthetic gum, resin, methylcellulose, sodium carboxymethyl cellulose, and known suspending agents.

The dosage of the present compound may vary according to various conditions such as patient's disease, age, body weight, sex, symptom, and administration route. Typically, the present compound is administered to an adult (body weight: 50 kg) at a dose of 0.1 to 1000 mg/day, preferably at a dose of 0.1 to 300 mg/day, which may be administered once a day or 2 or 3 times a day. In addition, the present compound may be administered once in several days to several weeks.

### EXAMPLES

Hereinafter, the invention is illustrated in more detail with Reference Examples, Examples, and Test Examples, but the invention should not be limited thereto. The compound names as shown in the following Reference Examples and Examples do not necessarily follow the IUPAC nomenclature system. Also, some abbreviations may be used herein for the sake of simplicity, and the abbreviations are as defined above.

The compound identification was performed with any methods such as proton nuclear magnetic resonance absorption spectrum (¹H-NMR) and LC-MS. Amino chromatography in Reference Examples and Examples was performed with the amino column manufactured by Yamazen Corporation. LC-MS measurement was performed under various conditions as shown in Table 1 below. Retention Time (R.T.) means the time when the mass spectral peak of a sample is detected in the LC-MS measurement.

TABLE 1

| | Condition A | Condition B |
|---|---|---|
| analyser | Waters ACQUITYTM | Shimadzu LCMS-2020 |
| | UltraPerformance LC | |
| column | Waters ACQUITY | Phenomenex Kinetex |
| | UPLC BEH | 1.7 μm |
| | Phenyl 1.7 μm, | C18 (50 mm × 2.10 mm) |
| | 2.1 × 50 mm | |
| solvent | A solution: 0.05% | A solution: 0.05% |
| | formic acid/H₂O | TFA/H₂O |
| | B solution: 0.05% | B solution: CH₃CN |
| | formic acid/CH₃CN | |
| gradient | 0.0 min: | 0.0 min: |
| condition | A/B = 90:10 | A/B = 99:1 |
| | 0.0-1.3 min: | 0.0-1.90 min: |
| | A/B = 90:10-1:99 | A/B = 99:1-1:99 |
| | (linear gradient) | (linear gradient) |
| | 1.3-1.5 min: | 1.91-3.00 min: |
| | A/B = 1:99 | A/B = 1:99 |
| | 1.5-2.0 min: | |
| | A/B = 90:10 | |
| flow rate | 0.75 mL/min | 0.5 mL/min |
| UV | 220 nm, 254 nm | 220 nm, 254 nm |
| column | 40° C. | 40° C. |
| temperature | | |

The following abbreviations may be used herein.

The following abbreviations are used in NMR data in Reference Examples and Examples.

Me group: methyl group

Et group: ethyl group

Boc group: tert-butoxycarbonyl group

tert-: tertiary-

s: singlet

brs: broad singlet

d: doublet

t: triplet

m: multiplet

br: broad

J: coupling constant

Hz: Hertz

CDCl₃: deuterochloroform

DMSO-d₆: deuterodimethylsulfoxide

### Example 1

### 5-Benzyl-2-(pyridin-2-yl)-4,5,6,7-tetrahydropyrazolo[1,5-a]pyrazine

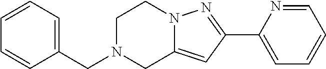 [SMILES: c1ccc(CN2CCn3nc(-c4ccccn4)cc3C2)cc1]

To a solution of the compound of Reference Example 1 (40 mg, 0.20 mmol) in dichloromethane (2 mL) were added benzaldehyde (20 μL, 0.20 mmol) and sodium triacetoxyborohydride (64 mg, 0.30 mmol). The mixture was stirred at room temperature for 2 hours, and saturated aqueous sodium hydrogen carbonate solution was added thereto, and then the mixture was extracted with ethyl acetate. The organic layer was washed with brine, dried over anhydrous sodium sulfate, filtered, and concentrated. The resulting residue was purified by amino silica gel column chromatography (n-hexane:ethyl acetate=1:1) to give the title compound (49 mg, 84%).

¹H-NMR (400 MHz, CDCl₃) δ: 2.97 (2H, t, J=5.4 Hz), 3.72 (2H, s), 3.73 (2H, s), 4.24 (2H, t, J=5.4 Hz), 6.61 (1H, s), 7.15-7.19 (1H, m), 7.28-7.40 (5H, m), 7.67-7.71 (1H, m), 7.88 (1H, d, J=8.3 Hz), 8.59 (1H, d, J=4.9 Hz).

### Examples 2-11

The compounds of Examples 2-11 were synthesized from the corresponding compounds of each Reference Example according to the process of Example 1.

| Example | Chemical Structure | Instrumental Analysis Data |
|---|---|---|
| 2 | 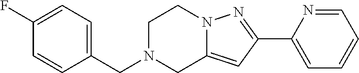 [SMILES: Fc1ccc(CN2CCn3nc(-c4ccccn4)cc3C2)cc1] | ¹H-NMR (400 MHz, CDCl₃) δ: 2.94 (2H, t, J = 5.6 Hz), 3.67 (2H, s), 3.70 (2H, s), 4.23 (2H, t, J = 5.6 Hz), |
| | | 6.58 (1H, s), 7.01 |
| | | (2H, t, J = 7.8 Hz), |
| | | 7.12-7.18 (1H, m), |
| | | 7.24-7.34 (2H, m), |
| | | 7.62-7.70 (1H, m), |
| | | 7.86 (1H, d, J = 7.8 |
| | | Hz), 8.56-8.60 (1H, m). |
| 3 | 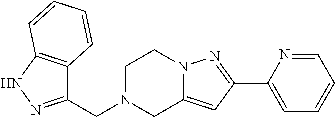 [SMILES: c1ccc(-c2cc3n(n2)CCN(Cc2n[nH]c4ccccc24)C3)nc1] | ¹H-NMR (400 MHz, CDCl₃) δ: 3.06 (2H, t, J = 5.4 Hz), 3.85 (2H, s), 4.17 (2H, s), 4.27 (2H, t, J = 5.4 Hz), 6.61 (1H, s), 7.13- 7.20 (2H, m), 7.36 (1H, t, J = 7.6 Hz), |
| | | 7.44 (1H, d, J = 8.3 |
| | | Hz), 7.67-7.72 (1H, |
| | | m), 7.89 (2H, t, J = |
| | | 8.3 Hz), 8.62 (1H, d, |
| | | J = 4.9 Hz). |
| 4 | 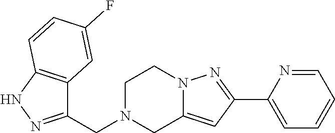 [SMILES: Fc1ccc2[nH]nc(CN3CCn4nc(-c5ccccn5)cc4C3)c2c1] | ¹H-NMR (400 MHz, CDCl₃) δ: 3.05 (2H, t, J = 5.6 Hz), 3.80 (2H, s), 4.12 (2H, s), 4.27 (2H, t, J = 5.6 Hz), 6.60 (1H, s), 7.11 (1H, dt, J = 9.0, 2.4 Hz), 7.17-7.21 (1H, m), 7.34 (1H, dt, J = |
| | | 9.0, 4.1 Hz), 7.48 |
| | | (1H, dd, J = 9.0, 2.4 |
| | | Hz), 7.71 (1H, dt, J = |
| | | 7.8, 1.6 Hz), 7.92 |
| | | (1H, d, J = 7.8 Hz), |
| | | 8.62 (1H, d, J = 4.1 Hz). |
| 5 | 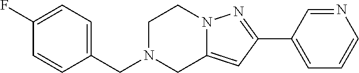 [SMILES: Fc1ccc(CN2CCn3nc(-c4cccnc4)cc3C2)cc1] | ¹H-NMR (400 MHz, CDCl₃) δ: 2.95 (2H, t, J = 5.5 Hz), 3.68 (4H, s), 4.21 (2H, t, J = 5.5 Hz), 6.27 (1H, s), |
| | | 7.02 (2H, t, J = 8.7 |
| | | Hz), 7.26-7.34 (3H, |
| | | m), 8.01-8.05 (1H, |
| | | m), 8.48-8.51 (1H, |
| | | m), 8.95 (1H, s). |
| 6 | 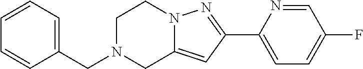 [SMILES: Fc1ccc(-c2cc3n(n2)CCN(Cc2ccccc2)C3)nc1] | ¹H-NMR (400 MHz, CDCl₃) δ: 2.98 (2H, t, J = 5.6 Hz), 3.73 (4H, s), 4.23 (2H, t, J = 5.6 Hz), 6.50 (1H, s), |
| | | 7.25-7.41 (6H, m), |
| | | 7.87 (1H, dd, J = 8.8, |
| | | 4.4 Hz), 8.43 (1H, d, |
| | | J = 2.9 Hz). |
| 7 | 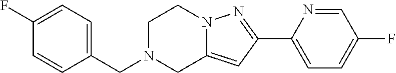 [SMILES: Fc1ccc(CN2CCn3nc(-c4ccc(F)cn4)cc3C2)cc1] | ¹H-NMR (400 MHz, CDCl₃) δ: 2.95 (2H, t, J = 5.6 Hz), 3.68 (2H, s), 3.70 (2H, s), 4.22 (2H, t, J = 5.6 Hz), |
| | | 6.50 (1H, s), 7.00- |
| | | 7.04 (2H, m), 7.30- |
| | | 7.34 (2H, m), 7.38 |
| | | (1H, dt, J = 8.8, 2.9 |
| | | Hz), 7.86 (1H, dd, J = |
| | | 8.8, 4.6 Hz), 8.42 |
| | | (1H, d, J = 2.9 Hz). |
| 8 | 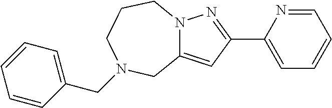 [SMILES: c1ccc(CN2CCCn3nc(-c4ccccn4)cc3C2)cc1] | ¹H-NMR (400 MHz, CDCl₃) δ: 1.82-1.89 (2H, m), 3.09 (2H, t, J = 5.4 Hz), 3.52 (2H, s), 3.79 (2H, s), 4.37- 4.40 (2H, m), 6.57 (1H, s), 7.08-7.12 |
| | | (1H, m), 7.17-7.29 |
| | | (5H, m), 7.62 (1H, dt, |
| | | J = 7.8, 1.5 Hz), 7.80 |
| | | (1H, d, J = 7.8 Hz), |
| | | 8.54 (1H, d, J = 4.9 Hz). |
| 9 | 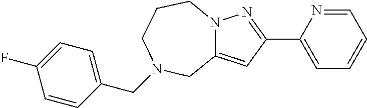 [SMILES: Fc1ccc(CN2CCCn3nc(-c4ccccn4)cc3C2)cc1] | ¹H-NMR (400 MHz, CDCl₃) δ: 2.11-2.17 (2H, m), 3.39 (2H, t, J = 5.1 Hz), 3.78 (2H, s), 4.07 (2H, s), 4.68- 4.70 (2H, m), 6.86 (1H, s), 7.21-7.27 |
| | | (2H, m), 7.38-7.43 |
| | | (1H, m), 7.47-7.53 |
| | | (2H, m), 7.93 (1H, dt, |
| | | J = 7.3, 2.0 Hz), 8.09- |
| | | 8.11 (1H, m), 8.84- |
| | | 8.85 (1H, m). |
| 10 | 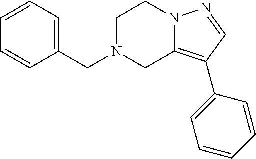 [SMILES: c1ccc(CN2CCn3ncc(-c4ccccc4)c3C2)cc1] | ¹H-NMR (400 MHz, CDCl₃) δ: 2.96 (2H, t, J = 5.6 Hz), 3.77 (2H, s), 3.90 (2H, s), 4.23 (2H, t, J = 5.6 Hz), 7.19-7.40 (10H, m), 7.69 (1H, s). |
| 11 | 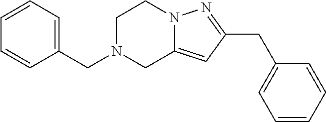 [SMILES: c1ccc(Cc2cc3n(n2)CCN(Cc2ccccc2)C3)cc1] | ¹H-NMR (400 MHz, CDCl₃) δ: 2.91 (2H, t, J = 5.6 Hz), 3.59 (2H, s), 3.68 (2H, s), 3.93 (2H, s), 4.14 (2H, t, J = 5.6 Hz), 5.67 (1H, s), 7.13-7.39 (10H, m). |

### Example 12

### 5-(2,3-Dihydro-1H-inden-2-ylmethyl)-2-(pyridin-2-yl)-4,5,6,7-tetrahydropyrazolo[1,5-a]pyrazine

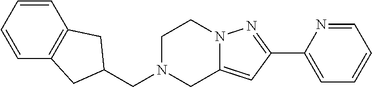 [SMILES: c1ccc(-c2cc3n(n2)CCN(CC2Cc4ccccc4C2)C3)nc1]

To a solution of the compound of Reference Example 1 (57.8 mg, 0.289 mmol) in dichloromethane (5.0 mL) were added 2,3-dihydro-1H-indene-2-carbaldehyde (44.0 mg, 0.301 mmol), acetic acid (0.10 mL), and then sodium triacetoxyborohydride (92.0 mg, 0.434 mmol). The mixture was stirred at room temperature for 24 hours, and ice-cooled. To the ice-cooled reaction mixture was then added saturated aqueous sodium hydrogen carbonate solution, and the mixture was extracted with chloroform. The organic layer was dried over anhydrous sodium sulfate, filtered, and concentrated in vacuo. The resulting concentrated residue was purified by silica gel chromatography (chloroform:methanol=9:1) to give the title compound (36 mg, 38%).

¹H-NMR (400 MHz, CDCl₃) δ: 2.62 (2H, d, J=7.3 Hz), 2.79 (3H, ddd, J=17.0, 10.1, 4.4 Hz), 3.01 (2H, t, J=5.7 Hz), 3.14-3.07 (2H, m), 3.78 (2H, s), 4.29 (2H, t, J=5.5 Hz), 6.66 (1H, s), 7.24-7.14 (5H, m), 7.74-7.71 (1H, m), 7.93-7.91 (1H, m), 8.63-8.62 (1H, m).

### Examples 13-21

The compounds of Examples 13-21 were synthesized from the corresponding compounds of each Reference Example according to the process of Example 12.

| Example | Chemical Structure | Instrumental Analysis Data |
|---|---|---|
| 13 | 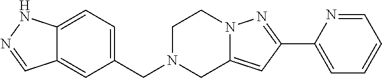 [SMILES: c1ccc(-c2cc3n(n2)CCN(Cc2ccc4[nH]ncc4c2)C3)nc1] | ¹H-NMR (300 MHz, CDCl₃) δ: 3.04 (2H, s, J = 5.5 Hz), 3.87 (2H, s), 3.79 (2H, s), 4.28 (2H, t, J = |
| | | 5.5 Hz), 6.81-6.68 |
| | | (1H, m), 7.24 (1H, |
| | | s), 7.52-7.47 (2H, |
| | | m), 7.81-7.72 (2H, |
| | | m), 7.98-7.93 (1H, |
| | | m), 8.07 (1H, s), |
| | | 8.64-8.61 (1H, m). |
| 14 | 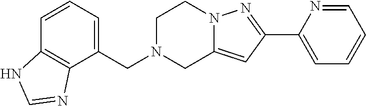 [SMILES: c1ccc(-c2cc3n(n2)CCN(Cc2cccc4[nH]cnc24)C3)nc1] | ¹H-NMR (300 MHz, CDCl₃) δ: 2.94-2.91 (2H, m), 3.69 (2H, brs), 4.02 (2H, s), 4.12-4.19 (2H, m), 6.51 (1H, s), 7.19- 7.13 (2H, m), 7.22 |
| | | (1H, dd, J = 8.0, 7.3 |
| | | Hz), 7.71-7.65 (2H, |
| | | m), 7.86-7.84 (1H, |
| | | m), 8.00 (1H, s), |
| | | 8.58-8.56 (1H, m). |
| 15 | 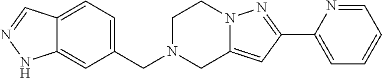 [SMILES: c1ccc(-c2cc3n(n2)CCN(Cc2ccc4cn[nH]c4c2)C3)nc1] | ¹H-NMR (400 MHz, CDCl₃) δ: 3.05 (2H, t, J = 5.6 Hz), 3.80 (2H, s), 3.90 (2H, s), 4.29 (2H, t, J = 5.6 Hz), 6.79 (1H, |
| | | brs), 7.22 (1H, dd, |
| | | J = 8.3, 1.2 Hz), 7.26- |
| | | 7.25 (1H, m), 7.55 |
| | | (1H, brs), 7.74 (1H, |
| | | dd, J = 8.3 Hz, 0.7 |
| | | Hz), 7.82-7.77 (1H, |
| | | m), 7.98-7.97 (1H, |
| | | m), 8.07 (1H, d, J = |
| | | 1.0 Hz), 8.64-8.62 |
| | | (1H, m). |
| 16 | 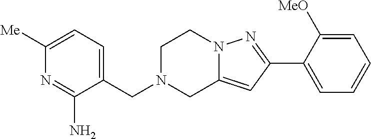 [SMILES: COc1ccccc1-c1cc2n(n1)CCN(Cc1ccc(C)nc1N)C2] | ¹H-NMR (300 MHz, CDCl₃) δ: 2.54 (3H, s), 2.97 (2H, t, J = 5.6 Hz), 3.68 (2H, s), 3.73 (2H, s), 3.89 (3H, s), 4.26 (2H, t, J = 5.6 Hz), 6.04 (2H, brs), 6.52 (1H, s), 6.95-7.04 |
| | | (2H, m), 7.30 (1H, |
| | | ddd, J = 8.3, 7.5, |
| | | 1.7 Hz), 7.85 (1H, |
| | | dd, J = 7.5, 1.7 Hz), |
| | | 8.02 (1H, s). |
| 17 | 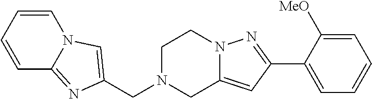 [SMILES: COc1ccccc1-c1cc2n(n1)CCN(Cc1cn3ccccc3n1)C2] | ¹H-NMR (300 MHz, CDCl₃) δ: 3.14 (2H, t, J = 5.5 Hz), 3.87 (3H, s), 3.89 (2H, s), 4.01 (2H, s), 4.30 (2H, t, J = 5.6 Hz), 6.49 (1H, s), |
| | | 6.85 (1H, ddd, J = |
| | | 6.8, 6.8, 1.1 Hz), |
| | | 6.93-7.03 (2H, m), |
| | | 7.21-7.31 (2H, m), |
| | | 7.62 (1H, brs), 7.65 |
| | | (1H, brd, 7 = 9.2 |
| | | Hz), 7.87 (1H, dd, J = |
| | | 7.7, 1.8 Hz), 8.12 |
| | | (1H, brd, J = 6.8 Hz). |
| 18 | 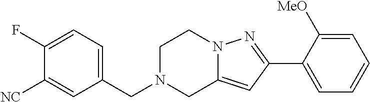 [SMILES: COc1ccccc1-c1cc2n(n1)CCN(Cc1ccc(F)c(C#N)c1)C2] | ¹H-NMR (300 MHz, CDCl₃) δ: 3.01 (2H, t, J = 5.6 Hz), 3.73 (2H, s), 3.75 (2H, s), 3.89 (3H, s), 4.28 (2H, t, J = 5.6 Hz), 6.50 (1H, s), |
| | | 6.95-7.04 (2H, m), |
| | | 7.19-7.33 (2H, m), |
| | | 7.64-7.70 (2H, m), |
| | | 7.87 (1H, dd, J = |
| | | 7.7, 1.8 Hz). |
| 19 | 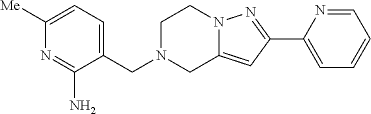 [SMILES: Cc1ccc(CN2CCn3nc(-c4ccccn4)cc3C2)c(N)n1] | ¹H-NMR (400 MHz, CDCl₃) δ: 2.40 (3H, s), 2.96 (2H, t, J = 5.6 Hz), 3.67 (2H, s), 3.73 (2H, s), 4.26 (2H, t, J = 5.6 Hz), 5.48 (2H, brs), 6.50 (1H, d, J = 7.2 |
| | | Hz), 6.60 (1H, s), |
| | | 7.18 (1H, ddd, J = |
| | | 7.7, 4.9, 1.2 Hz), |
| | | 7.21 (1H, d, J = 7.2 |
| | | Hz), 7.69 (1H, ddd, J = |
| | | 7.8, 7.7, 1.8 Hz), |
| | | 7.88 (1H, brd, J = |
| | | 7.8), 8.61 (1H, ddd, |
| | | J = 4.9, 1.8, 1.0 Hz). |
| 20 | 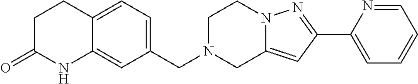 [SMILES: O=C1CCc2ccc(CN3CCn4nc(-c5ccccn5)cc4C3)cc2N1] | ¹H-NMR (400 MHz, CDCl₃) δ: 2.65 (2H, t, J = 7.4 Hz), 2.97 (2H, t, J = 7.4 Hz), 2.99 (2H, t, J = 5.7 Hz), 3.69 (2H, s), |
| | | 3.74 (2H, s), 4.26 |
| | | (2H, t, J = 5.7 Hz), |
| | | 6.65 (1H, brs), 6.79 |
| | | (1H, brs), 6.98- |
| | | 7.00 (1H, m), 7.15 |
| | | (1H, d, J = 7.6 Hz), |
| | | 7.19-7.23 (1H, m), |
| | | 7.63 (1H, brs), 7.70- |
| | | 7.76 (1H, m), 7.90- |
| | | 7.92 (1H, m), 8.61 |
| | | (1H, ddd, J = 4.9, |
| | | 1.0, 0.7 Hz). |
| 21 | 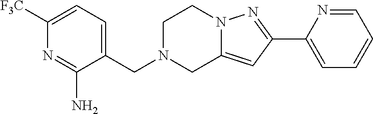 [SMILES: Nc1nc(C(F)(F)F)ccc1CN1CCn2nc(-c3ccccn3)cc2C1] | ¹H-NMR (400 MHz, CDCl₃) δ: 3.01 (2H, t, J = 5.6 Hz), 3.81- 3.71 (4H, m), 4.31 (2H, t, J = 5.6 Hz), 6.63 (1H, s), 7.68- 7.70 (2H, m), 7.84- 7.87 (2H, m), 8.59 |
| | | (1H, s), 8.60-8.61 |
| | | (2H, br). |

### Example 22

### 2-Methyl-5-{[2-(pyridin-2-yl)-6,7-dihydropyrazolo[1,5-a]pyrazin-5(4H)-yl]methyl}pyrimidine-4-amine

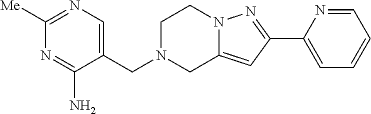 [SMILES: Cc1ncc(CN2CCn3nc(-c4ccccn4)cc3C2)c(N)n1]

To a solution of the compound of Reference Example 1 (50 mg, 0.25 mmol) in acetonitrile (3 mL) were added 5-(chloromethyl)-2-methylpyrimidine-4-amine (49 mg, 0.25 mmol), potassium iodide (42 mg, 0.25 mmol), and potassium carbonate (104 mg, 0.275 mmol). The mixture was stirred for 2 hours with heating under reflux, and saturated aqueous sodium hydrogen carbonate solution was added thereto, and then the mixture was extracted with ethyl acetate. The organic layer was washed with brine, dried over anhydrous sodium sulfate, filtered, and concentrated. The resulting residue was purified by amino silica gel column chromatography (n-hexane:ethyl acetate=1:2) to give the title compound (48 mg, 60%).

¹H-NMR (400 MHz, CDCl₃) δ: 2.52 (3H, s), 2.97 (2H, t, J=5.6 Hz), 3.67 (2H, s), 3.75 (2H, s), 4.27 (2H, t, J=5.6 Hz), 6.62 (1H, s), 7.18-7.21 (1H, m), 7.70 (1H, dt, J=7.7, 1.8 Hz), 7.88 (1H, d, J=7.7 Hz), 8.01 (1H, s), 8.61-8.62 (1H, m).

### Example 23

### 5-[(2-Methyl-2,3-dihydro-1H-isoindol-5-yl)methyl]-2-(pyridin-2-yl)-4,5,6,7-tetrahydropyrazolo[1,5-a]pyrazine

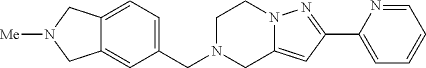 [SMILES: CN1Cc2ccc(CN3CCn4nc(-c5ccccn5)cc4C3)cc2C1]

To a solution of the compound of Reference Example 1 (244 mg, 1.22 mmol) in dichloromethane (5.0 mL) were added tert-butyl 5-formyl-1,3-dihydro-2H-isoindole-2-carboxylate (315 mg, 1.27 mmol) which can be synthesized according to the process of Bioorganic Medicinal Chemistry 17 (2009) 7850-7860, acetic acid (0.10 mL), and then sodium triacetoxyborohydride (388 mg, 1.83 mmol). The mixture was stirred at room temperature for 24 hours, and ice-cooled. To the ice-cooled reaction mixture was then added saturated aqueous sodium hydrogen carbonate solution, and the mixture was extracted with chloroform. The organic layer was dried over sodium sulfate, filtered, and concentrated in vacuo.

The resulting concentrated residue was purified by silica gel chromatography (chloroform:methanol=9:1). To the purified product (296 mg, 0.686 mmol) was added 4 mol/L hydrochloric acid/1,4-dioxane (5.0 mL), and then the mixture was stirred at room temperature for 20 minutes, and the reaction mixture was concentrated. The resulting concentrated residue was purified by amino column chromatography (chloroform:methanol=9:1).

To a solution of the purified product (202 mg, 0.609 mmol) in methanol (18 mL) were added paraformaldehyde (27.1 mg) and sodium borohydride (35.6 mg, 0.942 mmol). The mixture was stirred at room temperature for 24 hours, and brine was added to the reaction mixture with ice-cooling, and then the mixture was extracted with chloroform, dried over anhydrous sodium sulfate, filtered, and concentrated. The resulting concentrated residue was purified by silica gel column chromatography (chloroform:methanol=9:1) to give the title compound (114 mg, 27%).

¹H-NMR (300 MHz, CDCl₃) δ: 2.60 (3H, s), 2.92 (2H, t, J=5.5 Hz), 3.67 (4H, d, J=2.9 Hz), 3.94 (4H, s), 4.19 (2H, t, J=5.6 Hz), 6.54-6.47 (1H, m), 7.20-7.03 (4H, m), 7.71-7.58 (1H, m), 7.86-7.79 (1H, m), 8.58-8.51 (1H, m).

### Example 24

### 5-(2-Phenylethyl)-2-(pyridin-2-yl)-4,5,6,7-tetrahydropyrazolo[1,5-a]pyrazine

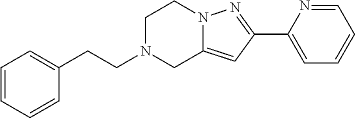 [SMILES: c1ccc(CCN2CCn3nc(-c4ccccn4)cc3C2)cc1]

To a solution of the compound of Reference Example 1 (95.8 mg, 0.478 mmol) in acetonitrile (5 mL) was added potassium carbonate (132 mg, 0.955 mmol) and 2-phenylethyl p-toluenesulfonate (132 mg, 0.478 mmol). The mixture was stirred for 24 hours with heating under reflux, and brine was added to the reaction mixture, and then the mixture was extracted with chloroform. The organic layer was dried over anhydrous sodium sulfate, filtered, and concentrated. The resulting concentrated residue was purified by silica gel chromatography (chloroform:methanol=9:1) to give the title compound (28.0 mg, 19%).

¹H-NMR (400 MHz, CDCl₃) δ: 2.85-2.74 (4H, m), 3.00-2.95 (2H, m), 3.75 (2H, s), 4.21 (2H, t, J=5.5 Hz), 6.55 (1H, s), 7.26-7.09 (6H, m), 7.64-7.60 (1H, m), 7.83-7.81 (1H, m), 8.54-8.54 (1H, m).

### Example 25

### 5-(2,4-Difluorobenzyl)-2-(2-methoxyphenyl)-4,5,6,7-tetrahydropyrazolo[1,5-a]pyrazine

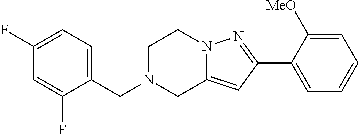 [SMILES: COc1ccccc1-c1cc2n(n1)CCN(Cc1ccc(F)cc1F)C2]

To a solution of the compound of Reference Example 8 (30.0 mg, 0.131 mmol) in N,N-dimethylformamide (1.0 mL) were added potassium carbonate (23.5 mg, 0.170 mmol) and 2,4-difluorobenzyl bromide (18.5 μL, 0.144 mmol). The mixture was stirred at room temperature for 23 hours, and water (6.0 mL) was added to the reaction mixture, and then the mixture was extracted with chloroform (4.0 mL). The organic layer was dried over anhydrous sodium sulfate, filtered, and concentrated. The resulting residue was purified by silica gel column chromatography (n-hexane:ethyl acetate=2:1) to give the title compound (40.2 mg, 86%).

¹H-NMR (300 MHz, CDCl₃) δ: 3.04 (2H, c, J=5.6 Hz), 3.79 (2H, s), 3.81 (2H, s), 3.89 (3H, s), 4.29 (2H, t, J=5.6 Hz), 6.51 (1H, s), 6.81-7.04 (4H, m), 7.25-7.32 (1H, m), 7.43-7.52 (1H, m), 7.87 (1H, dd, J=7.6, 1.7 Hz).

### Examples 26-29

The compounds of Examples 26-29 were synthesized from the corresponding compounds of each Reference Example according to the process of Example 25.

| Example | Chemical Structure | Instrumental Analysis Data |
|---|---|---|
| 26 | 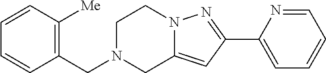 [SMILES: Cc1ccccc1CN1CCn2nc(-c3ccccn3)cc2C1] | ¹H-NMR (300 MHz, CDCl₃) δ: 2.37 (3H, s), 2.95 (2H, t, J = 5.5 Hz), 3.68 (2H, s), 3.73 (2H, s), 4.22 |
| | | (2H, t, J = 5.5 Hz), |
| | | 6.57 (1H, s), 7.11- |
| | | 7.31 (5H, m), 7.67 |
| | | (1H, ddd, J = 7.8, |
| | | 7.8, 1.7 Hz), 7.86 |
| | | (1H, brd, J = 8.1 Hz), |
| | | 8.56-8.60 (1H, m). |
| 27 | 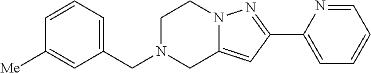 [SMILES: Cc1cccc(CN2CCn3nc(-c4ccccn4)cc3C2)c1] | ¹H-NMR (300 MHz, CDCl₃) δ: 2.37 (3H, s), 2.99 (2H, t, J = 5.6 Hz), 3.71 (2H, s), 3.75 (2H, s), 4.27 |
| | | (2H, t, J = 5.6 Hz), |
| | | 6.60 (1H, s), 7.08- |
| | | 7.31 (5H, m), 7.70 |
| | | (1H, ddd, J = 7.7, |
| | | 7.7, 1.8 Hz), 7.89 |
| | | (1H, d, J = 8.1 Hz), |
| | | 8.59-8.63 (1H, m). |
| 28 | 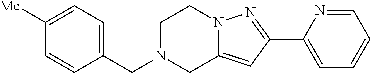 [SMILES: Cc1ccc(CN2CCn3nc(-c4ccccn4)cc3C2)cc1] | ¹H-NMR (300 MHz, CDCl₃) δ: 2.34 (3H, s), 2.95 (2H, t, J = 5.6 Hz), 3.68 (2H, s), 3.71 (2H, s), 4.23 |
| | | (2H, t, J = 5.6 Hz), |
| | | 6.56 (1H, s), 7.11- |
| | | 7.18 (3H, m), 7.20- |
| | | 7.28 (2H, m), 7.67 |
| | | (1H, ddd, J = 7.7, |
| | | 7.7, 1.8 Hz), 7.86 |
| | | (1H, ddd, J = 7.9, |
| | | 1.1, 1.1 Hz), 8.56- |
| | | 8.60 (1H, m). |
| 29 | 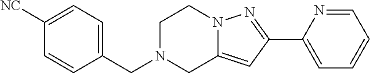 [SMILES: N#Cc1ccc(CN2CCn3nc(-c4ccccn4)cc3C2)cc1] | ¹H-NMR (300 MHz, CDCl₃) δ: 2.97 (2H, t, J = 5.6 Hz), 3.73 (2H, s), 3.78 (2H, s), 4.26 (2H, t, J = 5.6 Hz), |
| | | 6.57 (1H, s), 7.17 |
| | | (1H, ddd, J = 7.5, |
| | | 4.9, 1.2 Hz), 7.50 |
| | | (2H, d, J = 8.3 Hz), |
| | | 7.64 (2H, d, J = 8.3 |
| | | Hz), 7.68 (1H, ddd, J = |
| | | 7.8, 7.8, 1.8 Hz), |
| | | 7.86 (1H, d, J = 7.8 |
| | | Hz), 8.57-8.61 (1H, m). |

### Example 30

### 2-Methyl-5-{[2-(3-methylpyridin-2-yl)-6,7-dihydropyrazolo[1,5-a]pyrazin-5(4H)-yl]methyl}pyrimidine-4-amine

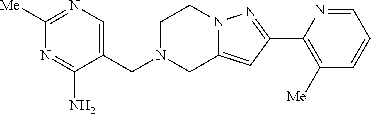 [SMILES: Cc1ncc(CN2CCn3nc(-c4ncccc4C)cc3C2)c(N)n1]

To a solution of the compound of Reference Example 12 (220 mg, 0.772 mmol) in dichloromethane (3.0 mL) were added 4-amino-2-methylpyrimidine-5-carbaldehyde (211 mg, 1.54 mmol), triethylamine (155 mg, 1.54 mmol), and then sodium triacetoxyborohydride (409 mg, 1.93 mmol). The mixture was stirred at room temperature for 16 hours, and then the reaction mixture was concentrated, and purified by preparative HPLC to give the title compound (34.8 mg, 13%).

¹H-NMR (400 MHz, CDCl₃) δ: 2.54 (3H, s), 2.60 (3H, s), 3.01 (2H, t, J=5.6 Hz), 3.70 (2H, s), 3.78 (2H, s), 4.31 (2H, t, J=5.6 Hz), 5.81 (2H, brs), 6.52 (1H, s), 7.15 (1H, dd, J=8.0, 4.2 Hz), 7.57 (1H, d, J=8.0 Hz), 8.04 (1H, s), 8.52 (1H, d, J=4.2 Hz).

### Example 31

### 5-{[2-(5-Fluoropyridin-2-yl)-6,7-dihydropyrazolo[1,5-a]pyrazin-5(4H)-yl]methyl}-2-methylpyrimidine-4-amine

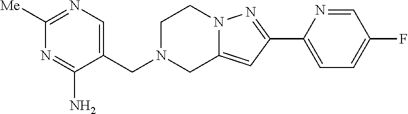 [SMILES: Cc1ncc(CN2CCn3nc(-c4ccc(F)cn4)cc3C2)c(N)n1]

The title compound was prepared from the compound of Reference Example 6 according to a similar process to that of Example 30 (yield: 18%).

¹H-NMR (400 MHz, CD₃OD) δ: 2.44 (3H, s), 3.03 (2H, t, J=5.4 Hz), 3.71 (2H, s), 3.77 (2H, s), 4.26 (2H, t, J=5.4 Hz), 6.62 (1H, s), 7.63-7.70 (1H, m), 7.92-8.02 (2H, m), 8.45 (1H, d, J=2.8 Hz).

### Example 32

### 5-Benzyl-3-methyl-2-(pyridin-2-yl)-5,6,7,8-tetrahydro-4H-pyrazolo[1,5-a][1,4]diazepine

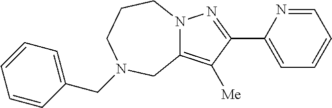 [SMILES: Cc1c(-c2ccccn2)nn2c1CN(Cc1ccccc1)CCC2]

To a solution of the compound of Reference Example 20 (100 mg, 0.379 mmol) in acetonitrile (5 mL) were added potassium carbonate (105 mg, 0.758 mmol) and benzyl bromide (65 mg, 0.379 mmol). The mixture was stirred for 16 hours with heating under reflux, and the reaction mixture was purified by preparative HPLC (with 0.1% aqueous ammonia) to give the title compound (23 mg, 19%).

¹H-NMR (400 MHz, CDCl₃) δ: 1.89-1.99 (2H, m), 2.07 (3H, s), 3.17 (2H, t, J=5.2 Hz), 3.59 (2H, s), 3.78 (2H, s), 4.43 (2H, t, J=5.2 Hz), 7.17 (1H, dd, J=5.2, 5.2 Hz), 7.22-7.37 (5H, m), 7.69 (1H, dd, J=6.3, 6.3 Hz), 7.80 (1H, d, J=8.0 Hz), 8.64 (1H, d, J=4.8 Hz)

### Example 33

### 5-Benzyl-3-fluoro-2-(pyridin-2-yl)-5,6,7,8-tetrahydro-4H-pyrazolo[1,5-a][1,4]diazepine

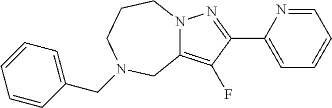 [SMILES: Fc1c(-c2ccccn2)nn2c1CN(Cc1ccccc1)CCC2]

The title compound was prepared from the compound of Reference Example 24 according to a similar process to that of Example 32 (yield: 47%).

¹H-NMR (400 MHz, CDCl₃) δ: 1.87-1.99 (2H, m), 3.17 (2H, t, J=4.8 Hz), 3.63 (2H, s), 3.89 (2H, s), 4.46 (2H, t, J=4.8 Hz), 7.20-7.40 (6H, m), 7.71-7.84 (2H, m), 8.73 (1H, d, J=4.4 Hz).

### Examples 34-52

The compounds of Examples 34-52 were synthesized from the corresponding compounds of each Reference Example according to the process of Example 1.

| Example | Chemical Structure | Instrumental Analysis Data |
|---|---|---|
| 34 | 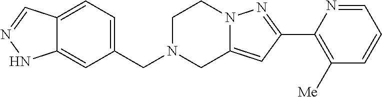 [SMILES: Cc1cccnc1-c1cc2n(n1)CCN(Cc1ccc3cn[nH]c3c1)C2] | ¹H-NMR (400 MHz, CDCl₃) δ: 2.61 (3H, s), 3.04 (2H, t, J = 5.5 Hz), 3.79 (2H, s), 3.88 (2H, s), 4.29 (2H, t, J = 5.6 Hz), 6.65 (1H, s), 7.53 (1H, s), 7.19- |
| | | 7.26 (3H, m), 7.65 (1H, |
| | | d, J = 7.6 Hz), 7.74 |
| | | (1H, d, J = 8.3 Hz), |
| | | 8.07 (1H, d, J = 1.0 |
| | | Hz), 8.55 (1H, d, J = 3.7 Hz). |
| 35 | 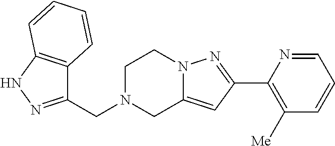 [SMILES: Cc1cccnc1-c1cc2n(n1)CCN(Cc1n[nH]c3ccccc13)C2] | ¹H-NMR (300 MHz, CDCl₃) δ: 2.56 (3H, s), 3.07 (2H, t, J = 5.5 Hz), 3.87 (2H, s), 4.18 (2H, s), 4.28 (2H, t, J = 5.5 Hz), 6.44 (1H, s), 7.11 (1H, dd, J = 7.7, 4.8 Hz), 7.15-7.21 (1H, m), 7.41 (1H, dd, J = 7.0, 7.0 Hz), 7.48 |
| | | (1H, d, J = 8.1 Hz), |
| | | 7.53 (1H, d, J = 8.8 |
| | | Hz), 7.92 (1H, d, J = |
| | | 8.1 Hz), 8.49 (1H, dd, |
| | | J = 4.4, 1.5 Hz), 9.96 |
| | | (1H, brs). |
| 36 | 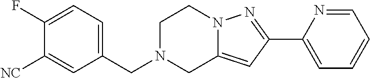 [SMILES: N#Cc1cc(CN2CCn3nc(-c4ccccn4)cc3C2)ccc1F] | ¹H-NMR (400 MHz, CDCl₃) δ: 3.02 (2H, t, J = 5.7 Hz), 3.76 (4H, s), 4.30 (2H, t, J = 5.7 Hz), 6.63 (1H, s), 7.23- 7.24 (2H, m), 7.66- |
| | | 7.75 (3H, m), 7.90- |
| | | 7.92 (1H, m), 8.63 |
| | | 8.63 (1H, m). |
| 37 | 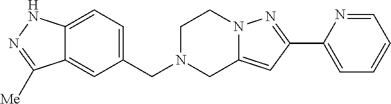 [SMILES: Cc1n[nH]c2ccc(CN3CCn4nc(-c5ccccn5)cc4C3)cc12] | ¹H-NMR (400 MHz, CDCl₃) δ: 2.62 (3H, s), 3.04 (2H, t, J = 5.5 Hz), 3.79 (2H, s), 3.87 (2H, s), 4.30 (2H, t, J = 5.7 Hz), 6.60 (1H, s), 7.19-7.21 (1H, m), |
| | | 7.43-7.46 (2H, m), |
| | | 7.67 (1H, s), 7.71- |
| | | 7.73 (2H, m), 7.90- |
| | | 7.93 (1H, m), 8.62 |
| | | 8.64 (1H, m). |
| 38 | 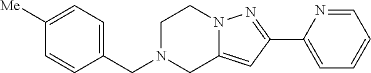 [SMILES: Cc1ccc(CN2CCn3nc(-c4ccccn4)cc3C2)cc1] | ¹H-NMR (400 MHz, CDCl₃) δ: 2.34 (3H, s), 2.99 (2H, t, J = 5.5 Hz), 3.72 (4H, d, J = 2.4 Hz), 4.24 (2H, t, J = 5.6 Hz), 6.29 (1H, s), |
| | | 7.16 (2H, d, J = 7.8 |
| | | Hz), 7.25 (2H, d, J = |
| | | 9.3 Hz), 7.36 (1H, dd, |
| | | J = 8.0, 4.9 Hz), 8.13 |
| | | (1H, dd, J = 7.9, 1.3 |
| | | Hz), 8.50 (1H, dd, J = |
| | | 4.9, 1.7 Hz), 8.95 (1H, |
| | | d, J = 2.2 Hz). |
| 39 | 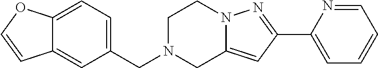 [SMILES: c1ccc(-c2cc3n(n2)CCN(Cc2ccc4occc4c2)C3)nc1] | ¹H-NMR (300 MHz, CDCl₃) δ: 2.98 (2H, t, J = 5.7 Hz), 3.74 (2H, s), 3.81 (2H, s), 4.24 (2H, t, J = 5.7 Hz), 6.55 (1H, s), 6.74 (1H, d, J = |
| | | 1.5 Hz), 7.12-7.18 |
| | | (1H, m), 7.30 (1H, dd, |
| | | J = 8.8, 1.5 Hz), 7.47 |
| | | (1H, d, J = 8.8 Hz), |
| | | 7.58 (1H, d, J = 1.5 |
| | | Hz), 7.62 (1H, d, J = |
| | | 2.2 Hz), 7.67 (1H, ddd, |
| | | J = 7.7, 7.7, 1.7 Hz), |
| | | 7.86 (1H, d, J = 8.1 |
| | | Hz), 8.58 (1H, d, J = 5.1 Hz). |
| 40 | 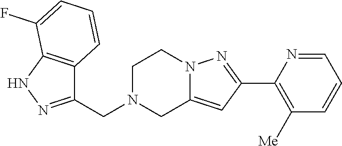 [SMILES: Cc1cccnc1-c1cc2n(n1)CCN(Cc1n[nH]c3c(F)cccc13)C2] | ¹H-NMR (300 MHz, CDCl₃) δ: 2.56 (3H, s), 3.06 (2H, t, J = 5.5 Hz), 3.85 (2H, s), 4.17 (2H, s), 4.27 (2H, t, J = 5.5 Hz), 6.45 (1H, s), 7.03-7.14 (3H, m), 7.53 (1H, d, J = 7.3 Hz), 7.67 (1H, dd, J = 5.9, 2.9 Hz), 8.49 (1H, |
| | | dd, J = 5.1, 1.5 Hz), |
| | | 10.88 (1H, brs). |
| 41 | 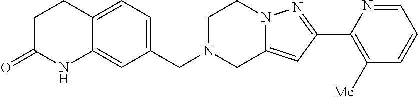 [SMILES: Cc1cccnc1-c1cc2n(n1)CCN(Cc1ccc3c(c1)NC(=O)CC3)C2] | ¹H-NMR (400 MHz, CDCl₃) δ: 2.65 (5H, dd, J = 8.3, 6.6 Hz), 3.02- 2.96 (4H, m), 3.70 (2H, s), 3.75 (2H, s), 4.29 (2H, t, J = 5.6 Hz), 6.79 (1H, d, J = 1.2 |
| | | Hz), 6.99 (1H, dd, J = |
| | | 7.7, 1.3 Hz), 7.15 (1H, |
| | | d, J = 7.6 Hz) 7.25- |
| | | 7.27 (1H, m), 7.59 (1H, |
| | | s), 7.69 (1H, s), 8.55- |
| | | 8.57 (1H, m). |
| 42 | 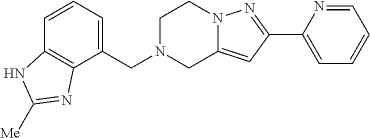 [SMILES: Cc1nc2c(CN3CCn4nc(-c5ccccn5)cc4C3)cccc2[nH]1] | ¹H-NMR (400 MHz, CDCl₃) δ: 2.63 (3H, s), 3.02 (2H, t, J = 5.5 Hz), 3.82 (2H, s), 4.07 (2H, s), 4.26 (2H, t, J = 5.5 Hz), 6.62 (1H, s), 7.11-7.13 (1H, m), 7.20-7.25 (2H, m), 7.64-7.66 (1H, m), |
| | | 7.73-7.75 (1H, m), |
| | | 7.90-7.92 (1H, m), |
| | | 8.63-8.64 (1H, m). |
| 43 | 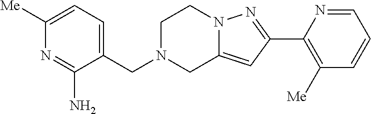 [SMILES: Cc1ccc(CN2CCn3nc(-c4ncccc4C)cc3C2)c(N)n1] | ¹H-NMR (400 MHz, CDCl₃) δ: 2.43 (3H, s), 2.57 (3H, s), 2.97 (2H, t, J = 5.6 Hz), 3.69 (2H, s), 3.75 (2H, s), 4.27 (2H, t, J = 5.5 Hz), 5.85 (1H, br s), 6.47 (1H, s), 6.50 (1H, d, |
| | | J = 7.3 Hz), 7.12 (1H, |
| | | dd, J = 7.7, 4.8 Hz), |
| | | 7.54 (1H, d, J = 7.6 |
| | | Hz), 8.49 (1H, d, J = 4.6 Hz). |
| 44 | 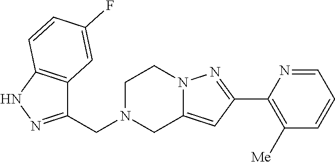 [SMILES: Cc1cccnc1-c1cc2n(n1)CCN(Cc1n[nH]c3ccc(F)cc13)C2] | ¹H-NMR (300 MHz, CDCl₃) δ: 2.57 (3H, s), 3.05 (2H, t, J = 5.5 Hz), 3.83 (2H, s), 4.12 (2H, s), 4.28 (2H, t, J = 5.5 Hz), 6.44 (1H, s), 7.09-7.20 (2H, m), 7.38 (1H, dd, J = 9.2, 4.0 Hz), 7.49-7.57 (2H, m), 8.49 (1H, d, J = 3.7 Hz). |
| 45 | 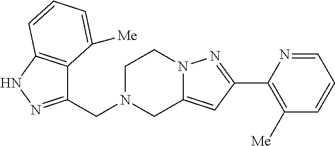 [SMILES: Cc1cccnc1-c1cc2n(n1)CCN(Cc1n[nH]c3cccc(C)c13)C2] | ¹H-NMR (400 MHz, CDCl₃) δ: 2.62 (3H, s), 2.77 (3H, s), 3.08 (2H, t, J = 5.5 Hz), 3.85 (2H, s), 4.12 (2H, s), 4.24 (2H, t, J = 5.5 Hz), 6.73 (1H, s), 6.91- 6.95 (1H, m), 7.21- 7.32 (3H, m), 7.67- 7.69 (1H, m), 8.56 (1H, |
| | | d, J = 3.7 Hz), 10.01 (1H, s). |
| 46 | 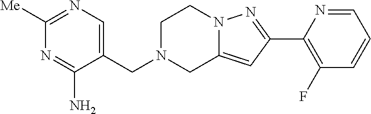 [SMILES: Cc1ncc(CN2CCn3nc(-c4ncccc4F)cc3C2)c(N)n1] | ¹H-NMR (400 MHz, CDCl₃) δ: 2.51 (3H, s), 3.00 (2H, t, J =0 5.6 Hz), 3.68 (2H, s), 3.76 (2H, s), 4.34 (2H, t, J = 5.6 Hz), 6.63 (1H, d, J = 3.6 Hz), 7.22-7.28 (1H, m), 7.47 (1H, dd, |
| | | J = 9.6, 8.4 Hz), 8.01 |
| | | (1H, s), 8.50 (1H, d, |
| | | J = 4.4 Hz). |
| 47 | 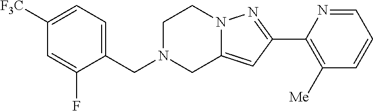 [SMILES: Cc1cccnc1-c1cc2n(n1)CCN(Cc1ccc(C(F)(F)F)cc1F)C2] | ¹H-NMR (400 MHz, CDCl₃) δ: 2.57 (3H, s), 3.04 (2H, t, J = 5.5 Hz), 3.81 (2H, s), 3.87 (2H, s), 4.30 (2H, t, J = 5.5 Hz), 6.46 (1H, s), 7.12 (1H, dd, J = 7.6, 4.8 Hz), 7.35-7.37 |
| | | (1H, m), 7.44-7.45 |
| | | (1H, m), 7.53-7.55 |
| | | (1H, m), 7.59-7.66 |
| | | (1H, m), 8.49-8.50 (1H, m). |
| 48 | 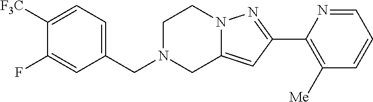 [SMILES: Cc1cccnc1-c1cc2n(n1)CCN(Cc1ccc(C(F)(F)F)c(F)c1)C2] | ¹H-NMR (400 MHz, CDCl₃) δ: 2.58 (3H, s), 3.01 (2H, t, J = 5.6 Hz), 3.78 (2H, s), 3.79 (2H, s), 4.30 (2H, t, J = 5.6 Hz), 6.47 (1H, s), 7.13 (1H, dd, J = 7.8, |
| | | 4.6 Hz), 7.27-7.32 |
| | | (2H, m), 7.52-7.56 |
| | | (1H, m), 7.59 (1H, dd, |
| | | J = 7.7, 7.7 Hz), 8.48- |
| | | 8.51 (1H, m). |
| 49 | 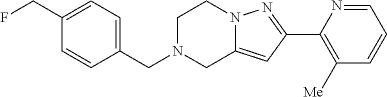 [SMILES: Cc1cccnc1-c1cc2n(n1)CCN(Cc1ccc(CF)cc1)C2] | ¹H-NMR (400 MHz, CDCl₃) δ: 2.58 (3H, s), 2.99 (2H, t, J = 5.6 Hz), 3.75 (2H, s), 3.76 (2H, s), 4.27 (2H, t, J = 5.6 Hz), 5.39 (2H, d, J = 47.8 Hz), 6.52 (1H, |
| | | s), 7.15 (1H, dd, J = |
| | | 7.4, 4.8 Hz), 7.37- |
| | | 7.44 (4H, m), 7.57 (1H, |
| | | d, J = 7.3 Hz), 8.51 |
| | | (1H, d, J = 4.6 Hz). |
| 50 | 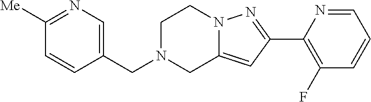 [SMILES: Cc1ccc(CN2CCn3nc(-c4ncccc4F)cc3C2)cn1] | ¹H-NMR (400 MHz, CDCl₃) δ: 2.58 (3H, s), 2.99 (2H, t, J = 5.6 Hz), 3.72 (2H, s), 3.75 (2H, s), 4.32 (2H, t, J = 5.6 Hz), 6.59 (1H, d, J = 3.7 Hz), 7.17-7.24 |
| | | (2H, m), 7.45 (1H, ddd, |
| | | J = 11.0, 8.3, 1.5 Hz), |
| | | 7.65 (1H, dd, J = 8.0, |
| | | 2.0 Hz), 8.48 (2H, dq, |
| | | J = 8.6, 2.4 Hz). |
| 51 | 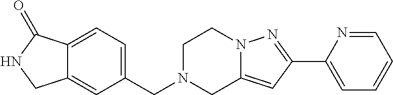 [SMILES: O=C1NCc2cc(CN3CCn4nc(-c5ccccn5)cc4C3)ccc21] | ¹H-NMR (400 MHz, CDCl₃) δ: 2.99 (2H, t, J = 5.6 Hz), 3.74 (2H, s), 3.82 (2H, s), 4.25 (2H, t, J = 5.6 Hz), 4.43 (2H, s), 6.54-6.56 (2H, m), 7.13-7.17 (1H, |
| | | m), 7.46 (1H, J = 7.6 |
| | | Hz,), 7.52 (1H, s), 7.64- |
| | | 7.69 (1H, m), 7.82- |
| | | 7.86 (2H, m), 8.56- |
| | | 8.58 (1H, m). |
| 52 | 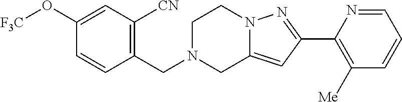 [SMILES: Cc1cccnc1-c1cc2n(n1)CCN(Cc1ccc(OC(F)(F)F)cc1C#N)C2] | ¹H-NMR (400 MHz, CDCl₃) δ: 2.61 (3H, s), 3.10 (2H, t, J = 5.6 Hz), 3.81 (2H, s), 3.96 (2H, s), 4.31 (2H, t, J = 5.6 Hz), 6.64 (1H, br s), 7.20 (1H, br s), |
| | | 7.46 (1H, d, J = 8.3 |
| | | Hz), 7.55 (2H, s), 7.69 |
| | | (1H, d, J = 8.5 Hz), |
| | | 8.53 (1H, d, J = 4.4 Hz). |

### Examples 53-83

The compounds of Examples 53-83 were synthesized from the corresponding compounds of each Reference Example according to the process of Example 25.

| Example | Chemical Structure | Instrumental Analysis Data |
|---|---|---|
| 53 | 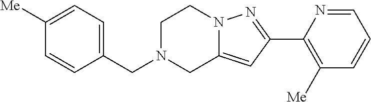 [SMILES: Cc1ccc(CN2CCn3nc(-c4ncccc4C)cc3C2)cc1] | ¹H-NMR (400 MHz, CDCl₃) δ: 2.36 (3H, s), 2.61 (3H, s), 2.99 (2H, t, J = 5.5 Hz), 3.72 (2H, s), 3.76 (2H, s), 4.27 (2H, t, J = 5.5 Hz), 6.64 (1H, s), 7.16- |
| | | 7.28 (5H, m), 7.64 (1H, |
| | | s), 8.54 (1H, s). |
| 54 | 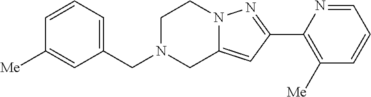 [SMILES: Cc1cccc(CN2CCn3nc(-c4ncccc4C)cc3C2)c1] | ¹H-NMR (400 MHz, CDCl₃) δ: 2.37 (3H, s), 2.63 (3H, s), 3.00 (2H, t, J = 5.6 Hz), 3.72 (2H, s), 3.77 (2H, s), 4.28 (2H, t, J = 5.5 Hz), 6.70-6.73 (1H, br m), |
| | | 7.11-7.29 (5H, m), |
| | | 7.68 (1H, br s), 8.54- |
| | | 8.57 (1H, m). |
| 55 | 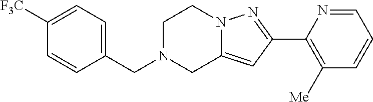 [SMILES: Cc1cccnc1-c1cc2n(n1)CCN(Cc1ccc(C(F)(F)F)cc1)C2] | ¹H-NMR (400 MHz, CDCl₃) δ: 2.61 (3H, s), 3.00 (2H, t, J = 5.6 Hz), 3.77 (2H, s), 3.80 (2H, s), 4.29 (2H, t, J = 5.6 Hz), 6.64 (1H, s), 7.20 (1H, dd, J = 6.8, |
| | | 5.4 Hz), 7.52 (2H, d, |
| | | J = 8.0 Hz), 7.62 (2H, d, |
| | | J = 8.0 Hz), 7.64 (1H, |
| | | br s), 8.54 (1H, d, J = |
| | | 4.4 Hz). |
| 56 | 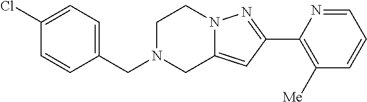 [SMILES: Cc1cccnc1-c1cc2n(n1)CCN(Cc1ccc(Cl)cc1)C2] | ¹H-NMR (400 MHz, CDCl₃) δ: 2.54 (3H, s), 2.91 (2H, t, J = 5.6 Hz), 3.64 (2H, s), 3.68 (2H, s), 4.20 (2H, t, J = 5.5 Hz), 6.55 (1H, s), 7.14-7.10 (1H, m), |
| | | 7.19 (2H, d, J = 0.5 |
| | | Hz), 7.26 (2H, d, J = |
| | | 0.5 Hz), 7.56 (1H, d, |
| | | J = 6.6 Hz), 8.47 (1H, d, |
| | | J = 4.1 Hz). |
| 57 | 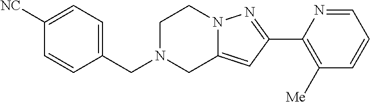 [SMILES: Cc1cccnc1-c1cc2n(n1)CCN(Cc1ccc(C#N)cc1)C2] | ¹H-NMR (400 MHz, CDCl₃) δ: 2.62 (3H, s), 3.00 (2H, t, J = 5.6 Hz), 3.77 (2H, s), 3.80 (2H, s), 4.29 (2H, t, J = 5.5 Hz), 6.66 (1H, s), 7.19-7.23 (1H, m), |
| | | 7.52 (2H, d, J = 7.8 |
| | | Hz), 7.66 (3H, d, J = |
| | | 7.8 Hz), 8.54 (1H, d, |
| | | J = 4.4 Hz). |
| 58 | 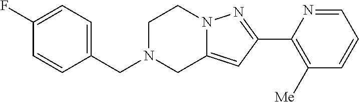 [SMILES: Cc1cccnc1-c1cc2n(n1)CCN(Cc1ccc(F)cc1)C2] | ¹H-NMR (400 MHz, CDCl₃) δ: 2.61 (3H, s), 2.98 (2H, t, J = 5.6 Hz), 3.71 (2H, s), 3.74 (2H, s), 4.27 (2H, t, J = 5.5 Hz), 6.62 (1H, br s), 7.05 (2H, t, J = |
| | | 8.5 Hz), 7.20 (1H, s), |
| | | 7.35 (2H, dd, J = 8.4, |
| | | 5.7 Hz), 7.61-7.65 |
| | | (1H, m), 8.54 (1H, d, |
| | | J = 4.9 Hz). |
| 59 | 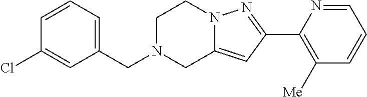 [SMILES: Cc1cccnc1-c1cc2n(n1)CCN(Cc1cccc(Cl)c1)C2] | ¹H-NMR (400 MHz, CDCl₃) δ: 2.62 (3H, s), 2.99 (2H, t, J = 5.6 Hz), 3.72 (2H, s), 3.76 (2H, s), 4.28 (2H, t, J = 5.6 Hz), 6.69 (1H, br s), 7.19-7.30 (4H, |
| | | m), 7.40 (1H, s), 7.66 |
| | | (1H, s), 8.55 (1H, d, |
| | | J = 4.4 Hz). |
| 60 | 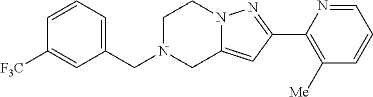 [SMILES: Cc1cccnc1-c1cc2n(n1)CCN(Cc1cccc(C(F)(F)F)c1)C2] | ¹H-NMR (400 MHz, CDCl₃) δ: 2.60 (3H, s), 3.00 (2H, t, J = 5.5 Hz), 3.77 (2H, s), 3.80 (2H, s), 4.29 (2H, t, J = 5.5 Hz), 6.60 (1H, br s), 7.216-7.20 (1H, |
| | | m), 7.48 (1H, t, J = |
| | | 7.6 Hz), 7.56-7.62 |
| | | (3H, m), 7.66 (1H, s), |
| | | 8.52 (1H, d, J = 4.6 Hz). |
| 61 | 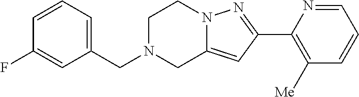 [SMILES: Cc1cccnc1-c1cc2n(n1)CCN(Cc1cccc(F)c1)C2] | ¹H-NMR (400 MHz, CDCl₃) δ: 2.60 (3H, s), 2.98 (2H, t, J = 5.6 Hz), 3.72 (2H, s), 3.75 (2H, s), 4.26 (2H, t, J = 5.6 Hz), 6.65 (1H, br s), 6.95-7.00 (1H, |
| | | m), 7.09-7.34 (4H, |
| | | m), 7.64 (1H, br s), |
| | | 8.53 (1H, d, J = 4.1 Hz). |
| 62 | 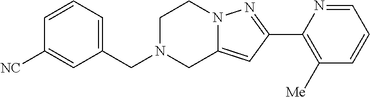 [SMILES: Cc1cccnc1-c1cc2n(n1)CCN(Cc1cccc(C#N)c1)C2] | ¹H-NMR (400 MHz, CDCl₃) δ: 2.60 (3H, s), 3.01 (2H, t, J = 5.5 Hz), 3.76 (2H, s), 3.78 (2H, s), 4.29 (2H, t, J = 5.5 Hz), 6.60 (1H, br s), 7.16-7.20 (1H, |
| | | m), 7.48 (1H, t, J = |
| | | 7.8 Hz), 7.60-7.66 |
| | | (3H, m), 7.71 (1H, s), |
| | | 8.52 (1H, d, J = 4.6 Hz). |
| 63 | 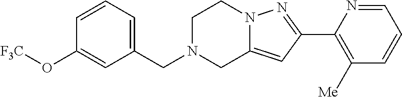 [SMILES: Cc1cccnc1-c1cc2n(n1)CCN(Cc1cccc(OC(F)(F)F)c1)C2] | ¹H-NMR (400 MHz, CDCl₃) δ: 2.58 (3H, s), 2.99 (2H, t, J = 5.6 Hz), 3.77 (4H, s), 4.28 (2H, t, J = 5.6 Hz), 6.46 (1H, s), 7.12 (1H, dd, J = 7.6, 4.6 Hz), 7.14- |
| | | 7.18 (1H, m), 7.27- |
| | | 7.29 (1H, m), 7.31- |
| | | 7.34 (1H, m), 7.38 (1H, |
| | | dd, J = 7.8, 7.8 Hz), |
| | | 7.52-7.55 (1H, m), |
| | | 8.48-8.51 (1H, m). |
| 64 | 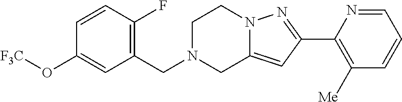 [SMILES: Cc1cccnc1-c1cc2n(n1)CCN(Cc1cc(OC(F)(F)F)ccc1F)C2] | ¹H-NMR (400 MHz, CDCl₃) δ: 2.58 (3H, s), 3.03 (2H, t, J = 5.6 Hz), 3.82 (4H, s), 4.29 (2H, t, J = 5.6 Hz), 6.47 (1H, s), 7.07-7.18 (3H, m), 7.37-7.41 |
| | | (1H, m), 7.52-7.56 |
| | | (1H, m), 8.48-8.51 (1H, m). |
| 65 | 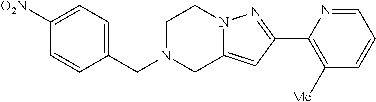 [SMILES: Cc1cccnc1-c1cc2n(n1)CCN(Cc1ccc([N+](=O)[O-])cc1)C2] | ¹H-NMR (400 MHz, CDCl₃) δ: 2.58 (3H, s), 3.02 (2H, t, J = 5.6 Hz), 3.78 (2H, s), 3.85 (2H, s), 4.30 (2H, t, J = 5.6 Hz), 6.46 (1H, s), 7.13 (1H, dd, J = 7.6, |
| | | 4.6 Hz), 7.52-7.56 |
| | | (1H, m), 7.59 (2H, d, |
| | | J = 8.6 Hz), 8.23 (2H, d, |
| | | J = 8.6 Hz), 8.48- |
| | | 8.51 (1H, m). |
| 66 | 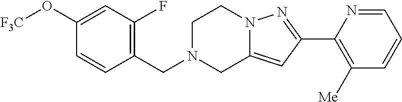 [SMILES: Cc1cccnc1-c1cc2n(n1)CCN(Cc1ccc(OC(F)(F)F)cc1F)C2] | ¹H-NMR (400 MHz, CDCl₃) δ: 2.57 (3H, s), 3.03 (2H, t, J = 5.6 Hz), 3.79 (2H, s), 3.81 (2H, s), 4.29 (2H, t, J = 5.5 Hz), 6.50 (1H, br s), 6.99 (1H, d, J = |
| | | 10.0 Hz), 7.04 (1H, d, |
| | | J = 8.5 Hz), 7.13 (1H, |
| | | dd, J = 7.6, 4.6 Hz), |
| | | 7.51 (1H, t, J = 8.3 |
| | | Hz), 7.55 (1H, d, J = |
| | | 7.6 Hz), 8.50 (1H, d, |
| | | J = 3.9 Hz). |
| 67 | 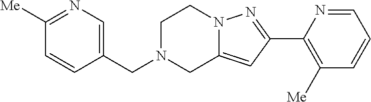 [SMILES: Cc1ccc(CN2CCn3nc(-c4ncccc4C)cc3C2)cn1] | ¹H-NMR (400 MHz, CDCl₃) δ: 2.56 (s, 3H), 2.57 (s, 3H), 2.98 (t, J = 5.6 Hz, 2H), 3.72 (s, 2H), 3.75 (s, 2H), 4.26 (t, J = 5.6 Hz, 2H), 6.44 (s, 1H), 7.12 (dd, |
| | | J = 7.8, 7.6 Hz, 1H), |
| | | 7.17 (d, J = 7.8 Hz, |
| | | 1H), 7.53 (d, J = 7.6 |
| | | Hz, 1H), 7.64 (dd, J = |
| | | 8.0, 7.8 Hz, 1H), 8.46- |
| | | 8.51 (m, 2H). |
| 68 | 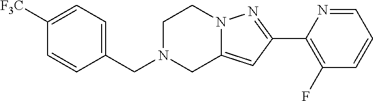 [SMILES: Fc1cccnc1-c1cc2n(n1)CCN(Cc1ccc(C(F)(F)F)cc1)C2] | ¹H-NMR (400 MHz, CDCl₃) δ: 3.04 (2H, br s), 3.80 (2H, s), 3.84 (2H, s), 4.36 (2H, t, J = 5.1 Hz), 6.63 (1H, s), 7.24-7.25 (1H, m), 7.47 (1H, ddd, J = 11.0, |
| | | 8.3, 1.0 Hz), 7.54 (2H, |
| | | d, J = 7.3 Hz), 7.63 |
| | | (2H, d, J = 8.0 Hz), |
| | | 8.51 (1H, br s). |
| 69 | 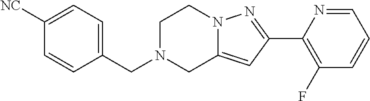 [SMILES: N#Cc1ccc(CN2CCn3nc(-c4ncccc4F)cc3C2)cc1] | ¹H-NMR (400 MHz, CDCl₃) δ: 3.04 (2H, br s), 3.80 (2H, s), 3.84 (2H, s), 4.36 (2H, t, J = 5.4 Hz), 6.63 (1H, br s), 7.24-7.26 (1H, br m), 7.45-7.55 (3H, |
| | | m), 7.67 (2H, d, J = |
| | | 8.0 Hz), 8.51 (1H, br s). |
| 70 | 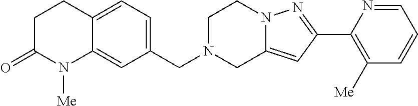 [SMILES: Cc1cccnc1-c1cc2n(n1)CCN(Cc1ccc3c(c1)N(C)C(=O)CC3)C2] | ¹H-NMR (400 MHz, CDCl₃) δ: 2.58 (3H, s), 2.64 (2H, dd, J = 8.7, 6.2 Hz), 2.89 (2H, t, J = 7.3 Hz), 2.99 (2H, t, J = 5.5 Hz), 3.35 (3H, s), 3.72 (2H, s), 3.75 |
| | | (2H, s), 4.26 (2H, t, |
| | | J = 5.5 Hz), 6.56 (1H, br |
| | | s), 6.98-7.02 (2H, |
| | | m), 7.11-7.17 (2H, |
| | | m), 7.58 (1H, br s), |
| | | 8.50 (1H, d, J = 4.6 Hz). |
| 71 | 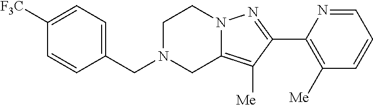 [SMILES: Cc1cccnc1-c1nn2c(c1C)CN(Cc1ccc(C(F)(F)F)cc1)CC2] | ¹H-NMR (400 MHz, CDCl₃) δ: 1.94 (3H, s), 2.39 (3H, s), 2.97 (2H, t, J = 5.0 Hz), 3.66 (2H, s), 3.83 (2H, s), 4.22 (2H, t, J = 5.5 Hz), 7.16 (1H, dd, J = 7.8, |
| | | 5.0 Hz), 7.54-7.56 |
| | | (3H, m), 7.62-7.64 |
| | | (2H, m), 8.50-8.51 (1H, m). |
| 72 | 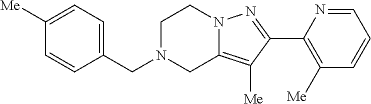 [SMILES: Cc1ccc(CN2CCn3nc(-c4ncccc4C)c(C)c3C2)cc1] | ¹H-NMR (400 MHz, CDCl₃) δ: 1.93 (3H, s), 2.36 (3H, s), 2.37 (3H, s), 2.92-2.94 (2H, m), 3.63 (2H, s), 3.72 (2H, s), 4.19 (2H, t, J = 5.5 Hz), 7.13-7.16 |
| | | (3H, m), 7.27-7.28 |
| | | (2H, m), 7.53-7.55 |
| | | (1H, m), 8.48-8.49 (1H, m). |
| 73 | 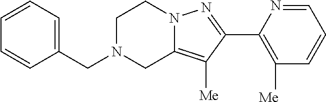 [SMILES: Cc1cccnc1-c1nn2c(c1C)CN(Cc1ccccc1)CC2] | ¹H-NMR (400 MHz, CDCl₃) δ: 1.94 (3H, s), 2.38 (3H, s), 2.96 (2H, t, J = 5.5 Hz), 3.66 (2H, s), 3.77 (2H, s), 4.21 (2H, t, J = 5.5 Hz), 7.14 (1H, dd, J = 7.6, |
| | | 4.8 Hz), 7.27-7.41 |
| | | (5H, m), 7.55 (1H, d, |
| | | J = 6.9 Hz), 8.50 (1H, d, |
| | | J = 3.7 Hz). |
| 74 | 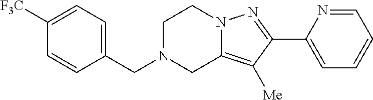 [SMILES: Cc1c(-c2ccccn2)nn2c1CN(Cc1ccc(C(F)(F)F)cc1)CC2] | ¹H-NMR (400 MHz, CDCl₃) δ: 2.25 (3H, s), 2.97 (2H, t, J = 5.5 Hz), 3.67 (2H, s), 3.82 (2H, s), 4.24 (2H, t, J = 5.5 Hz), 7.16-7.19 (1H, m), 7.58 (4H, dd, |
| | | J = 40.6, 8.0 Hz), 7.67- |
| | | 7.72 (1H, m), 7.81-7.83 |
| | | (1H, m), 8.64-8.65 (1H, m). |
| 75 | 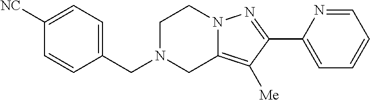 [SMILES: Cc1c(-c2ccccn2)nn2c1CN(Cc1ccc(C#N)cc1)CC2] | ¹H-NMR (400 MHz, CDCl₃) δ: 2.23 (3H, s), 2.96 (2H, t, J = 5.5 Hz), 3.64 (2H, s), 3.81 (2H, s), 4.23 (2H, t, J = 5.5 Hz), 7.15-7.18 (1H, m), 7.51-7.53 |
| | | (2H, m), 7.66-7.70 |
| | | (3H, m), 7.80-7.82 |
| | | (1H, m), 8.63-8.65 (1H, m). |
| 76 | 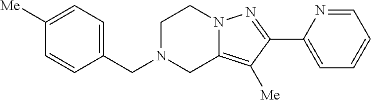 [SMILES: Cc1ccc(CN2CCn3nc(-c4ccccn4)c(C)c3C2)cc1] | ¹H-NMR (300 MHz, CDCl₃) δ: 2.24 (3H, s), 2.37 (3H, s), 2.94 (2H, t, J = 5.5 Hz), 3.64 (2H, s), 3.72 (2H, s), 4.21 (2H, t, J = 5.5 Hz), 7.13-7.21 (3H, m), |
| | | 7.27 (2H, d, J = 7.3 |
| | | Hz), 7.68 (1H, ddd, J = |
| | | 7.7, 7.7, 1.7 Hz), 7.81 |
| | | (1H, d, J = 7.3 Hz), |
| | | 8.64 (1H, d, J = 3.7 Hz). |
| 77 | 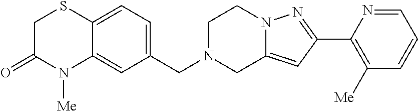 [SMILES: Cc1cccnc1-c1cc2n(n1)CCN(Cc1ccc3c(c1)N(C)C(=O)CS3)C2] | ¹H-NMR (400 MHz, CDCl₃) δ: 2.57 (3H, s), 3.01 (2H, t, J = 5.5 Hz), 3.42 (2H, s), 3.46 (3H, s), 3.75 (4H, d, J = 10.1 Hz), 4.29 (2H, t, J = 5.5 Hz), 6.46 (1H, s), 7.05 (1H, dd, J = |
| | | 8.0, 1.6 Hz), 7.11- |
| | | 7.15 (2H, m), 7.34 (1H, |
| | | d, J = 7.8 Hz), 7.54- |
| | | 7.55 (1H, m), 8.50 (1H, |
| | | dd, J = 4.6, 1.8 Hz). |
| 78 | 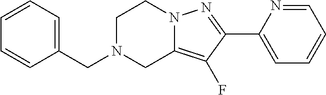 [SMILES: Fc1c(-c2ccccn2)nn2c1CN(Cc1ccccc1)CC2] | ¹H-NMR (400 MHz, CDCl₃) δ: 2.97 (2H, t, J = 5.6 Hz), 3.73 (2H, s), 3.77 (2H, s), 4.21 (2H, t, J = 5.6 Hz), 7.20 (1H, dd, J = 4.8, 4.8 Hz), 7.29-7.41 (5H, m), |
| | | 7.72 (1H, dd, J = 8.4, |
| | | 7.2 Hz), 7.79 (1H, d, |
| | | J = 8.4 Hz), 8.70 (1H, brs). |
| 79 | 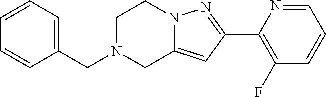 [SMILES: Fc1cccnc1-c1cc2n(n1)CCN(Cc1ccccc1)C2] | ¹H-NMR (400 MHz, CDCl₃) δ: 3.00 (2H, t, J = 5.4 Hz), 3.75 (4H, s), 4.33 (2H, t, J = 5.4 Hz), 6.59 (1H, d, J = 3.6 Hz), 7.20-7.25 (1H, m), 7.29-7.42 (5H, |
| | | m), 7.46 (1H, dd, J = |
| | | 6.2, 6.2 Hz), 8.50 (1H, |
| | | dd, J = 2.8, 2.8 Hz). |
| 80 | 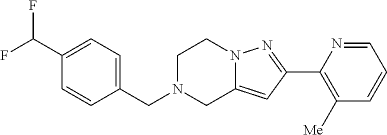 [SMILES: Cc1cccnc1-c1cc2n(n1)CCN(Cc1ccc(C(F)F)cc1)C2] | ¹H-NMR (300 MHz, CDCl₃) δ: 2.59 (3H, s), 3.00 (2H, t, J = 5.7 Hz), 3.76 (2H, s), 3.79 (2H, s), 4.28 (2H, t, J = 5.7 Hz), 6.53 (1H, s), 6.66 (1H, t, J = 56.5 Hz), 7.15 (1H, dd, J = 7.7, 4.8 Hz), 7.50 (4H, |
| | | s), 7.58 (1H, d, J = 8.1 |
| | | Hz), 8.51 (1H, d, J = 4.4 Hz). |
| 81 | 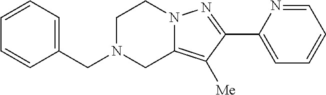 [SMILES: Cc1c(-c2ccccn2)nn2c1CN(Cc1ccccc1)CC2] | ¹H-NMR (400 MHz, CDCl₃) δ: 2.24 (3H, s), 2.96 (2H, t, J = 5.6 Hz), 3.66 (2H, s), 3.77 (2H, s), 4.22 (2H, t, J = 5.6 Hz), 7.16 (1H, dd, J = 6.9, 5.3 Hz), 7.27- |
| | | 7.43 (5H, m), 7.69 |
| | | (1H, dd, J = 9.6, 7.6 |
| | | Hz), 7.81 (1H, d, J = |
| | | 7.6 Hz), 8.64 (1H, d, J = |
| | | 4.8 Hz). |
| 82 | 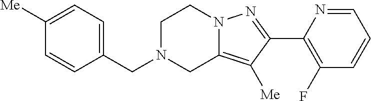 [SMILES: Cc1ccc(CN2CCn3nc(-c4ncccc4F)c(C)c3C2)cc1] | ¹H-NMR (300 MHz, CDCl₃) δ: 2.03 (3H, d, J = 1.5 Hz), 2.36 (3H, s), 2.95 (2H, t, J = 5.5 Hz), 3.65 (2H, s), 3.73 (2H, s), 4.24 (2H, t, J = 5.5 Hz), 7.17 (2H, d, J = |
| | | 8.1 Hz), 7.22-7.31 |
| | | (3H, m), 7.47 (1H, dd, |
| | | J = 8.8, 8.8 Hz), 8.50 |
| | | (1H, d, J = 4.4 Hz). |
| 83 | 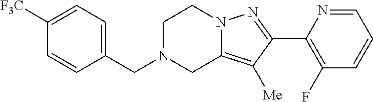 [SMILES: Cc1c(-c2ncccc2F)nn2c1CN(Cc1ccc(C(F)(F)F)cc1)CC2] | ¹H-NMR (300 MHz, CDCl₃) δ: 2.04 (3H, d, J = 1.5 Hz), 2.97 (2H, t, J = 5.5 Hz), 3.67 (2H, s), 3.82 (2H, s), 4.27 (2H, t, J = 5.5 Hz), 7.23- 7.31 (1H, m), 7.47 (1H, |
| | | dd, J = 10.3, 1.5 Hz), |
| | | 7.52 (2H, d, J = 8.1 Hz), |
| | | 7.62 (2H, d, J = 8.1 Hz), |
| | | 8.51 (1H, d, J = 5.1 Hz). |

### Example 84

### 5-{[2-(3-Methylpyridin-2-yl)-6,7-dihydropyrazolo[1,5-a]pyrazin-5(4H)-yl]methyl}-2-(trifluoromethyl)pyrimidine-4-amine

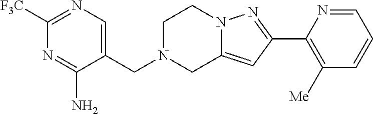 [SMILES: Cc1cccnc1-c1cc2n(n1)CCN(Cc1cnc(C(F)(F)F)nc1N)C2]

To a solution of the compound of Reference Example 12 (93.8 mg, 0.438 mmol) in methanol (2.0 mL) were added 4-amino-2-trifluoromethylpyrimidine-5-carbaldehyde (83.7 mg, 0.438 mmol), acetic acid (0.05 mL, 0.876 mmol), and then sodium cyanoborohydride (55.1 mg, 0.876 mmol). The mixture was stirred at room temperature for 16 hours, and then the reaction mixture was concentrated, and purified by preparative HPLC to give the title compound (18.5 mg, 11%).

¹H-NMR (400 MHz, CDCl₃) δ: 2.57 (s, 3H), 3.02 (t, J=5.6 Hz, 2H), 3.79 (d, J=6.8 Hz, 4H), 4.31 (t, J=5.6 Hz, 2H), 6.52 (s, 1H), 7.14 (dd, J=7.6, 7.6 Hz, 1H), 7.55 (d, J=7.6 Hz, 1H), 8.22 (s, 1H), 8.48-8.52 (m, 1H).

### Examples 85-105

The compounds of Examples 85-105 were synthesized from the corresponding compounds of each Reference Example according to the process of Example 84.

| | | Instrumental Analysis |
|---|---|---|
| Example | Chemical Structure | Data |
| 85 | 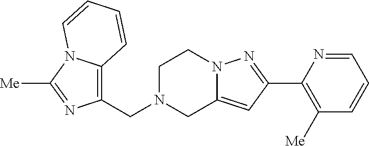 [SMILES: Cc1cccnc1-c1cc2n(n1)CCN(Cc1nc(C)n3ccccc13)C2] | ¹H-NMR (400 MHz, CDCl₃) δ: 2.54 (s, 3H), 2.66 (s, 3H), 3.06 (t, J = 5.6 Hz, 2H), 3.83 (s, 2H), 4.03 (s, 2H), 4.28 (t, J = 5.6 Hz, 2H), 6.42 (s, 1H), 6.53-6.60 (m, 1H), 6.63- 6.70 (m, 1H), 7.08-7.13 (m, 1H), 7.48- 7.54 (m, 2H), 7.62-7.68 (m, 1H), 8.46-8.51 (m, 1H). |
| 86 | 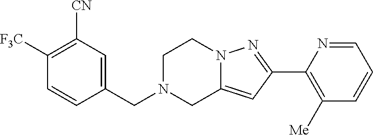 [SMILES: Cc1cccnc1-c1cc2n(n1)CCN(Cc1ccc(C(F)(F)F)c(C#N)c1)C2] | ¹H-NMR (400 MHz, CDCl₃) δ: 2.58 (s, 3H), 3.04 (t, J = 5.5 Hz, 2H), 3.78 (s, 2H), 3.85 (s, 2H), 4.31 (t, J = 5.5 Hz, 2H), 6.48 (s, 1H), 7.11-7.15 (m, 1H), 7.52-7.56 (m, 1H), 7.78-7.80 (m, 2H), 7.91-7.93 (m, 1H), 8.48-8.51 (m, 1H). |
| 87 | 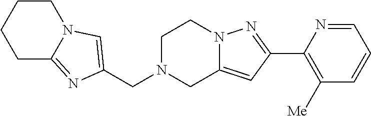 [SMILES: Cc1cccnc1-c1cc2n(n1)CCN(Cc1cn3c(n1)CCCC3)C2] | ¹H-NMR (400 MHz, CDCl₃) δ: 1.87-2.02 (m, 4H), 2.54 (s, 3H), 2.87 (t, J = 6.0 Hz, 2H), 3.07 (t, J = 5.6 Hz, 2H), 3.68 (s, 2H), 3.82 (s, 2H), 3.94 (t, J = 6.0 Hz, 2H), 4.28 (t, J = 5.6 Hz, 2H), 6.43 (s, 1H), 6.74 (s, 1H), 7.10 (dd, J = 7.6, 4.6 Hz, 1H), 7.52 (d, J = 7.6 Hz, 1H), 8.48 (d, J = 4.6 Hz, 1H). |
| 88 | 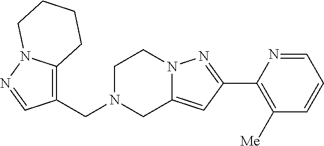 [SMILES: Cc1cccnc1-c1cc2n(n1)CCN(Cc1cnn3c1CCCC3)C2] | ¹H-NMR (400 MHz, CDCl₃) δ: 1.83-1.92 (m, 2H), 2.00-2.09 (m, 2H), 2.56 (s, 3H), 2.76 (t, J = 6.6 Hz, 2H), 2.94 (t, J = 5.6 Hz, 2H), 3.56 (s, 2H), 3.71 (s, 2H), 4.15 (t, J = 6.0 Hz, 2H), 4.24 (t, J = 5.6 Hz, 2H), 6.44 (s, 1H), 7.11 (dd, J = 7.8, 7.5 Hz, 1H), 7.43 (s, 1H), 7.53 (d, J = 6.8 Hz, 1H), 8.49 (d, J = 4.1 Hz, 1H). |
| 89 | 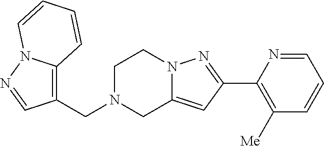 [SMILES: Cc1cccnc1-c1cc2n(n1)CCN(Cc1cnn3ccccc13)C2] | ¹H-NMR (400 MHz, CDCl₃) δ: 2.57 (s, 3H), 3.00 (t, J = 5.6 Hz, 2H), 3.78 (s, 2H), 3.94 (s, 2H), 4.25 (t, J = 5.6 Hz, 2H), 6.44 (s, 1H), 6.75-6.82 (m, 1H), 7.08-7.15 (m, 2H), 7.53 (d, J = 6.8 Hz, 1H), 7.65 (d, J = 8.8 Hz, 1H), 7.94 (s, 1H), 8.44-8.51 (m, 2H). |
| 90 | 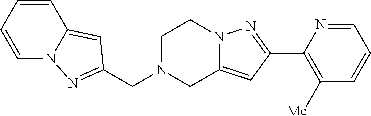 [SMILES: Cc1cccnc1-c1cc2n(n1)CCN(Cc1cc3ccccn3n1)C2] | ¹H-NMR (400 MHz, CDCl₃) δ: 2.55 (s, 3H), 3.09 (t, J = 5.6 Hz, 2H), 3.86 (s, 2H), 3.99 (s, 2H), 4.31 (t, J = 5.6 Hz, 2H), 6.44 (s, 1H), 6.52 (s, 1H), 6.71- 6.78 (m, 1H), 7.08-7.14 (m, 2H), 7.47-7.55 (m, 2H), 8.42-8.45 (m, 1H), 8.47-8.50 (m, 1H). |
| 91 | 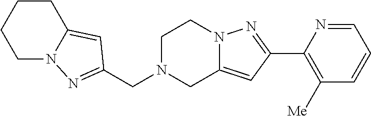 [SMILES: Cc1cccnc1-c1cc2n(n1)CCN(Cc1cc3n(n1)CCCC3)C2] | ¹H-NMR (400 MHz, CDCl₃) δ: 1.82-1.92 (m, 2H), 2.00-2.09 (m, 2H), 2.56 (s, 3H), 2.76 (t, J = 6.3 Hz, 2H), 2.94 (t, J = 5.6 Hz, 2H), 3.536 (s, 2H), 3.71 (s, 2H), 4.15 (t, J = 6.3 Hz, 2H), 4.24 (t, J = 5.6 Hz, 2H), 6.44 (s, 1H), 7.12 (dd, J = 7.5, 7.0 Hz, 1H), 7.43 (s, 1H), 7.53 (d, J = 7.0 Hz, 1H), 8.49 (d, J = 4.6 Hz, 1H). |
| 92 | 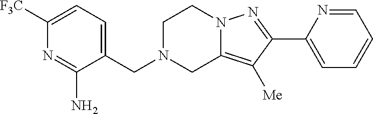 [SMILES: Cc1c(-c2ccccn2)nn2c1CN(Cc1ccc(C(F)(F)F)nc1N)CC2] | ¹H-NMR (400 MHz, CDCl₃) δ: 2.26 (3H, s), 2.95 (2H, t, J = 5.5 Hz), 3.66 (2H, s), 3.77 (2H, s), 4.24 (2H, t, J = 5.5 Hz), 5.79 (2H, s), 7.00 (1H, d, J = 7.3 Hz), 7.15-7.21 (1H, m), 7.44 (1H, d, J = 7.3 Hz), 7.70 (1H, ddd, J = 7.7, 7.7, 1.7 Hz), 7.81 (1H, d, J = 8.1 Hz), 8.64 (1H, d, J = 4.4 Hz). |
| 93 | 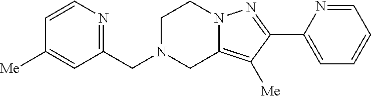 [SMILES: Cc1ccnc(CN2CCn3nc(-c4ccccn4)c(C)c3C2)c1] | ¹H-NMR (300 MHz, CDCl₃) δ: 2.24 (3H, s), 2.37 (3H, s), 3.03 (2H, t, J = 5.5 Hz), 3.72 (2H, s), 3.89 (2H, s), 4.26 (2H, t, J = 5.5 Hz), 7.05 (1H, d, J = 5.1 Hz), 7.13-7.19 (1H, m), 7.31 (1H, s), 7.68 (1H, ddd, J = 7.7, 7.7, 1.7 Hz), 7.81 (1H, d, J = 8.1 Hz), 8.45 (1H, d, J = 5.1 Hz), 8.63 (1H, d, J = 3.7 Hz). |
| 94 | 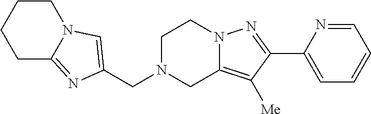 [SMILES: Cc1c(-c2ccccn2)nn2c1CN(Cc1cn3c(n1)CCCC3)CC2] | ¹H-NMR (300 MHz, CDCl₃) δ: 1.88-2.12 (4H, m), 2.23 (3H, s), 2.93-3.14 (4H, m), 3.72 (2H, s), 3.84 (2H, s), 4.00 (2H, brs), 4.22 (2H, brs), 6.86 (1H, s), 7.11-7.20 (1H, m), 7.62-7.73 (1H, m), 7.75-7.84 (1H, m), 8.62 (1H, brs). |
| 95 | 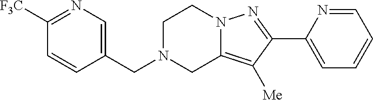 [SMILES: Cc1c(-c2ccccn2)nn2c1CN(Cc1ccc(C(F)(F)F)nc1)CC2] | ¹H-NMR (300 MHz, CDCl₃) δ: 2.24 (3H, s), 2.99 (2H, t, J = 5.5 Hz), 3.69 (2H, s), 3.87 (2H, s), 4.25 (2H, t, J = 5.5 Hz), 7.15-7.22 (1H, m), 7.66-7.75 (2H, m), 7.82 (1H, d, J = 7.3 Hz), 7.96 (1H, d, J = 7.3 Hz), 8.66 (1H, d, J = 4.4 Hz), 8.74 (1H, s). |
| 96 | 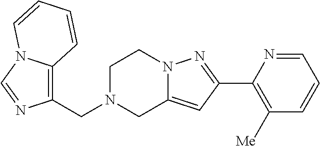 [SMILES: Cc1cccnc1-c1cc2n(n1)CCN(Cc1ncn3ccccc13)C2] | ¹H-NMR (400 MHz, CDCl₃) δ: 2.54 (s, 3H), 3.07 (t, J = 5.8 Hz, 2H), 3.84 (s, 2H), 4.07 (s, 2H), 4.28 (t, J = 5.8 Hz, 2H), 6.42 (s, 1H), 6.56 (t, J = 7.0 Hz, 1H), 6.68-6.74 (m, 1H), 7.11 (dd, J = 7.5 Hz, 7.5 Hz, 1H), 7.52 (d, J = 7.5 Hz, 1H), 7.56 (d, J = 9.0 Hz, 1H), 7.91 (d, J = 7.0 Hz, 1H), 8.09 (s, 1H), 8.46-8.50 (m, 1H). |
| 97 | 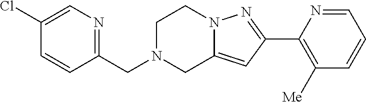 [SMILES: Cc1cccnc1-c1cc2n(n1)CCN(Cc1ccc(Cl)cn1)C2] | ¹H-NMR (400 MHz, CDCl₃) δ: 2.57 (s, 3H), 3.06 (t, J = Hz, 2H), 3.82 (s, 2H), 3.90 (s, 2H), 4.30 (t, J = 23.2 Hz, 2H), 6.45 (s, 1H), 7.12 (dd, J = 7.8, 7.5 Hz, 1H), 7.46 (d, J = 8.2 Hz, 1H), 7.53 (d, J = 7.5 Hz, 1H), 7.69 (dd, J = 8.2, 2.4 Hz, 1H), 8.47- 8.51 (m, 1H), 8.545 (d, J = 2.4 Hz, 1H). |
| 98 | 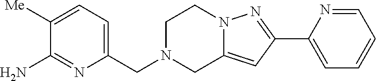 [SMILES: Cc1ccc(CN2CCn3nc(-c4ccccn4)cc3C2)nc1N] | ¹H-NMR (400 MHz, CDCl₃) δ: 2.14 (3H, s), 3.03 (2H, t, J = 5.6 Hz), 3.70 (2H, s), 3.81 (2H, s), 4.29 (2H, t, J = 5.6 Hz), 4.46 (2H, brs), 6.58 (1H, s), 6.72 (1H, d, J = 7.2 Hz), 7.16-7.19 (1H, m), 7.26 (1H, d, J = 7.2 Hz), 7.67-7.71 (1H, m), 7.88 (1H, J = 8.0 Hz), 8.61 (1H, J = 4.8 Hz). |
| 99 | 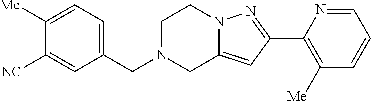 [SMILES: Cc1ccc(CN2CCn3nc(-c4ncccc4C)cc3C2)cc1C#N] | ¹H-NMR (400 MHz, CDCl₃) δ: 2.57 (3H, s), 2.60 (3H, s), 3.01 (2H, t, J = 7.6 Hz), 3.75 (2H, s), 3.76 (2H, s), 4.30 (2H, t, J = 7.6 Hz), 6.47 (1H, s), 7.12- 7.16 (1H, m), 7.31-7.35 (1H, m), 7.52-7.56 (2H, m), 7.65 (1H, s), 8.51 (1H, d, J = 6.4 Hz). |
| 100 | 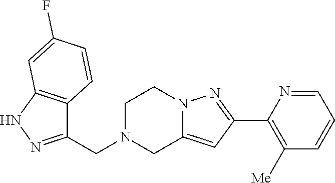 [SMILES: Cc1cccnc1-c1cc2n(n1)CCN(Cc1n[nH]c3cc(F)ccc13)C2] | ¹H-NMR (300 MHz, CDCl₃) δ: 2.54 (3H, s), 3.02 (2H, t, J = 5.7 Hz), 3.78 (2H, s), 4.10 (2H, s), 4.24 (2H, t, J = 5.7 Hz), 6.41 (1H, s), 6.84-6.91 (1H, m), 6.97-7.01 (1H, m), 7.09-7.14 (1H, m), 7.53 (1H, d, J = 8.1 Hz), 7.78-7.83 (1H, m), 8.48 (1H, d, J = 4.5 Hz). |
| 101 | 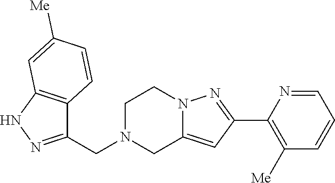 [SMILES: Cc1ccc2c(CN3CCn4nc(-c5ncccc5C)cc4C3)n[nH]c2c1] | ¹H-NMR (400 MHz, CDCl₃) δ: 2.50 (3H, s), 2.56 (3H, s), 3.06 (2H, t, J = 5.6 Hz), 3.87 (2H, s), 4.15 (2H, s), 4.27 (2H, t, J = 5.6 Hz), 6.44 (1H, s), 7.01 (1H, d, J = 8.4 Hz), 7.02-7.12 (1H, m), 7.25-7.26 (1H, m), 7.53 (1H, d, J = 8.0 Hz), 7.78 (1H, d, J = 8.0 Hz), 8.50 (1H, d, J = 4.0 Hz), 9.89 (1H, brs). |
| 102 | 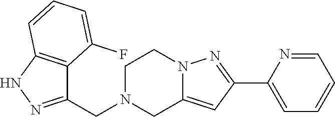 [SMILES: Fc1cccc2[nH]nc(CN3CCn4nc(-c5ccccn5)cc4C3)c12] | ¹H-NMR (400 MHz, CDCl₃) δ: 3.17 (2H, t, J = 5.6 Hz), 3.93 (2H, s), 4.24 (2H, s), 4.29 (2H, t, J = 5.6 Hz), 6.59 (1H, s), 6.80-6.85 (1H, m), 7.16-7.19 (1H, m), 7.24-7.36 (2H, m), 7.68-7.71 (1H, m), 7.87 (1H, d, J = 8.0 Hz), 8.61 (1H, d, J = 4.0 Hz), 10.2 (1H, brs). |
| 103 | 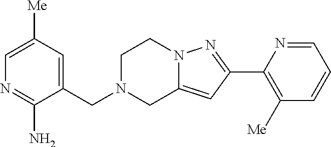 [SMILES: Cc1cnc(N)c(CN2CCn3nc(-c4ncccc4C)cc3C2)c1] | ¹H-NMR (400 MHz, CDCl₃) δ: 2.19 (3H, s), 2.56 (3H, s), 2.96 (2H, t, J = 7.6 Hz), 3.66 (2H, s), 3.73 (2H, s), 4.26 (2H, t, J = 7.6 Hz), 5.32 (2H, brs), 6.46 (1H, s), 7.08-7.14 (2H, m), 7.52 (1H, d, J = 9.2 Hz), 7.86 (1H, s), 8.48 (1H, d, J = 4.8 Hz). |
| 104 | 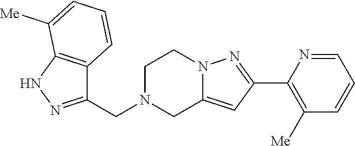 [SMILES: Cc1cccnc1-c1cc2n(n1)CCN(Cc1n[nH]c3c(C)cccc13)C2] | ¹H-NMR (400 MHz, CDCl₃) δ: 2.49 (3H, s), 2.53 (3H, s), 3.03 (2H, t, J = 5.4 Hz), 3.82 (2H, s), 4.14 (2H, s), 4.24 (2H, t, J = 5.4 Hz), 6.42 (1H, s), 7.03-7.15 (3H, m), 7.50 (1H, d, J = 7.6 Hz), 7.71 (1H, d, J = 8.0 Hz), 8.46 (1H, d, J = 5.2 Hz), 10.4 (1H, brs). |
| 105 | 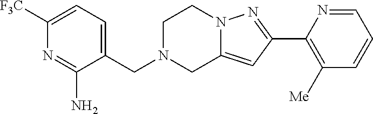 [SMILES: Cc1cccnc1-c1cc2n(n1)CCN(Cc1ccc(C(F)(F)F)nc1N)C2] | ¹H-NMR (400 MHz, CDCl₃) δ: 2.55 (3H, s), 2.96 (2H, t, J = 5.6 Hz), 3.73 (2H, s), 3.75 (2H, s), 4.27 (2H, t, J = 5.6 Hz), 5.77 (2H, brs), 6.47 (1H, s), 6.97 (1H, d, J = 7.2 Hz), 7.09-7.13 (1H, m), 7.41 (1H, d, J = 7.6 Hz), 7.52 (1H, d, J = 8.0 Hz), 8.46- 8.48 (1H, m). |

### Example 106

### 3-Chloro-2-(3-methylpyridin-2-yl)-5-[(5-methylpyridin-2-yl)methyl]-4,5,6,7-tetrahydropyrazolo[1,5-a]pyrazine

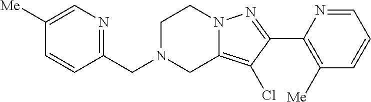 [SMILES: Cc1ccc(CN2CCn3nc(-c4ncccc4C)c(Cl)c3C2)nc1]

A mixture of the compound of Reference Example 48 (0.063 g, 0.253 mmol), 2-(chloromethyl)-5-methylpyridine monohydrochloride (0.050 g, 0.281 mmol), tetrabutylammonium bromide (0.008 g, 0.0248 mmol), 50% aqueous potassium carbonate solution (0.280 g), and tetrahydrofuran (3.0 mL) was stirred at 80° C. overnight. The reaction mixture was then diluted with water, and extracted with ethyl acetate. The combined organic layer was dried over sodium sulfate, filtered, and concentrated. The residue was purified by silica gel chromatography (chloroform:methanol=9:1) to give the title compound (0.058 g, 64%).

¹H-NMR (400 MHz, CDCl₃) δ: 2.34 (3H, s), 2.36 (3H, s), 3.03 (2H, t, J=5.5 Hz), 3.74 (2H, s), 3.90 (2H, s), 4.23 (2H, t, J=5.5 Hz), 7.20 (1H, dd, J=7.6, 4.8 Hz), 7.32-7.34 (1H, m), 7.51 (1H, dd, J=8.0, 1.6 Hz), 7.56 (1H, dd, J=7.8, 0.9 Hz), 8.43-8.43 (1H, m), 8.52-8.53 (1H, m).

### Examples 107-139

The compounds of Examples 107-139 were synthesized from the corresponding compounds of each Reference Example according to the process of Example 106.

| | | Instrumental Analysis |
|---|---|---|
| Example | Chemical Structure | Data |
| 107 | 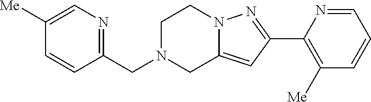 [SMILES: Cc1ccc(CN2CCn3nc(-c4ncccc4C)cc3C2)nc1] | ¹H-NMR (400 MHz, CDCl₃) δ: 2.35 (s, 3H), 2.56 (s, 3H), 3.05 (t, J = 5.6 Hz, 2H), 3.81 (s, 2H), 3.88 (s, 2H), 4.29 (t, J = 5.6 Hz, 2H), |
| | | 6.43 (s, 1H), 7.09- |
| | | 7.14 (m, 1H), 7.33- |
| | | 7.38 (m, 1H), 7.48- |
| | | 7.55 (m, 2H), 8.41- |
| | | 8.45 (m, 1H), 8.47- |
| | | 8.51 (m, 1H). |
| 108 | 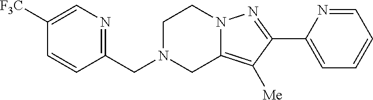 [SMILES: Cc1c(-c2ccccn2)nn2c1CN(Cc1ccc(C(F)(F)F)cn1)CC2] | ¹H-NMR (400 MHz, CDCl₃) δ: 2.25 (s, 3H), 3.05 (t, J = 5.6 Hz, 2H), 3.70-3.81 (m, 2H), 3.97-4.06 (m, 2H), 4.27 (t, J = 5.6 Hz, |
| | | 3H), 7.11-7.23 (m, |
| | | 1H), 7.63-7.74 (m, |
| | | 1H), 7.79-7.86 (m, |
| | | 1H), 7.93-7.99 (m, |
| | | 1H), 8.62-8.67 (m, |
| | | 1H), 8.87 (s, 1H). |
| 109 | 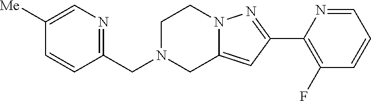 [SMILES: Cc1ccc(CN2CCn3nc(-c4ncccc4F)cc3C2)nc1] | ¹H-NMR (400 MHz, CDCl₃) δ: 2.35 (s, 3H), 3.05 (t, J = 5.6 Hz, 2H), 3.82 (s, 2H), 3.88 (s, 2H), 4.35 (t, J = 5.6 Hz, 2H), 6.57-6.61 |
| | | (m, 1H), 7.19-7.25 |
| | | (m, 1H), 7.33-7.37 |
| | | (m, 1H), 7.42-7.48 |
| | | (m, 1H), 7.49-7.53 |
| | | (m, 1H), 8.40-8.45 |
| | | (m, 1H), 8.47-8.53 |
| | | (m, 1H). |
| 110 | 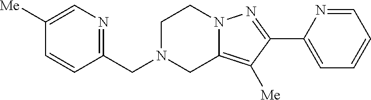 [SMILES: Cc1ccc(CN2CCn3nc(-c4ccccn4)c(C)c3C2)nc1] | ¹H-NMR (400 MHz, CDCl₃) δ: 2.23 (3H, s), 2.35 (3H, s), 3.02 (2H, t, J = 5.5 Hz), 3.70 (2H, s), 3.89 (2H, s), 4.24 (2H, t, J = 5.7 Hz), |
| | | 7.15-7.16 (1H, m), |
| | | 7.35-7.37 (1H, m), |
| | | 7.51 (1H, dd, J = 7.6, |
| | | 2.1 Hz), 7.68 (1H, td, |
| | | J = 7.8, 1.8 Hz), 7.81 |
| | | (1H, dd, J = 6.9, 0.9 |
| | | Hz), 8.43-8.43 (1H, |
| | | m), 8.63-8.64 (1H, |
| | | m). |
| 111 | 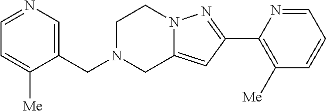 [SMILES: Cc1ccncc1CN1CCn2nc(-c3ncccc3C)cc2C1] | ¹H-NMR (400 MHz, CDCl₃) δ: 2.42 (s, 3H), 2.57 (s, 3H), 2.99 (t, J = 5.6 Hz, 2H), 3.73 (s, 2H), 3.75 (s, 2H), 4.24 (t, J = 5.6 Hz, 2H), 6.45 (s, 1H), 7.10- |
| | | 7.15 (m, 2H), 7.51- |
| | | 7.56 (m, 1H), 8.41- |
| | | 8.47 (m, 2H), 8.47- |
| | | 8.51 (m, 1H). |
| 112 | 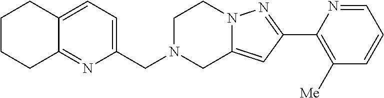 [SMILES: Cc1cccnc1-c1cc2n(n1)CCN(Cc1ccc3c(n1)CCCC3)C2] | ¹H-NMR (400 MHz, CDCl₃) δ: 1.81-1.89 (4H, m), 2.55 (3H, s), 2.76 (2H, t, J = 6.0 Hz), 2.92 (2H, t, J = 6.2 Hz), 3.03 (2H, t, J = 5.5 |
| | | Hz), 3.82 (4H, d, J = |
| | | 13.2 Hz), 4.27 (2H, t, |
| | | J = 5.5 Hz), 6.43 (1H, |
| | | s), 7.10 (1H, dd, J = |
| | | 7.6, 4.9 Hz), 7.21 (1H, |
| | | d, J = 7.8 Hz), 7.36 |
| | | (1H, d, J = 7.8 Hz), |
| | | 7.51 (1H, d, J = 7.6 |
| | | Hz), 8.48 (1H, d, J = |
| | | 4.4 Hz). |
| 113 | 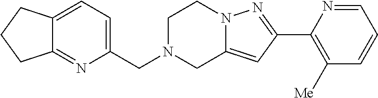 [SMILES: Cc1cccnc1-c1cc2n(n1)CCN(Cc1ccc3c(n1)CCC3)C2] | ¹H-NMR (400 MHz, CDCl₃) δ: 2.13-2.17 (2H, m), 2.56 (3H, s), 2.94 (2H, t, J = 7.3 Hz), 3.02- 3.06 (4H, m), 3.82 (2H, s), 3.87 (2H, s), 4.29 |
| | | (2H, t, J = 5.5 Hz), |
| | | 6.44 (1H, s), 7.11 (1H, |
| | | dd, J = 7.6, 4.8 Hz), |
| | | 7.21 (1H, d, J = 7.8 |
| | | Hz), 7.50-7.53 (2H, |
| | | m), 8.49 (1H, dd, J = |
| | | 4.6, 1.4 Hz). |
| 114 | 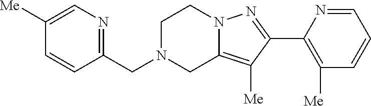 [SMILES: Cc1ccc(CN2CCn3nc(-c4ncccc4C)c(C)c3C2)nc1] | ¹H-NMR (400 MHz, CDCl₃) δ: 1.94 (3H, s), 2.36 (3H, s), 2.38 (3H, s), 3.04 (2H, t, J = 5.5 Hz), 3.72 (2H, s), 3.92 (2H, s), 4.24 (2H, t, J = 5.5 Hz), 7.13-7.22 |
| | | (1H, m), 7.39 (1H, d, J = |
| | | 7.8 Hz), 7.54 (1H, d, |
| | | J = 7.8 Hz), 7.59 (1H, |
| | | d, J = 7.8 Hz), 8.44 |
| | | (1H, s), 8.51 (1H, d, J = |
| | | 3.9 Hz). |
| 115 | 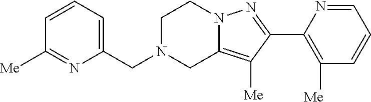 [SMILES: Cc1cccc(CN2CCn3nc(-c4ncccc4C)c(C)c3C2)n1] | ¹H-NMR (400 MHz, CDCl₃) δ: 1.94 (3H, s), 2.38 (3H, s), 2.60 (3H, s), 3.04 (2H, t, J = 5.5 Hz), 3.74 (2H, s), 3.93 (2H, s), 4.24 (2H, t, J = |
| | | 5.5 Hz), 7.10 (1H, d, |
| | | J = 7.8 Hz), 7.12- |
| | | 7.20 (1H, m), 7.33 (1H, |
| | | d, J = 7.8 Hz), 7.53- |
| | | 7.67 (2H, m), 8.50 (1H, |
| | | d, J = 4.2 Hz). |
| 116 | 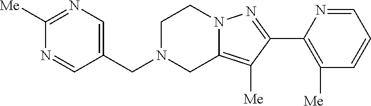 [SMILES: Cc1ncc(CN2CCn3nc(-c4ncccc4C)c(C)c3C2)cn1] | ¹H-NMR (400 MHz, CDCl₃) δ: 1.96 (3H, s), 2.40 (3H, s), 2.77 (3H, s), 2.98 (2H, t, J = 5.5 Hz), 3.67 (2H, s), 3.75 (2H, s), 4.22 (2H, t, J = |
| | | 5.5 Hz), 7.15-7.25 |
| | | (1H, m), 7.55-7.73 |
| | | (1H, m), 8.50-8.58 |
| | | (1H, m), 8.68 (2H, s). |
| 117 | 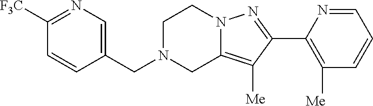 [SMILES: Cc1cccnc1-c1nn2c(c1C)CN(Cc1ccc(C(F)(F)F)nc1)CC2] | ¹H-NMR (400 MHz, CDCl₃) δ: 1.97 (3H, s), 2.41 (3H, s), 3.00 (2H, t, J = 5.5 Hz), 3.69 (2H, s), 3.88 (2H, s), 4.23 (2H, t, J = 5.5 Hz), 7.15-7.30 (1H, m), |
| | | 7.63 (1H, brs), 7.71 |
| | | (1H, d, J = 7.9 Hz), |
| | | 7.97 (1H, d, J = 7.9 |
| | | Hz), 8.53 (1H, s), 8.75 |
| | | (1H, s). |
| 118 | 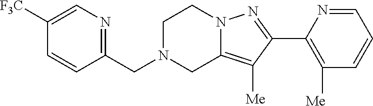 [SMILES: Cc1cccnc1-c1nn2c(c1C)CN(Cc1ccc(C(F)(F)F)cn1)CC2] | ¹H-NMR (400 MHz, CDCl₃) δ: 1.96 (3H, s), 2.40 (3H, s), 3.06 (2H, t, J = 5.5 Hz), 3.75 (2H, s), 4.02 (2H, s), 4.26 (2H, t, J = 5.5 Hz), |
| | | 7.15-7.25 (1H, m), |
| | | 7.55-7.67 (1H, m), |
| | | 7.68 (1H, d, J = 8.3 |
| | | Hz), 7.96 (1H, dd, J = |
| | | 8.3, 2.2 Hz), 8.53 (1H, |
| | | d, J = 4.6 Hz), 8.87 |
| | | (1H, s). |
| 119 | 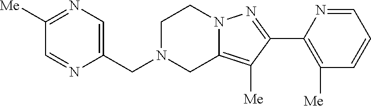 [SMILES: Cc1cnc(CN2CCn3nc(-c4ncccc4C)c(C)c3C2)cn1] | ¹H-NMR (400 MHz, CDCl₃) δ: 1.95 (3H, s), 2.38 (3H, s), 2.60 (3H, s), 3.05 (2H, t, J = 5.5 Hz), 3.74 (2H, s), 3.94 (2H, s), 4.24 (2H, t, J = 5.5 Hz), 7.13-7.24 |
| | | (1H, m), 7.60 (1H, d, J = |
| | | 7.3 Hz), 8.47 (1H, |
| | | s), 8.52 (1H, d, J = |
| | | 4.6 Hz), 8.62 (1H, d, J = |
| | | 1.2 Hz). |
| 120 | 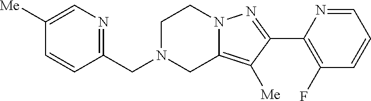 [SMILES: Cc1ccc(CN2CCn3nc(-c4ncccc4F)c(C)c3C2)nc1] | ¹H-NMR (400 MHz, CDCl₃) δ: 2.04 (s, 3H), 2.36 (s, 3H), 3.03 (t, J = 5.6 Hz, 2H), 3.71 (s, 2H), 3.90 (s, 2H), 4.28 (t, J = 5.6 Hz, 2H), |
| | | 7.23-7.29 (m, 1H), 7.37 |
| | | (d, J = 7.8 Hz, 1H), |
| | | 7.44-7.48 (m, 1H), |
| | | 7.48-7.53 (m, 1H), |
| | | 8.41-8.44 (m, 1H), |
| | | 8.49-8.52 (m, 1H). |
| 121 | 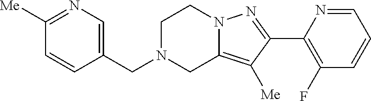 [SMILES: Cc1ccc(CN2CCn3nc(-c4ncccc4F)c(C)c3C2)cn1] | ¹H-NMR (400 MHz, CDCl₃) δ: 2.03-2.04 (m, 3H), 2.57 (s, 3H), 2.96 (t, J = 5.6 Hz, 2H), 3.65 (s, 2H), 3.74 (s, 2H), 4.25 (t, J = 5.6 Hz, |
| | | 2H), 7.18 (d, J = 7.8 |
| | | Hz, 1H), 7.24-7.28 (m, |
| | | 1H), 7.44-7.51 (m, |
| | | 1H), 7.62-7.66 (m, |
| | | 1H), 8.46-8.49 (m, |
| | | 1H), 8.49-8.52 (m, |
| | | 1H). |
| 122 | 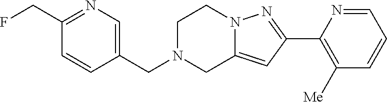 [SMILES: Cc1cccnc1-c1cc2n(n1)CCN(Cc1ccc(CF)nc1)C2] | ¹H-NMR (400 MHz, CDCl₃) δ: 2.56 (3H, s), 3.01 (2H, t, J = 5.6 Hz), 3.76-3.81 (4H, m), 4.28 (2H, t, J = 5.6 Hz), 5.50 (2H, d, J = |
| | | 47.0 Hz), 6.46 (1H, s), |
| | | 7.10-7.15 (1H, m), |
| | | 7.45-7.57 (2H, m), |
| | | 7.80-7.86 (1H, m), |
| | | 8.47-8.52 (1H, m), |
| | | 8.58 (1H, s). |
| 123 | 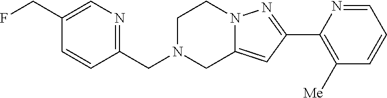 [SMILES: Cc1cccnc1-c1cc2n(n1)CCN(Cc1ccc(CF)cn1)C2] | ¹H-NMR (400 MHz, CDCl₃) δ: 2.58 (3H, s), 3.07 (2H, t, J = 5.5 Hz), 3.83 (2H, s), 3.94 (2H, s), 4.31 (2H, t, J = 5.5 Hz), 5.43 (2H, d, J = |
| | | 47.2 Hz), 6.51 (1H, |
| | | s), 7.22-7.31 (1H, |
| | | m), 7.50-7.65 (2H, |
| | | m), 7.73-7.82 (1H, |
| | | m), 8.46-8.55 (1H, |
| | | m), 8.59-8.64 (1H, |
| | | m). |
| 124 | 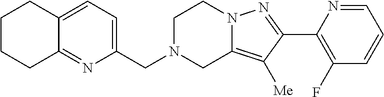 [SMILES: Cc1c(-c2ncccc2F)nn2c1CN(Cc1ccc3c(n1)CCCC3)CC2] | ¹H-NMR (400 MHz, CDCl₃) δ: 1.78-1.87 (m, 2H), 1.87-1.95 (m, 2H), 2.04 (s, 3H), 2.77 (t, J = 6.3 Hz, 2H), 2.94 (t, J = 6.3 Hz, 2H), |
| | | 3.02 (t, J = 5.6 Hz, |
| | | 2H), 3.73 (s, 2H), 3.86 |
| | | (s, 2H), 4.27 (t, J = |
| | | 5.6 Hz, 2H), 7.22 (d, J = |
| | | 7.8 Hz, 1H), 7.24- |
| | | 7.28 (m, 1H), 7.37 (d, |
| | | J = 7.8 Hz, 1H), 7.44- |
| | | 7.50 (m, 1H), 8.49- |
| | | 8.52 (m, 1H). |
| 125 | 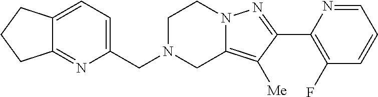 [SMILES: Cc1c(-c2ncccc2F)nn2c1CN(Cc1ccc3c(n1)CCC3)CC2] | ¹H-NMR (400 MHz, CDCl₃) δ: 2.01-2.06 (m, 3H), 2.10-2.22 (m, 2H), 2.94 (t, J = 7.3 Hz, 2H), 3.00-3.08 (m, 4H), 3.72 (s, 2H), 3.89 |
| | | (s, 2H), 4.28 (t, J = |
| | | 5.6 Hz, 2H), 7.21 (d, J = |
| | | 7.6 Hz, 1H), 7.23- |
| | | 7.29 (m, 1H), 7.44- |
| | | 7.48 (m, 1H), 7.48- |
| | | 7.53 (m, 1H), 8.49- |
| | | 8.52 (m, 1H). |
| 126 | 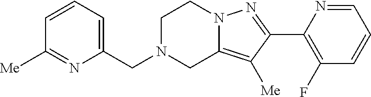 [SMILES: Cc1cccc(CN2CCn3nc(-c4ncccc4F)c(C)c3C2)n1] | ¹H-NMR (400 MHz, CDCl₃) δ: 2.02-2.07 (m, 3H), 2.58 (s, 3H), 3.00- 3.07 (m, 2H), 3.74 (s, 2H), 3.90 (s, 2H), 4.25- 4.31 (m, 2H), 7.06- |
| | | 7.10 (m, 1H), 7.44- |
| | | 7.51 (m, 1H), 7.51- |
| | | 7.56 (m, 2H), 7.56- |
| | | 7.62 (m, 1H), 8.49- |
| | | 8.57 (m, 1H). |
| 127 | 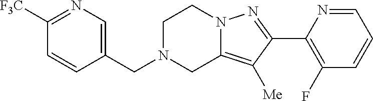 [SMILES: Cc1c(-c2ncccc2F)nn2c1CN(Cc1ccc(C(F)(F)F)nc1)CC2] | ¹H-NMR (400 MHz, CDCl₃) δ: 2.04 (s, 3H), 3.00 (t, J = 5.6 Hz, 2H), 3.69 (s, 2H), 3.88 (s, 2H), 4.28 (t, J = 5.6 Hz, 2H), 7.27-7.30 |
| | | (m, 1H), 7.45-7.53 |
| | | (m, 1H), 7.69-7.74 |
| | | (m, 1H), 7.94-7.98 |
| | | (m, 1H), 8.49-8.53 |
| | | (m, 1H), 8.71-8.75 |
| | | (m, 1H). |
| 128 | 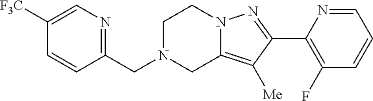 [SMILES: Cc1c(-c2ncccc2F)nn2c1CN(Cc1ccc(C(F)(F)F)cn1)CC2] | ¹H-NMR (400 MHz, CDCl₃) δ: 2.06 (s, 3H), 3.07 (t, J = 5.6 Hz, 2H), 3.75 (s, 2H), 4.02 (s, 2H), 4.31 (t, J = 5.6 Hz, 2H), 7.27-7.30 |
| | | (m, 1H), 7.45-7.52 |
| | | (m, 1H), 7.66-7.70 |
| | | (m, 1H), 7.93-7.99 |
| | | (m, 1H), 8.49-8.53 |
| | | (m, 1H), 8.84-8.88 |
| | | (m, 1H). |
| 129 | 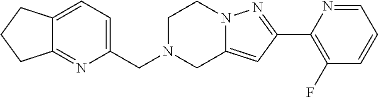 [SMILES: Fc1cccnc1-c1cc2n(n1)CCN(Cc1ccc3c(n1)CCC3)C2] | ¹H-NMR (400 MHz, CDCl₃) δ: 2.94 (2H, t, J = 7.6 Hz), 3.02-3.06 (4H, m), 3.82 (2H, s), 3.87 (2H, s), 4.00-4.03 (2H, m), 4.34 (2H, t, J = |
| | | 5.5 Hz), 6.59 (1H, d, |
| | | J = 3.7 Hz), 7.20- |
| | | 7.22 (2H, m), 7.45- |
| | | 7.48 (2H, m), 8.49- |
| | | 8.50 (1H, m). |
| 130 | 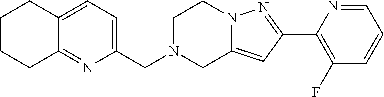 [SMILES: Fc1cccnc1-c1cc2n(n1)CCN(Cc1ccc3c(n1)CCCC3)C2] | ¹H-NMR (400 MHz, CDCl₃) δ: 1.76-1.91 (4H, m), 2.75 (2H, t, J = 6.0 Hz), 2.91 (2H, t, J = 6.4 Hz), 3.02 (2H, t, J = 5.5 Hz), 3.81 (4H, d, |
| | | J = 11.5 Hz), 4.31 (2H, |
| | | t, J = 5.5 Hz), 6.57 |
| | | (1H, d, J = 3.7 Hz), |
| | | 7.16-7.21 (2H, m), |
| | | 7.34-7.35 (1H, m), |
| | | 7.41-7.44 (1H, m), |
| | | 8.47-8.48 (1H, m). |
| 131 | 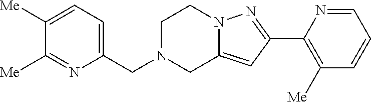 [SMILES: Cc1ccc(CN2CCn3nc(-c4ncccc4C)cc3C2)nc1C] | ¹H-NMR (400 MHz, CDCl₃) δ: 2.29 (3H, s), 2.52 (3H, s), 2.57 (3H, s), 3.05 (2H, t, J = 5.5 Hz), 3.82 (2H, s), 3.85 (2H, s), 4.29 (2H, t, J = |
| | | 5.7 Hz), 6.44 (1H, |
| | | s), 7.12 (1H, dd, J = |
| | | 7.8, 4.6 Hz), 7.21- |
| | | 7.23 (1H, m), 7.41-7.43 |
| | | (1H, m), 7.52-7.54 |
| | | (1H, m), 8.49 (1H, dd, |
| | | J = 4.8, 1.1 Hz). |
| 132 | 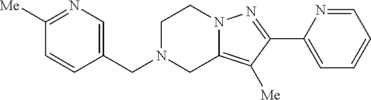 [SMILES: Cc1ccc(CN2CCn3nc(-c4ccccn4)c(C)c3C2)cn1] | ¹H-NMR (400 MHz, CDCl₃) δ: 2.23 (s, 3H), 2.35 (s, 3H), 3.01 (t, J = 5.6 Hz, 2H), 3.71 (s, 2H), 3.89 (s, 2H), 4.24 (t, J = 5.6 Hz, 2H), |
| | | 7.13-7.18 (m, 1H), |
| | | 7.36 (d, J = 8.0 Hz, |
| | | 1H), 7.51 (d, J = 7.8 |
| | | Hz, 1H), 7.68 (td, J = |
| | | 7.8, 1.9 Hz, 1H), 7.81 |
| | | (d, J = 8.0 Hz, 1H), |
| | | 8.41-8.45 (m, 1H), |
| | | 8.61-8.65 (m, 1H). |
| 133 | 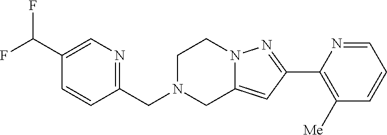 [SMILES: Cc1cccnc1-c1cc2n(n1)CCN(Cc1ccc(C(F)F)cn1)C2] | ¹H-NMR (400 MHz, CDCl₃) δ: 2.56 (s, 3H), 3.07 (t, J = 5.6 Hz, 2H), 3.84 (s, 2H), 3.97 (s, 2H), 4.31 (t, J = 5.6 Hz, 2H), 6.46 (s, 1H), 6.73 (t, J = 55.8 Hz, 1H), 7.12 (dd, J = 7.5, |
| | | 4.9 Hz, 1H), 7.53 (d, J = |
| | | 7.5 Hz, 1H), 7.62 (d, |
| | | J = 8.0 Hz, 1H), 7.87 |
| | | (d, J = 8.0 Hz, 1H), |
| | | 8.47-8.51 (m, 1H), |
| | | 8.69-8.75 (m, 1H). |
| 134 | 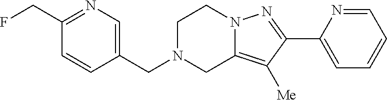 [SMILES: Cc1c(-c2ccccn2)nn2c1CN(Cc1ccc(CF)nc1)CC2] | ¹H-NMR (400 MHz, CDCl₃) δ: 2.24 (s, 3H), 2.97 (t, J = 5.6 Hz, 2H), 3.67 (s, 2H), 3.79 (s, 2H), 4.23 (t, J = 5.6 Hz, 2H), 5.52 (d, J = |
| | | 47.0 Hz, 2H),7.14- |
| | | 7.20 (m, 1H), 7.48 (d, |
| | | J = 8.0 Hz, 1H), 7.69 |
| | | (td, J = 7.5, 1.9 Hz, |
| | | 1H), 7.78-7.85 (m, |
| | | 2H), 8.56-8.59 (m, |
| | | 1H), 8.62-8.66 (m, |
| | | 1H). |
| 135 | 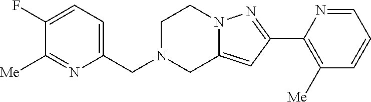 [SMILES: Cc1cccnc1-c1cc2n(n1)CCN(Cc1ccc(F)c(C)n1)C2] | ¹H-NMR (400 MHz, CDCl₃) δ: 2.54 (3H, d, J = 2.8 Hz), 2.57 (3H, s), 3.05 (2H, t, J = 5.5 Hz), 3.81 (2H, s), 3.85 (2H, s), 4.29 (2H, t, J = |
| | | 5.5 Hz), 6.45 (1H, s), |
| | | 7.12 (1H, dd, J = 7.6, |
| | | 4.8 Hz), 7.33 (2H, d, J = |
| | | 6.4 Hz), 7.53 (1H, d, |
| | | J = 7.8 Hz), 8.49 (1H, |
| | | d, J = 4.1 Hz). |
| 136 | 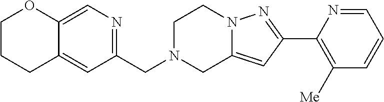 [SMILES: Cc1cccnc1-c1cc2n(n1)CCN(Cc1cc3c(cn1)OCCC3)C2] | ¹H-NMR (400 MHz, CDCl₃) δ: 2.00-2.08 (m, 2H), 2.56 (s, 3H), 2.79 (t, J = 6.3 Hz, 2H), 3.05 (t, J = 5.6 Hz, 2H), 3.77-3.81 (m, 4H), |
| | | 4.23 (t, J = 5.3 Hz, |
| | | 2H), 4.29 (t, J = 5.6 |
| | | Hz, 2H), 6.43 (s, 1H), |
| | | 7.08-7.14 (m, 2H), |
| | | 7.50-7.55 (m, 1H), |
| | | 8.13 (s, 1H), 8.47- |
| | | 8.51 (m, 1H). |
| 137 | 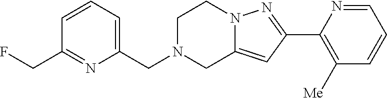 [SMILES: Cc1cccnc1-c1cc2n(n1)CCN(Cc1cccc(CF)n1)C2] | ¹H-NMR (400 MHz, CDCl₃) δ: 2.57 (3H, s), 3.07 (2H, t, J = 5.4 Hz), 3.83 (2H, s), 3.91 (2H, s), 4.30 (2H, t, J = 5.4 Hz), 5.45 (1H, s), |
| | | 5.56 (1H, s), 6.46 (1H, |
| | | s), 7.10-7.13 (1H, |
| | | m), 7.39 (1H, d, J = |
| | | 7.8 Hz), 7.45 (1H, d, J = |
| | | 7.8 Hz), 7.53-7.55 |
| | | (1H, m), 7.75-7.79 |
| | | (1H, m), 8.49 (1H, dd, |
| | | J = 4.6, 1.2 Hz). |
| 138 | 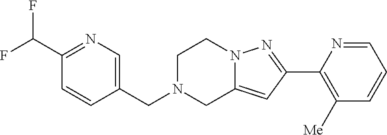 [SMILES: Cc1cccnc1-c1cc2n(n1)CCN(Cc1ccc(C(F)F)nc1)C2] | ¹H-NMR (400 MHz, CDCl₃) δ: 2.57 (3H, s), 3.01 (2H, t, J = 5.4 Hz), 3.78 (2H, s), 3.82 (2H, s), 4.29 (2H, t, J = 5.4 Hz), 6.47 (1H, s), 6.66 (1H, t, J = 55.4 Hz), 7.11-7.14 (1H, |
| | | m), 7.53-7.55 (1H, |
| | | m), 7.64-7.67 (1H, |
| | | m), 7.91 (1H, dd, J = |
| | | 8.0, 2.0 Hz), 8.49- |
| | | 8.50 (1H, m), 8.65 (1H, |
| | | s). |
| 139 | 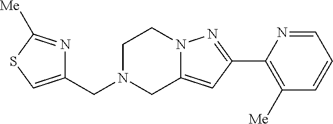 [SMILES: Cc1nc(CN2CCn3nc(-c4ncccc4C)cc3C2)cs1] | ¹H-NMR (400 MHz, CDCl₃) δ: 2.56 (3H, s), 2.73 (3H, s), 3.07 (2H, t, J = 5.5 Hz), 3.83 (2H, s), 3.86 (2H, s), 4.30 (2H, t, J = 5.5 Hz), 6.45 (1H, s), 7.05 (1H, s), 7.10-7.13 (1H, |
| | | m), 7.52-7.54 (1H, |
| | | m), 8.49 (1H, dd, J = |
| | | 4.6, 1.2 Hz). |

### Example 140

### 5-Benzyl-2-[3-(trifluoromethyl)pyridin-2-yl]-4,5,6,7-tetrahydropyrazolo[1,5-a]pyrazine

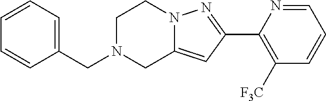 [SMILES: FC(F)(F)c1cccnc1-c1cc2n(n1)CCN(Cc1ccccc1)C2]

To a solution of the compound of Reference Example 31 (100 mg, 0.328 mmol) in methanol (1.5 mL) were added triethylamine (0.137 mL, 0.984 mmol) and then benzaldehyde (52.2 mg, 0.492 mmol). The mixture was stirred at room temperature for 30 minutes, and sodium cyanoborohydride (61.8 mg, 0.984 mmol) was added thereto. The mixture was stirred at room temperature for 12 hours, and methanol was removed from the reaction mixture. The residue was purified by preparative HPLC to give the title compound (22%).

¹H-NMR (400 MHz, CDCl₃) δ: 3.07 (2H, brs), 3.82 (4H, brs), 4.35 (2H, brs), 6.42 (1H, s), 7.27 (1H, s), 7.29-7.53 (5H, m), 8.06 (1H, d, J=6.4 Hz), 8.85 (1H, d, J=4.4 Hz).

### Examples 141-142

The compounds of Examples 141-142 were synthesized from the corresponding compounds of each Reference Example according to the process of Example 140.

| | | Instrumental Analysis |
|---|---|---|
| Example | Chemical Structure | Data |
| 141 | 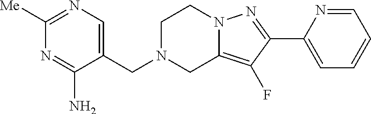 [SMILES: Cc1ncc(CN2CCn3nc(-c4ccccn4)c(F)c3C2)c(N)n1] | ¹H-NMR (400 MHz, CDCl₃) δ: 2.52 (3 H, s), 2.97 (2H, t, J = 5.4 Hz), 3.70 (2H, s), 3.74 (2H, s), 4.23 (2H, t, J = 5.4 Hz), 5.82 (2H, brs), 7.23 (1H, dd, J = |
| | | 6.0, 6.0 Hz), 7.74 (1H, |
| | | dd, J = 7.6, 7.6 Hz), |
| | | 7.78 (1H, dd, J = 8.0, |
| | | 8.0 Hz), 8.03 (1H, |
| | | s), 8.70 (1H, d, J = |
| | | 4.4 Hz). |
| 142 | 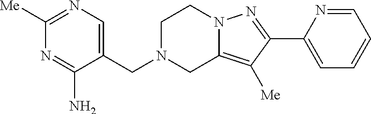 [SMILES: Cc1ncc(CN2CCn3nc(-c4ccccn4)c(C)c3C2)c(N)n1] | ¹H-NMR (400 MHz, CDCl₃) δ: 2.25 (3H, s), 2.53 (3H, s), 2.96 (2H, t, J = 5.4 Hz), 3.64 (2H, s), 3.69 (2H, s), 4.24 (2H, t, J = 5.4 Hz), 5.90 (2H, brs), 7.18 |
| | | (1H, dd, J = 6.4, 6.4 |
| | | Hz), 7.70 (1H, dd, J = |
| | | 7.6, 7.6 Hz), 7.80 |
| | | (1H, d, J = 8.0 Hz), 8.03 |
| | | (1H, s), 8.64 (1H, |
| | | d, J = 4.8 Hz). |

### Example 143

### 2-(3-Methylpyridin-2-yl)-5-{[6-(trifluoromethyl)pyridin-3-yl]methyl}-4,5,6,7-tetrahydropyrazolo[1,5-a]pyrazine

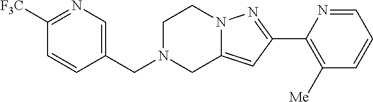 [SMILES: Cc1cccnc1-c1cc2n(n1)CCN(Cc1ccc(C(F)(F)F)nc1)C2]

To a solution of the compound of Reference Example 12 (100 mg, 0.467 mmol) in dichloroethane (2.0 mL) were added 6-(trifluoromethyl)pyridine-3-carboxyaldehyde (123 mg, 0.701 mmol), triethylamine (130 mL, 0.934 mmol), and then sodium triacetoxyborohydride (248 mg, 1.17 mmol). The mixture was stirred at 50° C. for 12 hours, and the reaction mixture was concentrated. The residue was purified by preparative HPLC to give the title compound (34.9 mg, 20%).

¹H-NMR (400 MHz, CDCl₃) δ: 2.59 (3H, s), 3.02 (2H, t, J=5.4 Hz), 3.77 (2H, s), 3.78 (2H, s), 4.30 (2H, t, J=5.4 Hz), 6.48 (1H, s), 6.97 (1H, d, J=8.4 Hz), 7.11-7.17 (1H, m), 7.56 (1H, d, J=7.6 Hz), 7.84-7.93 (1H, m), 8.21 (1H, s), 8.51 (1H, d, J=3.2 Hz).

### Examples 144-171

The compounds of Examples 144-171 were synthesized from the corresponding compounds of each Reference Example according to the process of Example 143.

| | | Instrumental Analysis |
|---|---|---|
| Example | Chemical Structure | Data |
| 144 | 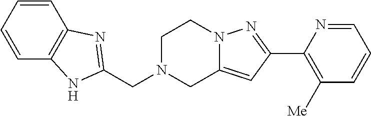 [SMILES: Cc1cccnc1-c1cc2n(n1)CCN(Cc1nc3ccccc3[nH]1)C2] | ¹H-NMR (400 MHz, CDCl₃) δ: 2.57 (3H, s), 3.01 (2H, t, J = 5.6 Hz), 3.74 (2H, s), 3.97 (2H, s), 4.20 (2H, t, J = 5.6 Hz), 6.44 (1H, s), |
| | | 7.10-7.17 (1H, m), |
| | | 7.20-7.30 (2H, m), |
| | | 7.47-7.77 (3H, m), |
| | | 8.47 (1H, d, J = 4.0 |
| | | Hz). |
| 145 | 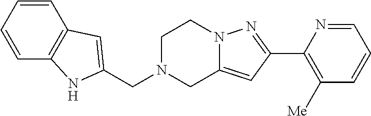 [SMILES: Cc1cccnc1-c1cc2n(n1)CCN(Cc1cc3ccccc3[nH]1)C2] | ¹H-NMR (400 MHz, DMSO-d₆) δ: 2.58 (3H, s), 3.01 (2H, t, J = 5.4 Hz), 3.74 (2H, s), 3.90 (2H, s), 4.20 (2H, t, J = 5.4 Hz), 6.39 (1H, s), |
| | | 6.57 (1H, s), 6.91- |
| | | 7.01 (1H, m), 7.01- |
| | | 7.09 (1H, m), 7.17- |
| | | 7.25 (1H, m), 7.34 |
| | | (1H, d, J = 8.0 Hz), |
| | | 7.48 (1H, d, J = 7.6 |
| | | Hz), 7.65 (1H, d, J = |
| | | 7.2 Hz), 8.42 (1H, d, |
| | | J = 4.0 Hz), 11.1 |
| | | (1H, s). |
| 146 | 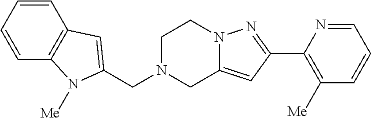 [SMILES: Cc1cccnc1-c1cc2n(n1)CCN(Cc1cc3ccccc3n1C)C2] | ¹H-NMR (400 MHz, CDCl₃) δ: 2.59 (3H, s), 3.03 (2H, t, J = 5.4 Hz), 3.81 (2H, s), 3.84 (3H, s), 3.91 (2H, s), 4.26 (2H, t, J = 5.4 Hz), |
| | | 6.47 (1H, s), 6.49 |
| | | (1H, s), 7.14 (2H, |
| | | dd, J = 6.0, 5.2 Hz), |
| | | 7.26 (1H, dd, J = |
| | | 5.0, 5.0 Hz), 7.35 |
| | | (1H, d, J = 3.4 Hz), |
| | | 7.55 (1H, d, J = 5.4 |
| | | Hz), 7.62 (1H, d, J = |
| | | 7.6 Hz), 8.52 (1H, d, |
| | | J = 4.0 Hz). |
| 147 | 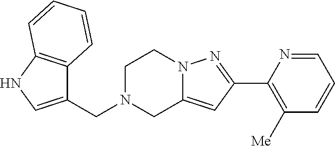 [SMILES: Cc1cccnc1-c1cc2n(n1)CCN(Cc1c[nH]c3ccccc13)C2] | ¹H-NMR (400 MHz, CDCl₃) δ: 2.58 (3H, s), 3.05 (2H, t, J = 5.6 Hz), 3.83 (2H, s), 3.97 (2H, s), 4.28 (2H, t, J = 5.6 H), 6.45 (1H, s), 7.08-7.20 (2H, m), 7.20-7.26 (2H, m), |
| | | 7.42 (1H, d, J = 8.0 |
| | | Hz), 7.55 (1H, d, J = |
| | | 7.2 Hz), 7.80 (1H, d, |
| | | J = 7.6 Hz), 8.25 |
| | | (1H, brs), 8.51 (1H, |
| | | d, J = 3.2 Hz). |
| 148 | 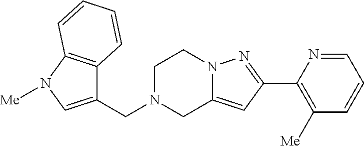 [SMILES: Cc1cccnc1-c1cc2n(n1)CCN(Cc1cn(C)c3ccccc13)C2] | ¹H-NMR (400 MHz, CDCl₃) δ: 2.57 (3H, s), 3.04 (2H, t, J = 5.4 Hz), 3.82 (5H, s), 3.96 (2H, s), 4.27 (2H, t, J = 5.4 Hz), 6.44 (1H, s), 7.06-7.20 (3H, m), 7.24-7.31 (1H, m), |
| | | 7.36 (1H, d, J = 8.4 |
| | | Hz), 7.54 (1H, d, J = |
| | | 7.6 Hz), 7.77 (1H, d, |
| | | J = 8.4 Hz), 8.51 |
| | | (1H, d, J = 3.6 Hz). |
| 149 | 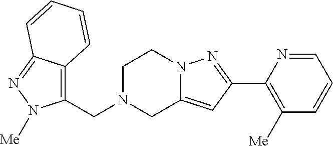 [SMILES: Cc1cccnc1-c1cc2n(n1)CCN(Cc1c3ccccc3nn1C)C2] | ¹H-NMR (400 MHz, CDCl₃) δ: 2.58 (3H, s), 3.02 (2H, brs), 3.83 (2H, s), 4.14 (2H, s), 4.28 (5H, brs), 6.47 (1H, s), 7.13 (2H, brs), 7.32 (1H, dd, J = 7.6, 7.6 Hz), 7.55 (1H, d, J = |
| | | 6.8 Hz), 7.66 (1H, d, |
| | | J = 8.0 Hz), 7.72 |
| | | (1H, d, J = 8.8 Hz), |
| | | 8.52 (1H, brs). |
| 150 | 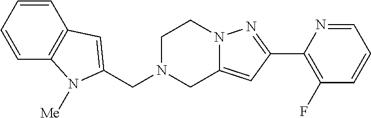 [SMILES: Cn1c(CN2CCn3nc(-c4ncccc4F)cc3C2)cc2ccccc21] | ¹H-NMR (400 MHz, CDCl₃) δ: 3.03 (2H, t, J = 5.4 Hz), 3.80 (3H, s), 3.81 (2H, s), 3.94 (2H, s), 4.31 (2H, t, J = 5.4 Hz), 6.58 (1H, d, J = |
| | | 3.2 Hz), 7.07 (1H, |
| | | s), 7.14 (1H, dd, J = |
| | | 6.8, 6.8 Hz), 7.17- |
| | | 7.29 (2H, m), 7.30 |
| | | (1H, d, J = 8.4 Hz), |
| | | 7.39-7.47 (1H, m), |
| | | 7.74 (1H, d, J = 8.4 |
| | | Hz), 8.50 (1H, d, J = |
| | | 4.8 Hz). |
| 151 | 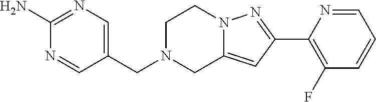 [SMILES: Nc1ncc(CN2CCn3nc(-c4ncccc4F)cc3C2)cn1] | ¹H-NMR (400 MHz, CDCL₃) δ: 2.99 (2H, t, J = 5.6 Hz), 3.60 (2H, s), 3.76 (2H, s), 4.33 (2H, t, J = 5.6 Hz), 5.05 (2H, |
| | | brs), 6.61 (1H, d, J = |
| | | 4.0 Hz), 7.20- |
| | | 7.26 (1H, m), 7.47 |
| | | (1H, dd, J = 9.6, 9.6 |
| | | Hz), 8.31 (2H, s), |
| | | 8.50 (1H, d, J = 4.4 |
| | | Hz). |
| 152 | 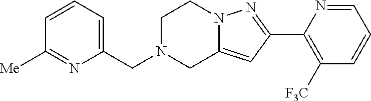 [SMILES: Cc1cccc(CN2CCn3nc(-c4ncccc4C(F)(F)F)cc3C2)n1] | ¹H-NMR (400 MHz, CDCl₃) δ: 2.59 (3H, s), 3.08 (2H, t, J = 5.4 Hz), 3.85 (2H, s), 3.90 (2H, s), 4.35 (2H, t, J = 5.4 |
| | | Hz), 6.41 (1H, s), |
| | | 7.10 (1H, d, J = 7.6 |
| | | Hz), 7.31 (1H, d, J = |
| | | 7.6 Hz), 7.37-7.44 |
| | | (1H, m), 7.61 (1H, |
| | | dd, J = 8.0, 8.0 Hz), |
| | | 8.07 (1H, dd, J = |
| | | 8.0, 0.8 Hz), 8.86 |
| | | (1H, d, J = 3.6 Hz). |
| 153 | 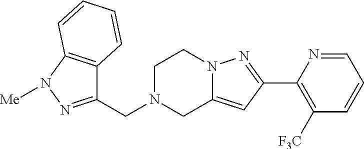 [SMILES: Cn1nc(CN2CCn3nc(-c4ncccc4C(F)(F)F)cc3C2)c2ccccc21] | ¹H-NMR (400 MHz, CDCl₃) δ: 3.10 (2H, t, J = 5.4 Hz), 3.89 (2H, s), 4.10 (3H, s), 4.17 (2H, s), 4.33 (2H, t, J = 5.4 Hz), 6.40 (1H, s), 7.18 (1H, dd, J = 8.0, 6.0 Hz), 7.35- |
| | | 7.47 (3H, m), 7.88 |
| | | (1H, d, J = 8.4 Hz), |
| | | 8.07 (1H, d, J = 8.0 |
| | | Hz), 8.86 (1H, d, J = |
| | | 4.8 Hz). |
| 154 | 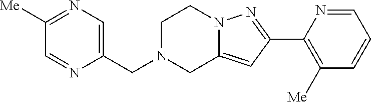 [SMILES: Cc1cnc(CN2CCn3nc(-c4ncccc4C)cc3C2)cn1] | ¹H-NMR (400 MHz, CDCl₃) δ: 2.58 (3H, s), 2.61 (3H, s), 3.09 (2H, t, J = 5.6 Hz), 3.86 (2H, s), 3.93 (2H, s), 4.32 |
| | | (2H, t, J = 5.6 Hz), |
| | | 6.47 (1H, s), 7.13 |
| | | (1H, dd, J = 7.6, 4.4 |
| | | Hz), 7.55 (1H, d, J = |
| | | 7.6 Hz), 8.48 (1H, |
| | | s), 8.51 (1H, d, J = |
| | | 3.6 Hz), 8.62 (1H, |
| | | s). |
| 155 | 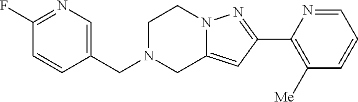 [SMILES: Cc1cccnc1-c1cc2n(n1)CCN(Cc1ccc(F)nc1)C2] | ¹H-NMR (400 MHz, CDCl₃) δ: 2.59 (3H, s), 3.04 (2H, t, J = 5.6 Hz), 3.81 (2H, s), 3.87 (2H, s), 4.31 (2H, t, J = 5.6 |
| | | Hz), 6.49 (1H, s), |
| | | 7.15 (1H, dd, J = |
| | | 7.2, 4.4 Hz), 7.56 |
| | | (1H, d, J = 7.6 Hz), |
| | | 7.72 (1H, d, J = 8.0 |
| | | Hz), 7.98 (1H, d, J = |
| | | 8.0 Hz), 8.51 (1H, d, |
| | | J = 4.4 Hz), 8.75 |
| | | (1H, s). |
| 156 | 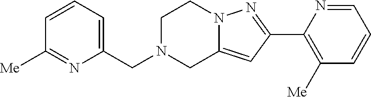 [SMILES: Cc1cccc(CN2CCn3nc(-c4ncccc4C)cc3C2)n1] | ¹H-NMR (400 MHz, CDCl₃) δ: 2.59 (3H, s), 2.59 (3H, s), 3.08 (2H, t, J = 5.2 Hz), 3.85 (2H, s), 3.90 (2H, s), 4.32 |
| | | (2H, t, J = 5.2 Hz), |
| | | 6.46 (1H, s), 7.06- |
| | | 7.18 (2H, m), 7.31 |
| | | (1H, d, J = 7.2 Hz), |
| | | 7.55 (1H, d, J = 7.2 |
| | | Hz), 7.63 (1H, dd, J = |
| | | 7.6, 7.6 Hz), 8.51 |
| | | (1H, d, J = 4.4 Hz). |
| 157 | 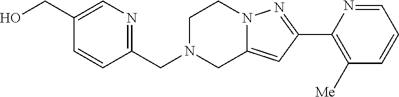 [SMILES: Cc1cccnc1-c1cc2n(n1)CCN(Cc1ccc(CO)cn1)C2] | ¹H-NMR (400 MHz, CDCl₃) δ: 2.57 (3H, s), 3.07 (2H, t, J = 5.6 Hz), 3.83 (2H, s), 3.92 (2H, s), 4.31 (2H, t, J = 5.6 |
| | | Hz), 4.76 (2H, s), |
| | | 6.45 (1H, s), 7.12 |
| | | (1H, dd, J = 7.6, 4.8 |
| | | Hz), 7.50 (1H, d, J = |
| | | 8.0 Hz), 7.54 (1H, d, |
| | | J = 8.4 Hz), 7.75 |
| | | (1H, d, J = 8.0 Hz), |
| | | 8.50 (1H, d, J = 4.8 |
| | | Hz), 8.60 (1H, s). |
| 158 | 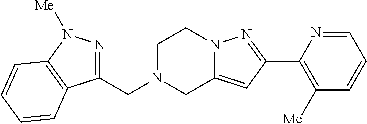 [SMILES: Cc1cccnc1-c1cc2n(n1)CCN(Cc1nn(C)c3ccccc13)C2] | ¹H-NMR (400 MHz, CDCl₃) δ: 2.57 (3H, s), 3.09 (2H, t, J = 5.6 Hz), 3.89 (2H, s), 4.10 (3H, s), 4.16 (2H, s), 4.29 (2H, t, J = 5.6 Hz), 6.45 (1H, s), 7.10- |
| | | 7.21 (2H, m), 7.38- |
| | | 7.47 (2H, m), 7.56 |
| | | (1H, d, J = 7.6 Hz), |
| | | 7.89 (1H, d, J = 8.4 |
| | | Hz), 8.50 (1H, d, J = |
| | | 3.2 Hz). |
| 159 | 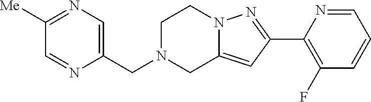 [SMILES: Cc1cnc(CN2CCn3nc(-c4ncccc4F)cc3C2)cn1] | ¹H-NMR (400 MHz, CDCl₃) δ: 2.59 (3H, s), 3.07 (2H, t, J = 5.6 Hz), 3.85 (2H, s), 3.92 (2H, s), 4.36 (2H, t, J = 5.6 |
| | | Hz), 6.60 (1H, d, J = |
| | | 3.6 Hz), 7.18-7.26 |
| | | (1H, m), 7.45 (1H, |
| | | dd, J = 8.8, 8.8 Hz), |
| | | 8.46 (1H, s), 8.50 |
| | | (1H, d, J = 2.8 Hz), |
| | | 8.60 (1H, s). |
| 160 | 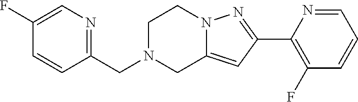 [SMILES: Fc1ccc(CN2CCn3nc(-c4ncccc4F)cc3C2)nc1] | ¹H-NMR (400 MHz, CDCl₃) δ: 3.08 (2H, t, J = 5.4 Hz), 3.84 (2H, s), 3.92 (2H, s), 4.38 (2H, t, J = 5.4 Hz), 6.62 (1H, d, |
| | | J = 3.6 Hz), 7.20- |
| | | 7.27 (1H, m), 7.39- |
| | | 7.56 (3H, m), 8.47 |
| | | (1H, d, J = 2.8 Hz), |
| | | 8.52 (1H, d, J = 4.4 |
| | | Hz). |
| 161 | 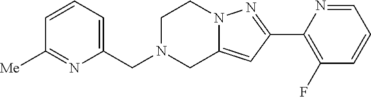 [SMILES: Cc1cccc(CN2CCn3nc(-c4ncccc4F)cc3C2)n1] | ¹H-NMR (400 MHz, CDCl₃) δ: 2.58 (3H, s), 3.06 (2H, t, J = 5.6 Hz), 3.84 (2H, s), 3.89 (2H, s), 4.36 (2H, t, J = 5.6 |
| | | Hz), 6.60 (1H, d, J = |
| | | 3.6 Hz), 7.08 (1H, d, |
| | | J = 7.6 Hz), 7.19- |
| | | 7.26 (1H, m), 7.29 |
| | | (1H, d, J = 7.6 Hz), |
| | | 7.41-7.51 (1H, m), |
| | | 7.59 (1H, d, J = 7.6 |
| | | Hz), 8.50 (1H, d, J = |
| | | 3.2 Hz). |
| 162 | 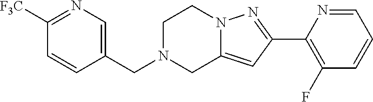 [SMILES: Fc1cccnc1-c1cc2n(n1)CCN(Cc1ccc(C(F)(F)F)nc1)C2] | ¹H-NMR (400 MHz, CDCl₃) δ: 3.04 (2H, t, J = 5.6 Hz), 3.80 (2H, s), 3.86 (2H, s), 4.35 (2H, t, J = 5.6 Hz), 6.62 (1H, d, |
| | | J = 3.2 Hz), 7.19- |
| | | 7.27 (1H, m), 7.47 |
| | | (1H, dd, J = 9.6, 7.2 |
| | | Hz), 7.71 (1H, d, J = |
| | | 8.0 Hz), 7.96 (1H, d, |
| | | J = 8.0 Hz), 8.51 |
| | | (1H, d, J = 1.2 Hz), |
| | | 8.73 (1H, s). |
| 163 | 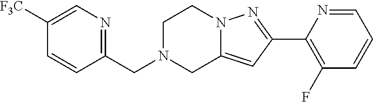 [SMILES: Fc1cccnc1-c1cc2n(n1)CCN(Cc1ccc(C(F)(F)F)cn1)C2] | ¹H-NMR (400 MHz, CDCl₃) δ: 3.09 (2H, t, J = 5.6 Hz), 3.86 (2H, s), 4.00 (2H, s), 4.38 (2H, t, J = 5.6 Hz), 6.62 (1H, d, |
| | | J = 3.6 Hz), 7.20- |
| | | 7.25 (1H, m), 7.47 |
| | | (1H, dd, J = 7.4, 6.2 |
| | | Hz), 7.67 (1H, d, J = |
| | | 8.0 Hz), 7.95 (1H, |
| | | dd, J = 8.4, 2.4 Hz), |
| | | 8.51 (1H, dd, J = |
| | | 4.8, 1.6 Hz), 8.86 |
| | | (1H, s). |
| 164 | 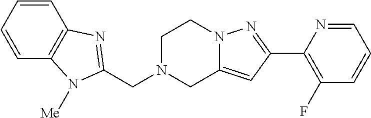 [SMILES: Cn1c(CN2CCn3nc(-c4ncccc4F)cc3C2)nc2ccccc21] | ¹H-NMR (400 MHz, CDCL₃) δ: 3.09 (2H, t, J = 5.4 Hz), 3.86 (2H, s), 3.88 (3H, s), 4.08 (2H, s), 4.32 (2H, t, J = 5.4 Hz), 6.60 (1H, d, J = |
| | | 3.6 Hz), 7.18-7.26 |
| | | (1H, m), 7.26-7.45 |
| | | (4H, m), 7.78 (1H, d, |
| | | J = 7.2 Hz), 8.50 |
| | | (1H, d, J = 4.4 Hz). |
| 165 | 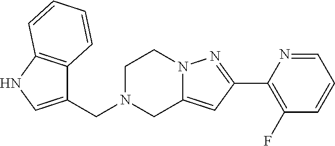 [SMILES: Fc1cccnc1-c1cc2n(n1)CCN(Cc1c[nH]c3ccccc13)C2] | ¹H-NMR (400 MHz, CDCl₃) δ: 3.04 (2H, t, J = 5.6 Hz), 3.83 (2H, s), 3.97 (2H, s), 4.32 (2H, t, J = 5.6 Hz), 6.59 (1H, d, J = 3.2 Hz), 7.10- 7.29 (4H, m), 7.36- 7.49 (2H, m), 7.77 |
| | | (1H, d, J = 7.6 Hz), |
| | | 8.17 (1H, brs), 8.50 |
| | | (1H, d, J = 4.8 Hz). |
| 166 | 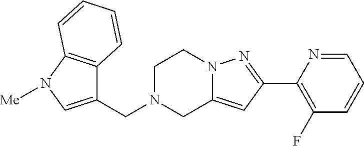 [SMILES: Cn1cc(CN2CCn3nc(-c4ncccc4F)cc3C2)c2ccccc21] | ¹H-NMR (400 MHz, CDCl₃) δ: 3.04 (2H, t, J = 5.4 Hz), 3.80 (3H, s), 3.82 (2H, s), 3.95 (2H, s), 4.31 (2H, t, J = 5.4 Hz), 6.58 (1H, d, J = 3.6 Hz), 7.07 (1H, s), 7.14 (1H, dd, J = |
| | | 7.6, 7.6 Hz), 7.18- |
| | | 7.29 (2H, m), 7.34 |
| | | (1H, d, J = 8.4 Hz), |
| | | 7.40-7.49 (1H, m), |
| | | 7.74 (1H, d, J = 8.0 |
| | | Hz), 8.49 (1H, d, J = |
| | | 3.2 Hz). |
| 167 | 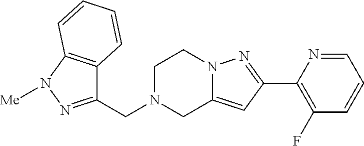 [SMILES: Cn1nc(CN2CCn3nc(-c4ncccc4F)cc3C2)c2ccccc21] | ¹H-NMR (400 MHz, CDCl₃) δ: 3.08 (2H, t, J = 5.6 Hz), 3.88 (2H, s), 4.08 (3H, s), 4.15 (2H, s), 4.33 (2H, t, J = 5.6 Hz), 6.59 (1H, d, J = 3.6 Hz), 7.11-7.26 (2H, m), 7.36-7.50 |
| | | (3H, m), 7.86 (1H, d, |
| | | J = 8.0 Hz), 8.50 |
| | | (1H, d, J = 1.6 Hz). |
| 168 | 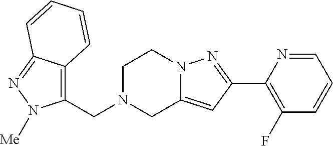 [SMILES: Cn1nc2ccccc2c1CN1CCn2nc(-c3ncccc3F)cc2C1] | ¹H-NMR (400 MHz, CDCl₃) δ: 3.02 (2H, t, J = 5.6 Hz), 3.80 (2H, s), 4.12 (2H, s), 4.25 (3H, s), 4.31 (2H, t, J = 5.6 Hz), 6.60 (1H, d, J = 3.6 Hz), 7.11 (1H, dd, J = 8.0, 8.0 Hz), |
| | | 7.20-7.27 (1H, m), |
| | | 7.31 (1H, dd, J = |
| | | 8.0, 8.0 Hz), 7.46 |
| | | (1H, dd, J = 6.4, 6.4 |
| | | Hz), 7.63 (1H, d, J = |
| | | 8.0 Hz), 7.70 (1H, d, |
| | | J = 8.8 Hz), 8.50 |
| | | (1H, d, J = 4.8 Hz). |
| 169 | 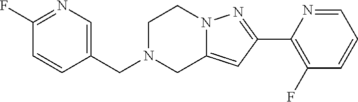 [SMILES: Fc1ccc(CN2CCn3nc(-c4ncccc4F)cc3C2)cn1] | ¹H-NMR (400 MHz, CDCl₃) δ: 3.00 (2H, t, J = 5.6 Hz), 3.75 (2H, s), 3.76 (2H, s), 4.33 (2H, t, J = 5.6 Hz), 6.60 (1H, d, |
| | | J = 4.0 Hz), 6.95 |
| | | (1H, dd, J = 8.4, 2.8 |
| | | Hz), 7.19-7.27 (1H, |
| | | m), 7.46 (1H, dd, J = |
| | | 10.8, 10.8 Hz), 7.87 |
| | | (1H, dd, J = 9.3, 7.3 |
| | | Hz), 8.19 (1H, s), |
| | | 8.50 (1H, d, J = 2.8 |
| | | Hz). |
| 170 | 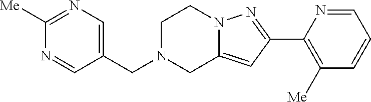 [SMILES: Cc1ncc(CN2CCn3nc(-c4ncccc4C)cc3C2)cn1] | ¹H-NMR (400 MHz, CDCl₃) δ: 2.59 (3H, s), 2.78 (3H, s), 3.02 (2H, t, J = 5.6 Hz), 3.75 (2H, s), 3.80 (2H, s), 4.30 |
| | | (2H, t, J = 5.6 Hz), |
| | | 6.49 (1H, s), 7.14 |
| | | (1H, dd, J = 8.0, 4.8 |
| | | Hz), 7.55 (1H, d, J = |
| | | 6.8 Hz), 8.51 (1H, d, |
| | | J = 3.6 Hz), 8.68 |
| | | (2H, s). |
| 171 | 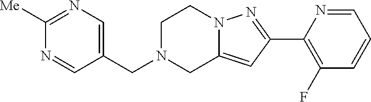 [SMILES: Cc1ncc(CN2CCn3nc(-c4ncccc4F)cc3C2)cn1] | ¹H-NMR (400 MHz, CDCl₃) δ: 2.76 (3H, s0, 3.01 (2H, t, J = 5.6 Hz), 3.73 (2H, s), 3.78 (2H, s), 4.34 (2H, t, J = 5.6 |
| | | Hz), 6.61 (1H, d, J = |
| | | 3.2 Hz), 7.20-7.27 |
| | | (1H, m), 7.43-7.50 |
| | | (1H, m), 8.50 (1H, d, |
| | | J = 1.6 Hz), 8.66 |
| | | (2H, s). |

### Example 172

### 5-{[3-Chloro-2-(pyridin-2-yl)-6,7-dihydropyrazolo[1,5-a]pyrazin-5 (4H)-yl]methyl}-2-methylpyrimidine-4-amine

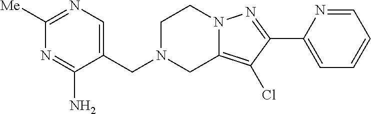 [SMILES: Cc1ncc(CN2CCn3nc(-c4ccccn4)c(Cl)c3C2)c(N)n1]

To a solution of the compound of Reference Example 44 (216 mg, 0.645 mmol) in methanol/water (3 mL/1 mL) was added concentrated hydrochloric acid (327 mg), and the mixture was stirred at 50° C. for 3 hours. To the reaction mixture was then added 15% aqueous sodium hydroxide solution (880 mg) with ice-cooling. The mixture was extracted with chloroform, dried over anhydrous sodium sulfate, filtered, and concentrated. The title compound was prepared from the resulting amine compound according to a similar process to that of Example 1 (124 mg, 74%)

¹H-NMR (400 MHz, CDCl₃) δ: 2.55 (3H, s), 3.00 (2H, t, J=5.5 Hz), 3.73 (4H, d, J=3.2 Hz), 4.28 (2H, t, J=5.5 Hz), 5.75-5.91 (2H, m), 7.27-7.30 (1H, m), 7.77-7.79 (1H, m), 7.96-7.98 (1H, m), 8.06 (1H, s), 8.74-8.75 (1H, m).

### Example 173

### 5-Benzyl-3-chloro-2-(pyridin-2-yl)-4,5,6,7-tetrahydropyrazolo[1,5-a]pyrazine

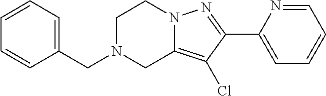 [SMILES: Clc1c(-c2ccccn2)nn2c1CN(Cc1ccccc1)CC2]

To a solution of the compound of Reference Example 44 (169 mg, 0.505 mmol) in methanol/water (3 mL/1 mL) was concentrated hydrochloric acid (256 mg), and the mixture was stirred at 50° C. for 3 hours. To the reaction mixture was then added 15% aqueous sodium hydroxide solution (689 mg) with ice-cooling. The mixture was extracted with chloroform, dried over anhydrous sodium sulfate, filtered, and concentrated. The title compound was prepared from the resulting amine compound according to a similar process to that of Example 25 (32.8 mg, 22%).

¹H-NMR (400 MHz, CDCl₃) δ: 2.97 (2H, t, =5.5 Hz), 3.71 (2H, s), 3.78 (2H, s), 4.24 (2H, t, J=5.4 Hz), 7.22-7.26 (2H, m), 7.37-7.38 (4H, m), 7.73-7.75 (1H, m), 7.94-7.97 (1H, m), 8.71-8.72 (1H, m).

### Examples 174-176

The compounds of Examples 174-176 were synthesized from the corresponding compounds of each Reference Example according to the process of Example 173.

| | | Instrumental |
|---|---|---|
| Example | Chemical Structure | Analysis Data |
| 174 | 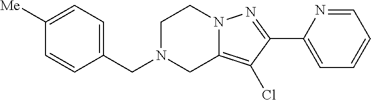 [SMILES: Cc1ccc(CN2CCn3nc(-c4ccccn4)c(Cl)c3C2)cc1] | ¹H-NMR (400 MHz, CDCl₃) δ: 2.97 (2H, t, J = 5.5 Hz), 3.71 (2H, s), 3.78 (2H, s), 4.24 (2H, t, J = 5.4 Hz), 7.22-7.26 (2H, m), 7.37-7.38 (4H, m), 7.73- 7.75 (1H, m), 7.94- |
| | | 7.97 (1H, m), 8.71-8.72 |
| | | (1H, m). |
| 175 | 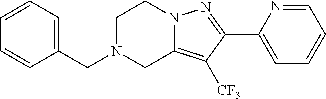 [SMILES: FC(F)(F)c1c(-c2ccccn2)nn2c1CN(Cc1ccccc1)CC2] | ¹H-NMR (400 MHz, CDCl₃) δ: 2.99 (2H, t, J = 5.6 Hz), 3.80 (2H, s), 3.93 (2H, s), 4.28 (2H, t, J = 5.6 Hz), 7.27-7.43 (6H, m), 7.76 (1H, d, J = 3.2 Hz), 8.73 (1H, d, |
| | | J = 4.8 Hz). |
| 176 | 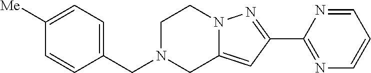 [SMILES: Cc1ccc(CN2CCn3nc(-c4ncccn4)cc3C2)cc1] | ¹H-NMR (400 MHz, DMSO-d₆) δ: 2.31 (3H, s), 2.95 (2H, t, J = 5.6 Hz), 3.65 (2H, s), 3.69 (2H, s), 4.18 (2H, t, J = 5.6 Hz), |
| | | 6.68 (1H, s), 7.18 (2H, |
| | | d, J = 8.0 Hz), 7.27 (2H, |
| | | d, J = 8.0 Hz), 7.37 (1H, |
| | | dd, J = 5.0, 5.0 Hz), |
| | | 8.80 (2H, d, J = 5.0 Hz). |

### Example 177

### 5-[3-Fluoro-4-(trifluoromethoxy)benzyl]-2-(3-methylpyridin-2-yl)-4,5,6,7-tetrahydropyrazolo[1,5-a]pyrazine monohydrochloride

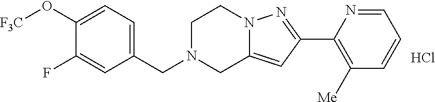 [SMILES: Cc1cccnc1-c1cc2n(n1)CCN(Cc1ccc(OC(F)(F)F)c(F)c1)C2 ; Cl]

To a solution of the compound of Reference Example 12 (0.043 g, 0.199 mmol) in N,N-dimethylformamide (2.0 mL) were added potassium carbonate (0.054 g, 0.398 mmol) and 3-fluoro-4-(trifluoromethoxy)benzyl bromide (0.060 g, 0.219 mmol). The mixture was stirred at room temperature for 3 hours, and water (20 mL) was added thereto. The mixture was then extracted with ethyl acetate (20 mL), dried over anhydrous sodium sulfate, filtered, and concentrated. The concentrated residue was purified by silica gel column chromatography (chloroform/methanol), and methanol (1.0 mL) and then 4 mol/L hydrochloric acid/cyclopentyl methyl ether (47 μL) were added thereto, and the mixture was concentrated. The concentrated residue was triturated with diethyl ether, and removed by filtration to give the title compound (0.045 g, 51%).

¹H-NMR (300 MHz, DMSO-d₆) δ: 2.61 (3H, s), 3.21-3.94 (4H, m), 4.15 (2H, brs), 4.42 (2H, brs), 6.87 (1H, s) 7.44-7.54 (1H, m), 7.57-7.77 (3H, m), 8.11-8.24 (1H, m), 8.57 (1H, d, J=4.4 Hz).

### Examples 178-188

The compounds of Examples 178-188 were synthesized from the corresponding compounds of each Reference Example according to the process of Example 106.

| | | Instrumental |
|---|---|---|
| Example | Chemical Structure | Analysis Data |
| 178 | 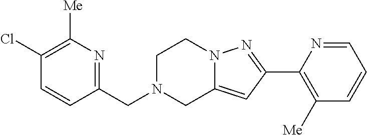 [SMILES: Cc1cccnc1-c1cc2n(n1)CCN(Cc1ccc(Cl)c(C)n1)C2] | ¹H-NMR (400 MHz, CDCl₃) δ: 2.57 (3H, s), 2.64 (3H, s), 3.05 (2H, t, J = 5.5 Hz), 3.82 (2H, s), 3.86 (2H, s), 4.29 (2H, t, J = 5.7 Hz), 6.45 (1H, s), 7.12 (1H, dd, J = 7.6, 4.8 Hz), 7.31 (1H, d, J = 8.3 Hz), 7.53- |
| | | 7.54 (1H, m), 7.63 (1H, |
| | | d, J = 8.3 Hz), 8.49 (1H, |
| | | dd, J = 4.6, 0.9 Hz). |
| 179 | 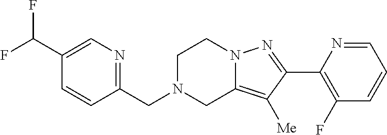 [SMILES: Cc1c(-c2ncccc2F)nn2c1CN(Cc1ccc(C(F)F)cn1)CC2] | ¹H-NMR (400 MHz, CDCl₃) δ: 2.04 (s, 3H), 3.00 (t, J = 5.6 Hz, 2H), 3.68 (s, 2H), 3.84 (s, 2H), 4.28 (t, J = 5.6 Hz, 2H), 6.66 (t, J = 55.6 Hz, 1H), 7.24-7.31 (m, 1H), 7.44- 7.53 (m, 1H), 7.66 (t, J = 8.1 Hz, 1H), 7.92 (d, |
| | | J = 8.1 Hz, 1H), 8.48- |
| | | 8.54 (m, 1H), 8.66 (s, 1H). |
| 180 | 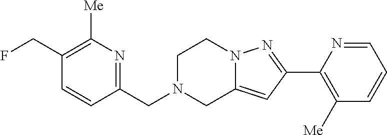 [SMILES: Cc1cccnc1-c1cc2n(n1)CCN(Cc1ccc(CF)c(C)n1)C2] | ¹H-NMR (400 MHz, CDCl₃) δ: 2.60 (3H, d, J = 1.8 Hz), 2.61 (3H, s), 3.08 (2H, t, J = 5.7 Hz), 3.84 (2H, s), 3.91-3.91 (2H, m), 4.31 (2H, t, J = 5.5 Hz), 5.44 (2H, d, J = 62.4 Hz), 6.62 (1H, s), 7.18 (1H, dd, J = 7.6, |
| | | 4.8 Hz), 7.38 (1H, d, J = |
| | | 7.8 Hz), 7.63-7.67 (2H, |
| | | m), 8.53 (1H, d, J = 3.7 Hz). |
| 181 | 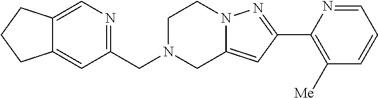 [SMILES: Cc1cccnc1-c1cc2n(n1)CCN(Cc1cc3c(cn1)CCC3)C2] | ¹H-NMR (400 MHz, CDCl₃) δ: 2.13-2.17 (2H, m), 2.58 (3H, s), 2.95-2.97 (4H, m), 3.09 (2H, t, J = 5.7 Hz), 3.82 (2H, s), 3.94 (2H, s), 4.31 (2H, |
| | | t, J = 5.7 Hz), 6.51 (1H, |
| | | s), 7.14 (1H, dd, J = |
| | | 7.8, 5.0 Hz), 7.42 (1H, |
| | | s), 7.57 (1H, d, J = 7.3 Hz), |
| | | 8.44 (1H, s), 8.50 |
| | | (1H, dd, J = 4.8, 1.1 Hz). |
| 182 | 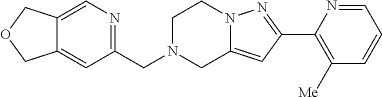 [SMILES: Cc1cccnc1-c1cc2n(n1)CCN(Cc1cc3c(cn1)COC3)C2] | ¹H-NMR (400 MHz, CDCl₃) δ: 2.13-2.17 (2H, m), 2.58 (3H, s), 2.95-2.97 (4H, m), 3.09 (2H, t, J = 5.7 Hz), 3.82 (2H, s), 3.94 (2H, s), 4.31 (2H, |
| | | t, J = 5.7 Hz), 6.51 (1H, |
| | | s), 7.14 (1H, dd, J = |
| | | 7.8, 5.0 Hz), 7.42 (1H, |
| | | s), 7.57 (1H, d, J = 7.3 Hz), |
| | | 8.44 (1H, s), 8.50 |
| | | (1H, dd, J = 4.8, 1.1 Hz). |
| 183 | 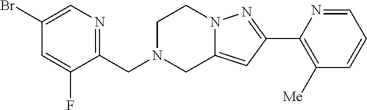 [SMILES: Cc1cccnc1-c1cc2n(n1)CCN(Cc1ncc(Br)cc1F)C2] | ¹H-NMR (400 MHz, CDCl₃) δ: 2.59 (3H, s), 3.12 (2H, t, J = 5.5 Hz), 3.89 (2H, s), 3.96 (2H, d, J = 2.8 Hz), 4.30 (2H, t, J = 5.5 Hz), 6.58-6.65 (1H, m), 7.17-7.23 (1H, m), |
| | | 7.62-7.66 (2H, m), 8.54- |
| | | 8.55 (2H, m). |
| 184 | 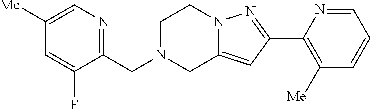 [SMILES: Cc1cnc(CN2CCn3nc(-c4ncccc4C)cc3C2)c(F)c1] | ¹H-NMR (400 MHz, CDCl₃) δ: 2.37 (3H, s), 2.55 (3H, s), 3.10 (2H, t, J = 5.5 Hz), 3.87 (2H, s), 3.95 (2H, d, J = 1.8 Hz), 4.28 (2H, t, J = 5.5 Hz), 6.48 (1H, s), 7.11-7.13 |
| | | (1H, m), 7.23 (1H, s), |
| | | 7.54 (1H, d, J = 7.3 Hz), |
| | | 8.28 (1H, s), 8.49 (1H, |
| | | d, J = 3.7 Hz). |
| 185 | 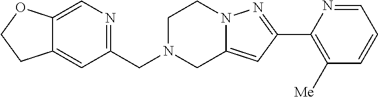 [SMILES: Cc1cccnc1-c1cc2n(n1)CCN(Cc1cc3c(cn1)OCC3)C2] | ¹H-NMR (400 MHz, CDCl₃) δ: 2.56 (s, 3H), 3.05 (t, J = 5.5 Hz, 2H), 3.25 (t, J = 8.8 Hz, 2H), 3.79 (s, 2H), 3.84 (s, 2H), 4.29 (t, J = 5.5 Hz, 2H), 4.63 |
| | | (t, J = 8.8 Hz, 2H), 6.44 |
| | | (s, 1H), 7.11 (dd, J = |
| | | 7.7, 4.5 Hz, 1H), 7.36 |
| | | (s, 1H), 7.53 (d, J = 7.7 Hz, |
| | | 1H), 8.11 (s, 1H), |
| | | 8.49 (d, J = 4.5 Hz, 1H). |
| 186 | 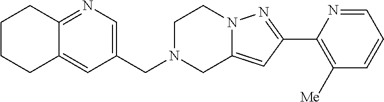 [SMILES: Cc1cccnc1-c1cc2n(n1)CCN(Cc1cnc3c(c1)CCCC3)C2] | ¹H-NMR (400 MHz, CDCl₃) δ: 1.80-1.94 (4H, m), 2.56 (3H, s), 2.78-2.79 (2H, m), 2.94-2.99 (4H, m), 3.69 (2H, s), 3.74 (2H, s), 4.27 (2H, t, J = 5.3 Hz), 6.45 (1H, s), |
| | | 7.12 (1H, t, J = 6.2 Hz), |
| | | 7.43 (1H, s), 7.53 (1H, |
| | | d, J = 7.3 Hz), 8.32 (1H, |
| | | s), 8.49 (1H, d, J = 4.6 Hz). |
| 187 | 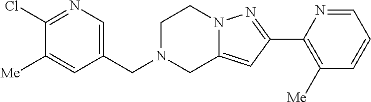 [SMILES: Cc1cc(CN2CCn3nc(-c4ncccc4C)cc3C2)cnc1Cl] | ¹H-NMR (300 MHz, CDCl₃) δ: 2.40 (s, 3H), 2.56 (s, 3H), 3.06 (t, J = 5.5 Hz, 2H), 3.80 (s, 2H), 3.85 (s, 2H), 4.30 (t, J = 24.2 Hz, 2H), 6.45 (s, 1H), 7.12 (dd, J = 7.7, |
| | | 4.6 Hz, 1H), 7.37 (s, |
| | | 1H), 7.53 (d, J = 7.7 Hz, |
| | | 1H), 8.45-8.51 (m, 2H). |
| 188 | 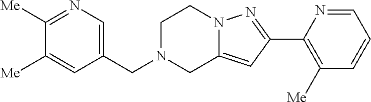 [SMILES: Cc1cc(CN2CCn3nc(-c4ncccc4C)cc3C2)cnc1C] | ¹H-NMR (400 MHz, CDCl₃) δ: 2.31 (3H, s), 2.55 (3H, s), 2.57 (3H, s), 2.99 (2H, t, J = 5.7 Hz), 3.71 (2H, s), 3.74 (2H, s), 4.27 (2H, t, J = 5.5 Hz), 6.48 (1H, s), 7.13 |
| | | (1H, dd, J = 7.6, 4.8 Hz), |
| | | 7.54-7.56 (2H, m), |
| | | 8.31 (1H, s), 8.49-8.50 |
| | | (1H, m). |

### Example 189

### 5-[(5-Chloro-6-methylpyridin-3-yl)methyl]-2-(3-methylpyridin-2-yl)-4,5,6,7-tetrahydropyrazolo[1,5-a]pyrazine

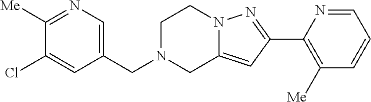 [SMILES: Cc1cccnc1-c1cc2n(n1)CCN(Cc1cnc(C)c(Cl)c1)C2]

To a solution of the compound (328 mg, 0.876 mmol) prepared from the compound of Reference Example 12 and 2,3-dichloro-5-(chloromethyl)pyridine according to a similar process to that of Example 106 in a mixture of tetrahydrofuran (3.0 mL) and N-methylpyrrolidone (0.30 mL) were added iron (III) acetylacetonate (15.4 mg, 0.0436 mmol) and a solution of 1.4 mol/L methylmagnesium bromide in toluene-tetrahydrofuran (3:1) (0.94 mL, 1.32 mmol), and the mixture was stirred at room temperature for 1 hour. To the reaction solution was added water, and the mixture was extracted with ethyl acetate. The organic layer was washed with brine, dried over sodium sulfate, filtered, and concentrated in vacuo. The resulting residue was purified by silica gel chromatography (chloroform:methanol=10:1) to give the title compound (66.6 mg, 21%).

¹H-NMR (400 MHz, CDCl₃) δ: 2.57 (3H, s), 2.64 (3H, s), 3.00 (2H, t, J=5.5 Hz), 3.72 (2H, s), 3.76 (2H, s), 4.28 (2H, t, J=5.5 Hz), 6.46 (1H, s), 7.11-7.13 (1H, m), 7.54 (1H, d, J=7.3 Hz), 7.72 (1H, s), 8.36 (1H, s), 8.49 (1H, d, J=3.7 Hz).

### Example 190

### 5-[(2,4-Dimethylpyrimidin-5-yl)methyl]-2-(3-methylpyridin-2-yl)-4,5,6,7-tetrahydropyrazolo[1,5-a]pyrazine

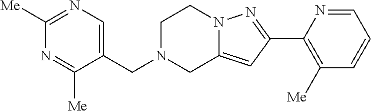 [SMILES: Cc1ncc(CN2CCn3nc(-c4ncccc4C)cc3C2)c(C)n1]

To a solution of (2,4-dimethylpyrimidin-5-yl)methanol (111 mg, 0.803 mmol) in tetrahydrofuran (2.0 mL) were added methanesulfonyl chloride (75 μL, 0.964 mmol) and triethylamine (0.271 mL, 1.93 mmol) with ice-cooling, and the mixture was stirred for 1 hour, and then the insoluble solid was removed by filtration. To the tetrahydrofuran solution of the filtrate were added the compound of Reference Example 12 (108 mg, 0.506 mmol), tetrabutylammonium bromide (16.3 mg, 0.0506 mmol), and 50% aqueous potassium carbonate solution (700 mg, 2.53 mmol), and the mixture was stirred at 75° C. overnight. The reaction solution was then diluted with brine, and extracted with ethyl acetate. The organic layer was dried over anhydrous sodium sulfate, filtered, and concentrated. The concentrated residue was purified by silica gel chromatography (chloroform:methanol=9:1) to give the title compound (120 mg, 71%).

¹H-NMR (400 MHz, CDCl₃) δ: 2.58 (3H, s), 2.60 (3H, s), 2.72 (3H, s), 2.98 (2H, t, J=5.5 Hz), 3.70 (2H, s), 3.78 (2H, s), 4.26 (2H, t, J=5.5 Hz), 6.58-6.58 (1H, m), 7.17-7.18 (1H, m), 7.60-7.60 (1H, m), 8.45 (1H, s), 8.52 (1H, d, J=4.6 Hz).

### Examples 191-194

The compounds of Examples 191-194 can be synthesized from the corresponding compounds of each Reference Example according to the process of Example 190.

| | | Instrumental |
|---|---|---|
| Example | Chemical Structure | Analysis Data |
| 191 | 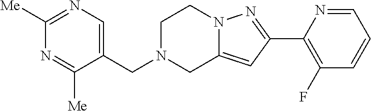 [SMILES: Cc1ncc(CN2CCn3nc(-c4ncccc4F)cc3C2)c(C)n1] | ¹H-NMR (400 MHz, CDCl₃) δ: 2.57 (3H, s), 2.71 (3H, s), 2.99 (2H, t, J = 5.5 Hz), 3.69 (2H, s), 3.76 (2H, s), 4.31 (2H, t, J = 5.5 Hz), 6.60 (1H, d, J = 3.7 Hz), |
| | | 7.20-7.25 (1H, |
| | | m), 7.46 (1H, dd, J = |
| | | 9.6, 9.6 Hz), 8.44 (1H, |
| | | s), 8.49-8.50 (1H, m). |
| 192 | 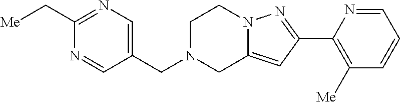 [SMILES: CCc1ncc(CN2CCn3nc(-c4ncccc4C)cc3C2)cn1] | ¹H-NMR (400 MHz, CDCl₃) δ: 1.39 (t, J = 7.6 Hz, 3H), 2.57 (s, 3H), 2.98- 3.06 (m, 4H), 3.73 (s, 2H), 3.78 (s, 2H), 4.28 (t, J = 5.4 Hz, |
| | | 2H), 6.47 (s, 1H), 7.12 |
| | | (dd, J = 7.6, 4.6 Hz, |
| | | 1H), 7.54 (d, J = 7.6 Hz, |
| | | 1H), 8.49 (d, J = |
| | | 4.6 Hz, 1H), 8.68 (s, 2H). |
| 193 | 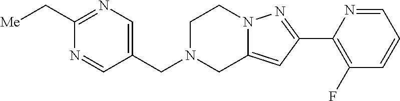 [SMILES: CCc1ncc(CN2CCn3nc(-c4ncccc4F)cc3C2)cn1] | ¹H-NMR (400 MHz, CDCl₃) δ: 1.39 (t, J = 8.0 Hz, 3H), 2.97-3.07 (m, 4H), 3.68-3.84 (m, 4H), 4.29-4.38 (m, 2H), 6.57-6.65 (m, 1H), 7.19-7.26 (m, |
| | | 1H), 7.41-7.51 (m, |
| | | 1H), 8.46-8.53 (m, |
| | | 1H), 8.64-8.70 (m, 2H). |
| 194 | 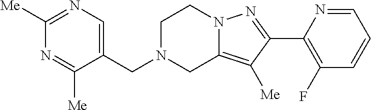 [SMILES: Cc1ncc(CN2CCn3nc(-c4ncccc4F)c(C)c3C2)c(C)n1] | |

### Examples 195-202

The compounds of Examples 195-202 were synthesized from the corresponding compounds of each Reference Example according to the process of Example 1.

| | | Instrumental |
|---|---|---|
| Example | Chemical Structure | Analysis Data |
| 195 | 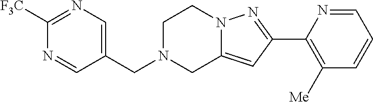 [SMILES: Cc1cccnc1-c1cc2n(n1)CCN(Cc1cnc(C(F)(F)F)nc1)C2] | ¹H-NMR (400 MHz, CDCl₃) δ: 2.56 (3H, s), 3.03 (2H, t, J = 5.5 Hz), 3.81 (2H, s), 3.86 (2H, s), 4.29 (2H, t, J = 5.5 Hz), 6.47 (1H, s), 7.10-7.12 (1H, m), |
| | | 7.51-7.54 (1H, m), |
| | | 8.47-8.48 (1H, m), |
| | | 8.92 (2H, s). |
| 196 | 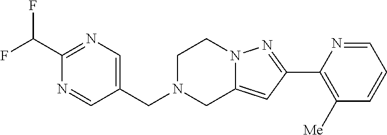 [SMILES: Cc1cccnc1-c1cc2n(n1)CCN(Cc1cnc(C(F)F)nc1)C2] | ¹H-NMR (400 MHz, CDCl₃) δ: 2.56 (3H, s), 3.02 (2H, t, J = 5.5 Hz), 3.80 (2H, s), 3.83 (2H, s), 4.28 (2H, t, J = 5.5 Hz), 6.47 (1H, s), 6.67 (1H, t, J = 54.7 Hz), 7.09-7.12 (1H, m), 7.51-7.53 (1H, |
| | | m), 8.47-8.48 (1H, |
| | | m), 8.88 (2H, s). |
| 197 | 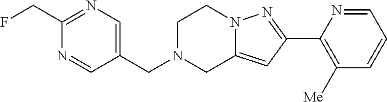 [SMILES: Cc1cccnc1-c1cc2n(n1)CCN(Cc1cnc(CF)nc1)C2] | ¹H-NMR (300 MHz, CDCl₃) δ: 2.55 (3H, s), 3.01 (2H, t, J = 5.7 Hz), 3.78 (4H, s), 4.27 (2H, t, J = 5.7 Hz), 5.55 (2H, d, J = 46.2 Hz), 6.46 (1H, s), 7.10 (1H, |
| | | dd, J = 7.7, 4.8 Hz), |
| | | 7.52 (1H, d, J = 6.6 Hz), |
| | | 8.47 (1H, dd, J = |
| | | 4.4, 1.5 Hz), 8.79 (2H, s). |
| 198 | 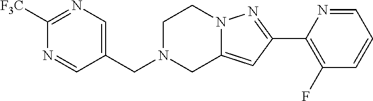 [SMILES: Fc1cccnc1-c1cc2n(n1)CCN(Cc1cnc(C(F)(F)F)nc1)C2] | ¹H-NMR (400 MHz, CDCl₃) δ: 3.04 (2H, t, J = 5.5 Hz), 3.82 (2H, s), 3.86 (2H, s), 4.35 (2H, t, J = 5.5 Hz), 6.61 (1H, d, J = 3.4 Hz), 7.21- 7.22 (1H, m), 7.44-7.46 |
| | | (1H, m), 8.48-8.50 |
| | | (1H, m), 8.92 (2H, s). |
| 199 | 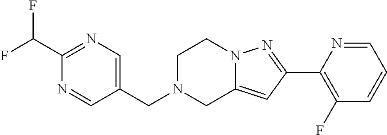 [SMILES: Fc1cccnc1-c1cc2n(n1)CCN(Cc1cnc(C(F)F)nc1)C2] | ¹H-NMR (400 MHz, CDCl₃) δ: 3.03 (2H, t, J = 5.5 Hz), 3.80 (2H, s), 3.83 (2H, s), 4.34 (2H, t, J = 5.5 Hz), 6.60-6.61 (1H, m), 6.67 (1H, t, J = 54.2 Hz), 7.19-7.22 (1H, m), 7.43-7.47 (1H, m), 8.48-8.49 |
| | | (1H, m), 8.88 (2H, s). |
| 200 | 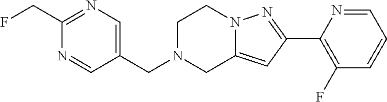 [SMILES: FCc1ncc(CN2CCn3nc(-c4ncccc4F)cc3C2)cn1] | ¹H-NMR (300 MHz, CDCl₃) δ: 3.01 (2H, t, J = 5.5 Hz), 3.78 (4H, s), 4.33 (2H, t, J = 5.5 Hz), 5.55 (2H, d, J = 47.0 Hz), 6.60 (1H, d, J = 2.9 Hz), 7.16-7.25 |
| | | (1H, m), 7.40-7.49 |
| | | (1H, m), 8.48 (1H, d, |
| | | J = 4.4 Hz), 8.79 (2H, s). |
| 201 | 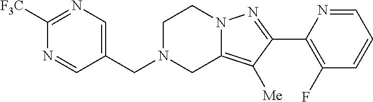 [SMILES: Cc1c(-c2ncccc2F)nn2c1CN(Cc1cnc(C(F)(F)F)nc1)CC2] | |
| 202 | 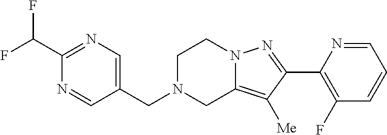 [SMILES: Cc1c(-c2ncccc2F)nn2c1CN(Cc1cnc(C(F)F)nc1)CC2] | |

### Examples 203-204

The compounds of Examples 202-204 can be synthesized from the corresponding compounds of each Reference Example according to the process of Example 106.

| | | Instrumental |
|---|---|---|
| Example | Chemical Structure | Analysis Data |
| 203 | 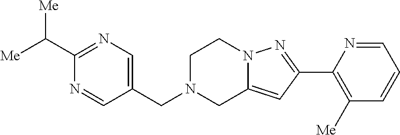 [SMILES: Cc1cccnc1-c1cc2n(n1)CCN(Cc1cnc(C(C)C)nc1)C2] | ¹H-NMR (400 MHz, CDCl₃) δ: 1.36 (6H, s), 1.38 (6H, s), 2.57 (3H, s), 3.01 (2H, t, J = 5.5 Hz), 3.22-3.29 (1H, m), 3.73 (2H, s), 3.79 (2H, s), 4.29 (2H, t, J = 5.5 Hz), 6.47 (1H, s), 7.12 (1H, dd, J = 7.8, |
| | | 4.6 Hz), 7.54 (1H, d, J = |
| | | 7.8 Hz), 8.49 (1H, d, |
| | | J = 3.7 Hz), 8.69 (2H, s). |
| 204 | 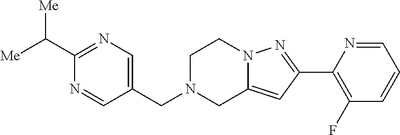 [SMILES: CC(C)c1ncc(CN2CCn3nc(-c4ncccc4F)cc3C2)cn1] | ¹H-NMR (400 MHz, CDCl₃) δ: 1.36 (6H, s), 1.38 (6H, s), 3.02 (2H, t, J = 5.5 Hz), 3.22-3.28 (1H, m), 3.74 (2H, s), 3.79 (2H, s), 4.34 (2H, t, J = 5.5 Hz), 6.62 (1H, d, J = 3.7 Hz), 7.21-7.25 (1H, m), |
| | | 7.44-7.49 (1H, m), |
| | | 8.49-8.50 (1H, m), 8.67- |
| | | 8.70 (2H, m). |

### Reference Example 1

### 2-(Pyridin-2-yl)-4,5,6,7-tetrahydropyrazolo[1,5-a]pyrazine

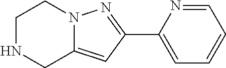 [SMILES: c1ccc(-c2cc3n(n2)CCNC3)nc1]

To a suspension of lithium aluminum hydride (2.1 g, 55 mmol) in tetrahydrofuran (100 mL) was added dropwise a suspension of the compound of Reference Example 2 (5.9 g, 27.5 mmol) in 1,4-dioxane (200 mL), and the mixture was stirred at 80° C. for 3 hours. The reaction mixture was cooled to 0° C., and water (3.14 mL), 4 mol/L aqueous sodium hydroxide solution (3.14 mL), and then water (9.42 mL) were added thereto. The resulting suspension was filtered through Celite®, and the filter cake was washed with 20% methanol/chloroform. The filtrate was concentrated in vacuo, and the resulting residue was purified by amino silica gel column chromatography (chloroform:methanol=1:0 to 9:1) to give the title compound (2.0 g, 37%).

¹H-NMR (300 MHz, CDCl₃) δ: 3.35 (2H, t, J=6.1 Hz), 4.13 (2H, s), 4.21 (2H, t, J=5.6 Hz), 6.61 (1H, s), 7.18 (1H, ddd, J=7.5, 5.0, 1.1 Hz), 7.70 (1H, dt, J=7.5, 7.5, 1.7 Hz), 7.89 (1H, d, J=8.1 Hz), 8.61 (1H, d, J=4.8 Hz).

### Reference Example 2

### 2-(Pyridin-2-yl)-6,7-dihydropyrazolo[1,5-a]pyrazin-4(5H)-one

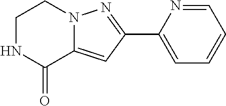 [SMILES: O=C1NCCn2nc(-c3ccccn3)cc21]

To a solution of the compound of Reference Example 3 (13.5 g, 37.5 mmol) in 1,4-dioxane (140 mL) was added 4 mol/L hydrochloric acid/1,4-dioxane solution (18.8 mL), and the mixture was stirred at 50° C. for 6 hours. The reaction solution was concentrated in vacuo to give a white solid. The white solid was dissolved in methanol (80 mL), and potassium carbonate (16 g) was added thereto, and then the mixture was stirred at room temperature for 16 hours. The reaction solution was filtered and concentrated in vacuo. To the resulting residue was added 20% methanol/chloroform, and the resulting white precipitate was removed through Celite®. The filtrate was purified by silica gel column chromatography (chloroform:methanol=1:0 to 9:1) to give the title compound (5.9 g, 73%).

¹H-NMR (400 MHz, CDCl₃) δ: 3.82-3.86 (2H, m), 4.49 (2H, t, J=6.1 Hz), 6.34 (1H, brs), 7.22-7.26 (1H, m), 7.45 (1H, s), 7.75 (1H, dt, J=7.8, 1.6 Hz), 7.87 (1H, d, J=7.8 Hz), 8.66-8.69 (1H, m).

### Reference Example 3

### Ethyl 1-{2-[(tert-butoxycarbonyl)amino]ethyl}-3-(pyridin-2-yl)-1H-pyrazole-5-carboxylate

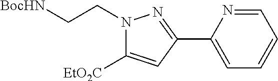 [SMILES: CCOC(=O)c1cc(-c2ccccn2)nn1CCNC(=O)OC(C)(C)C]

To a solution of the compound of Reference Example 4 (8.2 g, 37.8 mmol), N-(tert-butoxycarbonyl)ethanolamine (6.4 g, 39.7 mmol), and triphenylphosphine (10.4 g, 39.7 mmol) in anhydrous tetrahydrofuran (60 mL) was added dropwise diethyl azodicarboxylate (18 mL, 39.7 mmol, 2.2 mol/L toluene solution) at 0° C., and the mixture was stirred at room temperature for 2 hours. The reaction solution was concentrated in vacuo, and the resulting residue was purified by silica gel column chromatography (n-hexane:ethyl acetate=2:1 to 1:2) to give the title compound (13.5 g, 99%).

¹H-NMR (400 MHz, CDCl₃) δ: 1.37-1.42 (3H, m), 1.40 (s, 9H), 3.63-3.67 (2H, m), 4.36 (2H, q, J=7.2 Hz), 4.76 (2H, t, J=5.6 Hz), 7.23 (1H, ddd, J=7.5, 4.8, 1.0 Hz), 7.49 (1H, s), 7.74 (1H, dt, J=7.8, 1.8 Hz), 7.96 (1H, d, J=7.8 Hz), 8.62-8.65 (1H, m).

### Reference Example 4

### Ethyl 3-(pyridin-2-yl)-1H-pyrazole-5-carboxylate

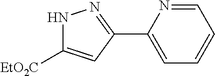 [SMILES: CCOC(=O)c1cc(-c2ccccn2)n[nH]1]

A solution of 2-ethynylpyridine (18.5 g, 179 mmol) and ethyl diazoacetate (30.7 g, 80% purity, 269 mmol) in toluene (200 mL) was stirred at 85° C. for 16 hours. The reaction solution was cooled to room temperature and concentrated in vacuo, and then the resulting solid was filtered and washed with hexane. The resulting solid was purified by silica gel column chromatography (chloroform:methanol=100:0 to 95:5) to give the title compound (5.3 g, 14%).

¹H-NMR (400 MHz, CDCl₃) δ: 1.43 (3H, t, J=7.2 Hz), 4.44 (2H, q, J=7.2 Hz), 7.27-7.30 (2H, m), 7.71-7.75 (1H, m), 7.77-7.80 (1H, m), 8.61-8.64 (1H, m), 11.3 (1H, brs).

### Reference Examples 5-7

The compounds of Reference Examples 5-7 were synthesized from ethyl diazoacetate according to the above processes of Reference Examples 1-4.

| Reference | | Instrumental |
|---|---|---|
| Example | Chemical Structure | Analysis Data |
| 5 | 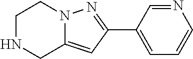 [SMILES: c1cncc(-c2cc3n(n2)CCNC3)c1] | ¹H-NMR (300 MHz, CDCl₃) δ: 3.35 (t, J = 5.6 Hz, 2H), 4.12 (s, 2H), 4.19 (t, J = 5.6 Hz, 2H), |
| | | 6.33 (s, 1H), 7.31 (ddd, |
| | | J = 8.1, 4.8, 0.7 Hz, |
| | | 1H), 8.07 (dt, J = 8.1, |
| | | 2.0, 2.0 Hz, 1H), 8.53 |
| | | (dd, J = 4.8, 1.6 Hz, 1H), |
| | | 8.99 (d, J = 1.5 Hz, 1H). |
| 6 | 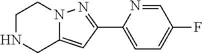 [SMILES: Fc1ccc(-c2cc3n(n2)CCNC3)nc1] | ¹H-NMR (300 MHz, CDCl₃) δ: 3.35 (t, J = 5.5 Hz, 2H), 4.12 (s, 2H), 4.20 (t, J = 5.5 Hz, 2H), |
| | | 6.55 (s, 1H), 7.42 (dt, J = |
| | | 8.2, 8.2, 2.8 Hz, 1H), |
| | | 7.90 (dd, J = 8.6, 4.4 Hz, |
| | | 1H), 8.46 (d, J = 2.8 Hz, |
| | | 1H). |
| 7 | 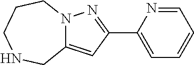 [SMILES: c1ccc(-c2cc3n(n2)CCCNC3)nc1] | ¹H-NMR (300 MHz, CDCl₃) δ: 1.89 (2H, m), 3.23 (2H, t, J = 5.3 Hz), 3.96 (2H, s), 4.48 (2H, t, J = 5.3 Hz), 6.68 (1H, s), |
| | | 7.17 (1H, ddd, J = 7.5, |
| | | 5.0, 1.1 Hz), 7.69 (1H, |
| | | ddd, J = 7.9, 7.5, 1.8 Hz), |
| | | 7.85 (1H, ddd, J = |
| | | 7.9, 1.1, 1.1 Hz), 8.61 |
| | | (1H, m). |

### Reference Example 8

### 2-(2-Methoxyphenyl)-4,5,6,7-tetrahydropyrazolo[1,5-a]pyrazine

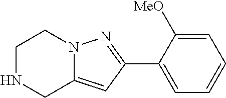 [SMILES: COc1ccccc1-c1cc2n(n1)CCNC2]

To a suspension of lithium aluminum hydride (0.275 g, 7.25 mmol) in tetrahydrofuran (10 mL) was added a solution of the compound of Reference Example 9 (1.47 g, 6.04 mmol) in tetrahydrofuran (20 mL). The mixture was heated under reflux for 8 hours, and lithium aluminum hydride (0.275 g, 7.25 mmol) was added thereto. The mixture was further heated under reflux for 8 hours. To the reaction solution was gradually added water (0.54 mL) with ice-cooling, and then 15% aqueous sodium hydroxide solution (0.54 mL) was gradually added thereto. To the mixture was further added water (1.62 mL), and the mixture was stirred for 30 minutes with ice-cooling. The reaction mixture was then filtered through Celite®, and the filtrate was concentrated. The resulting residue was purified by silica gel column chromatography (chloroform:methanol=90:10) to give the title compound (1.04 g, 75%).

¹H-NMR (300 MHz, CDCl₃) δ: 3.34 (2H, t, J=5.6 Hz), 3.90 (3H, s), 4.12 (2H, s), 4.19 (2H, t, J=5.6 Hz), 6.51 (1H, s), 6.94-7.05 (2H, m), 7.29 (1H, ddd, J=8.7, 7.0, 1.3 Hz), 7.88 (1H, dd, J=7.6, 1.7 Hz).

### Reference Example 9

### 2-(2-Methoxyphenyl)-6,7-dihydropyrazolo[1,5-a]pyrazin-4 (5H)-one

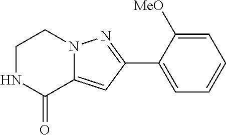 [SMILES: COc1ccccc1-c1cc2n(n1)CCNC2=O]

To a solution of the compound of Reference Example 10 (2.60 g, 7.98 mmol) in ethanol (30 mL) was added triethylamine (1.67 mL, 12.0 mmol). The mixture was stirred at room temperature for 23 hours, and water (150 mL) was added to the reaction mixture, and then the mixture was extracted with ethyl acetate (100 mL×3 times). The combined organic layer was dried over anhydrous magnesium sulfate, filtered, and concentrated. The resulting residue was purified by silica gel column chromatography (chloroform:methanol=95:5) to give the title compound (1.52 g, 78%).

¹H-NMR (300 MHz, CDCl₃) δ: 3.80-3.86 (2H, m), 3.92 (3H, s), 4.43-4.50 (2H, m), 6.44 (1H, brs), 6.98-7.07 (2H, m), 7.34 (1H, ddd, J=8.7, 7.0, 1.3 Hz), 7.45 (1H, s), 7.94 (1H, dd, J=7.6, 1.7 Hz).

### Reference Example 10

### Ethyl 1-(2-aminoethyl)-3-(2-methoxyphenyl)-1-pyrazole-5-carboxylate monohydrochloride

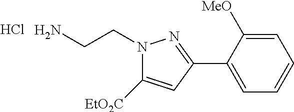 [SMILES: Cl ; CCOC(=O)c1cc(-c2ccccc2OC)nn1CCN]

To a solution of the compound of Reference Example 11 (3.20 g, 8.22 mmol) in chloroform (20 mL) was added 4 mol/L hydrochloric acid/1,4-dioxane (40 mL). The mixture was stirred at room temperature for 30 minutes, and the reaction mixture was concentrated to give the title compound (2.71 g, quantitative).

¹H-NMR (300 MHz, DMSO-D₆) δ: 1.33 (3H, t, J=7.2 Hz), 3.33 (2H, t, J=6.1 Hz), 3.88 (3H, s), 4.34 (2H, q, J=7.1 Hz), 4.77 (2H, t, J=6.1 Hz), 7.02 (1H, dd, J=7.2, 7.2 Hz), 7.14 (1H, d, J=7.5 Hz), 7.29 (1H, s), 7.36 (1H, ddd, J=7.7, 7.7, 2.1 Hz), 7.90 (2H, brs), 7.92 (1H, dd, J=7.7, 1.8 Hz).

### Reference Example 11

### Ethyl 1-{2-[(tert-butoxycarbonyl)amino]ethyl}-3-(2-methoxyphenyl)-1H-pyrazole-5-carboxylate

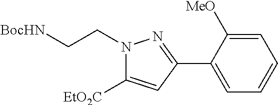 [SMILES: CCOC(=O)c1cc(-c2ccccc2OC)nn1CCNC(=O)OC(C)(C)C]

To a solution of ethyl 3-(2-methoxyphenyl)-1H-pyrazole-5-carboxylate (2.00 g, 8.12 mmol) which can be synthesized according to the process of WO 2007/061923 in tetrahydrofuran (20 mL) were added N-(tert-butoxycarbonyl)ethanolamine (1.44 g, 8.93 mmol) and triphenylphosphine (2.55 g, 9.74 mmol). To the mixture was then added a solution of 1.9 mol/L diisopropyl azodicarboxylate in toluene (5.13 mL, 9.74 mmol) with ice-cooling. The mixture was stirred at room temperature for 20 hours, and the reaction mixture was concentrated. The resulting residue was purified by silica gel column chromatography (hexane:ethyl acetate=69:31) to give the title compound (3.34 g, quantitative).

¹H-NMR (300 MHz, CDCl₃) δ: 1.41 (3H, t, J=7.2 Hz), 1.42 (9H, s), 3.60-3.69 (2H, m), 3.94 (3H, s), 4.38 (2H, q, J=7.2 Hz), 4.73 (2H, t, J=5.6 Hz), 5.07 (1H, br s), 6.97-7.07 (2H, m), 7.30-7.37 (2H, m), 7.95 (1H, dd, J=7.7, 1.5 Hz).

### Reference Example 12

### 2-(3-Methylpyridin-2-yl)-4,5,6,7-tetrahydropyrazolo[1,5-a]pyrazine

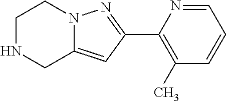 [SMILES: Cc1cccnc1-c1cc2n(n1)CCNC2]

To a solution of the compound of Reference Example 13 (318 mg, 1.01 mmol) in methanol (7.5 mL) was added trifluoroacetic acid (0.4 mL, 5.37 mmol) at 0° C. The mixture was stirred at room temperature for 1.5 hours, and trifluoroacetic acid (1.0 mL, 13.4 mmol) was added thereto at 0° C. The mixture was stirred at room temperature for 64.5 hours, and trifluoroacetic acid (2.0 mL, 26.8 mmol) was added thereto at 0° C. The mixture was stirred at room temperature for 2 hours and 20 minutes, and trifluoroacetic acid (3.4 mL, 45.6 mmol) was added thereto at 0° C. The mixture was stirred at room temperature for 2 hours and 45 minutes, and 12 mol/L hydrochloric acid (3.7 mL) was added thereto at 0° C. The mixture was stirred at room temperature for 19 hours, and acetonitrile (3 mL) and methanol (2 mL) were added thereto at 0° C. The mixture was stirred for 3 hours, and water and potassium carbonate were added thereto at 0° C. until the pH of the reaction solution became 8 to 9, and then the mixture was extracted with chloroform. The organic layer was dried over sodium sulfate, and filtered, and then the solvent therein was removed to give the title compound (208 mg, 0.97 mmol).

¹H-NMR (400 MHz, CDCl₃) δ: 2.57 (3H, s), 3.40 (2H, t, J=5.6 Hz), 4.18 (2H, s), 4.26 (2H, t, J=5.6 Hz), 6.51 (1H, s), 7.14 (1H, dd, J=7.7, 4.7 Hz), 7.56 (1H, d, J=7.7 Hz), 8.48 (1H, d, J=4.7 Hz).

### Reference Example 13

### Tert-butyl 2-(3-methylpyridin-2-yl)-6,7-dihydropyrazolo[1,5-a]pyrazine-5(4H)-carboxylate

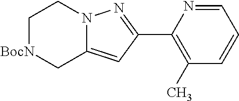 [SMILES: Cc1cccnc1-c1cc2n(n1)CCN(C(=O)OC(C)(C)C)C2]

To a solution of the compound of Reference Example 14 (1.02 g, 3.07 mmol) in dichloromethane (10 mL) were added triethylamine (0.65 mL, 4.66 mmol) and methanesulfonyl chloride (0.35 mL, 4.43 mmol) at 0° C., and the mixture was stirred at 0° C. for 1.5 hours. To the reaction mixture were then added triethylamine (0.21 mL, 1.51 mmol) and methanesulfonyl chloride (0.11 mL, 1.39 mmol) at 0° C. The mixture was stirred at 0° C. for 0.5 hour, and water was added thereto at 0° C., and then the mixture was extracted with chloroform. The organic layer was washed with brine, dried over sodium sulfate, and filtered, and then the solvent therein was removed. The resulting residue (1.31 g) was dissolved in N,N-dimethylformamide (6 mL), and potassium tert-butoxide (0.691 g, 3.49 mmol) was added thereto at 0° C., and then the mixture was stirred at room temperature for 18 hours. To the reaction mixture was added water, and the mixture was extracted with ethyl acetate. The organic layer was washed with brine, dried over sodium sulfate, and filtered, and then the solvent therein was removed. The resulting residue was purified by silica gel column chromatography (chloroform/methanol and hexane/ethyl acetate) to give the title compound (318 mg, 1.01 mmol).

¹H-NMR (400 MHz, CDCl₃) δ: 1.51 (9H, s), 2.60 (3H, s), 3.93 (2H, t, J=5.5 Hz), 4.26 (2H, t, J=5.5 Hz), 4.72 (2H, s), 6.63 (1H, brs), 7.14-7.20 (1H, m), 7.56-7.63 (1H, m), 8.52 (1H, brd, J=4.9 Hz).

### Reference Example 14

### Tert-butyl {2-[5-(hydroxymethyl)-3-(3-methylpyridin-2-yl)-1H-pyrazol-1-yl]ethyl}carbamate

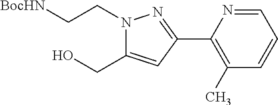 [SMILES: Cc1cccnc1-c1cc(CO)n(CCNC(=O)OC(C)(C)C)n1]

To a suspension of lithium aluminum hydride (0.42 g, 11.1 mmol) in tetrahydrofuran (20 mL) was added dropwise a solution of the compound of Reference Example 15 (3.76 g, 10.0 mmol) in tetrahydrofuran (30 mL) at −10° C. to 0° C. The mixture was stirred at 0° C. for 1.5 hours, and water (0.4 mL), 15% aqueous sodium hydroxide solution (0.4 mL), and then water (0.13 mL) were added thereto at −10° C. to 0° C., and the mixture was stirred overnight. The reaction mixture was filtered through Celite®, and the solvent therein was removed. The residue was purified by silica gel chromatography (chloroform/methanol) to give the title compound (3.19 g, 9.60 mmol).

¹H-NMR (400 MHz, CDCl₃) δ: 1.39 (9H, s), 2.60 (3H, s), 3.63 (2H, q, J=5.9 Hz), 4.34 (2H, t, J=5.9 Hz), 4.71 (2H, s), 5.32 (1H, brs), 6.75 (1H, s), 7.16 (1H, dd, J=7.7, 4.7 Hz), 7.59 (1H, dq, J=7.7, 0.8 Hz), 8.49 (1H, brd, J=4.7 Hz).

### Reference Example 15

### Ethyl 1-{2-[(tert-butoxycarbonyl)amino]ethyl}-3-(3-methylpyridin-2-yl)-1H-pyrazole-5-carboxylate

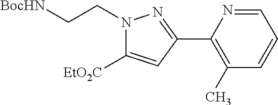 [SMILES: CCOC(=O)c1cc(-c2ncccc2C)nn1CCNC(=O)OC(C)(C)C]

To a solution of the compound of Reference Example 16 (3.04 g, 13.1 mmol) in N,N-dimethylformamide (22 mL) were added tert-butyl (2-bromoethyl)carbamate (3.26 g, 14.5 mmol) and potassium carbonate (2.21 g, 16.0 mmol), and the mixture was stirred at room temperature for 19 hours. To the reaction solution was then added water (60 mL) at 0° C., and the mixture was extracted with a mixture of hexane/ethyl acetate (4/1). The organic layer was washed with brine, dried over sodium sulfate, and filtered, and then the solvent therein was removed. The residue was purified by silica gel column chromatography (hexane/ethyl acetate) to give the title compound (3.76 g, 10.0 mmol).

¹H-NMR (400 MHz, CDCl₃) δ: 1.38 (3H, t, J=7.2 Hz), 1.40 (9H, s), 2.64 (3H, s), 3.64 (2H, brs), 4.36 (2H, q, J=7.2 Hz), 4.76 (2H, brt, J=5.5 Hz), 7.18 (2H, dd, J=7.4, 4.6 Hz), 7.46 (1H, s), 7.59 (1H, brd, J=7.4 Hz), 8.51 (1H, brd, J=4.6 Hz).

### Reference Example 16

### Ethyl 3-(3-methylpyridin-2-yl)-1H-pyrazole-5-carboxylate

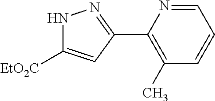 [SMILES: CCOC(=O)c1cc(-c2ncccc2C)n[nH]1]

The title compound was synthesized using 2-ethynyl-3-methylpyridine (1.92 g, 16.4 mmol) according to a similar process to that of Reference Example 4 (3.04 g, 13.1 mmol).

¹H-NMR (400 MHz, CDCl₃) δ: 1.44 (3H, t, J=7.1 Hz), 2.59 (3H, s), 4.45 (2H, q, J=7.1 Hz), 7.23-7.27 (2H, m), 7.64 (1H, brd, J=7.6 Hz), 8.50 (1H, brd, J=3.4 Hz).

### Reference Example 17

### 3-Phenyl-4,5,6,7-tetrahydropyrazolo[1,5-a]pyrazine

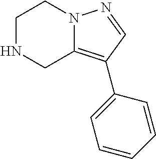 [SMILES: c1ccc(-c2cnn3c2CNCC3)cc1]

To a solution of the compound of Reference Example 18 (58 mg, 0.27 mmol) in tetrahydrofuran (5.8 mL) was added lithium aluminum hydride (110 mg, 2.9 mmol). The mixture was stirred at room temperature for 15 hours, and lithium aluminum hydride (110 mg, 2.9 mmol) was added thereto, and then the mixture was stirred at room temperature for 5 hours. To the mixture was then added additional lithium aluminum hydride (350 mg, 9.2 mmol), and the mixture was stirred at room temperature for 19 hours. To the reaction mixture was added saturated aqueous Rochelle salt solution, and the mixture was stirred for 1 day, and then the reaction mixture was extracted with a mixture of chloroform/methanol. The organic layer was dried over anhydrous sodium sulfate, filtered, and concentrated. The resulting residue was purified by silica gel column chromatography (chloroform/methanol) to give the title compound (39 mg, 72%).

LC-MS: Condition A R.T.=0.41 min ObsMS=200.2 [M+1]

### Reference Example 18

### 3-Phenyl-6,7-dihydropyrazolo[1,5-a]pyrazin-4(5H)-one

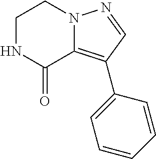 [SMILES: O=C1NCCn2ncc(-c3ccccc3)c21]

To a solution of the compound of Reference Example 19 (90 mg, 0.42 mmol) in tetrahydrofuran (1.4 mL) were added pinacol phenylboronate (85 mg, 0.42 mmol), tetrakis(triphenylphosphine)palladium (48 mg, 0.042 mmol), sodium carbonate (220 mg, 2.1 mmol), and water (0.70 mL) The mixture was stirred under nitrogen at 100° C. (in microwave) for 1.5 hours, and water was added thereto, and then the mixture was extracted with a mixture of chloroform/methanol. The organic layer was concentrated, and the resulting residue was purified by silica gel column chromatography (chloroform/methanol). The resulting crude product was further purified by amino silica gel column chromatography (chloroform/methanol) to give the title compound (58 mg, 65%).

LC-MS: Condition A R.T.=0.62 min ObsMS=214.1 [M+1]

### Reference Example 19

### 3-Bromo-6,7-dihydropyrazolo[1,5-a]pyrazin-4(5H)-one

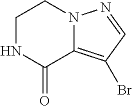 [SMILES: O=C1NCCn2ncc(Br)c21]

To a solution of 6,7-dihydropyrazolo[1,5-a]pyrazin-4(5H)-one (82 mg, 0.60 mmol) in N,N-dimethylformamide (0.80 mL) was added N-bromosuccinimide (12 mg, 0.66 mmol). The mixture was stirred at room temperature for 18 hours, and the reaction mixture was ice-cooled, and then water was added thereto. The resulting precipitate was collected by filtration, and dried in vacuo to give the title compound (96 mg, 74%).

¹H-NMR (300 MHz, CDCl₃) δ: 3.76-3.82 (2H, m), 4.36-4.44 (2H, m), 6.22 (1H, brs), 7.56 (1H, s).

### Reference Example 20

### 3-Methyl-2-(pyridin-2-yl)-5,6,7,8-tetrahydro-4H-pyrazolo[1,5-a][1,4]diazepine dihydrochloride

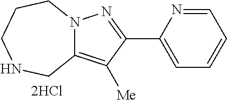 [SMILES: Cc1c(-c2ccccn2)nn2c1CNCCC2]

The compound of Reference Example 21 (650 mg, 1.98 mmol) was dissolved in 4 mol/L hydrochloric acid/1,4-dioxane (10 mL). The solution was stirred at room temperature for 48 hours, and the reaction solution was concentrated to give the title compound (523 mg, 100%).

¹H-NMR (400 MHz, DMSO-d₆) δ: 2.08 (2H, brs), 3.16 (3H, s), 3.44 (2H, brs), 4.57 (2H, brs), 4.58 (2H, brs), 7.72 (1H, dd, J=6.7, 6.7 Hz), 8.12 (1H, d, J=8.0 Hz), 8.33 (1H, dd, J=7.4, 7.4 Hz), 8.74 (1H, d, J=4.8 Hz), 9.64 (2H, brs).

### Reference Example 21

### Tert-butyl 3-methyl-2-(pyridin-2-yl)-7,8-dihydro-4H-pyrazolo[1,5-a][1,4]diazepine-5(6H)-carboxylate

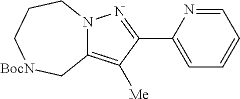 [SMILES: Cc1c(-c2ccccn2)nn2c1CN(C(=O)OC(C)(C)C)CCC2]

To a solution of the compound of Reference Example 22 (1.0 g, 2.55 mmol) in tetrahydrofuran (20 mL) was added a solution of 2.5 mmol/L n-butyllithium in hexane (3 mL, 7.65 mmol) at −78° C. The mixture was stirred at −78° C. for 1 hour, and methyl iodide (1.09 g, 7.65 mmol) was added thereto, and then the mixture was stirred at room temperature for 16 hours. To the reaction mixture was then added saturated aqueous ammonium chloride solution (30 mL), and the mixture was extracted with ethyl acetate (20 mL×3 times). The combined organic layer was dried over anhydrous sodium sulfate, filtered, and concentrated. The concentrated residue was purified by silica gel column chromatography (petroleum ether:ethyl acetate=3:1) to give the title compound (650 mg, 78%).

¹H-NMR (400 MHz, CDCl₃) δ: 1.47 (9H, s), 1.98 (2H, brs), 2.42 (3H, s), 3.78 (2H, brs), 4.43-4.55 (4H, m), 7.15-7.23 (1H, m), 7.72 (1H, dd, J=6.0, 6.0 Hz), 7.75-7.88 (1H, m), 8.67 (1H, d, J=4.0 Hz).

### Reference Example 22

### Tert-butyl 3-bromo-2-(pyridin-2-yl)-7,8-dihydro-4H-pyrazolo[1,5-a][1,4]diazepine-5(6H)-carboxylate

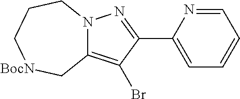 [SMILES: CC(C)(C)OC(=O)N1CCCn2nc(-c3ccccn3)c(Br)c2C1]

To a solution of the compound of Reference Example 23 (1.50 g, 4.78 mmol) in dichloromethane (15 mL) was added N-bromosuccinimide (850 mg, 4.78 mmol) in several portions with ice-cooling. The mixture was stirred at room temperature for 1 hour, and 1 mol/L aqueous sodium hydroxide solution (30 mL) was added thereto, and then the organic layer was separated and extracted. The organic layer was dried over sodium sulfate, filtered, and concentrated to give the title compound (1.80 g, 96%).

¹H-NMR (400 MHz, CDCl₃) δ: 1.48 (9H, s), 2.04 (2H, brs), 3.78 (2H, brs), 4.50-4.58 (2H, m), 4.61 (2H, s), 7.27 (1H, dd, J=7.2, 4.0 Hz), 7.77 (1H, dd, J=4.0, 4.0 Hz), 8.01 (1H, d, J=7.2 Hz), 8.74 (1H, d, J=4.0 Hz).

### Reference Example 23

### Tert-butyl 2-(pyridin-2-yl)-7,8-dihydro-4H-pyrazolo[1,5-a][1,4]diazepine-5(6H)-carboxylate

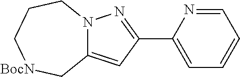 [SMILES: CC(C)(C)OC(=O)N1CCCn2nc(-c3ccccn3)cc2C1]

To a solution of the compound of Reference Example 7 (5.00 g, 23.4 mmol) in methanol (100 mL) was added di-tert-butyl dicarbonate (10.2 g, 46.8 mmol). The mixture was stirred at room temperature for 16 hours, and the reaction mixture was concentrated and purified by silica gel column chromatography (petroleum ether/ethyl acetate) to give the title compound (3.5 g, 48%).

¹H-NMR (400 MHz, CDCl₃) δ: 1.44 (9H, s), 2.00 (2H, brs), 3.75 (2H, brs), 4.45-4.60 (4H, m), 6.72-6.84 (1H, m), 7.20 (1H, dd, J=5.6, 5.6 Hz), 7.65-7.93 (2H, m), 8.64 (1H, d, J=4.4 Hz).

### Reference Example 24

### 3-Fluoro-2-(pyridin-2-yl)-5,6,7,8-tetrahydro-4H-pyrazolo[1,5-a][1,4]diazepine dihydrochloride

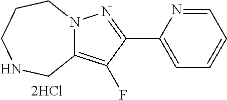 [SMILES: Fc1c(-c2ccccn2)nn2c1CNCCC2]

To the compound of Reference Example 25 (647 mg, 1.95 mmol) was added 4 mol/L hydrochloric acid/1,4-dioxane (10 mL). The mixture was stirred at room temperature for 16 hours, and the reaction mixture was concentrated to give the title compound (100%).

### Reference Example 25

### Tert-butyl 3-fluoro-2-(pyridin-2-yl)-7,8-dihydro-4H-pyrazolo[1,5-a][1,4]diazepine-5(6H)-carboxylate

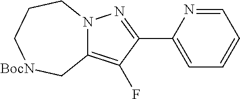 [SMILES: CC(C)(C)OC(=O)N1CCCn2nc(-c3ccccn3)c(F)c2C1]

To a solution of the compound of Reference Example 23 (1.0 g, 3.18 mmol) in acetonitrile (10 mL) was added 1-chloromethyl-4-fluoro-1,4-diazoniabicyclo[2.2.2]octane bis(tetrafluoroborate) (5.63 g, 15.9 mmol) in several portions. The mixture was stirred at room temperature for 16 hours, and the reaction mixture was purified by preparative HPLC (with 0.1% aqueous ammonia) to give the title compound (16%).

¹H-NMR (400 MHz, CDCl₃) δ: 1.44 (9H, s), 1.98 (2H, brs), 3.75 (2H, brs), 4.45-4.60 (4H, m), 7.23 (1H, dd, J=8.4, 8.4 Hz), 7.71-7.83 (2H, m), 8.72 (1H, d, J=4.4 Hz).

### Reference Example 26

### 2-Benzyl-4,5,6,7-tetrahydropyrazolo[1,5-a]pyrazine

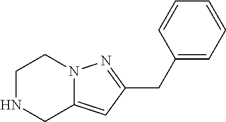 [SMILES: c1ccc(Cc2cc3n(n2)CCNC3)cc1]

To a solution of the compound of Reference Example 27 (190 mg, 0.84 mmol) in tetrahydrofuran (9.5 mL) was added lithium aluminum hydride (680 mg, 18 mmol), and the mixture was stirred at room temperature for 22.5 hours. To the reaction mixture was then added sodium sulfate decahydrate, and the mixture was stirred at room temperature overnight. The resulting suspension was filtered through Celite®. The filtrate was concentrated, and the residue was purified by silica gel column chromatography (chloroform/methanol) to give the title compound (43 mg, 24%).

LC-MS: Condition A R.T.=0.44 min ObsMS=214.0 [M+1]

### Reference Example 27

### 2-Benzyl-6,7-dihydropyrazolo[1,5-a]pyrazin-4(5H)-one

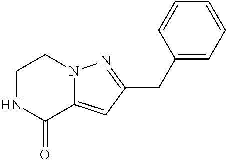 [SMILES: O=C1NCCn2nc(Cc3ccccc3)cc21]

To a solution of a mixture of the regioisomers of the compound of Reference Example 28 (600 mg, 1.9 mmol) in methanol (60 mL) was added cesium carbonate (1.4 g, 4.2 mmol). The mixture was stirred at room temperature for 11 hours, and water was added thereto, and then the mixture was extracted with chloroform. The organic layer was dried over anhydrous sodium sulfate, filtered, and concentrated. The resulting residue was purified by amino silica gel column chromatography (n-hexane/ethyl acetate) to give the title compound (190 mg).

LC-MS: Condition A R.T.=0.64 min ObsMS=228.2 [M+1]

### Reference Example 28

### Mixture of ethyl 1-(2-aminoethyl)-3-benzyl-1H-pyrazolo-5-carboxylate monohydrochloride and ethyl 1-(2-aminoethyl)-4-benzyl-1H-pyrazolo-5-carboxylate monohydrochloride

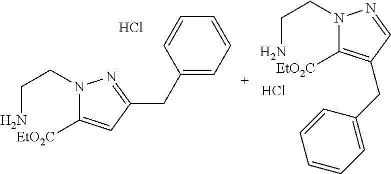 [SMILES: CCOC(=O)c1cc(Cc2ccccc2)nn1CCN ; Cl ; Cl ; CCOC(=O)c1c(Cc2ccccc2)cnn1CCN]

A mixture of the regioisomers of the compound of Reference Example 29 (720 mg, 1.9 mmol) was dissolved in 4 mol/L hydrochloric acid/ethyl acetate (14 mL), and the mixture was stirred at room temperature for 7 hours. The reaction mixture was concentrated to give the title regioisomer mixture (600 mg, quantitative).

LC-MS: Condition A R.T.=0.59 min ObsMS=274.9 [M+1]

### Reference Example 29

### Mixture of ethyl 3-benzyl-1-{2-[(tert-butoxycarbonyl)amino]ethyl}-1H-pyrazole-5-carboxylate and ethyl 4-benzyl-1-{2-[(tert-butoxycarbonyl)amino]ethyl}-1H-pyrazole-5-carboxylate

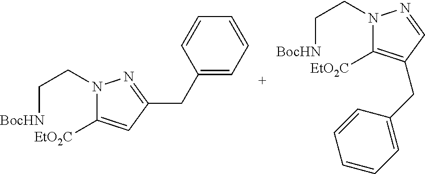 [SMILES: CCOC(=O)c1cc(Cc2ccccc2)nn1CCNC(=O)OC(C)(C)C ; CCOC(=O)c1c(Cc2ccccc2)cnn1CCNC(=O)OC(C)(C)C]

A mixture of the regioisomers of the compound of Reference Example 30 (690 mg, 3.0 mmol) and potassium carbonate (620 mg, 4.5 mmol) were mixed in N,N-dimethylformamide (14 mL), and tert-butyl (2-bromoethyl)carbamate (740 mg, 3.3 mmol) was added thereto with ice-cooling. The mixture was stirred at room temperature for 25 hours, and water was added thereto, and then the mixture was extracted with ethyl acetate. The organic layer was washed with water and brine, dried over anhydrous sodium sulfate, filtered, and concentrated. The resulting residue was purified by silica gel column chromatography (n-hexane/ethyl acetate) to give the title regioisomer mixture (720 mg, 64%).

LC-MS: Condition A R.T.=1.2 min ObsMS=374.2 [M+1]

### Reference Example 30

### Mixture of ethyl 5-benzyl-1H-pyrazole-3-carboxylate and ethyl 4-benzyl-1H-pyrazole-3-carboxylate

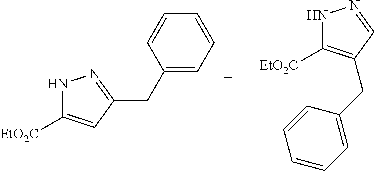 [SMILES: CCOC(=O)c1cc(Cc2ccccc2)n[nH]1 ; CCOC(=O)c1[nH]ncc1Cc1ccccc1]

3-Phenyl-1-propine (1.58 g, 13.6 mmol), ethyl diazoacetate (1.86 g, 16.3 mmol), and zinc trifluoromethanesulfonate (988 mg, 2.72 mmol) were mixed in triethylamine (2.8 mL), and the mixture was stirred at 100° C. for 44 hours. To the resulting reaction mixture was then added saturated aqueous sodium hydrogen carbonate solution, and the mixture was extracted with chloroform. The organic layer was dried over anhydrous magnesium sulfate, filtered, and concentrated. The resulting residue was purified by silica gel column chromatography (n-hexane/ethyl acetate) to give the title regioisomer mixture (1.35 g, 43%).

LC-MS: Condition A R.T.=0.87 min ObsMS=231.2 [M+1]

### Reference Examples 31-37

The compounds of Reference Examples 31-37 were synthesized from the corresponding compounds according to the above processes of Reference Examples 12-15.

| Reference | | Instrumental |
|---|---|---|
| Example | Chemical Structure | Analysis Data |
| 31 | 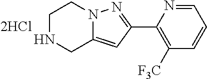 [SMILES: "2HCl" ; FC(F)(F)c1cccnc1-c1cc2n(n1)CCNC2] | ¹H-NMR (400 MHz, CDCl₃) δ: 3.34 (2H, t, J = 5.6 Hz), 4.12 (2H, s), 4.23 (2H, t, J = 5.6 Hz), 6.40 (1H, s), |
| | | 7.32-7.42 (1H, m), |
| | | 8.05 (1H, d, J = |
| | | 7.6 Hz), 8.83 (1H, |
| | | d, J = 4.0 Hz). |
| 32 | 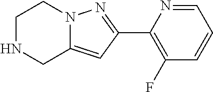 [SMILES: Fc1cccnc1-c1cc2n(n1)CCNC2] | ¹H-NMR (400 MHz, CDCl₃) δ: 3.37 (2H, t, J = 5.6 Hz), 4.16 (2H, s), 4.29 (2H, t, J = 5.6 Hz), 6.63 (1H, d, J = 3.4 Hz), 7.21- |
| | | 7.25 (1H, m), 7.47 |
| | | (1H, ddd, J = 11.0, |
| | | 8.3, 1.2 Hz), 8.49 (1H, |
| | | td, J = 3.1, 1.5 Hz). |
| 33 | 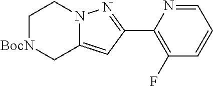 [SMILES: CC(C)(C)OC(=O)N1CCn2nc(-c3ncccc3F)cc2C1] | ¹H-NMR (400 MHz, CDCl₃) δ: 1.51 (9H, s), 3.93 (2H, t, J = 5.2 Hz), 4.32 (2H, t, J = 5.5 Hz), 4.72 (2H, s), 6.71 (1H, d, J = 3.4 Hz), 7.24- |
| | | 7.27 (1H, m), 7.49 |
| | | (1H, ddd, J = 10.9, |
| | | 8.4, 1.3 Hz), 8.52 (1H, |
| | | d, J = 4.4 Hz). |
| 36 | 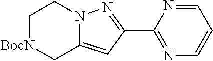 [SMILES: CC(C)(C)OC(=O)N1CCn2nc(-c3ncccn3)cc2C1] | ¹H-NMR (400 MHz, CDCl₃) δ: 1.51 (9H, s), 3.94 (2H, t, J = 5.6 Hz), 4.32 (2H, t, J = 5.6 Hz), |
| | | 4.73 (2H, s), 6.85 (1H, |
| | | s), 7.20 (1H, dd, J = 5.0, |
| | | 5.0 Hz), 8.80 (2H, d, |
| | | J = 5.0 Hz). |
| 37 | 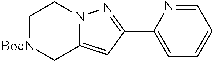 [SMILES: CC(C)(C)OC(=O)N1CCn2nc(-c3ccccn3)cc2C1] | ¹H-NMR (400 MHz, CDCl₃) δ: 1.50 (9H, s), 3.93 (2H, t, J = 5.4 Hz), 4.26 (2H, t, J = 5.4 Hz), |
| | | 4.70 (2H, s), 6.68 (1H, |
| | | s), 7.17-7.22 (1H, m), |
| | | 7.71 (1H, dd, J = 7.7, |
| | | 7.7, 1.9 Hz), 7.87 (1H, |
| | | d, J = 7.8 Hz), 8.60- |
| | | 8.63 (1H, m). |

### Reference Example 38

### Ethyl 3-[3-(trifluoromethyl)pyridin-2-yl]-1H-pyrazole-5-carboxylate

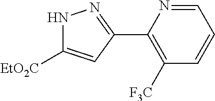 [SMILES: CCOC(=O)c1cc(-c2ncccc2C(F)(F)F)n[nH]1]

To a solution of the compound of Reference Example 39 (1.10 g, 3.80 mmol) in ethanol (15 mL) was added hydrazine monohydrate (0.209 g, 4.18 mmol), and the mixture was stirred at room temperature for 15 minutes and then at 50° C. for 1 hour. The reaction mixture was then concentrated, and the residue was washed with water to give the title compound (0.986 g, 91%).

¹H-NMR (400 MHz, CDCl₃) δ: 1.45 (3H, t, J=7.2 Hz), 4.46 (2H, q, J=7.2 Hz), 7.44 (1H, s), 7.47 (1H, dd, J=8.0, 4.8 z), 8.14 (1H, d, J=7.6 Hz), 8.83 (1, d, J=4.0 Hz).

### Reference Example 39

### Ethyl 2,4-dioxo-4-[3-(trifluoromethyl)pyridin-2-yl]butanoate

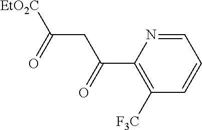 [SMILES: CCOC(=O)C(=O)CC(=O)c1ncccc1C(F)(F)F]

To a solution of 1-[3-(trifluoromethyl)pyridin-2-yl]ethan-1-one (1.00 g, 5.29 mmol) in tetrahydrofuran (15 mL) was added dropwise a solution of 1 mol/L lithium bis(trimethylsilyl)amide in tetrahydrofuran (6.35 mL, 6.35 mmol) at −20° C. The mixture was stirred at −20° C. for 20 minutes, and diethyl oxalate (0.928 g, 6.35 mol) was added thereto, and then the mixture was stirred at room temperature for 1 hour. To the reaction mixture was added water (200 mL) at 0° C., and then 1 mol/L hydrochloric acid until the pH of the mixture became 6. The mixture was then extracted with ethyl acetate (200 mL×3 times), and then the combined organic layer was dried over anhydrous sodium sulfate, filtered, and concentrated. The concentrated residue was washed with petroleum ether/ethyl acetate (5/1) to give the title compound (1.10 g, 72%).

¹H-NMR (400 MHz, CDCl₃) δ: 1.26 (3H, t, J=7.2 Hz), 2.63 (2H, brs), 4.21 (2H, q, J=7.2 Hz), 7.40 (1H, dd, J=8.0, 4.8 Hz), 7.99 (1H, d, J=8.4 Hz), 8.68 (1H, d, J=4.4 Hz).

### Reference Example 40

### Ethyl 3-(3-fluoropyridin-2-yl)-1H-pyrazole-5-carboxylate

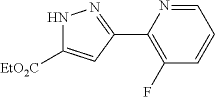 [SMILES: CCOC(=O)c1cc(-c2ncccc2F)n[nH]1]

The title compound was prepared from 2-ethynyl-3-fluoropyridine according to a similar process to that of Reference Example 4.

¹H-NMR (400 MHz, CDCl₃) δ: 1.43 (3H, t, J=7.1 Hz), 4.44 (2H, q, J=7.2 Hz), 7.32-7.36 (1H, m), 7.43 (1H, d, J=3.9 Hz), 7.56 (1H, ddd, J=10.6, 8.3, 1.0 Hz), 8.47 (1H, td, J=3.0, 1.5 Hz).

### Reference Example 41

### Ethyl 3-(pyrimidin-2-yl)-1H-pyrazole-5-carboxylate

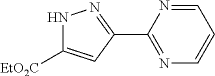 [SMILES: CCOC(=O)c1cc(-c2ncccn2)n[nH]1]

The title compound was prepared from 2-acetylpyrimidine according to similar processes to those of Reference Examples 38 to 39.

¹H-NMR (400 MHz, CDCl₃) δ: 1.43 (3H, t, J=7.2 Hz), 4.44 (2H, q, J=7.2 Hz), 7.20-7.30 (1H, m), 7.58 (1H, s), 8.81 (2H, d, J=4.8 Hz), 11.4 (1H, brs).

### Reference Example 42

### 3-Fluoro-2-(pyridin-2-yl)-4,5,6,7-tetrahydropyrazolo[1,5-a]pyrazine dihydrochloride

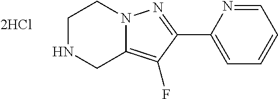 [SMILES: Fc1c(-c2ccccn2)nn2c1CNCC2]

The title compound was prepared from the compound of Reference Example 37 according to similar processes to those of Reference Examples 24 to 25.

¹H-NMR (400 MHz, DMSO-d₆) δ: 3.60 (2H, brs), 4.40 (4H, brs), 7.37 (1H, brs), 7.66-8.04 (2H, m), 8.59 (1H, brs), 10.3 (2H, brs).

### Reference Example 43

### 3-Methyl-2-(pyridin-2-yl)-4,5,6,7-tetrahydropyrazolo[1,5-a]pyrazine dihydrochloride

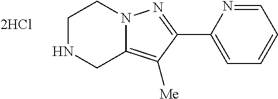 [SMILES: Cc1c(-c2ccccn2)nn2c1CNCC2]

The title compound was prepared from the compound of Reference Example 37 according to similar processes to those of Reference Examples 20 to 22.

¹H-NMR (400 MHz, DMSO-d₆) δ: 2.27 (3H, s), 3.67 (2H, brs), 4.37-4.48 (4H, m), 7.60 (1H, dd, J=6.4, 6.4 Hz), 8.05 (1H, d, J=8.4 Hz), 8.19 (1H, dd, J=7.6, 7.6 Hz), 8.69 (1H, d, J=4.4 Hz), 10.3 (2H, brs).

### Reference Example 44

### Tert-butyl 3-chloro-2-(pyridin-2-yl)-6,7-dihydropyrazolo[1,5-a]pyrazine-5(4H)-carboxylate

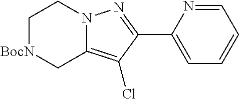 [SMILES: CC(C)(C)OC(=O)N1CCn2nc(-c3ccccn3)c(Cl)c2C1]

To a solution of the compound of Reference Example 37 (362 mg, 1.21 mmol) in tetrahydrofuran (5 mL) was added N-chlorosuccinimide (177 mg, 1.33 mmol), and the mixture was stirred at room temperature overnight. The reaction solution was then concentrated, and the concentrated residue was purified by silica gel chromatography (hexane:ethyl acetate=1:1) to give the title compound (237 mg, 59%).

¹H-NMR (400 MHz, CDCl₃) δ: 1.53 (9H, s), 3.94 (2H, t, J=5.0 Hz), 4.26 (2H, t, J=5.3 Hz), 4.65 (2H, s), 7.26-7.29 (1H, m), 7.77 (1H, td, J=7.8, 1.8 Hz), 7.98 (1H, d, J=7.8 Hz), 8.73-8.75 (1H, m).

### Reference Example 45

### Tert-butyl 2-(pyridin-2-yl)-3-(trifluoromethyl)-6,7-dihydropyrazolo[1,5-a]pyrazine-5(4H)-carboxylate

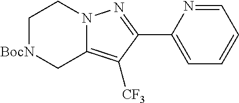 [SMILES: CC(C)(C)OC(=O)N1CCn2nc(-c3ccccn3)c(C(F)(F)F)c2C1]

To a solution of the compound of Reference Example 37 (601 mg, 2.00 mmol) in acetonitrile (10 mL) was added N-iodosuccinimide (675 mg, 3.00 mmol). The mixture was stirred at 30° C. for 2 hours, and the resulting solid was collected by filtration. To a solution of the resulting solid in N,N-dimethylformamide (10 mL) were added copper iodide (282 mg, 2.96 mmol) and methyl 2,2-difluoro-2-(fluorosulfonyl)acetate (711 mg, 3.70 mmol), and the mixture was stirred at 75° C. for 12 hours. To the reaction mixture was then added 2 mol/L aqueous sodium hydrogen carbonate solution (20 mL), and the mixture was extracted with dichloromethane and concentrated. The concentrated residue was purified by preparative HPLC to give the title compound.

¹H-NMR (400 MHz, CDCl₃) δ: 1.52 (9H, s), 3.94 (2H, t, J=5.2 Hz), 4.28 (2H, t, J=5.2 Hz), 4.82 (2H, s), 7.32 (1H, brs), 7.77 (2H, brs), 8.73 (1H, brs).

### Reference Example 46

### 3-Methyl-2-(3-methylpyridin-2-yl)-4,5,6,7-tetrahydropyrazolo[1,5-a]pyrazine

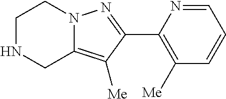 [SMILES: Cc1cccnc1-c1nn2c(c1C)CNCC2]

The title compound was prepared from the compound of Reference Example 13 according to similar processes to those of Reference Examples 20 to 22.

¹H-NMR (400 MHz, CDCl₃) δ: 1.97 (3H, s), 2.38 (3H, s), 3.33 (2H, t, J=5.5 Hz), 4.03 (2H, s), 4.15 (2H, t, J=5.5 Hz), 7.10-7.21 (1H, m), 7.56 (1H, d, J=7.3 Hz), 8.49 (1H, d, J=3.6 Hz).

### Reference Example 47

### 2-(3-Fluoropyridin-2-yl)-3-methyl-4,5,6,7-tetrahydropyrazolo[1,5-a]pyrazine

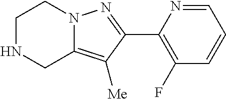 [SMILES: Cc1c(-c2ncccc2F)nn2c1CNCC2]

The title compound was prepared from the compound of Reference Example 33 according to similar processes to those of Reference Examples 20 to 22.

¹H-NMR (400 MHz, CDCl₃) δ: 2.04 (s, 3H), 3.30 (t, J=5.6 Hz, 2H), 4.01 (s, 2H), 4.18 (t, J=5.6 Hz, 2H), 7.21-7.29 (m, 1H), 7.42-7.51 (m, 1H), 8.46-8.53 (m, 1H).

### Reference Example 48

### 3-Chloro-2-(3-methylpyridin-2-yl)-4,5,6,7-tetrahydropyrazolo[1,5-a]pyrazine

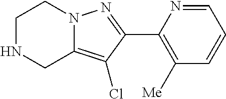 [SMILES: Cc1cccnc1-c1nn2c(c1Cl)CNCC2]

The title compound was prepared from the compound of Reference Example 13 according to similar processes to those of Reference Examples 12 and 44.

¹H-NMR (400 MHz, DMSO-d₆) δ: 2.30 (3H, s), 3.14 (2H, t, J=5.5 Hz), 3.85 (2H, s), 3.98 (2H, t, J=5.5 Hz), 7.29 (1H, dd, J=7.6, 4.8 Hz), 7.69-7.71 (1H, m), 8.44-8.46 (1H, m).

### Reference Example 49

### 2-Formyl-5-(trifluoromethoxy)benzonitrile

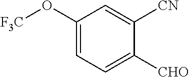 [SMILES: N#Cc1cc(OC(F)(F)F)ccc1C=O]

The compound of Reference Example 50 (0.231 g, 0.74 mmol) was dissolved in DMF solution (3.0 mL). To the reaction solution were added zinc cyanide (0.181 q, 1.54 mmol) and tert-butylphosphinepalladium (0.074 g, 0.14 mmol), and the mixture was irradiated with microwave under nitrogen atmosphere at 130° C. for 2 hours. To the reaction mixture was then added water, and the mixture was extracted with ethyl acetate/hexane solution (1:1). The resulting organic layer was washed with water, dried over sodium sulfate, filtered, and concentrated. To the resulting residue was added 1 mol/L hydrochloric acid, and the mixture was heated to 60° C. and stirred overnight. To the reaction mixture was then added saturated aqueous sodium hydrogen carbonate solution, and the mixture was extracted with chloroform, dried over sodium sulfate, filtered, and concentrated to give the title compound (0.099 g, 62%).

¹H-NMR (400 MHz, CDCl₃) δ: 7.56-7.70 (2H, m), 8.13 (1H, d, J=8.5 Hz), 10.34 (1H, s).

### Reference Example 50

### 2-[2-Bromo-4-(trifluoromethoxy)phenyl]-1,3-dioxolane

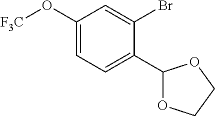 [SMILES: FC(F)(F)Oc1ccc(C2OCCO2)c(Br)c1]

A mixture of 2-bromo-4-(trifluoromethoxy)benzaldehyde (0.219 g, 0.81 mmol), ethylene glycol (0.159 g, 2.56 mmol), p-toluenesulfonic acid (0.022 g, 0.12 mmol), and toluene (4.0 mL) was heated under reflux for 1 hour. To the reaction mixture was then added ethylene glycol (0.256 g, 4.12 mmol), and the mixture was heated under reflux for 1 hour. To the reaction mixture was further added ethylene glycol (0.256 g, 4.12 mmol), and the mixture was heated under reflux for 4 hours. After cooling, saturated aqueous sodium hydrogen carbonate solution was added to the reaction mixture. The mixture was extracted with ethyl acetate, and the organic layer was dried over sodium sulfate, filtered, and concentrated. The resulting residue was purified by silica gel column chromatography (n-hexane/ethyl acetate) to give the title compound (0.231 g, 91%).

¹H-NMR (400 MHz, CDCl₃) δ: 4.04-4.18 (4H, m), 6.07 (1H, d, J=5.1 Hz) 7.21 (1H, dd, J=8.5, 1.2 Hz), 7.45 (1H, dd, J=2.3, 0.9 Hz), 7.64 (1H, d, J=8.5 Hz).

### Reference Example 51

### 5-Formyl-2-(trifluoromethyl)benzonitrile

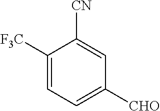 [SMILES: N#Cc1cc(C=O)ccc1C(F)(F)F]

The compound of Reference Example 52 (0.106 g, 0.526 mmol) and manganese dioxide (0.229 g, 2.63 mmol) were mixed in methylene chloride (5.0 mL), and the mixture was stirred at room temperature for 20 hours. The resulting reaction solution was filtered and concentrated. The resulting residue was purified by silica gel column chromatography (n-hexane/ethyl acetate) to give the title compound (0.153 g, 71%).

¹H-NMR (400 MHz, CDCl₃) δ: 7.95-8.10 (1H, m), 8.16-8.29 (1H, m), 8.36 (1H, s), 10.12 (1H, s).

### Reference Example 52

### 5-(Hydroxymethyl)-2-(trifluoromethyl)benzonitrile

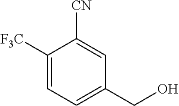 [SMILES: N#Cc1cc(CO)ccc1C(F)(F)F]

3-Bromo-4-trifluoromethylphenylmethanol (0.300 g, 1.17 mmol), zinc cyanide (0.276 g, 2.35 mmol), and bistributylphosphinepalladium (60.2 mg, 0.118 mmol) were mixed in N,N-dimethylformamide (2.5 mL), and the mixture was stirred at 130° C. in microwave for 2 hours. To the resulting reaction mixture was added water, and the mixture was extracted with ethyl acetate. The organic layer was dried over anhydrous sodium sulfate, filtered, and concentrated. The resulting residue was purified by silica gel column chromatography (n-hexane/ethyl acetate) to give the title compound (0.112 g, 47%).

¹H-NMR (400 MHz, CDCl₃) δ: 4.84 (2H, s), 7.70-7.74 (1H, m), 7.77-7.80 (1H, m), 7.87 (1H, s).

### Reference Example 53

### 6-(Chloromethyl)-3,4-dihydro-2H-pyrano[2,3-c]pyridine monohydrochloride

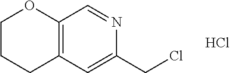 [SMILES: ClCc1cc2c(cn1)OCCC2 ; Cl]

To a solution of 2H,3H,4H-pyrano[2,3-c]pyridin-6-ylmethanol (0.200 g, 1.21 mmol) in methylene chloride (2.0 mL) was added dropwise thionyl chloride (0.19 mL, 2.48 mmol) with ice-cooling, and the mixture was stirred at room temperature for 2 hours. The reaction solution was concentrated to give the title compound (0.266 g, 99%).

¹H-NMR (400 MHz, DMSO-d₆) δ: 1.90-1.98 (2H, m), 2.80 (2H, t, J=6.9 Hz), 4.23 (2H, t, J=6.9 Hz), 4.70-4.75 (2H, m), 7.39 (1H, s), 8.14 (1H, s).

### Reference Example 54

### 2-(Chloromethyl)-6-(fluoromethyl)pyridine monohydrochloride

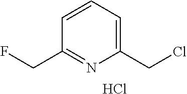

To a solution of the compound of Reference Example 55 (998 mg, 7.07 mmol) in toluene (15 mL) was added thionyl chloride (1.03 mL, 14.14 mmol), and the mixture was stirred at 65° C. for 2 hours. The reaction solution was cooled, and the solvent therein was removed in vacuo to give the title compound (1.19 g, 86%).

¹H-NMR (400 MHz, DMSO-d₆) δ: 4.78 (2H, s), 5.42 (1H, s), 5.53 (1H, s), 7.46-7.48 (1H, m), 7.54-7.56 (1H, m), 7.92-7.96 (1H, m).

### Reference Example 55

### [6-(Fluoromethyl)pyridin-2-yl]methanol

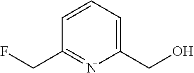

To a solution of 6-bromomethyl-2-pyridinemethanol (2.11 g, 10.4 mmol) in acetonitrile (20 mL) were added potassium fluoride (7.28 g, 125 mmol) and 18-crown-6 (0.828 g, 3.13 mmol), and the mixture was heated under reflux for 2 days. The mixture was cooled to room temperature, and water was added to the reaction solution, and then the mixture was extracted with ethyl acetate 3 times. The combined organic layer was washed with brine, dried over anhydrous sodium sulfate, filtered, and concentrated in vacuo. The resulting residue was purified by silica gel column chromatography (hexane/ethyl acetate) to give the title compound (0.998 g, 68%).

¹H-NMR (400 MHz, CDCl₃) δ: 3.65 (1H, brs), 4.74 (2H, s), 5.41 (1H, s), 5.53 (1H, s), 7.17-7.19 (1H, m), 7.34-7.36 (1H, m), 7.71-7.75 (1H, m).

### Reference Example 56

### 5-(Chloromethyl)-2-(difluoromethyl)pyridine

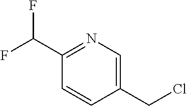 [SMILES: FC(F)c1ccc(CCl)cn1]

To a solution of the compound of Reference Example 57 (276 mg, 1.74 mmol) in tetrahydrofuran (5.0 mL) were added triethylamine (0.85 mL, 6.09 mmol) and methanesulfonyl chloride (0.34 mL, 4.35 mmol), and the mixture was heated under reflux for 1.5 hours. The reaction solution was cooled, and saturated aqueous ammonium chloride solution was added thereto, and then the mixture was extracted with ethyl acetate. The organic layer was washed with saturated aqueous sodium hydrogen carbonate solution and brine, dried over anhydrous sodium sulfate, filtered, and concentrated in vacuo. The resulting residue was purified by silica gel column chromatography (hexane/ethyl acetate) to give the title compound (231 mg, 75%).

¹H-NMR (400 MHz, CDCl₃) δ: 4.63 (2H, s), 6.65 (1H, t, J=55.4 Hz), 7.66 (1H, d, J=8.0 Hz), 7.90 (1H, dd, J=2.0, 8.0 Hz), 8.66 (1H, d, J=2.0 Hz).

### Reference Example 57

### [6-(Difluoromethyl)pyridin-3-yl]methanol

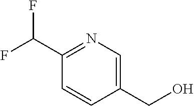 [SMILES: OCc1ccc(C(F)F)nc1]

To a suspension of lithium aluminum hydride (77.9 mg, 2.23 mmol) in tetrahydrofuran (6.0 mL) was added dropwise a solution of the compound of Reference Example 58 (348 mg, 1.86 mmol) in THF (2.0 mL) in an ice bath. The mixture was stirred at 0° C. for 1 hour, and saturated aqueous Rochelle salt solution was added to the reaction solution, and then the mixture was stirred for 3 hours. The reaction mixture was extracted with chloroform 3 times, and the combined organic layer was dried over anhydrous sodium sulfate, filtered, and concentrated in vacuo. The resulting residue was purified by silica gel column chromatography (hexane/ethyl acetate) to give the title compound (276 mg, 93%).

¹H-NMR (400 MHz, CDCl₃) δ: 4.80 (2H, s), 6.64 (1H, t, J=55.4 Hz), 7.63 (1H, d, J=8.0 Hz), 7.86 (1H, dd, J=1.7, 8.0 Hz), 8.61 (1H, d, J=1.7 Hz).

### Reference Example 58

### Methyl 6-(difluoromethyl)pyridine-3-carboxylate

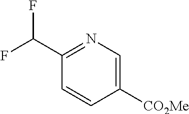 [SMILES: COC(=O)c1ccc(C(F)F)nc1]

To a solution of methyl 6-(hydroxymethyl)nicotinate (511 mg, 3.06 mmol) in dichloromethane (10 mL) was added manganese dioxide (1.33 g, 15.3 mmol), and the mixture was stirred at room temperature for 4.5 hours. The reaction solution was then filtered through Celite®. The filtrate was concentrated in vacuo to give methyl 6-formylnicotinate. To a solution of the resulting methyl 6-formylnicotinate in dichloromethane (5.0 mL) was added diethylaminosulfur trifluoride (1.60 mL, 12.24 mmol) in an ice bath. The mixture was stirred in the ice bath for 1 hour, and saturated aqueous sodium hydrogen carbonate solution was added to the reaction solution, and then the mixture was extracted with ethyl acetate. The organic layer was washed with brine, dried over anhydrous sodium sulfate, filtered, and concentrated in vacuo. The resulting residue was silica gel column chromatography (hexane/ethyl acetate) to give the title compound (361 mg, 63%).

¹H-NMR (400 MHz, CDCl₃) δ: 3.99 (3H, s), 6.68 (1H, t, J=55.2 Hz), 7.73 (1H, d, J=8.1 Hz), 8.45 (1H, dd, J=2.2, 8.1 Hz), 9.25 (1H, m).

### Reference Example 59

### 6-(Chloromethyl)-4-methyl-2H-pyrido[3,2-b][1,4]thiazin-3(4H)-one

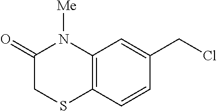 [SMILES: CN1C(=O)CSc2ccc(CCl)cc21]

To a solution of the compound of Reference Example 60 (130 mg, 0.621 mmol) in dichloromethane (2.0 mL) was added thionyl chloride (50 μL) with ice-cooling, and the mixture was stirred at room temperature for 70 minutes. The reaction mixture was then concentrated, and the residue was purified by silica gel column chromatography (hexane:ethyl acetate=1:1) to give the title compound (119 mg, 84%).

¹H-NMR (400 MHz, CDCl₃) δ: 3.41 (2H, s), 3.47 (3H, s), 4.59 (2H, s), 7.05 (1H, dd, J=8.0, 1.6 Hz), 7.11 (1H, d, J=1.4 Hz), 7.35 (1H, d, J=8.3 Hz).

### Reference Example 60

### 6-(Hydroxymethyl)-4-methyl-2H-pyrido[3,2-b][1,4]thiazin-3(4H)-one

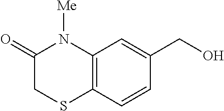 [SMILES: CN1C(=O)CSc2ccc(CO)cc21]

To a mixture of methyl 4-methyl-3-oxo-3,4-dihydro-2H-1,4-benzothiazine-6-carboxylate (475 mg, 2.00 mmol), sodium borohydride (151 mg, 3.99 mmol), and tetrahydrofuran (2.0 mL) was added dropwise methanol (640 mg) at 40° C. The mixture was stirred at 40° C. for 1 hour, diluted with 1 mol/L hydrochloric acid with ice-cooling, and extracted with ethyl acetate. The combined organic layer was dried over sodium sulfate, filtered, and concentrated. The residue was purified by silica gel column chromatography (hexane:ethyl acetate=1:1) to give the title compound (130 mg, 31%).

¹H-NMR (400 MHz, CDCl₃) δ: 3.40 (2H, s), 3.46 (3H, s), 4.72 (2H, d, J=5.5 Hz), 7.02 (1H, dd, J=7.8, 1.8 Hz), 7.13 (1H, d, J=1.4 Hz), 7.35 (1H, d, J=7.8 Hz).

### Reference Example 61

### 7-(Chloromethyl)-1-methyl-3,4-dihydroquinolin-2(1H)-one

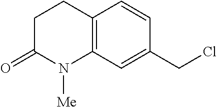 [SMILES: CN1C(=O)CCc2ccc(CCl)cc21]

To a solution of the compound of Reference Example 62 (80.2 mg, 0.42 mmol) in methylene chloride (2.1 mL) was added dropwise thionyl chloride (0.037 mL, 0.51 mmol) at 0° C. The mixture was stirred at 0° C. for 1 hour and 30 minutes, and the reaction mixture was concentrated to give the title compound (85.1 mg, 0.41 mmol).

¹H-NMR (400 MHz, CDCl₃) δ: 2.65 (2H, dd, J=8.4, 6.2 Hz), 2.90 (2H, t, J=7.4 Hz), 3.37 (3H, s), 4.59 (2H, s), 7.02 (2H, t, J=6.3 Hz), 7.15 (1H, d, J=7.6 Hz).

### Reference Example 62

### 7-(Hydroxymethyl)-1-methyl-3,4-dihydroquinolin-2(1H)-one

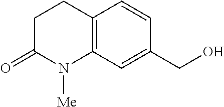 [SMILES: CN1C(=O)CCc2ccc(CO)cc21]

To a solution of the compound of Reference Example 63 (228 mg, 0.75 mmol) in tetrahydrofuran (3.7 mL) was added 1 mol/L hydrochloric acid (1.5 mL) with ice-cooling. The mixture was stirred at room temperature for 1 hour, and saturated sodium hydrogen carbonate solution was added thereto with ice-cooling, and then the mixture was extracted with chloroform. The organic layer was washed with brine, dried over sodium sulfate, filtered, and concentrated to give the title compound (138 mg, 0.72 mmol).

¹H-NMR (400 MHz, CDCl₃) δ: 1.69 (1H, s), 2.64 (2H, dd, J=8.4, 6.2 Hz), 2.90 (2H, dd, J=8.4, 6.2 Hz), 3.37 (3H, s), 4.70 (2H, s), 6.99-7.02 (2H, m), 7.15 (1H, d, J=7.6 Hz).

### Reference Example 63

### 7-({[Tert-butyl(dimethyl)silyl]oxy}methyl)-1-methyl-3,4-dihydroquinolin-2(1H)-one

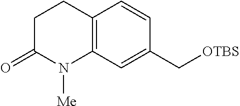 [SMILES: CN1C(=O)CCc2ccc(CO[Si](C)(C)C(C)(C)C)cc21]

To a suspension of sodium hydride (188 mg, 4.71 mmol) in N,N-dimethylformamide (10 mL) was added dropwise a solution of the compound of Reference Example 65 (921 mg, 3.11 mmol) in N,N-dimethylformamide (6.0 mL) at 0° C. The mixture was stirred at 0° C. for 30 minutes, and methyl iodide (0.39 mL, 6.26 mmol) was added thereto with ice-cooling, and then the mixture was stirred at room temperature for 1 hour and 30 minutes. To the reaction mixture was then added saturated aqueous ammonium chloride solution, and the mixture was extracted with ethyl acetate. The organic layer was washed with water 4 times and brine once, dried over sodium sulfate, filtered, and concentrated to give the title compound (904 mg, 2.96 mmol).

¹H-NMR (400 MHz, CDCl₃) δ: 0.11 (6H, s), 0.95 (9H, s), 2.64 (2H, dd, J=8.4, 6.2 Hz), 2.88 (2H, dd, J=8.7, 6.0 Hz), 3.36 (3H, s), 4.73 (2H, s), 6.94 (1H, d, J=7.6 Hz), 7.00 (1H, s), 7.11 (1H, d, J=7.8 Hz).

### Reference Example 64

### 7-({[tert-butyl(dimethyl)silyl]oxy}methyl)-3,4-dihydroquinolin-2(1H)-one

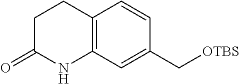 [SMILES: CC(C)(C)[Si](C)(C)OCc1ccc2c(c1)NC(=O)CC2]

To a solution of 3,4-dihydro-7-(hydroxymethyl)-2(1H)-quinolinone (551 mg, 3.11 mmol) in N,N-dimethylformamide (3.1 mL) were added imidazole (428 mg, 6.28 mmol) and tert-butyldimethylsilyl chloride (567 mg, 3.76 mol) with ice-cooling, and the mixture was stirred at 0° C. for 2 hours. To the reaction mixture was then added water, and the mixture was extracted with ethyl acetate. The organic layer was washed with water 3 times and brine once, dried over sodium sulfate, filtered, and concentrated to give the title compound (921 mg, 3.11 mol).

¹H-NMR (400 MHz, CDCl₃) δ: 0.10 (6H, s), 0.94 (9H, s), 2.63 (2H, dd, J=8.4, 6.7 Hz), 2.95 (2H, t, J=7.6 Hz), 4.68 (2H, s), 6.73 (1H, s), 6.92 (1H, d, J=7.6 Hz), 7.11 (1H, d, J=7.8 Hz), 7.69-7.77 (1H, br m).

### Reference Example 65

### 2-(Chloromethyl)-5-(fluoromethyl)pyridine

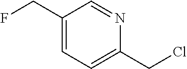

To a solution of the compound of Reference Example 66 (846 mg, 5.37 mmol) in dichloromethane (10 mL) was added diethylaminosulfur trifluoride (1.4 mL, 10.7 mmol) with ice-cooling, and the mixture was stirred for 30 minutes. To the reaction mixture was then added saturated aqueous sodium hydrogen carbonate solution, and the mixture was extracted with chloroform. The combined organic layer was dried over anhydrous sodium sulfate, filtered, and concentrated. The concentrated residue was purified by silica gel column chromatography (hexane:ethyl acetate=1:1) to give the title compound (303 mg, 35%).

¹H-NMR (400 MHz, CDCl₃) δ: 4.69 (2H, s), 5.43 (2H, dd, J=47.5, 2.1 Hz), 7.53 (1H, d, J=7.8 Hz), 7.77 (1H, dd, J=8.3, 1.8 Hz), 8.59 (1H, d, J=1.4 Hz).

### Reference Example 66

### [6-(Chloromethyl)pyridin-3-yl]methanol

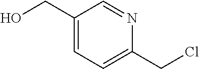

To a solution of methyl 6-(hydroxymethyl)nicotinate (1.16 g, 7.36 mmol) in dichloromethane (10 mL) was added thionyl chloride (1.0 mL, 14.7 mmol) with ice-cooling, and the mixture was stirred for 15 minutes. The reaction mixture was then diluted with saturated aqueous sodium hydrogen carbonate solution, and extracted with chloroform. The combined organic layer was dried and concentrated. To a solution of the concentrated residue in tetrahydrofuran (10 mL) was added a solution of 1.0 mol/L diisobutylaluminium hydride in toluene (16.2 mL, 16.2 mmol) at −78° C., and the mixture was stirred for 2 hours. The reaction mixture was then poured into aqueous potassium sodium tartrate solution. The mixture was stirred at room temperature overnight, and the reaction solution was extracted with ethyl acetate. The combined organic layer was dried over anhydrous sodium sulfate, filtered, and concentrated. The concentrated residue was purified by silica gel column chromatography (chloroform:methanol=9:1) to give the title compound (846 mg, 74%).

### Reference Example 67

### 6-(Chloromethyl)-3-(fluoromethyl)-2-methylpyridine

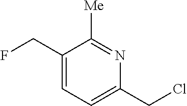 [SMILES: Cc1nc(CCl)ccc1CF]

To a solution of the compound of Reference Example 68 (127 mg, 0.820 mmol) in dichloromethane (2.0 mL) was added thionyl chloride (119 μL, 1.64 mmol) with ice-cooling, and the mixture was stirred for 1 hour. The reaction mixture was diluted with saturated aqueous sodium hydrogen carbonate solution, and extracted with chloroform. The organic layer was dried and concentrated. The concentrated residue was purified by silica gel chromatography (hexane:ethyl acetate=4:1) to give the title compound (98.0 mg, 69%).

¹H-NMR (400 MHz, CDCl₃) δ: 2.57 (3H, s), 4.65 (2H, s), 5.43 (2H, d, J=47.2 Hz), 7.36 (1H, d, J=7.8 Hz), 7.68 (1H, d, J=7.8 Hz).

### Reference Example 68

### [5-(Fluoromethyl)-6-methylpyridin-2-yl]methanol

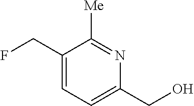 [SMILES: Cc1nc(CO)ccc1CF]

To a solution of the compound of Reference Example 69 (169 mg, 0.923 mmol) in tetrahydrofuran (2.0 mL) was added a solution of 1.0 mol/L diisobutylaluminium hydride in toluene (2.78 mL, 2.78 mmol) at −78° C., and the mixture was stirred for 2 hours. The reaction mixture was diluted with aqueous Rochelle salt solution, and stirred at room temperature for 2 hours. The reaction solution was extracted with ethyl acetate, dried over anhydrous sodium sulfate, filtered, and concentrated. To a solution of the concentrated residue in methanol (3.0 mL) was added sodium borohydride (80 mg, 2.11 mmol) with ice-cooling, and the mixture was stirred for 20 minutes. To the reaction mixture was then added 1 mol/L hydrochloric acid (3.0 mL), and the mixture was basified with 1 mol/L aqueous sodium hydroxide solution, and extracted with chloroform. The combined organic layer was dried anhydrous sodium sulfate, filtered, and concentrated to give the title compound (127 mg, 89%).

¹H-NMR (400 MHz, CDCl₃) δ: 2.60 (3H, s), 4.75 (2H, d, J=1.8 Hz), 5.44 (2H, d, J=47.7 Hz), 7.12 (1H, d, J=7.8 Hz), 7.66 (1H, d, J=7.8 Hz).

### Reference Example 69

### Methyl 5-(fluoromethyl)-6-methylpyridine-2-carboxylate

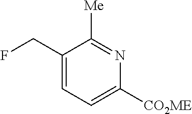

To a solution of the compound of Reference Example 70 (470 mg, 2.43 mmol) in methanol (5.0 mL) was added thionyl chloride (0.706 mL, 9.72 mmol), and the mixture was stirred overnight with heating under reflux. The reaction mixture was cooled to room temperature, concentrated in vacuo, diluted with saturated aqueous sodium hydrogen carbonate solution, and extracted with chloroform. The organic layer was dried over anhydrous sodium sulfate, filtered, and concentrated. The concentrated residue was purified by silica gel chromatography (hexane:ethyl acetate=2:1) to give the title compound (169 mg, 27%).

¹H-NMR (400 MHz, CDCl₃) δ: 2.63 (3H, s), 4.01 (3H, d, J=1.8 Hz), 5.50 (2H, d, J=47.2 Hz), 7.82 (1H, d, J=7.3 Hz), 8.03 (1H, d, J=7.8 Hz).

### Reference Example 70

### 5-(Fluoromethyl)-6-methylpyridine-2-carboxylic acid

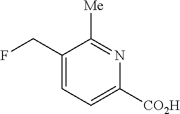 [SMILES: Cc1nc(C(=O)O)ccc1CF]

To a solution of the compound of Reference Example 71 (365 mg, 2.43 mmol) in ethanol (3.0 mL) was added 2 mol/L aqueous sodium hydroxide solution (3.0 mL), and the mixture was stirred at 85° C. for 1 hour. The reaction mixture was then acidified with 1 mol/L hydrochloric acid, and extracted with chloroform. The combined organic layer was dried over anhydrous sodium sulfate, filtered, and concentrated to give the title compound (470 mg).

LC-MS: Condition B R.T.=0.51 min ObsMS=170.1 [M+1]

### Reference Example 71

### 5-(Fluoromethyl)-6-methylpyridine-2-carbonitrile

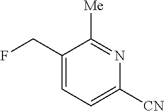 [SMILES: Cc1nc(C#N)ccc1CF]

To a solution of the compound of Reference Example 72 (538 mg, 3.81 mmol) in dichloromethane (5.0 mL) were added dimethylcarbamoyl chloride (512 mg, 4.76 mmol) and trimethylsilyl cyanide (472 mg, 4.76 mmol), and the mixture was stirred at room temperature overnight. The reaction mixture was then diluted with saturated aqueous sodium hydrogen carbonate solution, and extracted with chloroform. The organic layer was dried over anhydrous sodium sulfate, filtered, and concentrated. The concentrated residue was purified by silica gel chromatography (chloroform:methanol=9:1) to give the title compound (365 mg, 27%).

LC-MS: Condition B R.T.=1.32 min ObsM S=151.2 [M+1]

### Reference Example 72

### 3-(Fluoromethyl)-2-methylpyridine 1-oxide

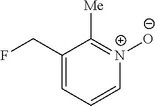

To a mixture of the compound of Reference Example 73 (578 mg, 4.62 mmol), dichloromethane (6.0 mL), and water (6.0 mL) were added sodium hydrogen carbonate (1.20 g, 13.9 mmol) and methachloroperoxybenzoic acid (1.81 g, 5.54 mmol) with ice-cooling, and the mixture was stirred at room temperature overnight. The reaction mixture was then diluted with saturated aqueous sodium hydrogen carbonate solution, and extracted with chloroform. The organic layer was dried over anhydrous sodium sulfate, filtered, and concentrated to give the title compound (538 mg, 83%).

### Reference Example 73

### 3-(Fluoromethyl)-2-methylpyridine

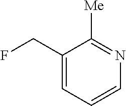

To a solution of (2-methylpyridin-3-yl)methanol (1.96 g, 15.9 mmol) in dichloromethane (20 mL) was added diethylaminosulfur trifluoride (2.29 mL, 17.5 mmol), and the mixture was stirred at room temperature for 4 hours. To the reaction mixture were then added aqueous sodium hydroxide solution and saturated aqueous sodium hydrogen carbonate solution, and extracted with chloroform. The combined organic layer was dried over anhydrous sodium sulfate, filtered, and concentrated. The concentrated residue was purified by silica gel chromatography (hexane:ethyl acetate=1:1) to give the title compound (540 mg, 27%).

¹H-NMR (400 MHz, CDCl₃) δ: 2.58 (3H, s), 5.42 (2H, d, J=47.2 Hz), 7.17 (1H, dd, J=7.8, 5.0 Hz), 7.65 (1H, d, J=7.3 Hz), 8.49 (1H, d, J=5.0 Hz).

### Reference Example 74

### 3-(Chloromethyl)-5,6,7,8-tetrahydroquinoline monohydrochloride

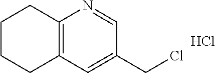 [SMILES: ClCc1cnc2c(c1)CCCC2 ; Cl]

To a solution of (5,6,7,8-tetrahydroquinolin-3-yl)methanol (705 mg, 4.72 mmol) in dichloromethane (5.0 mL) was added thionyl chloride (0.630 mL, 9.44 mmol) with ice-cooling, and the mixture was stirred for 1 hour. The reaction mixture was then concentrated to give the title compound (697 mg, 74%).

¹H-NMR (400 MHz, DMSO-d₆) δ: 1.76-1.83 (4H, m), 2.86 (2H, t, J=6.2 Hz), 3.04 (2H, t, J=6.2 Hz), 4.88 (2H, s), 8.30-8.30 (1H, m), 8.71-8.71 (1H, m).

### Reference Example 75

### 5-(Chloromethyl)-2,3-dihydrofuro[2,3-c]pyridine monohydrochloride

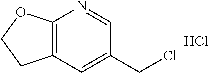 [SMILES: ClCc1cnc2c(c1)CCO2 ; Cl]

To a solution of 2H,3H,-furano[2,3-c]pyridin-6-ylmethanol (0.200 g, 1.32 mmol) in methylene chloride (2.0 mL was added dropwise thionyl chloride (0.19 mL, 2.64 mmol) with ice-cooling, and the mixture was stirred at room temperature for 2 hours. The reaction solution was concentrated to give the title compound (0.702 g, 99%).

¹H-NMR (300 MHz, CD₃OD) δ: 3.59 (t, J=8.8 Hz, 2H), 4.88-4.96 (m, 4H), 7.99 (s, 1H), 8.30 (s, 1H).

### Reference Example 76

### 2-(Trifluoromethyl)pyrimidine-5-carbaldehyde

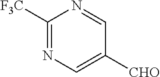 [SMILES: O=Cc1cnc(C(F)(F)F)nc1]

To a solution of the compound of Reference Example 77 (50.0 mg, 0.227 mmol) in toluene (0.8 mL) was added a solution of 1 mol/L diisobutylaluminium hydride in toluene (0.25 mL, 0.25 mmoL) at −78° C., and the mixture was stirred for 15 minutes. To the reaction mixture was then added saturated aqueous Rochelle salt solution, and the mixture was stirred for 1 hour. The mixture was extracted with ethyl acetate, and the organic layer was dried over anhydrous sodium sulfate, filtered, and concentrated in vacuo. The resulting residue was purified by silica gel column chromatography (hexane/ethyl acetate) to give the title compound (30.0 mg, 75%).

¹H-NMR (400 MHz, CDCl₃) δ: 9.33 (2H, s), 10.24 (1H, s).

### Reference Example 77

### Ethyl 2-(trifluoromethyl)pyrimidine-5-carboxylate

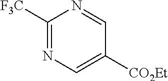 [SMILES: CCOC(=O)c1cnc(C(F)(F)F)nc1]

To a solution of ethyl 4-chloro-2-(trifluoromethyl)pyrimidine-5-carboxylate (1.99 g, 7.82 mmol) in ethanol (30 mL) were added diisopropylethylamine (2.43 g, 18.8 mmol) and 10% palladium-carbon (200 mg), and the mixture was stirred under hydrogen atmosphere at room temperature for 3.5 hours. The reaction mixture was then filtered through Celite®, and concentrated in vacuo. The resulting residue was purified by silica gel column chromatography (hexane/ethyl acetate) to give the title compound (1.36 g, 79%).

¹H-NMR (400 MHz, CDCl₃) δ: 1.46 (3H, t, J=7.2 Hz), 4.51 (2H, q, J=7.2 Hz), 9.43 (2H, s).

### Reference Example 78

### 2-(Difluoromethyl)pyrimidine-5-carbaldehyde

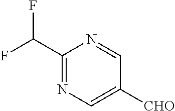 [SMILES: O=Cc1cnc(C(F)F)nc1]

The title compound was prepared from the compound of Reference Example 79 according to a similar process to that of Reference Example 76.

¹H-NMR (400 MHz, CDCl₃) δ: 6.72 (1H, t, J=54.1 Hz), 9.29 (2H, s), 10.22 (1H, s).

### Reference Example 79

### Ethyl 2-(difluoromethyl)pyrimidine-5-carboxylate

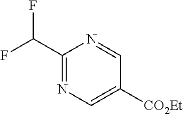 [SMILES: CCOC(=O)c1cnc(C(F)F)nc1]

The title compound was prepared from the compound of Reference Example 80 according to a similar process to that of Reference Example 58.

¹H-NMR (400 MHz, CDCl₃) δ: 1.43 (3H, t, J=7.3 Hz), 4.47 (2H, q, J=7.3 Hz), 6.70 (1H, t, J=54.3 Hz), 9.37 (2H, s).

### Reference Example 80

### Ethyl 2-formylpyrimidine-5-carboxylate

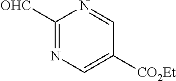 [SMILES: CCOC(=O)c1cnc(C=O)nc1]

The title compound was prepared from the compound of Reference Example 81 according to a similar process to that of Reference Example 51.

LC-MS: Condition B R.T.=0.52 min ObsMS=181.1 [M+1]

### Reference Example 81

### Ethyl 2-(hydroxymethyl)pyrimidine-5-carboxylate

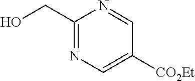 [SMILES: CCOC(=O)c1cnc(CO)nc1]

To a solution of the compound of Reference Example 82 (224 mg, 1.14 mmol) in dichloromethane (3.0 mL) was added a solution of 1.0 mol/L boron tribromide in dichloromethane (2.2 mL, 2.2 mmol) in an ice bath. The mixture was stirred for 1 hour in the ice bath, and saturated aqueous sodium hydrogen carbonate solution was added to the reaction solution, and then the mixture was extracted with chloroform. The organic layer was washed with brine, dried over anhydrous sodium sulfate, filtered, and concentrated in vacuo. The resulting residue was purified by silica gel column chromatography (hexane/ethyl acetate) to give the title compound (160.4 mg, 77%).

¹H-NMR (400 MHz, CDCl₃) δ: 1.44 (3H, t, J=7.1 Hz), 3.69 (1H, brs), 4.47 (2H, q, J=7.1 Hz), 4.93 (2H, s), 9.28 (2H, s).

### Reference Example 82

### Ethyl 2-(methoxymethyl)pyrimidine-5-carboxylate

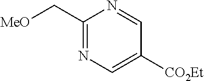 [SMILES: CCOC(=O)c1cnc(COC)nc1]

The title compound was prepared from the compound of Reference Example 83 according to a similar process to that of Reference Example 76.

¹H-NMR (400 MHz, CDCl₃) δ: 1.43 (3H, t, J=7.1 Hz), 3.58 (3H, s), 4.45 (2H, q, J=7.1 Hz), 4.79 (2H, s), 9.28 (2H, s)

### Reference Example 83

### Ethyl 4-chloro-2-(methoxymethyl)pyrimidine-5-carboxylate

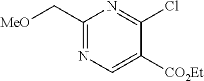 [SMILES: CCOC(=O)c1cnc(COC)nc1Cl]

To a solution of ethyl 4-hydroxy-2-methoxymethyl-pyrimidine-5-carboxylate (2.25 g, 10.6 mmol) in dichloromethane (50 mL) were added oxalyl chloride (1.75 g, 13.79 mmol) and DMF (0.2 mL) at room temperature, and the mixture was stirred at room temperature for 2 hours. To the reaction mixture was then added water, and the mixture was extracted with ethyl acetate. The organic layer was washed with brine, dried over anhydrous sodium sulfate, filtered, and concentrated in vacuo. The resulting residue was purified by silica gel column chromatography (hexane/ethyl acetate) to give the title compound (1.88 g, 77%).

¹H-NMR (400 MHz, CDCl₃) δ: 1.43 (3H, t, J=7.2 Hz), 3.57 (3H, s), 4.46 (2H, q, J=7.2 Hz), 4.73 (2H, s), 9.13 (1H, s).

### Reference Example 84

### 2-(Fluoromethyl)pyrimidine-5-carbaldehyde

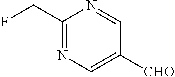

The title compound was prepared from the compound of Reference Example 85 according to a similar process to that of Reference Example 76.

¹H-NMR (400 MHz, CDCl₃) δ: 5.55 (1H, s), 5.70 (1H, s), 9.20 (2H, s), 10.16 (1H, s).

### Reference Example 85

### Ethyl 2-(fluoromethyl)pyrimidine-5-carboxylate

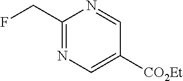 [SMILES: CCOC(=O)c1cnc(CF)nc1]

The title compound was prepared from the compound of Reference Example 81 according to a similar process to that of Reference Example 69.

¹H-NMR (400 MHz, CDCl₃) δ: 1.41 (3H, t, J=7.3 Hz), 4.44 (2H, q, J=7.3 Hz), 5.52 (1H, s), 5.67 (1H, s), 9.28 (2H, s).

### Test Example 1

### Evaluation of Agonistic Activity and Selectivity for D₄ Receptor

### Effect of the Present Compound on G Protein Dependent Pathway in Dopamine D₄ Receptor

The G protein dependent pathway is a pathway for transmitting signals in cells via a second messenger by stimulating a G protein through the binding of guanosine triphosphate (GTP) to the G protein. When GPCRs are activated by the binding of a ligand, a G protein binds to the GPCRs, and thereby GTP binds to a Gα subunit that is one of G protein subunits and a Gγβ subunit is dissociated. The activated Gα subunit regulates intracellular cAMP levels by the activation or inhibition of an adenylate cyclase, and regulates intracellular calcium levels by the activation of phospholipase C to transmit signals in cells. Hence, the activation of the G protein dependent pathway can be evaluated by measuring intracellular cAMP levels and intracellular calcium levels.

In this study, the effects of the present compounds on dopamine D₄ receptor in the G protein dependent pathway were measured.

Preparation of Expressing Cell Lines

Plasmids expressing human brain-derived dopamine D₄ receptor gene (Gene Bank Accession No: NM_000797), calcium-binding photo protein aequorin, and chimeric G protein such as Gα16 and Gqi5 were prepared, and the plasmids were transfected into CHO cells (Chinese Hamster Ovary cells) or HEK293 cells (Human Embryonic Kidney 293 cells) to prepare expressing cell lines.

Measurement of Activity in G Protein Dependent Pathway

The agonistic activities of the present compounds in the G protein dependent pathway were evaluated on the basis of intracellular calcium levels as follows. CHO-K1 or HEK293 cell lines transfected with D₄ receptor genes were seeded in a 384-well plate, and incubated in a CO₂ incubator at 37° C. for 24 hours, and then a solution of the present compound in DMSO was added into cells preloaded with coelenterazine to measure the change in luminescent signals with a FDSS (Hamamatsu Photonics K.K.). The luminescent signals in the wells without the present compound were defined as 0%, and the luminescent signals in the wells with 1 μM of an endogenous ligand (dopamine) were defined as 100%. The agonistic activity of the present compound was calculated according to the measured luminescent signals, as the maximal activity (Emax) of the present compound. EC₅₀ value was calculated as the concentration of the present compound required to achieve a 50% response of Emax.

The results obtained by the test method of Test Example 1 are shown in Table below.

| Examples | Maximal activity on D₄ receptor (%) | EC₅₀ (nM) |
|---|---|---|
| Dopamine | 100 | |
| 1 | 71 | 20 |
| 2 | 83 | 28 |
| 3 | 60 | 19 |
| 4 | 60 | 17 |
| 8 | 37 | 105 |
| 9 | 34 | 89 |
| 13 | 65 | 116 |
| 14 | 46 | 84 |
| 15 | 62 | 9.2 |
| 16 | 51 | 138 |
| 17 | 52 | 25 |
| 18 | 83 | 60 |
| 19 | 63 | 54 |
| 20 | 42 | 51 |
| 21 | 59 | 47 |
| 22 | 62 | 109 |
| 23 | 71 | 25 |
| 25 | 57 | 100 |
| 26 | 49 | 61 |
| 27 | 62 | 0.5 |
| 28 | 56 | 0.8 |
| 29 | 48 | 38 |
| 30 | 53 | 79 |
| 32 | 52 | 19 |
| 33 | 41 | 63 |
| 34 | 73 | 5.3 |
| 35 | 87 | 4.3 |
| 36 | 77 | 40 |
| 37 | 54 | 62 |
| 38 | 43 | 57 |
| 39 | 69 | 35 |
| 40 | 70 | 11 |
| 41 | 54 | 46 |
| 42 | 50 | 100 |
| 43 | 77 | 15 |
| 44 | 98 | 26 |
| 45 | 54 | 157 |
| 46 | 59 | 45 |
| 47 | 65 | 71 |
| 48 | 50 | 96 |
| 49 | 84 | 19 |
| 50 | 41 | 61 |
| 51 | 32 | 70 |
| 52 | 58 | 34 |
| 53 | 99 | 10 |
| 54 | 80 | 7.0 |
| 55 | 92 | 67 |
| 56 | 78 | 12 |
| 57 | 77 | 83 |
| 58 | 81 | 22 |
| 59 | 58 | 36 |
| 60 | 59 | 99 |
| 61 | 57 | 34 |
| 62 | 89 | 25 |
| 63 | 40 | 102 |
| 64 | 42 | 108 |
| 65 | 69 | 20 |
| 66 | 52 | 140 |
| 67 | 64 | 27 |
| 68 | 64 | 69 |
| 69 | 50 | 59 |
| 70 | 45 | 85 |
| 71 | 53 | 88 |
| 72 | 65 | 33 |
| 73 | 58 | 30 |
| 74 | 65 | 73 |
| 75 | 61 | 37 |
| 76 | 59 | 44 |
| 77 | 62 | 58 |
| 78 | 51 | 11 |
| 79 | 83 | 3.4 |
| 80 | 70 | 18 |
| 81 | 89 | 0.7 |
| 82 | 91 | 46 |
| 83 | 72 | 84 |
| 84 | 68 | 118 |
| 85 | 78 | 78 |
| 86 | 85 | 52 |
| 87 | 47 | 62 |
| 88 | 54 | 93 |
| 89 | 39 | 47 |
| 90 | 55 | 29 |
| 91 | 44 | 49 |
| 92 | 64 | 60 |
| 93 | 56 | 23 |
| 94 | 49 | 42 |
| 95 | 56 | 51 |
| 96 | 57 | 37 |
| 97 | 85 | 49 |
| 98 | 54 | 20 |
| 99 | 77 | 1.1 |
| 100 | 79 | 20 |
| 101 | 67 | 78 |
| 102 | 48 | 116 |
| 103 | 53 | 127 |
| 104 | 67 | 18 |
| 105 | 79 | 69 |
| 106 | 94 | 66 |
| 107 | 61 | 13 |
| 108 | 66 | 1.0 |
| 109 | 68 | 36 |
| 110 | 99 | 18 |
| 111 | 22 | 159 |
| 112 | 66 | 41 |
| 113 | 104 | 13 |
| 114 | 93 | 64 |
| 115 | 105 | 57 |
| 116 | 67 | 131 |
| 117 | 51 | 241 |
| 118 | 59 | 106 |
| 119 | 71 | 145 |
| 120 | 98 | 23 |
| 121 | 84 | 46 |
| 122 | 47 | 50 |
| 123 | 65 | 32 |
| 124 | 76 | 47 |
| 125 | 98 | 13 |
| 126 | 91 | 11 |
| 127 | 89 | 56 |
| 128 | 86 | 36 |
| 129 | 91 | 14 |
| 130 | 71 | 20 |
| 131 | 66 | 22 |
| 132 | 64 | 28 |
| 133 | 47 | 5.0 |
| 134 | 62 | 42 |
| 135 | 54 | 49 |
| 136 | 52 | 63 |
| 137 | 82 | 10 |
| 138 | 49 | 50 |
| 139 | 56 | 0.6 |
| 140 | 85 | 86 |
| 141 | 56 | 87 |
| 142 | 82 | 68 |
| 143 | 48 | 80 |
| 144 | 49 | 41 |
| 145 | 52 | 45 |
| 146 | 48 | 90 |
| 147 | 59 | 33 |
| 148 | 71 | 47 |
| 149 | 53 | 107 |
| 150 | 71 | 38 |
| 151 | 69 | 103 |
| 152 | 74 | 107 |
| 153 | 57 | 94 |
| 154 | 57 | 101 |
| 155 | 61 | 85 |
| 156 | 70 | 55 |
| 157 | 50 | 127 |
| 158 | 74 | 65 |
| 159 | 64 | 92 |
| 160 | 53 | 111 |
| 161 | 74 | 26 |
| 162 | 62 | 59 |
| 163 | 57 | 55 |
| 164 | 64 | 55 |
| 165 | 65 | 22 |
| 166 | 85 | 44 |
| 167 | 69 | 17 |
| 168 | 65 | 82 |
| 169 | 80 | 51 |
| 170 | 48 | 152 |
| 171 | 48 | 252 |
| 172 | 53 | 90 |
| 173 | 63 | 12 |
| 174 | 52 | 42 |
| 175 | 50 | 92 |
| 176 | 39 | 41 |
| 177 | 39 | 141 |
| 178 | 50 | 32 |
| 179 | 38 | 64 |
| 180 | 63 | 38 |
| 181 | 43 | 43 |
| 182 | 47 | 79 |
| 183 | 49 | 108 |
| 184 | 51 | 116 |
| 185 | 51 | 82 |
| 186 | 44 | 51 |
| 187 | 66 | 32 |
| 188 | 63 | 44 |
| 189 | 49 | 57 |
| 190 | 61 | 60 |
| 191 | 76 | 43 |
| 195 | 35 | 84 |
| 196 | 29 | 55 |
| 198 | 33 | 45 |
| 199 | 36 | 101 |
| 203 | 54 | 37 |
| 204 | 50 | 44 |

### Test Example 2

### Evaluation of Bioavailability PK Study in Rat

In this study, the pharmacokinetics of the present compounds can be evaluated. Specifically, a 7-week-old SD-type or WKY-type rat receives the intravenous administration of a solution of the present compound in saline or the oral administration of a suspension of the present compound in carboxymethylcellulose or methylcellulose. Blood is collected from the rat at each time below.

Intravenous administration: 5 minutes, 15 minutes, 30 minutes, 1 hour, 2 hours, 4 hours, 6 hours, and 24 hours after administration

Oral administration: 15 minutes, 30 minutes, 1 hour, 2 hours, 4 hours, 6 hours, and 24 hours after administration

Plasma is collected from the blood, and the concentration of the present compound in the plasma is measured by a LC-MS method. The area under the plasma concentration-time curve is calculated on the basis of the concentration changes to calculate the bioavailability of the present compound according to the following formula.

Bioavailabity (%)=AUC after oral administration/AUC after intravenous administration×100

### Test Example 3

### Evaluation of Brain Penetration Brain Penetration Study in Rat

In this study, the brain penetration of the present compounds can be evaluated. Specifically, a 7-week-old SD-type or WKY-type rat received the subcutaneous administration of a solution of the present compound in saline or the oral administration of a suspension of the present compound in methylcellulose. Plasma and brain were collected from the treated rat 0.5 hour, 1 hour or 2 hours after the administration, and then the concentrations of the present compound in the plasma and the brain were measured by a LC-MS method.

The protein binding ratios of the present compound to the plasma and the brain were measured by an equilibrium dialysis method.

The concentrations of the present compound in the plasma and the brain as well as the plasma and brain protein binding ratios are applied to the following formula, and thus Kp,uu,brain (Unbound brain-to-plasma drug concentration ratio) can be calculated.

Kp,uu,brain=(the concentration of the present compound in the brain×(100−the brain protein binding ratio (%))/100)/(the concentration of the present compound in the plasma×(100−the plasma protein binding ratio (%))/100)

The results of Test Example 3 are shown in Table below.

| | Kp,uu,brain |
|---|---|
| Example 22 | 0.20 |
| Example 113 | 0.77 |
| Example 116 | 1.42 |
| Example 128 | 0.60 |
| Example 129 | 0.58 |
| Example 144 | 0.79 |

### Test Example 4

### Evaluation of Risk for Hepatotoxicity

Dansyl Glutathione (dGSH) Trapping Assay

The present compound was metabolized to a metabolite thereof by hepatic microsome, and the resulting metabolite was tested to detect a reactive metabolite therein which can react with dansyl glutathione (dGSH) and quantify the reactive metabolite. The metabolism was induced with a screening robot (Tecan), and the level of a metabolite-dGSH conjugate was measured with a fluorescence detection UPLC system (Waters).

(Preparation of Solution)

The present compound was dissolved in DMSO to prepare 10 mmol/L of a test substance solution. 7.6 mL of potassium phosphate buffer (500 mmol/L, pH 7.4), 1.9 mL of human hepatic microsome (Xenotech, 20 mg protein/mL), and 1.27 mL of pure water were mixed to prepare a microsome solution. To 3.78 mL of the microsome solution was added 0.67 mL of pure water to prepare a microsome (dGSH (−)) solution. To 6.48 mL of the microsome solution was added 1.14 mL of a dGSH solution (20 mmol/L) to prepare a microsome (dGSH (+)) solution. 80.9 mg of NADPH was dissolved in 30 mL of pure water to prepare a cofactor solution. 33 mg of tris(2-carboxyethyl)phosphine (TCEP) was dissolved in 115 mL of methanol to prepare a reaction-stopping solution.

(Reaction)

12 μL of the test substance solution was mixed with 388 μL of pure water, and then the mixture was put into 6 wells in an amount of 50 μL per well in a 96-well plate. The 6 wells were classified into 3 groups by 2 wells, and each group was defined as “reacted group”, “unreacted group”, and “group without dGSH”. The microsome (dGSH (+)) solution was added into the wells of the “reacted group” and the “unreacted group” in an amount of 50 μL per well, and the microsome (dGSH (−)) solution was added in the wells of the “group without dGSH” in an amount of 50 μL per well. The cofactor solution was added into the wells of the “reacted group” and the “group without dGSH” in an amount of 50 μL per well, and pure water was added into the wells of the “unreacted group” in an amount of 50 μL per well. The groups were incubated at 37° C. for 60 minutes, and the reaction-stopping solution was added into the wells of the groups in an amount of 450 μL per well to stop the reaction. Pure water was added into the wells of the “reacted group” and the “group without dGSH” in an amount of 50 μL per well, the cofactor solution was added into the wells of the “unreacted group” in an amount of 50 μL per well, and the plate was cooled at −20° C. for 1 hour, and then centrifuged (4000 rpm, 10 minutes). The supernatant was collected into another plate, and the plate was analyzed.

(Analysis)

The concentration of the metabolite-dGSH conjugate was measured with a fluorescence detection UPLC system (Waters) under the following conditions.

Column: Waters ACQUITY UPLC BEHC 18 1.7 μm 2.1×10 mm

Elution Solvent: A, 0.2% formic acid/40% methanol; B, 0.2% formic acid/methanol

Gradient: B, 0% (0 min)=>83.3% (9.33 min)=>83.3% (10.63 min)=>0% (10.64 min)=>0% (13 min)

Fluorescence intensity varied according to organic solvent compositions, and thus was corrected with that of the organic solvent composition at the time of elution.

The results of Test Example 4 are shown in Table below.

| | Concentration of metabolite-dGSH |
|---|---|
| | conjugate (μM) |
| Example 22 | 0 |
| Example 113 | 0 |
| Example 116 | 0 |
| Example 128 | 0 |
| Example 129 | 0 |
| Example 144 | 0 |

### Test Example 5

### Evaluation of Pharmacological Effect of the Present Compound on Hyperactivity in SHR Rat

A juvenile SHR rat has been widely used as an ADHD model with high validity. The inhibitory effects of the present compounds on hyperactivity in the rat can be evaluated in an open-field environment. Specifically, a 7-week-old SHR rat receives the oral administration of the present compound, and the locomotor activity level in the rat is measured for 90 minutes from 30 minutes after the administration. The measurement is performed with SuperMex (Muromachi Kikai Co., Ltd.). The total locomotor activity level for 90 minutes after the administration of the present compound is statistically expressed as an inhibition ratio (%) in the range of 0 to 100 on the basis of the locomotor activity level in the vehicle administration group.

### Test Example 6

### Evaluation of Pharmacological Effect of the Present Compound on Inattention in SHR Rat

The pharmacological effects of the present compounds on attention function can be evaluated by the pre-treatment of the rat with the present compounds. It is found that the rat has a lower spontaneous alternation behavior ratio than a WKY rat which is a background animal through the Y-shaped maze test. In this study, a Y-shaped maze device (black acrylic: 450 mm×100 mm×350 mm, Horikawa Manufacturing Co., Ltd.) is used. Specifically, a 4-week-old SHR rat receives the oral administration of the present compound, and a spontaneous alternation behavior ratio in the rat is measured for 8 minutes from 30 minutes after the administration. The improved spontaneous alternation behavior ratio (%) is calculated on the basis of the spontaneous alternation behavior ratio in the vehicle administration group.

### Test Example 7

### Evaluation of Pharmacological Effect of the Present Compound on Social Impairments in Rat after Prenatal Exposure to Valproic Acid

The improved effects of the present compounds on social cognition can be evaluated by the pre-treatment of the rat with the present compounds. A rat exposed to valproic acid on the 12.5 days of fetal life has been widely used as an autistic rat model with high validity. It is found that the rat has a social cognitive disorder through the three-chamber test which is a test for evaluating sociability. In this study, a sociability cage (600 mm×400 mm×220 mm, Muromachi Kikai Co., Ltd.) is used. Specifically, a 3-week-old rat after prenatal exposure to valproic acid receives the oral administration of the present compound, and the time that the rat stays in close to another rat side or a novel object is measured for 10 minutes from 30 minutes after the administration. The ratio of the time for another rat side to the time for a novel object that is defined as 100% is calculated to evaluate the improvement ratio (%) of the present compound on the basis of the result of the vehicle-treated group.

### INDUSTRIAL APPLICABILITY

As described above, the present compound is a dopamine D₄ receptor agonist, and thus is useful for treating a disease such as attention deficit hyperactivity disorder.

1. A compound of formula (1): 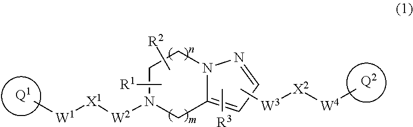
or a pharmaceutically acceptable salt thereof, wherein
n and m are independently 1 or 2;
W¹, W³, and W⁴ are independently single bond or optionally-substituted C₁₋₄ alkylene group;
W² is C₁₋₄ alkylene group;
R¹ and R² are independently hydrogen atom, halogen atom, or optionally-substituted C₁₋₆ alkyl group, or R¹ and R² may be combined with the carbon atom(s) to which they are attached to form 3- to 8-membered cycloalkane ring;
R³ is hydrogen atom, halogen atom, cyano group, optionally-substituted C₁₋₆ alkyl group, optionally-substituted C₁₋₆ alkoxy group, optionally-substituted C₁₋₆ alkylcarbonyl group, or optionally-substituted aminocarbonyl group;
X¹ and X² are independently single bond, oxygen atom, sulfur atom, —C(O)—, —NR⁴⁰—, or —C(O)NR⁴⁰—, wherein said R⁴⁰ is hydrogen atom or C₁₋₆ alkyl group;
ring Q¹ is optionally-substituted C₆₋₁₀ aryl group, optionally-substituted 5- to 10-membered heteroaryl group, optionally-substituted C₅₋₁₀ cycloalkyl group, or optionally-substituted 5- to 10-membered cyclic amino group; and
ring Q² is optionally-substituted phenyl group, optionally-substituted 6-membered heteroaryl group, optionally-substituted 5- or 6-membered saturated heterocyclyl group, or optionally-substituted 5- or 6-membered cyclic amino group.

2. The compound according to claim 1 or a pharmaceutically acceptable salt thereof, wherein
n and m are independently 1 or 2;
W¹, W³, and W⁴ are independently single bond or C₁₋₄ alkylene group which may be optionally substituted with the same or different 1 or 2 halogen atoms;
W² is C₁₋₄ alkylene group;
R¹ and R² are independently hydrogen atom, halogen atom, or C₁₋₆ alkyl group which may be optionally substituted with the same or different 1 to 3 halogen atoms, or R¹ and R² may be combined with the carbon atom(s) to which they are attached to form 3- to 8-membered cycloalkane ring;
R³ is
(1) hydrogen atom,
(2) halogen atom,
(3) cyano group,
(4) C₁₋₆ alkyl group which may be optionally substituted with the same or different 1 to 3 halogen atoms,
(5) C₁₋₆ alkoxy group which may be optionally substituted with the same or different 1 to 3 halogen atoms,
(6) C₁₋₆ alkylcarbonyl group which may be optionally substituted with the same or different 1 to 3 halogen atoms, or
(7) aminocarbonyl group wherein the amino moiety thereof may be optionally substituted with the same or different 1 or 2 groups selected independently from the group consisting of C₁₋₆ alkyl and C₃₋₇ cycloalkyl group;
X¹ and X² are independently single bond, oxygen atom, sulfur atom, —C(O)—, —NR⁴⁰—, or —C(O)NR⁴⁰—, wherein said R⁴⁰ is hydrogen atom or C₁₋₆ alkyl group;
ring Q¹ is
(8) C₆₋₁₀ aryl group which may be optionally substituted with the same or different 1 to 4 groups selected independently from the group consisting of
(a) halogen atom,
(b) C₁₋₆ alkyl group which may be optionally substituted with the same or different 1 to 3 groups selected independently from the group consisting of halogen atom and hydroxy group,
(c) C₁₋₆ alkoxy group which may be optionally substituted with the same or different 1 to 3 halogen atoms,
(d) cyano group, and
(e) amino group which may be optionally substituted with the same or different 1 or 2 groups selected independently from the group consisting of C₁₋₆ alkyl group and C₃₋₇ cycloalkyl group,
(9) 5- to 10-membered heteroaryl group which may be optionally substituted with the same or different 1 to 4 groups selected independently from the group consisting of (a) to (e) defined in the above (8),
(10) C₅₋₁₀ cycloalkyl group which may be optionally substituted with the same or different 1 to 4 groups selected independently from the group consisting of (a) to (e) defined in the above (8), or
(11) 5- to 10-membered cyclic amino group which may be optionally substituted with the same or different 1 to 4 groups selected independently from the group consisting of (a) to (e) defined in the above (8); and
ring Q² is
(12) phenyl group which may be optionally substituted with the same or different 1 to 4 groups selected independently from the group consisting of (a) to (e) defined in the above (8),
(13) 6-membered heteroaryl group which may be optionally substituted with the same or different 1 to 4 groups selected independently from the group consisting of (a) to (e) defined in the above (8),
(14) 5- or 6-membered saturated heterocyclyl group which may be optionally substituted with the same or different 1 to 4 groups selected independently from the group consisting of (a) to (e) defined in the above (8), or
(15) 5- or 6-membered cyclic amino group which may be optionally substituted with the same or different 1 to 4 groups selected independently from the group consisting of (a) to (e) defined in the above (8).

3. The compound according to claim 1 or a pharmaceutically acceptable salt thereof, wherein W³, X¹, and X² are single bond.

4. The compound according to claim 1 or a pharmaceutically acceptable salt thereof, wherein the compound is a compound of formula (1a): 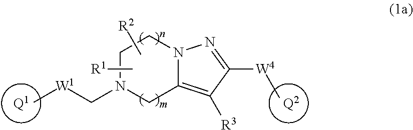
wherein n, m, W¹, W⁴, R¹, R², R³, ring Q¹, and ring Q² are as defined in claim 1 or 2.

5. The compound of claim 4 or a pharmaceutically acceptable salt thereof, wherein
n and m are independently 1 or 2;
W¹ and W⁴ are independently single bond or C₁₋₄ alkylene group which may be optionally substituted with the same or different 1 or 2 halogen atoms;
R¹ and R² are independently hydrogen atom, halogen atom, or C₁₋₆ alkyl group which may be optionally substituted with the same or different 1 to 3 halogen atoms, or R¹ and R² may be combined with the carbon atom(s) to which they are attached to form 3- to 8-membered cycloalkane ring;
R³ is
(1) hydrogen atom,
(2) halogen atom,
(3) cyano group,
(4) C₁₋₆ alkyl group which may be optionally substituted with the same or different 1 to 3 halogen atoms, or
(5) C₁₋₆ alkoxy group which may be optionally substituted with the same or different 1 to 3 halogen atoms;
ring Q¹ is
(6) 5- to 10-membered heteroaryl group which may be optionally substituted with the same or different 1 to 4 groups selected independently from the group consisting of
(a) halogen atom,
(b) C₁₋₆ alkyl group which may be optionally substituted with the same or different 1 to 3 groups selected independently from the group consisting of halogen atom and hydroxy group,
(c) C₁₋₆ alkoxy group which may be optionally substituted with the same or different 1 to 3 halogen atoms,
(d) cyano group, and
(e) amino group which may be optionally substituted with the same or different 1 or 2 groups selected independently from the group consisting of C₁₋₆ alkyl group and C₃₋₇ cycloalkyl group,
(7) C₆₋₁₀ aryl group which may be optionally substituted with the same or different 1 to 4 groups selected independently from the group consisting of (a) to (e) defined in the above (6), or
(8) C₅₋₁₀ cycloalkyl group which may be optionally substituted with the same or different 1 to 4 groups selected independently from the group consisting of (a) to (e) defined in the above (6);
ring Q² is
(9) phenyl group which may be optionally substituted with the same or different 1 to 4 groups selected independently from the group consisting of (a) to (e) defined in the above (6),
(10) 6-membered heteroaryl group which may be optionally substituted with the same or different 1 to 4 groups selected independently from the group consisting of (a) to (e) defined in the above (6), or
(11) 5- or 6-membered saturated heterocyclyl group which may be optionally substituted with the same or different 1 to 4 groups selected independently from the group consisting of (a) to (e) defined in the above (6).

6. The compound according to claim 5 or a pharmaceutically acceptable salt thereof, wherein the ring Q² is
(1) phenyl group which may be optionally substituted with the same or different 1 to 4 groups selected independently from the group consisting of
(a) halogen atom,
(b) C₁₋₆ alkyl group which may be optionally substituted with the same or different 1 to 3 halogen atoms,
(c) C₁₋₆ alkoxy group which may be optionally substituted with the same or different 1 to 3 halogen atoms,
(d) cyano group, and
(e) amino group which may be optionally substituted with the same or different 1 or 2 groups selected independently from the group consisting of C₁₋₆ alkyl group and C₃₋₇ cycloalkyl group, or
(2) 6-membered heteroaryl group containing 1 to 3 nitrogen atoms which may be optionally substituted with 1 to 4 groups selected independently from the group consisting of (a) to (e) defined in the above (1).

7. The compound according to claim 4 or a pharmaceutically acceptable salt thereof, wherein
n is 1 or 2;
m is 1;
both W¹ and W⁴ are single bond;
R¹, R², and R³ are independently hydrogen atom, halogen atom, or C₁₋₆ alkyl group which may be optionally substituted with the same or different 1 to 3 halogen atoms;
ring Q¹ is
(1) 5- to 10-membered heteroaryl group containing 1 to 3 nitrogen atoms which may be optionally substituted with the same or different 1 to 4 groups selected independently from the group consisting of
(a) halogen atom,
(b) C₁₋₆ alkyl group which may be optionally substituted with the same or different 1 to 3 halogen atoms,
(c) C₁₋₆ alkoxy group which may be optionally substituted with the same or different 1 to 3 halogen atoms,
(d) cyano group, and
(e) amino group which may be optionally substituted with the same or different 1 or 2 groups selected independently from the group consisting of C₁₋₆ alkyl group and C₃₋₇ cycloalkyl group, or
(2) C₆₋₁₀ aryl group which may be optionally substituted with the same or different 1 to 4 groups selected independently from the group consisting of (a) to (e) defined in the above (1);
ring Q² is
(3) pyridyl group which may be optionally substituted with the same or different 1 to 4 groups selected independently from the group consisting of (a) to (e) defined in the above (1), or
(4) phenyl group which may be optionally substituted with the same or different 1 to 4 groups selected independently from the group consisting of (a) to (e) defined in the above (1).

8. The compound according to claim 4 or a pharmaceutically acceptable salt thereof, wherein ring Q¹ is 5- to 10-membered heteroaryl group containing 1 to 3 nitrogen atoms which may be optionally substituted with the same or different 1 to 4 groups selected independently from the group consisting of
(a) halogen atom,
(b) C₁₋₆ alkyl group which may be optionally substituted with the same or different 1 to 3 groups selected independently from the group consisting of halogen atom and hydroxy group,
(c) C₁₋₆ alkoxy group which may be optionally substituted with the same or different 1 to 3 halogen atoms,
(d) cyano group, and
(e) amino group which may be optionally substituted with the same or different 1 or 2 groups selected independently from the group consisting of C₁₋₆ alkyl group and C₃₋₇ cycloalkyl group.

9. The compound according to claim 4 or a pharmaceutically acceptable salt thereof, wherein ring Q¹ is
(1) 6-membered heteroaryl group containing 1 to 3 nitrogen atoms which may be optionally substituted with the same or different 1 to 4 groups selected independently from the group consisting of
(a) halogen atom,
(b) C₁₋₆ alkyl group which may be optionally substituted with the same or different 1 to 3 halogen atoms,
(c) C₁₋₆ alkoxy group which may be optionally substituted with the same or different 1 to 3 halogen atoms,
(d) cyano group, and
(e) amino group which may be optionally substituted with the same or different 1 or 2 groups selected independently from the group consisting of C₁₋₆ alkyl group and C₃₋₇ cycloalkyl group, or
(2) phenyl group which may be optionally substituted with the same or different 1 to 4 groups selected independently from the group consisting of (a) to (e) defined the above (1).

10. The compound according to claim 4 or a pharmaceutically acceptable salt thereof, wherein ring Q¹ is a group of the following formula (2a) or (2b): 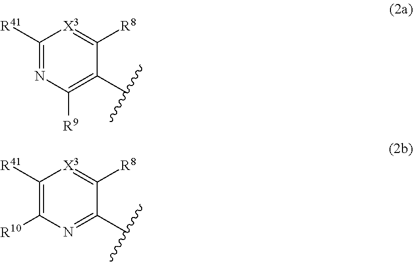
wherein X³ is N or CR⁷;
R⁴¹ is halogen atom or C₁₋₆ alkyl group which may be optionally substituted with the same or different 1 to 3 groups selected independently from the group consisting of halogen atom and hydroxy group;
R⁷, R⁸, R⁹, and R¹⁰ are independently hydrogen atom, halogen atom, C₁₋₆ alkyl group which may be optionally substituted with the same or different 1 to 3 halogen atoms, or amino group which may be optionally substituted with the same or different 1 or 2 C₁₋₆ alkyl groups;
or R⁴¹ and R¹⁰, or R⁴¹ and R⁷ may be combined with the carbon atom(s) to which they are attached to form 3- to 8-membered cycloalkane ring or 5- to 8-membered cycloalkene ring.

11. The compound according to claim 4 or a pharmaceutically acceptable salt thereof, wherein ring Q² is a group of the following formula (3): 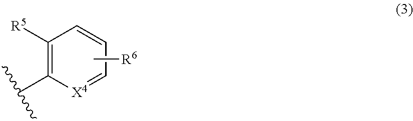
wherein X⁴ is N or CH;
R⁵ is halogen atom, C₁₋₆ alkyl group which may be optionally substituted with the same or different 1 to 3 halogen atoms, or C₁₋₆ alkoxy group which may be optionally substituted with the same or different 1 to 3 halogen atoms;
R⁶ is hydrogen atom, halogen atom, C₁₋₆ alkyl group which may be optionally substituted with the same or different 1 to 3 halogen atoms, or C₁₋₆ alkoxy group which may be optionally substituted with the same or different 1 to 3 halogen atoms.

12. The compound according to claim 11 or a pharmaceutically acceptable salt thereof, wherein X⁴ is N.

13. The compound according to claim 1 or a pharmaceutically acceptable salt thereof, wherein both R¹ and R² are hydrogen atom.

14. The compound according to claim 1 or a pharmaceutically acceptable salt thereof, wherein the compound is a compound of formula (1b): 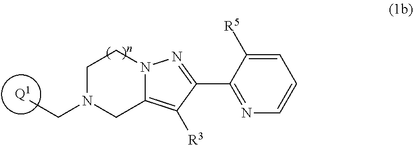
wherein n is 1 or 2;
ring Q¹ is a group of the following formula (2c) or (2d): 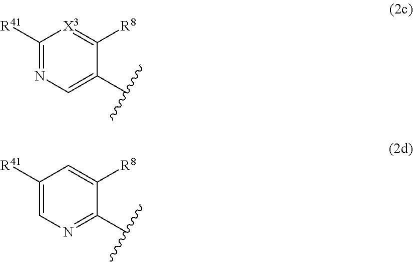
wherein X³ is N or CH;
R⁴¹ is halogen atom or C₁₋₆ alkyl group which may be optionally substituted with the same or different 1 to 3 halogen atoms; and
R⁸ is hydrogen atom, halogen atom, or C₁₋₆ alkyl group which may be optionally substituted with the same or different 1 to 3 halogen atoms;
R³ is hydrogen atom, halogen atom, or C₁₋₆ alkyl group which may be optionally substituted with the same or different 1 to 3 halogen atoms; and
R⁵ is halogen atom or C₁₋₆ alkyl group which may be optionally substituted with the same or different 1 to 3 halogen atoms.

15. The compound according to claim 14 or a pharmaceutically acceptable salt thereof, wherein the ring Q¹ is a group of formula (2c).

16. The compound according to claim 15 or a pharmaceutically acceptable salt thereof, wherein X³ is CH.

17. The compound according to claim 15 or a pharmaceutically acceptable salt thereof, wherein X³ is N.

18. The compound according to claim 14 or a pharmaceutically acceptable salt thereof, wherein the ring Q¹ is a group of formula (2d).

19. The compound according to claim 1 or a pharmaceutically acceptable salt thereof, wherein
n is 1; and
R³ is hydrogen atom or C₁₋₆ alkyl group.

20. The compound according to claim 10 or a pharmaceutically acceptable salt thereof, wherein R⁸ is hydrogen atom.

21. The compound according to claim 10 or a pharmaceutically acceptable salt thereof, wherein R⁴¹ is C₁₋₄ alkyl group substituted with 1 to 3 fluorine atoms.

22. A compound selected from the group consisting of the following formulae: 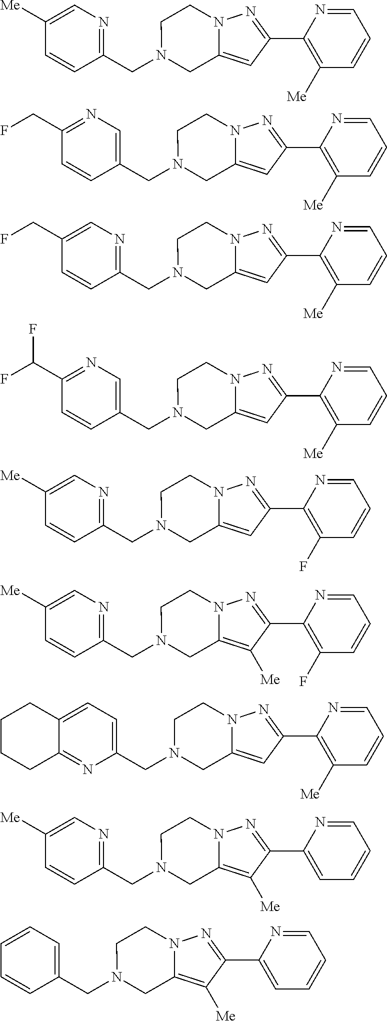
or a pharmaceutically acceptable salt thereof.

23. A pharmaceutical product comprising the compound according to claim 1 or a pharmaceutically acceptable salt thereof as an active ingredient.

24. A medicament for treating attention deficit hyperactivity disorder, comprising the compound according to claim 1 or a pharmaceutically acceptable salt thereof as an active ingredient.

25. The medicament according to claim 24, wherein the attention deficit hyperactivity disorder is a disorder with inattention as a predominant symptom.

26. The medicament according to claim 24, wherein the attention deficit hyperactivity disorder is a disorder with hyperactivity as a predominant symptom.

27. The medicament according to claim 24, wherein the attention deficit hyperactivity disorder is a disorder with impulsivity as a predominant symptom.

28. A medicament for treating autistic spectrum disorder, comprising the compound according to claim 1 or a pharmaceutically acceptable salt thereof as an active ingredient.

29. The medicament according to claim 28, wherein the autistic spectrum disorder is a disorder with persistent deficits in social communication and social interaction as a predominant symptom.

30. The medicament according to claim 28, wherein the autistic spectrum disorder is a disorder with restricted repetitive behaviors, interests, or activities.

31. A method for treating a central nervous system disease selected from the group consisting of attention deficit hyperactivity disorder, autistic spectrum disorder, schizophrenia, mood disorder, and cognitive dysfunction, which comprises administering a therapeutically effective amount of the compound according to claim 1 or a pharmaceutically acceptable salt thereof to a patient in need thereof.
